(19) United States
(12) Patent Application Publication
(10) Pub. No.: US 2024/0165158 A1
(43) Pub. Date: May 23, 2024

(54) MAJOR HISTOCOMPATIBILITY COMPLEX-BASED CHIMERIC RECEPTORS AND USES THEREOF FOR TREATING AUTOIMMUNE DISEASES

(71) Applicant: Jura Bio, Inc., Boston, MA (US)

(72) Inventors: Julie Norville, Boston, MA (US); Elizabeth Wood, Boston, MA (US)

(73) Assignee: Jura Bio, Inc., Boston, MA (US)

(21) Appl. No.: 18/490,445

(22) Filed: Oct. 19, 2023

Related U.S. Application Data
(62) Division of application No. 16/762,723, filed on May 8, 2020, now Pat. No. 11,826,385.
(60) Provisional application No. 62/584,449, filed on Nov. 10, 2017.

(51) Int. Cl. A61K 35/17; C07K 14/705; C07K 14/725; C07K 14/74; C12N 15/85

(52) U.S. Cl. CPC A61K 35/17; C07K 14/7051; C07K 14/70521; C07K 14/70539; C07K 14/70596; C12N 15/85

(57) ABSTRACT

Major histocompatibility complex-based chimeric receptors (MHC-CAR) for use in targeting autoreactive immune cells. Also provided herewith are genetically engineered immune cells expressing the MHC-CAR for use in treating autoimmune diseases such as multiple sclerosis.

### RELATED APPLICATION

This application claims the benefit of the filing date of U.S. Provisional Application Ser. No. 62/584,449, filed on Nov. 10, 2017. The entire contents of this referenced application are incorporated by reference herein.

### BACKGROUND OF THE INVENTION

Autoimmune diseases are characterized by abnormal immune responses against self-antigens, leading to damage or disruption of tissues. Multiple sclerosis (MS) is a central nervous system autoimmune disease, in which activated autoreactive T cells invade the blood brain barrier, initiating an inflammatory response that leads to myelin destruction and axonal loss. Although the etiology of MS, the mechanisms associated with its onset and progression, and determination of its outcome remains unelucidated, all available evidence suggests that therapies specifically targeting the pathologic immune cells responsible for MS would have improved therapeutic outcomes over available therapies. Reinhard et al., Proceedings of the National Academy of Sciences, 101(suppl 2):14599-14606; 2004. This strategy could be extended to other immune disorders with similar mechanisms, including rheumatoid arthritis. Carol et al., Nature Reviews Immunology, 2(2):85-95, 2002.

The major histocompatibility complex (MHC), known as human leukocytes (HLA) in humans, is a set of cell surface proteins essential for the immune system to recognize foreign agents. MHC complexes bind to antigens derived from pathogens and display such to T cells, which are then activated, leading to elimination of cells displaying foreign antigens. MHC complexes may also display intact, and in some cases misfolded, host-derived proteins to B cells thereby inducing the autoantibody responses characteristic of autoimmune disorders. Jiang et al., International immunology, 25(4):235-246 (2013), and Busch et al., The EMBO journal, 15(2):418, (1996).

### SUMMARY OF THE INVENTION

In aspect, the disclosure features a major histocompatibility complex (MHC)-based chimeric receptor (CAR), comprising: (i) an extracellular domain of a MHC molecule conjugated to an antigenic peptide from an antigen involved in an autoimmune disease; and (ii) a cytoplasmic signaling domain, at least one co-stimulatory domain, or a combination thereof. The MHC-based CAR may further comprises a hinge domain located between (i) and (ii). The antigenic peptide is dependent on the autoimmune disorder and may be from myelin basic protein (MBP), proteolipid protein (PLP), insulin, glutamate decarboxylase, or the additional exemplary self-antigens as described in Table 1.

In some examples, the MHC-based chimeric receptor comprises at least one co-stimulatory domain, which may be a co-stimulatory domain from 4-1BB (CD137), a co-stimulatory domain from CD28, or a combination thereof. In other examples, the MHC-based chimeric receptor as described herein may be free of a cytoplasmic signaling domain. Alternatively or in addition, the MHC-CAR comprises a cytoplasmic signaling domain of CD3ζ.

In some embodiments, the MHC molecule in the MHC-CAR is a class I MHC, for example, a human class I MHC. In some instances, the extracellular domain of the chimeric receptor comprises an extracellular domain of the alpha chain of the class I MHC, which is fused to the antigenic peptide. For example, the chimeric receptor may be a fusion polypeptide comprising (i) the extracellular domain of the class I MHC molecule, and (ii) the cytoplasmic domain, the at least one co-stimulatory domain, or the combination thereof. In one example, the chimeric receptor is a fusion polypeptide, which comprises, from N-terminus to C-terminus, a signal peptide, a first peptide linker, the antigenic peptide, a second peptide linker, an extracellular domain of macroglobulin, a third peptide linker, the class I MHC molecule, a transmembrane domain, the at least one co-stimulatory domain, and CD3ζ.

In other embodiments, the MHC-based chimeric receptor as described herein comprises a class II MHC (e.g., a human MHC II) or a portion thereof. Such a chimeric receptor may comprise a first polypeptide, which comprises an extracellular domain of a first MHC class II, and a second polypeptide, which comprises an extracellular domain of a beta chain of a second MHC class II, and wherein the antigenic peptide is fused to either the first polypeptide or the second polypeptide, and wherein either the first polypeptide or the second polypeptide further comprises the cytoplasmic signaling domain, the at least one co-stimulatory domain, or the combination thereof. In some examples, the chimeric receptor can be a fusion polypeptide comprising (i) an extracellular domain of the alpha chain of a first MHC class II molecule, (ii) an extracellular domain of the beta chain of a second MHC class II molecule, (iii) the antigenic peptide, and (iv) the cytoplasmic signaling domain, the at least one co-stimulatory domain, or the combination thereof. In some examples the antigenic protein may not be linked to the MHC class II and may instead be expressed as a separate fusion polypeptide with an alternative signal peptide (such as that from CD150, i.e., MDPKGLLSLTFVLFLSLAFG (SEQ ID NO: 388)). In some examples, the first MHC class II is HLA-DRA*1010. Alternatively or in addition, the second MHC class II is HLA-DRB1*1501.

In another aspect, the present disclosure features a nucleic acid or a nucleic acid set, which collectively encodes any of the MHC-based chimeric receptors described herein. In some instances, the nucleic acid or nucleic acid set can be located in one or more vectors, for example, viral vector(s).

Further, the present disclosure provides a genetically modified immune cell (e.g., a T cell), which expresses any of the MHC-based chimeric receptors described herein. In some instances, the activity of the endogenous T cell receptor (TCR) can be suppressed, which may be achieved by mutating or deleting the alpha chain of the endogenous TCR, the beta chain of the endogenous TCR, or both to disrupt surface expression of the endogenous TCR. Alternatively or in addition, the expression of the endogenous CD52 can be disrupted.

In some embodiments, the genetically modified immune cell as described herein may further express a suicide gene (e.g., RQR8), a marker gene (e.g., GFP), or both. When necessary, the immune cell can be further modified for lymph node or tertiary lymphoid organ delivery and retention. For example, the immune cell can be further engineered to overexpress VAP-1, L-selectin. CCR7, CXCR5, or a combination thereof. In some instances, the expression of endogenous sphingosine-1-phosphate receptor 1 can be disrupted in the genetically modified immune cell.

In some embodiments, the immune cell can be engineered to travel to the site of inflammation, for instance using a chemokine receptor such as CCR6 (e.g., to the site of Th17 cells), CXCR3 or CXCR4 (e.g., to the site of plasma cells), or through a membrane linked, antigen targeted antibody. Alternatively or in addition, the genetically modified immune cell may further comprise a genetic modification that results in blockade of PD-1 signaling. If needed and the disorder is especially severe the MHC-CAR cells can also be designed to remove or inactive bystander B cells (with a CD19 or CD20-CAR) or plasma cells (with a CS 1-CAR and/or CS1 knockout).

In some embodiments, the genetically modified immune cell as described herein may be a regulatory T cell, which can be CD25+, and optionally CD4+. In some instances, the regulatory T cell can be derived from CD25++CD45R+ T cells isolated from peripheral blood mononuclear cells or from cord blood. In other instance, the regulatory T cell may comprise a transgene coding for CD25. Any of the Treg cells disclosed herein may further express a chimeric receptor specific to CD19, a chimeric receptor specific to CS-1, or both. Alternatively or in addition, the regulatory T cell may further express CCR6, CXCR5, PD-1, or a combination thereof. In some examples, the regulatory T cell may display an antibody specific to MOG.

In yet another aspect, the present disclosure provides a method for suppressing autoreactive immune cells in a subject having an autoimmune disease (e.g., multiple sclerosis). The method may comprise administering to the subject an effective amount of genetically modified immune cells as described herein, which can be T cells.

In some embodiments, the genetically modified immune cells are autologous. In other embodiments, the genetically modified immune cells are allogeneic. Any of the genetically modified immune cells may be administered to a lymph node of the subject. In some instances, the subject is undergoing a therapy comprising an antibody specific to CD52.

In some embodiments, the subject is a human patient having or at risk for multiple sclerosis and the genetically modified T cells are Treg cells or cytotoxic lymphocytes (CTLS) as described herein.

In some examples, the human patient is an early-stage MS patient and the Treg cells express the MHC-CAR and have one or more of the following genetic modifications: (i) PD-L1 and/or PD-1 knockout; (ii) surface expression of CCR6 and/or CXCR5; (iii) surface display of an antibody or an antigen-binding fragment thereof that is specific to MOG; and (iv) surface expression of a chimeric receptor targeting CD19. In some examples the patient may first, simultaneously, or alternatively be treated with cytotoxic CTLs with modifications of the same type.

In some examples, the human patient has relapsing-remitting MS or early-stage progressive MS and the Treg cells express the MHC-CAR and have one or more of the following modifications: (i) surface display of an antibody or antigen binding fragment that is specific to MOG; and (ii) surface expression of CCR6. In some examples the patient may first, simultaneously, or alternatively be treated with cytotoxic CTLs with modifications of the same type.

In some examples, the human patient has relapsing-remitting MS or early-stage progressive MS and the Treg cells express the MHC-CAR and have one or more of the following modifications: (i) surface expression of a chimeric receptor targeting CD19; and (ii) surface expression of CXCR5. In some examples the patient may first, simultaneously, or alternatively be treated with CTLs with the same modifications.

In some examples, the human patient has MS in chronic progressive form and the Treg cells express the MHC-CAR and have one or more of the following modifications: (i) surface expression of a chimeric receptor targeting CS-1; and (ii) surface expression of an agent CXCR4, CCR6, and/or CXCR5. In some examples the patient may first, simultaneously, or alternatively be treated with CTLs with the same modifications.

In some embodiments, the subject is a human patient having or at risk for systemic lupus erythematosus, rheumatoid arthritis, juvenile idiopathic arthritis (also known as juvenile idiopathic arthritis), Sjögren's syndrome, systemic sclerosis, ankylosing spondylitis, Type 1 diabetes, autoimmune thyroid diseases (Grave's and Hashimoto's), multiple sclerosis myasthenia gravis, inflammatory bowel disease (Crohn's or ulcerative colitis), Psoriasis, or a diseases mentioned in Table 1 and the genetically modified T cells are Treg cells and/or CTLs as described herein.

In some examples, the human patient is an early-stage patient of any of the autoimmune disorders described herein (e.g., those listed in Table 1) and the Treg cells express the MHC-CAR and have one or more of the following genetic modifications: (i) PD-L1 and/or PD-1 knockout; (ii) surface expression of CCR6 and/or CXCR5; (iii) surface display of an antibody or an antigen-binding fragment thereof that is specific to a relevant protein described as an autoantigen in Table 1 for that autoimmune disorder; and (iv) surface expression of a chimeric receptor targeting CD19. In some examples the patient may first, simultaneously, or alternatively be treated with CTLs with the same modifications.

In some examples, the human patient has moderately severe disease state of any of the autoimmune disorders as described herein (e.g., those listed in Table 1) and the Treg cells express the MHC-CAR and have one or more of the following modifications: (i) surface display of an antibody or antigen binding fragment that is specific to a relevant protein described as an autoantigen in Table 1 for that autoimmune disorder; and (ii) surface expression of CCR6. In some examples the patient may first, simultaneously, or alternatively be treated with CTLs with the same modifications.

In some examples, the human patient has moderately severe disease state of any of the autoimmune disorders as described herein (e.g., those listed in Table 1) and the Treg cells express the MHC-CAR and have one or more of the following modifications: (i) surface expression of a chimeric receptor targeting CD19; and (ii) surface expression of CXCR5. In some examples the patient may first, simultaneously, or alternatively be treated with CTLs with the same modifications.

In some examples, the human patient has severe disease state of any of the autoimmune disorders described herein (e.g., those listed in Table 1) and the Treg cells express the MHC-CAR and have one or more of the following modifications: (i) surface expression of a chimeric receptor targeting CS-1; and (ii) surface expression of an agent targeting CXCR4, CCR6, and/or CXCR5. In some examples the patient may first, simultaneously, or alternatively be treated with CTLs with the same modifications.

Also within the scope of the present disclosure are pharmaceutical compositions for use in treating an autoimmune disease, the composition comprising genetically modified immune cells expression MHC-CAR as described herein such as Treg cells and a pharmaceutically acceptable carrier, and uses of such genetically modified immune cells for manufacturing a medicament for use in treating the target autoimmune disease.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

FIG. 1 is a schematic illustration of an exemplary design of a lentiviral expression vector for expression of an antigen-specific T cell receptor (TCR). mRNA and multicistronic mRNA designs are similar.

FIG. 2 is a schematic illustration of an MHC-CAR T cell, which expresses various designs of MHC-CAR as indicated in conjugation with a myelin basic protein (MBP) peptide for targeting pathologic T cells involved in multiple sclerosis (MS). Optionally, the MHC-CAR T cell may have the endogenous TCR and/or CD52 knocked out. The MHC-CAR T cell may further express RQR8 on the cell surface.

FIG. 3 is a schematic illustration of exemplary designs for various MHC-CAR constructs. The exemplary MHC-CAR constructs may have two subunits: an α-chain containing a leader sequence, a DRA*1010 domain, and a cytoplasmic domain, and a 0-chain that includes a leader sequence from HLA-DRB1*1501, a peptide from MBP, and a domain from HLA-DRB1*1501. The DNA used to create mRNA contains either single chains or are multicistronic and separated by orthogonal 2A sequences. RQR8 and eBFP (or GFP) are used for both cell control and labeling.

FIG. 4 is a schematic illustration of exemplary designs of expression cassettes for various MHC-CAR constructs containing a MBP peptides, which may further include eBFP (or GFP) or RQR8.

FIG. 5 is a schematic illustration of an exemplary design of a lentiviral expression construct for MHC-CARs and optionally label proteins such as eBFP and/or RQR8. Such an expression cassette would be sufficiently small to be included in one lentiviral package.

FIG. 6 is a schematic illustration of an exemplary design of MHC-CAR, which may include a number of sites for tagging. Site 1 is an HLA-DR antibody binding site for cases where naïve HLA-DR is either not expressed or due to CIITA editing. Sites 2 and 3 are potential insertion sites for polyhistidine-tag motifs. Sites 4 and 5 represent RQR and RQR8, respectively.

FIG. 7 is a schematic illustration of exemplary designs of MHC Class II moieties linked to antigenic peptides. “N” refers to the N-terminus of a polypeptide. Circled black dots refer to the antigenic peptides.

FIG. 8A and FIG. 8B are schematic illustrations of MHC Class I exemplary constructs. FIG. 8A depicts exemplary designs of MHC Class I moieties linked to antigenic peptides. “N” refers to the N-terminus of a polypeptide. Circled black dots refer to the antigenic peptides. FIG. 8B depicts an exemplary expression cassette for a MHC Class I CAR construct.

FIG. 9 depicts exemplary co-stimulatory domains and combinations thereof for constructing the MHC-CAR and considerations for co-expression of cytokines.

FIG. 10 depicts exemplary single-chain and multi-chain MHC Class I and Class 11 MHC-CAR constructs, including multi-chain MHC-CAR constructs containing both MHC Class I and Class II components.

FIG. 11 is a plot showing CD3 expression on primary human stimulated CD3+ T cells (TCELL-0028) transfected with an mRNA encoding Cas9 (“Cas9 only”) or an mRNA encoding Cas9 (“Cas9 only”) and sgRNAs targeting the gene encoding T cell receptor alpha domain (TRAC) (“Cas9+TRAC gRNA”).

FIG. 12 is a plot showing relative cell viability in a killing assay. HEK-293 cells transfected with Construct 1, Construct 2, or both Constructs 1 and 2 were incubated with media only, rituximab, complement, or both rituximab and complement.

### DETAILED DESCRIPTION OF THE INVENTION

Autoreactive T cells, (e.g., those for myelin components involved in multiple sclerosis) exist in normal individuals. The majority of determinant of disease induction is in the class of immune response that occurs when these autoreactive T cells are triggered in autoimmune patients (e.g., in MS patients). Generation of pathologic autoreactive T cells is favored both by specific major histocompatibility complex (MHC) and non-MHC genes, which determine the protein sequences an individual reacts against and the class of the immune response.

Once an immune attack begins on an initial autoantigen (for example, a myelin antigen in MS), there is a spreading of reactivity to other autoantigens; that is, if a T cell attacks one autoantigen (for example, a brain protein in MS), other structures are damaged and they can sensitize additional T cells to attack other targets in a process called “epitope-spreading”, a process that is shared by all autoimmune disorders and common to disease response in general.

B cells are ordinary components of the immune reaction in the early disease lesion caused by initial autoreactive attacks, for example, active MS lesion as well. B cell accumulation occurs as packed aggregates or ectopic B cell follicles. Serafini et al., Brain Pathol. 14: 164-144 (2004); Wekerle, Autoimmunity, 50:1, 57-60 (2017); and Pröbstel, et al., International journal of molecular sciences, 16(7), pp. 16576-16592 (2015). In MS, B cells were reported to be found in the brain and spinal cord of RR-, SP-, and β-stage MS patients. Therapeutic treatments that target B cells either directly or indirectly have proven beneficial in treatment of autoimmune diseases such as MS. Wekerle, 2017.

Both CD4+ and CD8+ T cells are present in MS lesions and are believed to play a central role in disease development. Increased frequencies of myelin-reactive (MBP, PLP, and MOG) CD4 and CD8 cells are found in MS patients compared to healthy controls. Cao, et al., Sci. Transl. Med. 7 (287), 287rα74 (2014); Martin, et al., J. Exp. Med. 173 (Jan. 1, 1991), Ota, et al., Nature 346, 183 (Jul. 12, 1990); Pette, et al., Neurology 40, 1770 (1990): and Raddassi, et al., J. Immunol. 187, 1039 (2011).

Th1 cells producing IFN-gamma and Th17 cells are uniquely pathogenic. Factors that favor the development of Th1 cells are elevated in MS patients and are also triggered by viral infections: gamma interferon; IL-12—in almost all treatments that affect the immune system and help MS, almost all decrease Th1 response and increase Th2 and TH3 response. Th17 cells are present at sites of tissue inflammation and are implicated in autoimmune/chronic inflammatory conditions. Th17 producing CD4 and CD8 cells are increased in the lesions, blood, and CSF of patients. [Tzartos 2008; Matusevicius 1999: Bruchlacher-Waldert 2009]. The CCR6 and CD161 on Th17 cells are hypothesized to be homing molecules to inflamed tissues [Cosmi, 2008].

Th17 cells are also implicated in a number of other autoimmune diseases such as systemic lupus erythematosus, rheumatoid arthritis, juvenile idiopathic arthritis (also known as juvenile idiopathic arthritis), Sjögren's syndrome, systemic sclerosis, ankylosing spondylitis, Type 1 diabetes, autoimmune thyroid diseases (Grave's and Hashimoto's), myasthenia gravis, inflammatory bowel disease (Crohn's or ulcerative colitis), and psoriasis. Tabarkiewicz et al., Archivum immunologiae et therapiae experimentalis, 63(6):435-449 (2015).

The ultimate goal of any treatment for autoimmune disease is a complete suppression of pathology. In the case of multiple sclerosis and other autoimmune disorders, pathologic lymphocytes (both B and T cells, and if necessary plasma cells for very severe cases) are expected to be eliminated or controlled to halt the disease course, and interventions at different stages of disease progression require different cellular targets and therefore therapeutic cells.

Disclosed herein are major histocompatibility complex (MHC)-based chimeric receptors (MHC-CAR) for targeting autoreactive immune cells such as autoreactive T cells. A MHC-CAR as described herein comprises one or more MHC polypeptides or an extracellular domain thereof and one or more cell signaling domains, for example, a cytoplasmic signaling domain (e.g., that from CD3ζ), at least one co-stimulatory domain (e.g., that from 4-1BB or CD28), or both. The MHC-CAR may further comprise an antigenic peptide from an autoantigen or a foreign antigen that mimics an autoantigen in eliciting autoimmune responses. Also herein are nucleic acids encoding the MHC-CAR, vectors carrying such, and genetically engineered immune cells such as T cell and natural killer (NK) cells expressing the MHC-CAR. Such genetically engineered immune cells can be used to target autoreactive immune cells, thereby benefiting treatment of autoimmune diseases involving the autoreactive immune cells.

Also disclosed herein are genetically modified regulatory T (Treg) cells expressing an MHC-based chimeric receptor as disclosed herein. Such Treg cells may be further modified with chimeric receptor(s) targeting T cell and/or B cell surface markers, as well as additional genetic engineering for, e.g., targeting specific tissue sites (e.g., lymph nodes or inflammation sites) or modulating immune responses (e.g., checkpoint modulation). The genetically modified Treg cells may be used to inhibit pathogenicity at an early stage of a target disease, to control disease progression at a middle stage of the disease (e.g., relapsing or remitting MS), or to suppress pathology via, e.g., inducing cytotoxicity of pathologic CD8+ T cells at a late stage of the disease (e.g., chronic progressive MS).

I. Major histocompatibility complex (MHC)-based chimeric receptors (MHC-CARs)

The MHC based chimeric receptor (MHC-CAR) described herein comprises an MHC moiety, which is conjugated to an antigenic peptide (e.g., a misfolded one), and at least one cell signaling moiety, which can be a cytoplasmic signaling domain (e.g., that of CD3), one or more co-stimulatory domains (e.g., that of 4-1BB or CD28), or a combination thereof. In some instances, the antigenic peptide can be part of a fusion polypeptide of the MHC-CAR. In other instances, the antigenic peptide does not form a fusion polypeptide with the MHC-CAR but forms a complex with the MHC-CAR As used herein, the term “conjugated” means that at least two components are physically associated, either via covalent bonds or via non-covalent interactions.

In some examples, the MHC-CAR can be a single fusion polypeptide containing the MHC moiety, the antigenic peptide, and the at least one cell signaling moiety. Such a single fusion polypeptide may form complexes with endogenous cell membrane proteins (e.g., β-microglobulin) when expressed in a suitable immune cells.

In other examples, the MHC-CAR described herein may be a multi-chain protein complex, for example, a heterodimer, comprising one polypeptide that comprises the antigenic peptide. In some instances, the antigenic peptide or polypeptide may be expressed as a separate polypeptide, which may form a complex (e.g., a trimer) with the MHC components. The antigenic polypeptide can be a misfolded antigenic protein that binds to the MHC. Optionally, the MHC-CAR may further comprise a hinge domain, which may be adjacent to the antigenic peptide and/or the MHC moiety, a signal peptide at the N-terminus, and/or one or more tagging sites, for example, a histidine protein tag and/or an RQR domain that additionally acts as a kill-switch site.

(i) Components oJMHC-CARs

(a) MHC Moiety

The MHC-CAR constructs disclosed herein comprise an MHC moiety, which may comprise one or more MHC polypeptides or an extracellular domain thereof. The MHC moiety may be derived from a suitable source, for example, human or a non-human mammal (e.g., monkey, mouse, rat, rabbit, pig, etc.) In some instances, the MHC moiety is from a human MHC molecule (also known as HLA). In some instances the domains that interact with molecules from other cells (TCR or BCR) are from a human MHC molecule. There are primarily two classes of MHC molecules, MHC class I molecules and MHC class II molecules, both of which can be used for constructing the MHC-CARs described herein. Sequences of MHC class I and class II molecules of various species (e.g., human, non-human primates, canids, fish, ovids, bovines, equids, suids, murids, and gallus) are available from public gene datasets, for example, the IPD-MHC database and the IMGT/IHLA database provided by EMBL-EBI and the dbMHC database provided by National Center for Biotechnology Information (NCBI).

MHC class I molecules are heterodimers containing an alpha chain and β-microglobulin. The extracellular domain of an alpha chain includes three subdomains, α1, α2, and α3. In some embodiments, the MHC moiety may include the alpha chain of a MHC class I molecule, or an extracellular domain thereof, for example, the α1 domain, the α2 domain, the α3 domain, or a combination thereof. The MHC class I molecule may be a human HLA-A molecule, a human HLA-B molecule, or a human HLA-C molecule. In some instances, the alpha chain of the MHC class I molecule may be fused with β-microglobulin to produce a single chain fusion protein. In some examples, the MHC Class I moiety is from HLA A3, which can be co-used with a PLP peptide. Honma et al., J. Neuroimmunol, 73:7-14 (1997). In other examples, the MHC Class I is from HLA A2, which can be used with the same PLP peptide and display of a viral peptide such as TAX. TAX is from the protein tax or p40 (Genbank accession no. BAB20130.1) that is a molecular mimic of a human neuronal protein and from the HTLV-1 virus, which is implicated in diseases such as rheumatoid arthritis, system lupus erythematosus, and Sjogren's syndrome. Garboczi, et al. The Journal of Immunology. 157(12):5403-5410, 1996. Quaresma, et al., 2015. Viruses, 8(1):5 2015. The class I protein and peptide may additionally contain modifications to enable more robust peptide loading such replacement of the invariant tyrosine at position 84 of the heavy chain with alanine; or alternatively the position 84 tyrosine can be replaced with cysteine as can the second position of the peptide-02m linker to create a disulfide trap. Hansen et al. Trends in immunology, 3/(10):363 (2010).

Like MHC class 1 molecules, MHC class II molecules are also heterodimers consisting of two homogenous peptides, an α-chain and a β-chain. The extracellular domain of each of the α-chain and the β-chain contains two subdomains α1/α2, and β1/β2. When a MHC class II molecule is used for constructing a MHC-CAR, the MHC moiety may include two subunits, one including the α-chain or a portion thereof, for example, an extracellular domain thereof (e.g., α1, α2, or both), the other including the b-chain or a portion thereof, for example, an extracellular domain thereof (e.g., β1, β2, or both). In cases where only the region that interacts with other cell types is used (i.e., α1 and β1), specific amino acid modifications may be required to enhance the folding of the mini-MHC, see mini-sequence with shaded regions and Bimbaum et al. The MHC class II molecule may be a human HLA DP molecule, a human HLA DM molecule, a human HLA DOA molecule, a human HLA DOB molecule, a human HLA DQ molecule, or a human HLA DR molecule. In some examples, the MHC class II molecule is a human HLA DR molecule, for example HLA DR*1501.

(b) Antigenic Peptides

The antigenic peptides of the MHC-CAR described herein are an antigenic peptide that is recognizable by pathogenic immune cells (e.g., autoreactive T cells or B cells) involved in an autoimmune disease. When presented by a suitable MHC molecule, such an antigenic peptide would interact with the antigen-specific T cell receptors of pathogenic T cells, leading to downstream immune responses.

In some instances, a specific antigenic peptide can be designed for a specific autoimmune disease patient such as an MS patient, using methods known in the art. Programs like NetMHC enable personalized design of antigenic peptides that are specific to the patients MHC, and have been used to develop personalized cancer vaccines. Hacohen et al., Cancer immunology research, 1(1):11-15 (2013). Also within the scope of the present disclosure are personalized CAR T and Treg therapies for autoimmune disorders. For disorders with very strong MHC associations (such as MS), a personalized therapy can be utilized to treat a large patient class at different stages of the disease. Recent studies have also demonstrated that Class II MHCs and specifically the HLAs implicated in autoimmune disorders can display entire antigenic proteins rather than just processed peptides. Jiang et al., International immunology, 25(4):235-246, (2013). These MHC-protein complexes appear to induce autoantibody production in autoimmune disorders, including antibodies that do not bind to properly folded proteins as well as autoantibodies that are specific to those with specific autoimmune disorders. The inventors impute that display of antigenic proteins in MHC-CAR can provide a specific route to remove or deactivate autoimmune specific B cells, such as those in MS which produce oligoclonal bands whose specificity to proteins has not been unraveled, despite many rigorous attempts. Owens et al., Annals of neurology, 65(60):639-649, 2009; Chastre et al., New England Journal of Medicine, 374(15):1495-1496, 2016; Housley et al., Clinical Immunology, 161(1):51-58, 2015; Larman et al., 2013. Journal of autoimmunity, 43:1-9, 2013. In the event that the antigenic protein does not bind the MHC, then that specific MHC-CAR will not be expressed, but as a Treg or CTL it can still play a bystander role in modifying the immune response depending upon its other characteristics and as part of a patient specific population of MHC-CAR T and Treg cells with different specificities.

The antigenic peptides used herein may be fragments of autoantigens involved in autoimmune diseases, for example, myelin basic protein (MBP), myelin oligodendrocyte glycoprotein (MOG), and proteolipid protein (PLP) involved in multiple sclerosis, insulin and glutamate decarboxylase (GAD) involved in type I diabetes, tryptase involved in rheumatoid arthritis (RA), and the proteins included in Table 1 below. Alternatively, the antigenic peptide can be a fragment of a pathogen protein such as a viral or a bacterial protein that is highly homologous to a self-antigen involved in an autoimmune disease. Such an antigenic peptide also can target pathogenic T cells. If needed, the antigenic peptide can be a (typically misfolded) antigenic protein or protein fragment that can be expressed separately and binds directly to the MHC moiety of a MHC-CAR described herein. In their natural state (attached to an MHC rather than an MHC moiety of a MHC-CAR), such antigenic protein/MHC complexes stimulate pathogenic B cells to produce autoantibodies. For proteins such as IgGH or rheumatoid factor in rheumatoid arthritis (in et al., Proceedings of the National Academy of Sciences, 111(10):3787-3792, 2014), 02-glycoprotein I in antiphospholipid syndrome (Tanimura et al., Blood, 125(18):2835-2844, 2015) and recurrent miscarriage (Tanimura et al., Placenta, 46:108, 2016), GM-CSF in autoimmune pulmonary alveolar proteinosis (Hamano et al., AVEOLAR MACROPHAGE BIOLOGY B32: A3147-A3147, 2016), tyrosinase in vitiligo (Arase et al. Journal of Dermatological Science, 84(1):e87, 2016), and myeloperoxidase in microscopic polyangiitis (Hiwa et al., Arthritis & Rheumatology. 69(10):2069-2080, 2017), HLA mediated surface display and in some cases autoantibody binding of misfolded variant/HLA complex can occur.

The antigenic peptides for use in the MHC-CAR described herein may contain up to 20 amino acid residues, the extracellular domain of the antigenic protein, or the full length antigenic protein. When co-used with a MHC class I moiety, the antigenic peptide may be 8-10 amino acid-long. Such antigenic peptides would fit well into the peptide binding site of a MHC class I molecule. Antigenic peptides to be co-used with MHC class II moieties can be longer, for example, containing 15-24 amino acid residues or up to the full length of the antigenic protein, since the antigen-binding groove of MHC class II molecules is open at both ends, while the corresponding antigen-binding groove on class I molecules is usually closed at each end. The open antigen-binding groove of MHC class II molecules implicated in autoimmune disorders can also frequently display intact (e.g., yet misfolded) antigenic proteins or splice variants. Jiang et al., International immunology, 25(4):235-246, 2013.

In some examples, a fragment of human MBP is used for constructing the MHC-CARs described herein. An exemplary amino acid sequence of a human MBP is provided below:

(SEQ ID NO: 1)
MASQKRPSQRHGSKYLATASTMDHARHGFLPRHRDTGILDSIGRFFGGDR
GAPKRGSGKVPWLKPGRSPLPSHARSQPGLCNMYKDSHHPARTAHYGSLP
QKSHGRTQDENPVVHFFKNIVTPRTPPPSQGKGRGLSLSRFSWGAEGQRP
GFGYGGRASDYKSAHKGFKGVDAQGTLSKIFKLGGRDSRSGSPMARRHHH
HHH

Exemplary MBP antigenic peptides include, but are not limited to:

(SEQ ID NO: 2)
GSKYLATASTMDHARHGFLPRHRDTGILDSIGRFFGGDRG,
(SEQ ID NO: 3)
KYLATASTMDHARHGFLPRH,
(SEQ ID NO: 4)
ATASTMDHARHGFLPRHRDTGIL,
(SEQ ID NO: 5)
RDTGILDSIGRFFGGDRGAP,
(SEQ ID NO: 6)
IGRFFGGDRGAPKRGSGKDSHHPARTAHY,
(SEQ ID NO: 7)
APKRGSGKDSHHAARTAHY,
(SEQ ID NO: 8)
GSGKDSHHPARTAHYGSLPQ,
(SEQ ID NO: 9)
HHPARTAHYGSLPQKSHGR,
(SEQ ID NO: 10)
HAARTAHYGSLPQKSQGHR,
(SEQ ID NO: 11)
SLPQSHGRTQDENPVVHF,
(SEQ ID NO: 12)
PQDENPVVHFFKNIVTPRTP,
(SEQ ID NO: 13)
TQDENPVVHFFKNIVTPRTP,
(SEQ ID NO: 14)
QDENPVVHFFKNIVTPRTP,
(SEQ ID NO: 15)
DENPVVHFFKNIVTPRTPP,
(SEQ ID NO: 16)
ENPVVHFFKNIVTPR,
(SEQ ID NO: 17)
ENPVVHFFKNIVTPRTP,
(SEQ ID NO: 18)
ENPVVHFFKNIVTP,
(SEQ ID NO: 19)
NPVVHFFKNIVTPRTPPPSQ,
(SEQ ID NO: 20)
VVHFFKNIVTPRT,
(SEQ ID NO: 21)
VVHFFKNIVTPRTPPPSQGK,
(SEQ ID NO: 22)
KNIVTPRTPPPSQGKGRGL,
(SEQ ID NO: 23)
PSQGKGRGLSLSRFSWGAE,
(SEQ ID NO: 24)
GKGRGLSLSRFSWGAEGQRP,
(SEQ ID NO: 25)
LSRFSWGAEGQRPGFGYGG,
(SEQ ID NO: 26)
QRPGFGYGGRASDYKSAHK,
(SEQ ID NO: 27)
ASDYKSAHKGFKGVDAQGT,
(SEQ ID NO: 28)
FKGVDAQGTLSKIFKLGGR,
(SEQ ID NO: 29)
VDAQGTLSKIFKLGGRDSRS,
and
(SEQ ID NO: 30)
SKIFKLGGRDSRSGSPMARR.

An example nucleic acid sequence encoding the MBP antigenic peptide of SEQ ID NO: 15 is provided below:

(SEQ ID NO: 411)
GATGAGAATCCCGTGGTTCATTTTTTTAAGAACATCGTCACACCGCGCAC
CCCACCTG

Specific examples include MBP13-32, MBP89-101, MBP83-99, MBP111-129, or MBP146-170.

Exemplary amino acid sequences for human myelin oligodendrocyte glycoprotein, proteolipid protein, and myelin associated glycoprotein are provided below:

>CAA52617.1 myelin oligodendrocyte glycoprotein [Homo sapiens]

(SEQ ID NO: 31)
MASLSRPSLPSCLCSFLLLLLLQVSSSYAGQFRVIGPRHPIRALVGDEVE
LPCRISPGKNATGMEVGWYPRRFSRVVHLYRNGKDQDGDQAPEYRGRTEL
LKDAIGEGKVTLRIRNVRFSDEGGFTCFFRDHSYQEEAAMELKVEDPFYW
VSPGVLVLLAVLPVLLLQITVGLVFLCLQYRLRGKLRAEIENLHRTFDPH
FLRVPCWKITLFVIVPVLGPLVALIICYNWLHRRLAGQFLEELRNPF

Exemplary MOG antigenic peptides include MOG1-20 or MOG35-55.

>AAA60117.1 proteolipid protein [Homo sapiens]

(SEQ ID NO: 32)
MGLLECCARCLVGAPFASLVATGLCFFGVALFCGCGHEALTGTEKLIETY
FSKNYQDYEYLINVIHAFQYVIYGTASFFFLYGALLLAEGFYTTGAVRQI
FGDYKTTICGKGLSATVTGGQKGRGSRGQHQAHSLERVCTCLGKWLGHPD
KFVGITYALTVVWLLVFACSAVPVYIYFNTWTTCQSIAFPSKTSASIGSL
CADARMYGVLPWNAFPGKVCGSNLLSICKTAEFQMTFHLFIAAFVGAAAT
LVSLLTFMIAATYNFAVLKLMGRGTKF

An exemplary antigenic fragment of PLP is underlined and in boldface. Other examples include PLP139-151(4) or PLP178-191.

>AAH93045.1 Myelin associated glycoprotein Homo sapiens|

(SEQ ID NO: 33)
MIFLTALPLFWIMISASRGGHWGAWMPSSISAFEGTCVSIPCRFDFPDEL
RPAVVHGVWYFNSPYPKNYPPVVFKSRTQVVHESFQGRSRLLGDLGLRNC
TLLLSNVSPELGGKYYFRGDLGGYNQYTFSEHSVLDIVNTPNIVVPPEVV
AGTEVEVSCMVPDNCPELRPELSWLGHEGLGEPAVLGRLREDEGTWVQVS
LLHFVPTREANGHRLGCQASFPNTTLQFEGYASMDVKYPPVIVEMNSSVE
AIEGSHVSLLCGADSNPPPLLTWMRDGTVLREAVAESLLLELEEVTPAED
GVYACLAENAYGQDNRTVGLSVMYAPWKPTVNGTMVAVEGETVSILCSTQ
SNPDPILTIFKEKQILSTVIYESELQLELPAVSPEDDGEYWCVAENQYGQ
RATAFNLSVEFAPVLLLESHCAAARDTVQCLCVVKSNPEPSVAFELPSRN
VTVNESEREFVYSERSGLVLTSILTLRGQAQAPPRVICTARNLYGAKSLE
LPFQGAHRLMWAKIGPVGAVVAFAILIAIVCYITQTRRKKNVTESPSFSA
GDNPPVLFSSDFRISGAPEKYESKEVSTLESH

Table 1 below provides additional exemplary autoantigens associated with other autoimmune diseases.

TABLE 1
Autoantigens of Various Autoimmune Disorders

| | GenBank | Associated Autoimmune |
|---|---|---|
| Autoantigen | Accession No. | Disease |
| Dopachrome tautomerase | AAH28311.1 | Alopecia areata |
| Melanoma antigen gp100 | AAC60634.1 | Alopecia areata |
| Melanocyte protein Pmel | NP_001186983.1 | Alopecia areata |
| Melanocyte-stimulating | NP_002377.4 | Alopecia areata |
| hormone receptor | | |
| Trichohyalin | AAA65582.1 | Alopecia areata |
| Tyrosine 3-monooxygenase | NP_954986.2 | Alopecia areata |
| Amyloid beta A4 protein | NP_000475.1 | Alzheimer's |
| Vasoactive intestinal | NP_004615.2 | Ankylosing spondylitis |
| polypeptide receptor 1 | | |
| Latent membrane protein 2 | CAA57360.1 | Ankylosing spondylitis |
| Nitrogenase iron protein | ART03999.1 | Ankylosing spondylitis |
| Aggrecan core protein | NP_001126.3 | Ankylosing spondylitis |
| Beta-2-glycoprotein 1 | NP_000033.2 | Antiphospholipid syndrome |
| M protein precursor | AAA26918.1 | Antiphospholipid syndrome |
| Large tegument protein | ACL51127.1 | Antiphospholipid syndrome |
| Steroid 21-hydroxylase | NP_000491.4 | Autoimmune adrenalitis |
| Steroid 17-alpha-hydroxylase/17,20 | NP_000093.1 | Autoimmune adrenalitis |
| lyase | | |
| Potassium-transporting | AAB50172.1 | Autoimmune gastritis |
| ATPase alpha chain | | |
| Potassium-transporting | AAA35987.1 | Autoimmune gastritis |
| ATPase beta chain | | |
| Cytochrome P450 2D6 | ABB77909.1 | Autoimmune hepatitis |
| Genome polyprotein | S35630 | Autoimmune hepatitis |
| O-phosphoseryl-tRNA(Sec) | NP_058651.3 | Autoimmune hepatitis |
| selenium transferase | | |
| Asialoglycoprotein receptor | AAB58308.1 | Autoimmune hepatitis |
| Glutathione S-transferase | CAA48637.1 | Autoimmune hepatitis |
| Cytokeratin 8 | AAB18966.1 | Autoimmune hepatitis |
| M protein | AAA26918.1 | Autoimmune myocarditis |
| Myosin-7 | NP_000248.2 | Autoimmune myocarditis |
| Cardiac myosin light chain 1 | AAF91089.1 | Autoimmune myocarditis |
| Cardiac myosin light chain 2 | AAA91832.1 | Autoimmune myocarditis |
| Cardiac actin | NP_005150.1 | Autoimmune myocarditis |
| Troponin I | AC14461.1 | Autoimmune myocarditis |
| Thyroid peroxidase | AAA61217.2 | Autoimmune thyroiditis |
| Thyrotropin receptor | AAB23390.2 | Autoimmune thyroiditis |
| Thyroglobulin | NP_003226.4 | Autoimmune thyroiditis |
| S-arrestin | NP_000532.2 | Autoimmune uvelitis |
| LAMP2 | AAB67314.1 | Autoimmune vasculitis |
| Myeloperoxidase | AAA59863.1 | Autoimmune vasculitis |
| Myeloblastin | NP_002768.3 | Autoimmune vasculitis |
| Alpha-gliadin | AFX69628.1 | Coeliac disease |
| Protein-glutamine | NP_004604.2 | Coeliac disease |
| gamma-glutamyltransferase 2 | | |
| 75k gamma secalin | ADP95479.1 | Coeliac disease |
| Gamma 1 hordein | AFM77738.1 | Coeliac disease |
| Avenin-3-like | ADA62372.1 | Coeliac disease |
| Glycosyltransferase | ANR93567.1 | Crohn's disease |
| 60 kDa heat shock protein, | NP_002147.2 | Crohn's disease |
| mitochondrial | | |
| Transmembrane protein UO-44D | NP_002147.2 | Crohn's disease |
| GM-CSF | AAA52578.1 | Crohn's disease |
| Sucrase-isomaltase, intestinal | NP_001032.2 | Crohn's disease |
| Glutathione peroxidase 2 | NP_002074.2 | Crohn's disease |
| 60 kDa chaperonin 2 | ARX70571.1 | Crohn's disease |
| Pancreatic secretory | NP_001493.2 | Crohn's disease |
| glycoprotein 2 | | |
| 60 kDa chaperonin 2 | OMH58317.1 | Crohn's disease |
| Cytoskeleton-associated | EAW67976.1 | Crohn's disease |
| protein 5 | | |
| AhpC | ETZ42359.1 | Crohn's disease |
| Leukotriene B4 receptor 2 | NP_062813.2 | Crohn's disease |
| Chromodomain-helicase-DNA-binding | NP_001264.2 | Dermatomyositis |
| protein 4 | | |
| Chromodomain-helicase-DNA-binding | NP_001005273.1 | Dermatomyositis |
| protein 3 | | |
| Beta-1 adrenergic receptor | NP_000675.1 | Dialated cardiomyopathy |
| Muscarinic acetylcholine | NP_001006633.1 | Dialated cardiomyopathy |
| receptor M2 | | |
| Collagen alpha-3(IV) chain | CAA56335.1 | Goodpasture's syndrome |
| Thyrotropin receptor | AAB23390.2 | Grave's disease |
| Thyroid peroxidase | AAA61217.2 | Grave's disease |
| Thyroglobulin | CAA29104.1 | Grave's disease |
| Glutamate decarboxylase 2 | NP_000809.1 | Grave's disease |
| TSHR protein | AAI27629.1 | Grave's disease |
| Thyroid peroxidase | AAA61217.2 | Hashimoto's thyroiditis |
| Thyroglobin | CAA29104.1 | Hashimoto's thyroiditis |
| Thyroid stimulating | AAI41971.1 | Hashimoto's thyroiditis |
| hormone receptor | | |
| Insulin | AAA59172.1 | Hypogycemia |
| Insulin receptor | AAA59452.1 | Hypogycemia |
| Integrin beta-3 | NP_000203.2 | Immune thrombocytopenic purpura |
| Integrin alpha-IIb | NP_000410.2 | Immune thrombocytopenic purpura |
| Platelet glycoprotein Ib | NP_000164.5 | Immune thrombocytopenic purpura |
| alpha chain | | |
| Platelet glycoprotein IIIa | AAA52600.1 | Immune thrombocytopenic purpura |
| Thrombopoietin | AAB03393.1 | Immune thrombocytopenic purpura |
| Insulin receptor | AAA59452.1 | Insulin resistant diabetes |
| Phospholipase A2 | NP_000919.1 | Membranous nephritis |
| Myelin basic protein | AAC41944.1 | Multiple sclerosis |
| Myelin proteolipid protein | AAA59565.1 | Multiple sclerosis |
| Myelin-oligodendrocyte | CAA52617.1 | Multiple sclerosis |
| glycoprotein | | |
| Epstein-Barr nuclear antigen 1 | Q1HVF7.1 | Multiple sclerosis |
| DNA polymerase catalytic subunit | AMD82168.1 | Multiple sclerosis |
| 2′,3′-cyclic-nucleotide | AAB24298.2 | Multiple sclerosis |
| 3′-phosphodiesterase | | |
| Oligodendrocyte-myelin | AAA59970.1 | Multiple sclerosis |
| glycoprotein | | |
| Aquaporin-4 | AAH22286.1 | Multiple sclerosis |
| Actin, cytoplasmic 1 | NP_001092.1 | Multiple sclerosis |
| Transposase, mutator family | EUA40098.1 | Multiple sclerosis |
| protein | | |
| E4 gene product | YP_002640224.1 | Multiple sclerosis |
| Protein BOLF1 | AIE89051.1 | Multiple sclerosis |
| Myelin-associated glycoprotein | AAH93045.1 | Multiple sclerosis |
| Transaldolase | NP_006746.1 | Multiple sclerosis |
| Possible transposase | CCP46656.1 | Multiple sclerosis |
| Claudin-11 | NP_005593.2 | Multiple sclerosis |
| Interferon beta | AAC41702.1 | Multiple sclerosis |
| Alpha-crystallin B chain | ACA05949.1 | Multiple sclerosis |
| Apolipoprotein E | AAB59518.1 | Multiple sclerosis |
| Epstein-Barr nuclear antigen 6 | AAA45895.1 | Multiple sclerosis |
| Trans-activator protein BZLF1 | BAP94413.1 | Multiple sclerosis |
| Hemagglutinin | ALB07770.1 | Multiple sclerosis |
| Protein S100-B | NP_006263.1 | Multiple sclerosis |
| DNA polymerase catalytic subunit | SCL76875.1 | Multiple sclerosis |
| Tripartite terminase subunit UL15 | SCL76864.1 | Multiple sclerosis |
| Glyceraldehyde-3-phosphate | CAA25833.1 | Multiple sclerosis |
| dehydrogenase | | |
| Alpha-enolase | CAA34360.1 | Multiple sclerosis |
| Neurofilament light polypeptide | NP_006149.2 | Multiple sclerosis |
| Connexin 43 | AAA52131.1 | Multiple sclerosis |
| Neurofilament medium polypeptide | NP_005373.2 | Multiple sclerosis |
| POTE ankyrin domain family | NP_001264335.1 | Multiple sclerosis |
| member I | | |
| 60 kDa heat shock protein, | NP_002147.2 | Multiple sclerosis |
| mitochondrial | | |
| Epstein-Barr nuclear antigen 3 | BAP94411.1 | Multiple sclerosis |
| Putative HTLV-1-related | CAA34646.1 | Multiple sclerosis |
| endogenous sequence | | |
| Glial fibrillary acidic protein | AAB22581.1 | Multiple sclerosis |
| Phosphomannomutase/phosphoglucomutase | OPA62825.1 | Multiple sclerosis |
| Minor capsid protein L2 | P36745.1 | Multiple sclerosis |
| N-acetylmuramoyl-L-alanine | KIX84070.1 | Multiple sclerosis |
| amidase CwlH | | |
| ATP-sensitive inward rectifier | NP_002232.2 | Multiple sclerosis |
| potassium channel 10 | | |
| mRNA export factor ICP27 homolog | YP_401659.1 | Multiple sclerosis |
| Acetylcholine receptor subunit alpha | NP_001034612.1 | Myasthenia gravis |
| Acetylcholine receptor subunit gamma | NP_005190.4 | Myasthenia gravis |
| Acetylcholine receptor subunit delta | NP_000742.1 | Myasthenia gravis |
| Acetylcholine receptor subunit epsilon | NP_000071.1 | Myasthenia gravis |
| Muscarinic receptor | AAB95158.1 | Myasthenia gravis-MUSC |
| Aquaporin 4 | AAH22286.1 | Neuromyelitis optica |
| Alpha-synuclein | NP_000336.1 | Parkinson's disease |
| DNA polymerase processivity factor | SBO07788.1 | Parkinson's disease |
| Desmoglein-3 | NP_001935.2 | Phemphigus |
| Collagen alpha-1(XVII) chain | NP_000485.3 | Phemphigus |
| Desmoglein-1 | NP_001933.2 | Phemphigus |
| Glutamate decarboxylase 2 | NP_000809.1 | Prediabetes |
| 60 kDa heat shock protein, | NP_002147.2 | Prediabetes |
| mitochondrial | | |
| Insulin | AAA59172.1 | Prediabetes |
| Insulin, isoform 2 | NP_001035835.1 | Prediabetes |
| Islet cell antigen | NP_002837.1 | Prediabetes |
| Dihydrolipoyllysine-residue | NP_001922.2 | Primary biliary cirrhosis |
| acetyltransferase component of | | |
| pyruvate dehydrogenase complex | | |
| Dihydrolipoyllysine-residue | OAF98393.1 | Primary biliary cirrhosis |
| acetyltransferase component of | | |
| pyruvate dehydrogenase complex | | |
| Dihydrolipoyllysine-residue | WP_032229692.1 | Primary biliary cirrhosis |
| succinyltransferase component of | | |
| 2-oxoglutarate dehydrogenase complex | | |
| Glycogen phosphorylase | AAC18079.1 | Primary biliary cirrhosis |
| Nuclear pore glycoprotein 210 | NP_079199.2 | Primary biliary cirrhosis |
| Sarcosine dehydrogenase | AAD32214.1 | Primary biliary cirrhosis |
| Sulfite oxidase | AAA74886.1 | Primary biliary cirrhosis |
| Transglutaminase | BAA14329.1 | Primary biliary cirrhosis |
| Nuclear autoantigen Sp-100 | NP_001073860.1 | Primary biliary cirrhosis |
| Dihydrolipoyllysine-residue | NP_001924.2 | Primary biliary cirrhosis |
| succinyltransferase component of | | |
| 2-oxoglutarate dehydrogenase | | |
| complex, mitochondrial | | |
| Nuclear pore p62 | AAA59990.1 | Primary biliary cirrhosis |
| M protein precursor | AAA26918.1 | Psoriasis |
| Keratin, type I cytoskeletal 16 | NP_005548.2 | Psoriasis |
| Keratin, type I cytoskeletal 17 | NP_000413.1 | Psoriasis |
| ADAMTS-like protein 5 | NP_998769.2 | Psoriasrs |
| Transcriptional activator | AHF70996.1 | Psoriatic arthritis |
| Fibrinogen alpha chain | AAI01936.1 | Psoriatic arthritis |
| Vimentin | NP_003371.2 | Psoriatic arthritis |
| Nebulin-related-anchoring | AI26408.1 | Psoriatic arthritis |
| protein | | |
| M protein | CAM31002.1 | Rheumatic fever |
| Myosin-2 | NP_060004.3 | Rheumatic fever |
| Fibrinogen beta chain | AAI06761.1 | Rheumatoid arthritis |
| Vimentin | NP_003371.2 | Rheumatoid arthritis |
| Rheumatoid factor (IgG) | AAH73766.1 | Rheumatoid arthritis |
| Glucose-6-phosphate isomerase | ARJ36701.1 | Rheumatoid arthritis |
| Collagen alpha-1(II) chain | NP_001835.3 | Rheumatoid arthritis |
| Fibrinogen alpha chain | AAI01936.1 | Rheumatoid arthritis |
| Alpha-enolase | CAA34360.1 | Rheumatoid arthritis |
| Tryptase precursor | AAA86934.1 | Rheumatoid arthritis |
| Filaggrin | NP_002007.1 | Rheumatoid arthritis |
| Aggrecan core protein | NP_001126.3 | Rheumatoid arthritis |
| Small nuclear ribonucleoprotein | NP_008869.1 | Rheumatoid arthritis |
| Sm D1 | | |
| Ribosomal protein L23a | AAB17510.1 | Rheumatoid arthritis |
| 60 kDa chaperonin 2 | OMH58317.1 | Rheumatoid arthritis |
| Trans-activator protein BZLF1 | BAP94413.1 | Rheumatoid arthritis |
| Epstein-Barr nuclear antigen 1 | YP_401677.1 | Rheumatoid arthritis |
| Chaperone protein DnaJ | EDV64758.1 | Rheumatoid arthritis |
| 60 kDa heat shock protein, | NP_002147.2 | Rheumatoid arthritis |
| mitochondrial | | |
| Chitinase-3-like protein 1 | NP_001267.2 | Rheumatoid arthritis |
| mRNA export factor ICP27 homolog | YP_401659.1 | Rheumatoid arthritis |
| Arrestin | AAC50992.1 | Rheumatoid arthritis, iritis |
| Protein BOLF1 | AIE89051.1 | Rheumatoid arthritis, juvenile |
| 60 kDa heat shock protein, | NP_002147.2 | Rheumatoid arthritis, juvenile |
| mitochondrial | | |
| Major DNA-binding protein | BAX36606.1 | Rheumatoid arthritis, juvenile |
| Keratin, type II cytoskeletal 3 | NP_476429.2 | Rheumatoid arthritis, juvenile |
| Fibrillin 1 | BAD16739.1 | Rheumatoid arthritis, juvenile |
| Tenascin precursor | NP_002151.2 | Rheumatoid arthritis, juvenile |
| Stromelysin-1 preproprotein | NP_002413.1 | Rheumatoid arthritis, juvenile |
| Interstitial collagenase | NP_002412.1 | Rheumatoid arthritis, juvenile |
| OspA | CAA32579.1 | Rheumatoid arthritis, Lyme |
| Integrin alpha-L | NP_002200.2 | Rheumatoid arthritis, Lyme |
| DNA topoisomerase 1 | NP_003277.1 | Scleroderma/Systemic sclerosis |
| Histone H3-like centromeric | NP_001800.1 | Scleroderma/Systemic sclerosis |
| protein A | | |
| Small nuclear ribonucleoprotein | NP_008869.1 | Scleroderma/Systemic sclerosis |
| Sm D1 | | |
| Major centromere autoantigen B | NP_001801.1 | Scleroderma/Systemic sclerosis |
| E3 ubiquitin-protein ligase | NP_003132.2 | Scleroderma/Systemic sclerosis |
| TRIM21 | | |
| Epstein-Barr nuclear antigen 1 | YP_401677.1 | Scleroderma/Systemic sclerosis |
| U11/U12 snRNP | Q6IEG0 | Scleroderma/Systemic sclerosis |
| rRNA 2′-O-methyltransferase | NP_001427.2 | Scleroderma/Systemic sclerosis |
| fibrillarin | | |
| Ribonuclease P protein subunit p25 | NP_060263.2 | Scleroderma/Systemic sclerosis |
| 60 kDa SS-A/Ro ribonucleoprotein | NP_001166995.1 | Sjogren's syndrome |
| Lupus La protein | NP_003133.1 | Sjogren's syndrome |
| E3 ubiquitin-protein ligase | NP_003132.2 | Sjogren's syndrome |
| TRIM21 | | |
| Muscarinic acetylcholine | NP_000731.1 | Sjogren's syndrome |
| receptor M3 | | |
| Small nuclear ribonucleoprotein | NP_008869.1 | Sjogren's syndrome |
| Sm D1 | | |
| U1 small nuclear | NP_004587.1 | Sjogren's syndrome |
| ribonucleoprotein A | | |
| Putative HTLV-1-related | CAA34646.1 | Sjogren's syndrome |
| endogenous sequence | | |
| Calreticulin | AAB51176.1 | Sjogren's syndrome |
| Spectrin alpha chain, | NP_001123910.1 | Sjogren's syndrome |
| non-erythrocytic 1 | | |
| Beta-tubulin | AAB59507.1 | Sydenham's chorea |
| Dopamine receptor 1 | NP_000785.1 | Sydenham's chorea |
| Dopamine receptor 2 | NP_000786.1 | Sydenham's chorea |
| 60 kDa SS-A/Ro ribonucleoprotein | NP_001166995.1 | Systemic lupus erythematosis |
| Small nuclear ribonucleoprotein | NP_008869.1 | Systemic lupus erythematosis |
| Sm D1 | | |
| U1 small nuclear | NP_003080.2 | Systemic lupus erythematosis |
| ribonucleoprotein 70 kDa | | |
| Natural killer group protein 2-A | AAC17488.1 | Systemic lupus erythematosis |
| Small nuclear ribonucleoprotein-associated | NP_937859.1 | Systemic lupus erythematosis |
| proteins B and B′ | | |
| Small nuclear ribonucleoprotein-associated | NP_001336393.1 | Systemic lupus erythematosis |
| protein N | | |
| E3 ubiquitin-protein ligase | NP_003132.2 | Systemic lupus erythematosis |
| TRIM21 | | |
| Epstein-Barr nuclear antigen 1 | YP_401677.1 | Systemic lupus erythematosis |
| U1 small nuclear | NP_003084.1 | Systemic lupus erythematosis |
| ribonucleoprotein C | | |
| NHP2-like protein 1 | NP_001003796 | Systemic lupus erythematosis |
| 60S acidic ribosomal | XP_805182.1 | Systemic lupus erythematosis |
| protein P2 | | |
| Histone H1.4 | NP_005312.1 | Systemic lupus erythematosis |
| Glutamate decarboxylase 2 | NP_000809.1 | Type 1 diabetes |
| Insulin | AAA59172.1 | Type 1 diabetes |
| Islet cell antigen | NP_002837.1 | Type 1 diabetes |
| Glucose-6-phosphatase 2 | NP_066999.1 | Type 1 diabetes |
| 60 kDa heat shock protein, | AAH02676.1 | Type 1 diabetes |
| mitochondrial | | |
| Zinc transporter 8 | AAP44332.1 | Type 1 diabetes |
| Insulin, isoform 2 | NP_001035835.1 | Type 1 diabetes |
| Genome polyprotein | AAX23962.1 | Type 1 diabetes |
| Islet amyloid polypeptide | NP_000406.1 | Type 1 diabetes |
| Hemagglutinin | ALB07770.1 | Type 1 diabetes |
| Islet amyloid polypeptide | NP_000406.1 | Type 2 diabetes |
| Zinc transporter 8 | AAP44332.1 | Type 2 diabetes |
| Pancreatic secretory glycoprotein 2 | NP_001493.2 | Ulcerative colitis |
| GM-CSF | AAA52578.1 | Ulcerative colitis |
| Myeloblastin | NP_002768.3 | Ulcerative colitis |
| Type VII collagen | AAA96439.1 | Ulcerative colitis |
| Melanocyte protein PMEL | NP_001186983.1 | Vitiligo |
| Melanin-concentrating hormone | NP_05288.3 | Vitiligo |
| receptor 1 | | |
| Tyrosine 3-monooxygenase | NP_954986.2 | Vitiligo |
| Tyrosinase | NP_000363.1 | Vitiligo |
| L-dopachrome tautomerase | NP_001913.2 | Vitiligo |
| TrpC1 | NP_001238774.1 | Vitiligo |
| Myeloblastin | NP_02768.3 | Wegener's granulomatosis |
| Collagen alpha-1(II) chain | NP_001835.3 | Wegener's granulomatosis |

Table 2 below provides HLA and classes commonly associated with autoimmune disorders though in the exemplary case the HLA or a portion of the HLA will be patient specific and derived from high resolution sequence of the patient suffering from the disorder or a serological equivalent.

TABLE 2
HLA types and classes commonly associated with autoimmune disease

| Common HLA | Common | Common HLA | Common | Associated |
|---|---|---|---|---|
| Class II | Class II | Class I | Class I | Autoimmune |
| Serotypes | Variants | Serotypes | Variants | Disease |
| DR4 (e,g,, | DRB1*04:01; | A2 (e.g., A*02) | A*02; | Alopecia areata |
| DRB1*04); and | DRB1*11; and | | A*02:01 and | |
| DR5 (e.g., | DRB1*11:04 | | B*07:02 | |
| DRB1*11 and | | | | |
| DRB1*12) | | | | |
| | | A2 | | Alzheimer's |
| | | B27 (e.g., | B*27:02; | Ankylosing |
| | | B*2701-2759); | B*27:05; | spondylitis |
| | | B40 (e,g,, | B*40:01; | |
| | | B*40); | B*52; and | |
| | | B27-B40; and | B*38 | |
| | | B7 (e.g., B*07) | | |
| DR7 (e.g., | DRB1*09; | | | Antiphospholipid |
| DRB1*0701-0705); | DRB1*09:01; | | | syndrome |
| DR4; DR5; and | DRB1*04; | | | |
| DR12 (e.g., | DRB1*04:05; and | | | |
| DRB1*1201-3 and | DRB1*14 | | | |
| DRB1*1206) | | | | |
| DR17 (e.g., | DRB1*03:01; | | | Autoimmune |
| DRB1*0301 and | DRB1*04; and | | | adrenalitis |
| DRB1*0304); | DRB1*04:04 | | | |
| DR4; DR4/DR3; | | | | |
| DQ2 (e.g., | | | | |
| DQB1*02); and DQ8 | | | | |
| (e.g., DQB1*0302) | | | | |
| DR2 (e.g., | | | | Autoimmune |
| DRB1*15 and | | | | gastritis |
| DRB1 | | | | |
| DR4; DR5; | | | | |
| DR2/DR4; and | | | | |
| DR4/DR5 | | | | |
| | DRB1*03:01: | | | Autoimmune |
| | DRB3*01:01; | | | hepatitis |
| | DRB1*04:01; | | | |
| | DRB1*04:05; | | | |
| | DRB1*07; and | | | |
| | DRB1*13:01 | | | |
| DR7; DR4; | DRB1*04:09; | | | Autoimmune |
| DR11 (e.g., | DRB1*07; and | | | myocarditis |
| DRB1*1101 to | DRB1*04 | | | |
| DRB1*1110); | | | | |
| DR3 (e.g., | | | | |
| DRB1*03); and | | | | |
| DR11-DQ7.5 | | | | |
| | | B27; | A*29:02; and | Autoimmune |
| | | A29 (e.g., | B*57:01 | uvelitis |
| | | A*29); and | | |
| | | B51 (e.g., | | |
| | | B*51) | | |
| DQ2; DQ8; | DQA1*05:01/ | | | Coeliac disease |
| DR12-DQ7.5; and | DQB1*02:01; | | | |
| DR7-DQ2.2 | DQA1*03/ | | | |
| | DQB1*03:02; and | | | |
| | DQA1*0505/ | | | |
| | DQBA1*0301 | | | |
| DR1 (e.g., | DRB1*07; | B27 | | Crohn's disease |
| DRB1*01); and | DRB1*01:03; | | | |
| DR3 | DRB1*0301; | | | |
| | DRB1*0302; and | | | |
| | DRB3*0301/ | | | |
| | DRB1*1302 | | | |
| | DRB1*0301; and | | | Dermatomyositis |
| | DRB1*0302 | | | |
| DR4 | DRB1*0302 | | | Dilated cardiomyopathy |
| DR2 | | B8 (e.g., B*08) | | Goodpasture's syndrome |
| DR17; DR52 (e.g., | DRB1*03:01; | | | Grave's disease |
| DRB3*); and DR7 | DRB1*04:01; | | | |
| | DRB3*01; and | | | |
| | DRB3*0202 | | | |
| DR3; and DR5 | | | | Hashimoto's thyroiditis |
| DR4 | | | | Immune thrombocytopenic |
| | | | | purpura |
| DR3; DR4; and | | | | Insulin resistant |
| DR3/DR4 | | | | diabetes |
| DR3 | DRB1*01:02 | | | Membranous nephritis |
| DR2; DR15 (e.g., | DRB1*15:01; | A3 (A*03); and | | Multiple sclerosis |
| DRB1*1505-5 and | DRB1*15:01/ | B7 | | |
| DRB1*1507); and | DRB1*15:01; | | | |
| DR53 (DRB4*) | DRB1*15:01/ | | | |
| | DRB5*01:01; | | | |
| | DRB1*15; | | | |
| | DRB5*01:01; | | | |
| | DPw2; | | | |
| | DRB1*04:01; | | | |
| | DRB1*04:04; | | | |
| | DPA1*01:03/ | | | |
| | DPB1*02:01; | | | |
| | DPA1*01:03/ | | | |
| | DPB1*04:01; | | | |
| | DQA1*01:02/ | | | |
| | DQB1*05:02; | | | |
| | DQB1*06; and | | | |
| | DQB1*06:02 | | | |
| DR17; DR3; and DR7 | DRB1*03:01 | | | Myasthenia gravis |
| DR14-DQ5 | | | | Myasthenia |
| | | | | gravis-MUSC |
| DR3 | | | | Neuromyelitis optica |
| DR patient specific | | | | Parkinson's disease |
| DR4; and DR6 | DRB1*01:01; and | | | Phemphigus |
| | DRB1*04:02 | | | |
| DR3; DR4; and | DRB1*03:01; | | | Prediabetes |
| DR3/DR4 | DRB1*04:01; and | | | |
| | DRB1*03: | | | |
| | 01/*04:01 | | | |
| DR8 (e.g., | DRB1*0801; and | | | Primary biliary |
| DRB1*0801-*0807 and | DRB1*0803 | | | cirrhosis |
| DRB1*0810-*0812) | | | | |
| DR7 | DRB1*0102 | B27; and Cw6 | | Psoriasis |
| | | (C*06:02 and | | |
| | | C*06:05) | | |
| | | B16 (e.g., | | Psoriatic arthritis |
| | | B38 and B39); | | |
| | | B17 (e.g., B57 and | | |
| | | B58); B27; | | |
| | | B39 (e.g., | | |
| | | B*39); and Cw6 | | |
| DR7 | | | | Rheumatic fever |
| DR4; DR4-DQ8; | DRB1*01:01; | | | Rheumatoid arthritis |
| DR1; DR12; and | DRB1*01:02; | | | |
| DR18 (e.g., | DRB1*04:01; | | | |
| DRB1*0302 and | DRB1*04:02; | | | |
| DRB1*0303) | DRB1*04:03; | | | |
| | DRB1*04:04; | | | |
| | DRB1*04:05; | | | |
| | DRB1*04:06; | | | |
| | DRB1*04:07; | | | |
| | DRB1*04:08; | | | |
| | DRB1*04:09; | | | |
| | DRB1*04:10; | | | |
| | DRB1*04:11; | | | |
| | DRB1*04:12; | | | |
| | DRB1*04:13; | | | |
| | homozygous | | | |
| | for the above;; | | | |
| | DRB1*01:01/*04: | | | |
| | 04; and | | | |
| | DRB1*01:01/*04: | | | |
| | 01 | | | |
| DR4; DR5; DR14 (e.g., | DRB1*04:01; | | | Rheumatoid arthritis, |
| DRB1*1401-*1408 and | DRB1*04:04; | | | juvenile |
| DRB1*1410-*1408); and | DRB1*04:05; | | | |
| DR15 | DRB1*14:02; and | | | |
| | DRB1*12:01 | | | |
| DR5; and DR11 | DRB1*04:01; | | | Rheumatoid arthritis, |
| | DRBl*10:01: and | | | Lyme |
| | DRB1*11:02 | | | |
| DR11; and DR8 | DRB1*11:04 | | | Rheumatoid arthritis, |
| | | | | pauciarticular |
| | | | | (juvenile) |
| DR5 | DRB1*12:01 | B35 | | Rheumatoid arthritis, |
| | | | | iritis |
| DR5 | DQB1*05:01; | | | Scleroderma/Systemic |
| | DRB1*11; | | | sclerosis |
| | DRB1*11:04; | | | |
| | DRB1*15:02; | | | |
| | DRB1*13:02; | | | |
| | DRB1*04:06; | | | |
| | DRB1*03 | | | Sjogren's syndrome |
| | DRB1*15; | | | |
| | DRB1*03:01/ | | | |
| | DRB1*15:01 | | | |
| DR1 | | B49 (e.g., B*49) | | Sydenham's chorea |
| DR11; and | DRB1*03:01; | | | Systemic lupus |
| DR53-DR7 | DRB1*15:01; | | | erythematosis |
| | DRB1*04:02; | | | |
| | DRB1*04:03; | | | |
| | DRB1*04:06; | | | |
| | DRB1*11:01; and | | | |
| | DRB3*03:01 | | | |
| DR3 | DRB1*03:02; | | | Type 1 diabetes |
| | DRB1*04; | | | |
| | DRB1*04:01; | | | |
| | DRB1*04:02; | | | |
| | DRB1*04:05; | | | |
| | DRB1*03:01; and | | | |
| | DRB1*03:01/ | | | |
| | DRB1*04:01 | | | |
| DR4 | | | | Type 2 diabetes |
| DR1 | DRB1*01:03; and | B27 | | Ulcerative colitis |
| | DRB1*15:02 | | | |
| | DRB1*07:01 | | A*02:01 | Vitiligo |
| | DPB1*04 | | | Wegener's |
| | | | | granulomatosis |

In some embodiments, the antigenic peptides used herein are associated with HLA-DR*1501, for example, GAD peptide TYEIAPVFVLLFYVTLKKMR (SEQ ID NO: 34) (involved in Type I diabetes), the MBP peptides listed above, the following MPP peptides (involved in MS)

(SEQ ID NO: 35)
LLECCARCLVGAPFASLVATGLCFFGVALFC,
(SEQ ID NO: 36)
LVGAPFASLVATGLCFFGVA,
(SEQ ID NO: 37)
FGVALFCGCEVEALTGTEKLIETYFSKNYQD,
(SEQ ID NO: 38)
LFCGCGHEALTGTEKLIETY,
(SEQ ID NO: 39)
TGTEKLIETYFSKNYQDYEY,
(SEQ ID NO: 40)
TGTEKLIETYFSKNYQDYEYL,
(SEQ ID NO: 41)
YFSKNYQDYEYLINVIHAFQYVIYGTASFFFL,
(SEQ ID NO: 42)
GTASFFFLYGALLLAYGFYTTGAVRQIFGDYK,
(SEQ ID NO: 43)
LYGALLLAEGFYTTGAVRQI,
(SEQ ID NO: 44)
FYYTTGAVRQIFGDYKTTICG,
(SEQ ID NO: 45)
AVRQIFGDYKTTICGKGLSATV,
(SEQ ID NO: 46)
RQIFGDYKTTCGKGLSATVTGGQKGRGSRGQ,
(SEQ ID NO: 47)
KGLSATVTGGQKGRGYRGQH,
(SEQ ID NO: 48)
QKGRGSRGQHQAHSLERVCH,
(SEQ ID NO: 49)
KGRGSRGQHQAHSLERVCHCLGCWLGHPDKFV,
(SEQ ID NO: 50)
LGHPDKFVGITY ALTVVWLLVFACSAVPVYIY,
(SEQ ID NO: 51)
SAVPVYIYFNTWTTCQSIAAPCKTSASIGTLC,
(SEQ ID NO: 52)
AVPVYIYFNTWTTCQSIAFP,
(SEQ ID NO: 53)
WTTCQSIAFPSKTSASIGSL,
(SEQ ID NO: 54)
SASIGTLCADARMYGVLPWNAFFGKVCGSNLL,
(SEQ ID NO: 55)
KVCGSNLLSICKTAEFQMTFHLFIAAFVGAAA,
(SEQ ID NO: 56)
AAFVGAAATLVSLLTFMIAATYNFAVLKLMGR,
(SEQ ID NO: 57)
MIAATYNFAVLKLMGRGTKF,
and
(SEQ ID NO: 58)
MAATYNFAVLKLMGRFTKF.

In some embodiments, the antigenic peptides or antigenic polypeptides are patient specific and designed for the patient's MHC. For example, a physician can diagnose the patient with an autoimmune disorder and determine the severity of the disease. The patient's Class I (HLA-A, B, and C) and II (HLA-DR, DQ, DP) regions can be typed, which can now be performed at high resolution using DNA sequencing and with comparison to a reference database (www.ebi.ac.uk/ipd/imgt/hla/). The patient's Class I and II MHC with the strongest evidence of autoimmune involvement can be identified for the disorder. Those known to be associated with a particular autoimmune disorder can be used as references. See, e.g., Tables 1 and 2. The strongest evidence based antigens are identified for the disorder (iedb.org/) and Table 1. A set of personalized peptide (cbs.dtu.dk/services/NetMHC/or cbs.dtu.dk/services,NetMHCII/) and protein targets (for Class II) that are expected to bind the patient autoimmune implicated MHC can be identified.

Personalized MHC-CARs lentivirus or mRNA can be prepared for the patient to enable targeting of pathogenic immune cells. The personalized lentivirus is used to prepare autologous or allogeneic T cells (CTL and/or Tregs) that can be combined with receptor or cellular modifications to allow co-treatment with additional therapeutics, desired interactions with pathogenic cells, routing to a desired location (for interaction with pathogenic inflammatory or inflammation generating cells), or secretion of cytokines (to reduce inflammation).

(c) Co-Stimulatorv Signaling Domains

Many immune cells require co-stimulation, in addition to stimulation of an antigen-specific signal, to promote cell proliferation, differentiation and survival, as well as to activate effector functions of the cell. The MHC-CAR described herein may comprise one or more co-stimulatory signaling domain. The term “co-stimulatory signaling domain,” as used herein, refers to at least a portion of a protein that mediates signal transduction within a cell to induce an immune response such as an effector function. The co-stimulatory signaling domain of the MHC-CAR described herein can be a cytoplasmic signaling domain from a co-stimulatory protein, which transduces a signal and modulates responses mediated by immune cells, such as T cells, NK cells, macrophages, neutrophils, or eosinophils.

Activation of a co-stimulatory signaling domain in a host cell (e.g., an immune cell) may induce the cell to increase or decrease the production and secretion of cytokines, phagocytic properties, proliferation, differentiation, survival, and/or cytotoxicity. The co-stimulatory signaling domain of any co-stimulatory molecule may be compatible for use in the MHC-CAR described herein. Examples of co-stimulatory signaling domains for use in the chimeric receptors can be the cytoplasmic signaling domain of co-stimulatory proteins, including, without limitation, members of the B7/CD28 family (e.g., B7-1/CD80, B7-2/CD86, B7-H1/PD-L1, B7-H2, CD28, CTLA-4, ICOS/CD278, or PD-1): members of the TNF superfamily (e.g., 4-1BB.TNFSF9/CD137, 4-1BB Ligand/TNFSF9, CD40/TNFRSF5, CD40 Ligand/TNFSF5, DR3/TNFRSF25, OX40/fNFRSF4, OX40 Ligand/TNFSF4, or TNF-alpha): and other molecules, such as FRB, and FKBF, that allow co-stimulation to be induced only in the presence of a specific drug molecule (but here in association with a the unique heterodimeric MHC-CAR). Wu et al., Science, 350(6258):aab4077, 2015. In some embodiments, any of the cytoplasmic signaling domains of co-stimulatory proteins may be used in receptors targeting inactive bystander B cells (e.g., with a CD19 or CD20-CAR) or plasma cells (e.g., with a CS1-CAR and/or CSI knockout).

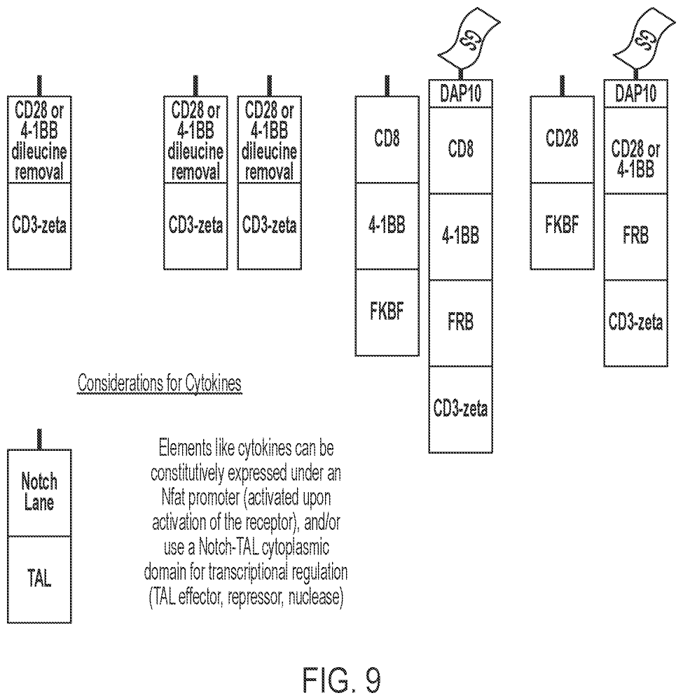

In some instances, the MHC-CAR may comprise a combination (e.g., 2 or 3) co-stimulatory domains, which may be from the same co-stimulatory receptor or from different co-stimulatory receptors. Examples include: CD28+4-1BB, CD28+FRB, CD28+FKBF, or 4-1BB+FRB. See also FIG. 9. In some examples, the MHC-CAR comprises a co-stimulatory domain from CD28, a co-stimulatory domain from 4-1BB, or both. In some embodiments, the co-stimulatory domain is preceded by a short linker. For example, for a class II MHC-CAR, the short linker may be TS (i.e., a MHC internal Linker): for a class I MHC-CAR, the short linker may be PG.

In some instances, the MHC-CAR constructs described herein may include no co-stimulatory domain. Alternatively, it may contain a non-traditional element such as a TALEN nuclease, activators, or repressors which may now be implemented in a clinically applicable lentiviral form using a recoded or non-repeat containing TAL domain and would be linked to a single chain MHC-CAR through a membrane domain derived from Notch.

Exemplary co-stimulatory domains for use in the MHC-CAR described herein include, but are not limited to:

41BB intracellular domain:
(SEQ ID NO: 59)
KRGRKKLLYIFKQPFMRPVQTTQEEDGCSCRFPEEEEGGCEL
41BBe intracellular domain:
(SEQ ID NO: 60)
pgKRGRKKLLYIFKQPFMRPVQTTQEEDGCSCRFPEEEEGGCELaha
CD28 intracellular domain:
(SEQ ID NO: 61)
RSKRSRGGHSDYMNMTPRRPGPTRKHYQPYAPPRDFAAYRS
CD28e intracellular domain:
(SEQ ID NO: 62)
pgRSKRSRGGHSDYMNMTPRRPGPTRKHYQPYAPPRDFAAYRSaha
FRB:
(SEQ ID NO: 63)
EMWHEGLEEASRLYFGERNVKGMFEVLEPLHAMMERGPQTLKETSFNQAY
GRDLMEAQEWDRKYMKSGNVKDLLQAWDLYYHVFRRI
FBP-with linkers
(SEQ ID NO: 64)
(GSSS)₄-EMWHEGLEEASRLYFGERNVKGMFEVLEPLHAMMERGPQTLK
ETSFNQAYGRDLMEAQEWCRKYMKSGNVKDLLQAWDLYYHVFRRI-
(GSSS)₃
FKRB
(SEQ ID NO: 65)
GVQVETISPGDGRTFPKRGQTCVVHYTGMLEDGKKFDSSRDRNKPFKFML
GKQEVIRGWEEGVAQMSVGQRAKLTISPDYAYGATGHPGIIPPHATLVFD
VELLKLE
FKBP-with linkers
(SEQ ID NO: 66)
GVQVETISPGDGRTFPKRGQTCVVHYTGMLEDGKKFDSSRDRNKPFKFML
GKQEVIRGWEEGVAQMSVGQRAKLTISPDYAYGATGHPGIIPPHATLVFD
VELLKLE-(GSSS)₃

(d) Cytoplasmic Signaling Domain

Any cytoplasmic signaling domain comprising an immunoreceptor tyrosine-based activation motif (ITAM) can be used to construct the chimeric receptors described herein. An “ITAM,” as used herein, is a conserved protein motif that is generally present in the tail portion of signaling molecules expressed in many immune cells. The motif may comprises two repeats of the amino acid sequence YxxLI separated by 6-8 amino acids, wherein each x is independently any amino acid, producing the conserved motif YxxL/Ix(6-8)YxxL/I. In some examples, the cytoplasmic signaling domain comprising an ITAM is of CD3ζ. In some examples, the MHC-CAR does not comprise a co-stimulatory domain and the cytoplasmic signaling domain is preceded by a short linker. For example, for a class II MHC-CAR, the short linker may be TS (i.e., a MHC internal Linker). For example for a class 1 MHC-CAR, the short linker may be PG. In some cases the linker may be AHA or absent, such as certain instances when a co-stimulatory domain occurs before a signaling domain.

In some embodiments, the MHC-CAR may include no cytoplasmic signaling domain, for example, that of CD3ζ. Such CD3ζ-free MHC-CAR would have suppressive effects against target cells or induce target cell death. Moisini, et al., The Journal of Immunology, 180(5), pp. 3601-3611.

Provided below is an exemplary cytoplasmic signaling domain from CD3ζ:

(SEQ ID NO: 67)
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

Provided below are exemplary nucleic acid sequences encoding a cytoplasmic signaling domain from CD3ζ:

(SEQ ID NO: 410)
AGAGTAAAGTTTTCCCGAAGTGCGGACGCTCCCGCGTATCAGCAAGGTCA
AAACCAGCTTTACAACGAACTGAACTTGGGACGACGCGAAGAGTACGATG
TTCTTGATAAGCGGAGAGGGCGCGATCCCGAAATGGGGGGAAAGCCTCGG
AGGAAGAACCCACAAGAAGGCCTTTATAATGAACTGCAGAAGGACAAGAT
GGCGGAGGCGTATTCCGAAATAGGCATGAAGGGTGAACGGAGGAGAGGAA
AGGGACATGACGGACTTTATCAAGGATTGTCTACCGCAACTAAAGAaACC
TATGACGCGTTGCACATGCAGGCTCTCCCTCCGAGA
(SEQ ID NO: 422)
CGGGTCAAATTTAGCAGATCCGCTGACGCACCGGCCTACCAGCAGGGCCA
GAACCAACTCTACAACGAGCTGAATCTCGGCCGACGGGAAGAGTATGACG
TACTCGACAAGCGGAGAGGTCGAGACCCTGAGATGGGCGGTAAACCGAGA
CGGAAAAATCCCCAAGAGGGTCTTTATAATGAACTCCAGAAGGATAAGAT
GGCTGAAGCCTATTCTGAGATAGGGATGAAAGGCGAGCGGCGGAGGGGTA
AGGGCCATGATGGCCTTTACCAGGGACTCTCCACGGCAACCAAAGATACT
TACGACGCCCTTCACATGCAAGCCCTCCCGCCACGC

(e) Additional Components

The MHC-CAR described herein may optionally further include one or more of the following components: a hinge domain, a transmembrane domain, a signal (leader) peptide, and a peptide linker.

In some instances, the antigenic peptide may be linked to a hinge peptide to enhance immune targeting activity of the resultant MHC-CAR and/or to reduce antibody responses by the target cell to the MHC-TCR complex. In some examples, a MHC-CAR containing a hinge peptide may not include a cytoplasmic domain (for example, free of a CD3ζdomain). A MHC-CAR construct that contains a hinge peptide may also include a MHC class I moiety. The hinge domain may contain about 10-100 amino acids, e.g., 15-75 amino acids, 20-50 amino acids, or 30-60 amino acids. In some embodiments, the hinge domain may be of 10, 11, 12, 13, 14,15, 16, 17, 18, 19, 20, 21, 22, 23, 24, 25, 26, 27, 28, 29, 30, 35, 40, 45, 50, 55, 60, 65, 70, or 75 amino acids in length.

In some examples, the following peptide linkers can be used in a class I MHC-CAR:

MHCILinker 1:
(SEQ ID NO: 68)
GGGGSGGGGSGGGGS
MHCILinker 2:
(SEQ ID NO: 69)
GGGGGGSGGSGGSGG
MHCILinker 3:
(SEQ ID NO: 70)
GGGGSGGGGSGGGGSGGGGS
MHCILinker 4:
(SEQ ID NO: 68)
GGGGSGGGGSGGGGS

Exemplary peptide linkers for a class II MHC-CAR can be GSGSGSGS (MHCII Linker1; SEQ ID NO: 72), GGGGSGGGGSGGGGS (MHC II LinkerII; SEQ ID NO: 68), GGGGSGGGGSGGS (SEQ ID NO: 400), or those described herein as MHCI Linkers (i.e., MHCI Linkers 1-4). An exemplary pre-peptide linker for a class II MHC-CAR can be AS or GS or one or two copies of either AS or GS.

An example nucleic acid sequence encoding the peptide linker provided by SED ID NO: 400 is provided below

(SEQ ID NO: 401)
GGGGGAGGCGGATCTGGCGGAGGCGGGAGTGGAGGCTCA

A hinge peptide for use in the MHC-CAR described herein may be derived from a naturally-occurring receptor. Hinge domains of any protein known in the art to comprise a hinge domain are compatible for use in the chimeric receptors described herein. In some embodiments, the hinge domain is a portion of the hinge domain of CD8α, e.g., a fragment containing at least 15 (e.g., 20, 25, 30, 35, or 40) consecutive amino acids of the hinge domain of CD8α. Alternatively, it may be a synthetic peptide.

Exemplary hinge domains include: IYIWAPLAGTCGVLLLSLVIT (SEQ ID NO: 73), and IWAPLAGICVALLLSLIITLI (SEQ ID NO: 74). Additional examples are provided below:

FKBP/FRB-CD8 hinge:

FKBP/FRB-CD8 hinge:
(SEQ ID NO: 75)
GVQVETISPGDGRTFPKRGQTCVVHYTGMLEDGKKFDSSRDRNKPFKFML
GKQEVIRGWEEGVAQMSVGQRAKLTISPDYAYGATGHPGIIPPHATLVFD
VELLKLEEAAAREAAAREAAAREAAARGRVAILWHEMWHEGLEEASRLYF
GERNVKGMFEVLEPLHAMMERGPQTLKETSFNQAYGRDLMEAQEWCRKYH
KSGNVKDLLQAWDLYYHVFRRITTTPAPRPPTPAPTIASQPLSLRPEACR
PAAGGAVHTRGLDFACD
GS short hinge:
(SEQ ID NO: 68)
GGGGSGGGGSGGGGS
GS long hinge:
(SEQ ID NO: 76)
GGGGSGGGGSGGGGSGGGGSGGGGSGGGGSGGGGSGGGGSGGGGS
H2-Kb hinge:
(SEQ ID NO: 77)
LRWEPPPSTVSNM
HLA-A2 hinge:
(SEQ ID NO: 78)
LRWEPSSQPTIPI
HLA-A3 hinge:
(SEQ ID NO: 79)
LRWELSSQPTIPI
DAP10 hinge:
(SEQ ID NO: 80)
QTTPGERSSLPAFYPGTSGSCSGCGSLSL
DAP10 hinge with linker:
(SEQ ID NO: 81)
(GSSS)₄QTTPGERSSLPAFYPGTSGSCSGCGSLSLP
DAP12 hinge:
(SEQ ID NO: 82)
LRPVQAQAQSDCSCSTVS
DAP12 hinge with linker:
(SEQ ID NO: 83)
(GSSS)₄LRPVQAQAQSDCSCSTVSP
FcIgGIIIa hinge:
(SEQ ID NO: 84)
GLAVSTISSFFPPGYQ
CD8α hinge:
(SEQ ID NO: 85)
TTTPAPRPPTPAPTIASQPLSLRPEACRPAAGGAVHTRGLDFACD
IgG1 hinge:
(SEQ ID NO: 86)
EPKSPDKTHTCPPCPAPPVAGPSVFLFPPKPKDTLMIARTPEVTCVVVDV
SHEDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSR
WQQGNVFSCSVMHEALHNHYTQKSLSLSPGK
DRA*0101 hinge:
(SEQ ID NO: 87)
EFDAPSPLPETTE
DRB1*1501 hinge:
(SEQ ID NO: 88)
VEWRARSESAQSK

An example nucleic acid sequence encoding a DRA*0101 hinge is provided below.

(SEQ ID NO: 417)
GAGTTCGACGCCCCATCACCGCTTCCAGAAACGACTGAA

An example nucleic acid sequence encoding a DRB1*1501 hinge is provided below.

(SEQ ID NO: 404)
GTTGAGTGGAGGGCGCGGTCAGAGAGCGCACAATCTAAA

In some embodiments, the MHC-CAR constructs described herein further comprise a transmembrane domain. Any transmembrane domain for use in the MHC-CAR can be in any form known in the art. As used herein, a “transmembrane domain” refers to any protein structure that is thermodynamically stable in a cell membrane, preferably a eukaryotic cell membrane. Transmembrane domains compatible for use in the chimeric receptors used herein may be obtained from a naturally-occurring protein. Alternatively, it can be a synthetic, non-naturally occurring protein segment, e.g., a hydrophobic protein segment that is thermodynamically stable in a cell membrane.

Transmembrane domains are classified based on the three dimensional structure of the transmembrane domain. For example, transmembrane domains may form an alpha helix, a complex of more than one alpha helix, a beta-barrel, or any other stable structure capable of spanning the phospholipid bilayer of a cell. Furthermore, transmembrane domains may also or alternatively be classified based on the transmembrane domain topology, including the number of passes that the transmembrane domain makes across the membrane and the orientation of the protein. For example, single-pass membrane proteins cross the cell membrane once, and multi-pass membrane proteins cross the cell membrane at least twice (e.g., 2, 3, 4, 5, 6, 7 or more times).

Membrane proteins may be defined as Type I, Type II or Type III depending upon the topology of their termini and membrane-passing segment(s) relative to the inside and outside of the cell. Type I membrane proteins have a single membrane-spanning region and are oriented such that the N-terminus of the protein is present on the extracellular side of the lipid bilayer of the cell and the C-terminus of the protein is present on the cytoplasmic side. Type II membrane proteins also have a single membrane-spanning region but are oriented such that the C-terminus of the protein is present on the extracellular side of the lipid bilayer of the cell and the N-terminus of the protein is present on the cytoplasmic side. Type III membrane proteins have multiple membrane-spanning segments and may be further sub-classified based on the number of transmembrane segments and the location of N- and C-termini.

In some embodiments, the transmembrane domain of the MHC-CAR described herein is derived from a Type I single-pass membrane protein, e.g., CD8α, CD8β, 4-1BB/CD137, or CD28. Transmembrane domains from multi-pass membrane proteins may also be compatible for use in the chimeric receptors described herein. Multi-pass membrane proteins may comprise a complex (at least 2, 3, 4, 5, 6, 7 or more) alpha helices or a beta sheet structure. Preferably, the N-terminus and the C-terminus of a multi-pass membrane protein are present on opposing sides of the lipid bilayer. e.g., the N-terminus of the protein is present on the cytoplasmic side of the lipid bilayerand the C-terminus of the protein is present on the extracellular side. Either one or multiple helix passes from a multi-pass membrane protein can be used for constructing the chimeric receptor variant described herein.

Exemplary transmembrane domains for use in constructing the MHC-CAR constructs described herein are provided below:

CD8a transmembrane domain:
(SEQ ID NO: 89)
IYIKAFLAGTCGVLLLSLVITLYC
HLA-A2 transmembrane domain:
(SEQ ID NO: 90)
VGIIAGLVLFGAVITGAVVAAVMW
HLA-A3 transmembrane domain:
(SEQ ID NO: 91)
VGIIAGLVLLGAVITGAVVAAVMW
Cd3zeta transmembrane domain:
(SEQ ID NO: 92)
LCYLLDGILFIYGVILTALFL
DR*1501 transmembrane domain:
(SEQ ID NO: 93)
MLSGVGGFVLGLLFLGAGLFI
DR*1501e transmembrane domain:
(SEQ ID NO: 94)
MLSGVGGFVLGLLFLGAGLFIYFRNQ
DRA*0101 transmembrane domain:
(SEQ ID NO: 416)
NVVCALGLTVGLVGIIIGTIFII
DRA*0101e transmembrane domain:
(SEQ ID NO: 418)
NVVCALGLTVGLVGIIIGTIFIIKGL

An example nucleic acid sequence encoding the DR*1501e transmembrane domain is provided below:

(SEQ ID NO: 406)
ATGCTGTCAGGAGTAGGCGGATTTGTACTCGGACTCCTTTTTGGGCGCTG
GGTTGTTTATCTACTTTAGAAACCAA

An example nucleic acid sequence encoding the DRA*0101e transmembrane domain is provided below:

(SEQ ID NO: 419)
AACGTTGTCTGCGCTCTTGGCCTGACAGTGGGCCTGGTAGGCATTATTAT
CGGGACCATCTTTATCATCAAAGGTTTG

Notch transmembrane domain:

(SEQ ID NO: 95)
ILDYSFTGGAGRDIPPPQIEEACELPECQVDAGNKVCNLQCNNHACGWDG
GDCSLNFNDPWKNCTQSLQCWKYFSDGHCDSQCNSAGCLFDGFDCQLTEG
QCNPLYDQYCKDHFSDGHCDQGCNSAECEWDGLDCAEHVPERLAAGTLVL
VVLLPPDQLRNNSFHFLRELSHVLHTNVVFKRDAQGQQMIFPYYGHEEEL
RKHPIKRSTVGWATSSLLPGTSGGRQRRELDPMDIRGSIVYLEIDNRQCV
QSSSQCFQSATDVAAFLGALASLGSLNIPYKIEAVKSEPVEPPLPSQLHL
MYVAAAAFVLLBTVGCGVLLSRKRRR

Notch 2 transmembrane domain:

(SEQ ID NO: 96)
PCVGSNPCYNQGTCEPTSENFFYRCLCPAKFNGLLCMILDYSFTGGAGRD
IPPPQIEEACELPECQVDAGNKVCNLQCNNHACGWDGGDCSLNFNDPWKN
CTQSLQCWKYFSDGHCDSQCNSAGCLFDGFDCQLTEGQCNPLYDQYCKDH
FSDGHCDQGCNSAECEWDGLDCAEHVPESLAAGTLVLVVLLPPDQLRNNS
FHFLRELSHVLHTNVVFKRDAQGQQMIFPYYGHEEELRKHPIKRSTVGWA
TSSLLPGTSGGRQRRELDPMDIRGSIVYLEIDNRQCVQSSSQCFQSATDV
AAFLGALASLGSLNIPYKIEAVKSEPVEPPLPSQLHLMYVAAAAFVLLFF
VGCGVLLSRKRRR

In some embodiments, the MHC-CAR may also comprise a signal peptide (also known as a signal sequence or a leader peptide) at the N-terminus of the polypeptide. In general, signal sequences are peptide sequences that target a polypeptide to the desired site in a cell. In some embodiments, the signal sequence targets the MHC-CAR to the secretory pathway of the cell and will allow for integration and anchor ing of the MHC-CAR into the lipid bilayer. Signal sequences including signal sequences of naturally occurring proteins or synthetic, non-naturally occurring signal sequences, that are compatible for use in the chimeric receptors described herein will be evident to one of skill in the art. In some embodiments, the signal sequence from CD8α. In some embodiments, the signal sequence is from CD28 (e.g., MLRLLLALNLFPSIQVTG (SEQ ID NO: 97)).

Exemplary signal peptides include, but are not limited to, Beta-2-microglobulin signal peptide (e.g., MSRSVALAVLALLSLSGLEA (SEQ ID NO: 98)), HLA A3 signal peptide (e.g., MAVMAPRTLLLLLSGALALTQTWA (SEQ ID NO: 99) or), DRA*0101 signal peptide (e.g., MAISGVPVLGFFIIAVLMSAQESWA (SEQ ID NO: 100)), DRB1*1501 signal peptide (e.g., MVCLKLPGGSCMTALTVTLMVLSSPLAL (SEQ ID NO: 101)), and DRB5 signal peptide (e.g., MVCLKLPGGSYMAKLTVTLMVLSSPLALA (SEQ ID NO: 102)). Exemplary signal peptides may be followed by flexible pre-peptide linkers such as AS, GS, ASAS, GSGS. In some embodiments, a flexible pre-peptide linker is used when the signal peptide is class II and followed by an introduced peptide. Any of the constructs encoding the MHC-CARs described herein may comprise a nucleic acid sequence encoding any of the pre-peptide linkers above, e.g. AS may be encoded by the nucleic acid sequence GCATCT, TS may be encoded by the nucleic acid sequence ACAAGT.

Example nucleic acid sequence encoding beta-2-microglobulin signal peptides are provided below:

(SEQ ID NO: 397)
ATGGTATGCTTGAAGCTCCCGGGCGGGTCCTGCATGACCGCTCTCACTGT
TACTCTTATGGTCCTTAGTTCACCGCTTGCCCTG
(SEQ ID NO: 414)
ATGGCAATATCTGGTGTTCCTGTCCTCGGGTTTTTTATCATAGCCGTACT
GATGTCAGCACAGGAATCATGGGCG

In some embodiments, the MHC-CAR described herein may include one or more peptide linkers between the other components as described herein. Examples include a (GlyₓSer)ₙ linker, wherein x and n, independently can be an integer between 3 and 12, including 3, 4, 5, 6, 7, 8, 9, 10, 11, 12, or more. In some examples, the peptide linker can be (Gly₄Ser)ₙ (SEQ ID NO: 103), wherein n can be an integer between 3 and 20. Specific examples include (Gly₄Ser)₃ (SEQ ID NO: 68),(Gly₄Ser)₆ (SEQ ID NO: 69). (Gly₄Ser)k (SEQ ID NO: 76),(Gly₄Ser)₁₂ (SEQ ID NO: 105), and (Gly₄Ser)₁₅ (SEQ ID NO: 106).

(ii) Configuration of MHC-CARs

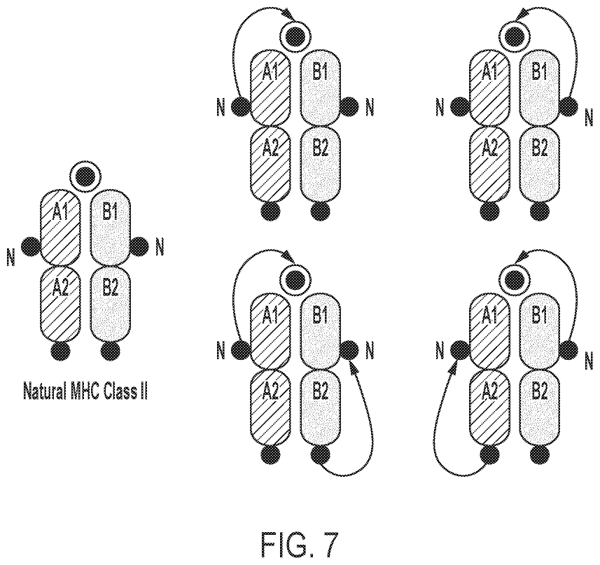

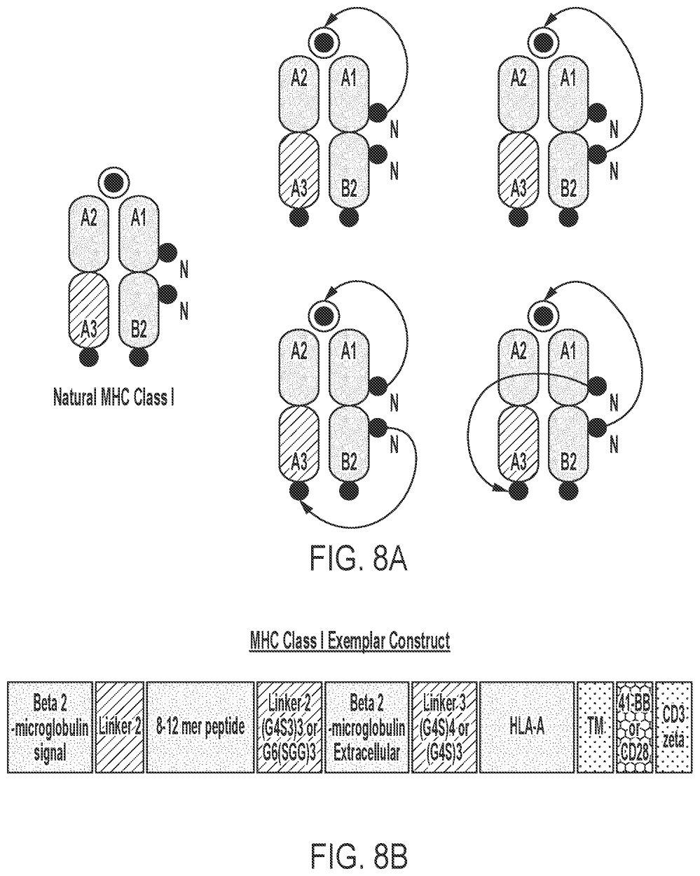

The MHC-CAR constructs disclosed herein, comprising one or more components described herein, may be configured in any suitable format. Exemplary MHC class I constructs and MHC class II constructs are provided in FIGS. 7 and 8.

A MHC-CAR construct containing a MHC class I moiety as described herein may be a single fusion polypeptide that comprise the MHC class I moiety, the antigenic peptide, and a signaling domain (e.g., a co-stimulatory domain, a cytoplasmic signaling domain, or a combination thereof), and optionally one or more of the additional components described herein. See, e.g., FIG. 8. In some examples, a MHC Class I CAR construct contains a hinge domain adjacent to the antigenic peptide. A MHC class I CAR may not contain 02-microglobulin (b2m). When expressed on cell surface, such a MHC-CAR may form a heterodimer with endogenous b2m. Alternatively, a MHC class I CAR may also include b2m, which may be fused with the alpha chain to produce a single polypeptide. In some instances, a MHC class I CAR may contain two subunits, one including the alpha chain or a portion thereof (e.g., an extracellular domain), and the other including b2m or a portion thereof (e.g., an extracellular domain). In some examples, the antigenic peptide may be fused to the alpha chain. In other examples, the antigenic peptide may be fused to b2m. Optionally, a MHC class I CAR may contain peptide linkers between two components. One example is provided in FIG. 8B.

In some examples, the MHC-CAR comprises a class 1 molecule or a portion thereof, for example, HLA A3 or HLA A2, and a antigenic peptide suitable for presentation by the class I molecule (e.g., the PLP fragment KLIETYFSK (SEQ ID NO: 107) or the TAX fragment LLFGYPVYV (SEQ ID NO: 108)). Optionally, the MHC-CAR may further comprise b2m. Alternatively, the b2m molecule may be expressed separately from the class I MHC-CAR. Examples of the class I molecules and b2m sequences are provided below:

HLA A2:
(SEQ ID NO: 109)
GSHSMRYFFTSVSRPGRGEPRFIAVGYVDDTQFVRFDSDAASQRMEPRAP
WIEQSGPEYWDGETRKVKAHSQTHRVDLGTLRGYYNQSEAGSHTVQRMYG
CDVGSDWRFLRGYHQYAYDGKDYIALKEDLRSWTAADMAAQTTKHKWEAA
HVAEQLRAYLEGTCVEWLRRYLENGKETLQRTDAPKTHMTHHAVSDHEAT
LRCWALSFYPAEITLTWQRDGSDQTQDTELVETRPAGDGTFQKWAAVVVP
SGQEQRYTCHVQHEGLPKPLT
HLA A3:
(SEQ ID NO: 110)
GSHSMRYFFTSVSRPGRGEPRFIAVGYVDDTQFVRFDSDAASQRMEPRAP
WIEQEGPEYWDQSTRNVKAQSQTDRVDLGTLRGYYNQSSAGSHTIQIMYG
CDVGSDGRFLRGYRQDAYDGKDYIALNEDLRSWTAADMAAQITKRKWEAA
HEAEQLPAYLDGTCVEWLRRYLENGKETLQRTDPPKTHMTHHPISDHEAT
LRCWALGFYPAEITLTWQRDGEDQTQDTELVETRPAGDGTFQKWAAVVVP
SGEEQRYTCHVQHEGLPKPLT
Microextension for above:
(SEQ ID NO: 111)
LRWE
HLA A2 with H-2K^{b} alpha3 domain
(underlined/italicized)
(SEQ ID NO: 112)
GSHSMRYFFTSVSRPGRGEPRFIAVGYVDDTQFVRFDSDAASQRMSPRAP
WIEQSGPEYWDGETRKVKAHSQTHRVDLGTLRGYYNQSEAGSHTVQRMYG
CDVGSDWRFLRGYHQYAYDGKDYIALKEDLRSWTAADMAAQTTKHKWEAA
HVAEQLRAYLEGTCVEWLRRYLENGKETLQRTDSPKAHVTHHSRPEDKVT
LRCWALGFYPADITLTWQLNGEELIQDMELVETRPAGDGTFQKWASVVVP
LGKEQYYTCHVYHQGLPEPLT
HLA A3 with H-2K^{b} alpha3 domain
(underlined/italicized)
(SEQ ID NO: 113)
GSHSMRYFFTSVSRPGRGEPRFIAVGYVDDTQFVRFDSDAASQRMEPRAP
WIEQEGPEYWDQETRNVKAQSQTDRVDLGTLRGYYNQSSAGSHTIQIMYG
CDVGSDGRFLRGYRQDAYDGKDYIALNEDLRSWTAADMAAQITKRKWEAA
HEASQLRAYLDGTCVEWLRRYLENGKETLQRTDSPKAHVTHHSRPEDKVT
LRCWALGFYPADITLTWQLNGEELIQDMELVETRPAGDGTFQKWASVVVP
LGKEQYYTCHVYHQGLPEPLT
Microextension for above:
(SEQ ID NO: 111)
LRWE
Beta-2-microglobulin (human):
(SEQ ID NO: 114)
IQRTPKIQVYSRHPAENGKSNFLNCYVSGFHPSDIEVDLLKNGERISKVE
HSDLSFSKDWSFYLLYYTEFTPTEKDEYACRVNHVTLSQPKIVKWDRDM
Beta-2-microglobuiin (mouse):
(SEQ ID NO: 115)
IQKTPQIQVYSRHPPENGKPNILNCYVTQFHPPHIEIQMLKNGKKIPKVE
MSDMSFSKDWSFYILAHTEFTPTETDTYACRVKHASMAEPKTVYWDRDM

FIG. 7 provides a number of exemplary designs of MHC class II CAR constructs. Typically, a MHC class II CAR construct contains two subunits, one including the alpha chain or a portion thereof (e.g., an extracellular domain) and the other including the beta chain or a portion thereof (e.g., an extracellular domain). The antigenic peptide can be fused to either the alpha chain or the beta chain. In some instances, a MHC class II CAR can also be in a single fusion polypeptide format, in which the alpha and beta chains are fused to form a single polypeptide. The alpha chain and beta chain of a MHC class II CAR may be derived from the same MHC class II molecule. Alternatively, they may be from different MHC class II molecules. For example, a MHC class II CAR may contain an alpha chain from HLA DRA*1010 and a beta chain from HLA DRB1*1501, which may be fused with an antigenic peptide, such as an MBP peptide.

In some examples, the MHC-CAR comprises a class II molecule or a portion thereof, for example, DRB1*1501 or DRA*O101, and a antigenic peptide suitable for presentation by the class II molecule (e.g., the MBP fragment DENPVVHFFKNIVTPRTPP (SEQ ID NO: 15)). Examples of the class II molecule sequences are provided below;

DRB1*1501:
(SEQ ID NO: 117)
GDTRPRFLWQPKRSCHFFNGTERVRFLDRYFYNQEESVRFDSDVGSFRAV
TELGRPDAEYWNSQKDILEQARAAVDTYCRHNYGVVESFTVQRRVQPKVT
VYPSKTQPLQHHNLLVCSVSGFYPGSIEVRWFLNGQEEKAGMVSTGLIQN
GDWTFQTLVMLETVPRSGEVYTCQVEHPSVTSPLT
DRA*0101
(SEQ ID NO: 118)
IKEEHVIIQAEFYLNPDQSGEFMFDFDGDEIFHVDMAKKETVWRLESFGR
FASFEAQGALANIAVDKANLEIMTKRSNYTPITNVPPEVTVLTNSPVELR
EPNVLICFIDKFTPPVVNVTWLRNGKPVTTGVSETVFLPREDHLFRKFHY
LPFLPSTEDVYDCRVEHWGLDSPLLKHW
DRB1*1501 human/IA-Dbeta mouse (mutated
residues in boldface and underlined)
(SEQ ID NO: 119)
IKEEHVIIQAESYLNPDQSGEFKFDFDGDEIFHVDMAKKETVWRLEEFGR
FASFEAQGALANIAVDKANLEIMTKRSNYTPIEETEVPTSLRRLEQPNVA
ISLSRTEALNHHNTLVCSVTDFYPAKIKVRWFRNGQEETVGVSSTQLIRN
GDWTFQVLVMLEMTPHQGEVYTCHVEWPSLKSPIT
DRA*0101 human/IA-Dalpha mouse (mutated
residues in boldface and underlined)
(SEQ ID NO: 120)
IKEEHVIIQAESYLNPDQSGEFKFDFDGDEIFHVDMAKKETVWRLESFGR
FASFEAQGALANIAVDKANLEIMTKRSNYTPIATNEAPQATVFPKSPVLL
GQPHTLICFVDNIFPPVINITWLRNSKSVTDGVYETSFLVNRDHSFHKLS
YLTFIPSDDDIYDCKVEHWGLEEPVLKHWEPEI
DR-2_{beta} mini (mutated residue in boldface
and underlined)
(SEQ ID NO: 121)
RPRFLWQSKRECHFFNGTERVRFLDRYFYNQEESVRFDSDVGEFRAVTEL
GRPDAEYWNSQKDILEQARAAVDTYCRHNYGVVESFTVQR
DR-2ₐₗₚₕₐ mini
(SEQ ID NO: 122)
IKEEHVIIQAESYLNPDQSGEFKFDFDGDEIFHVDMAKKETVWRLEEFGR
FASFEAQGALANIAVDKANLSIMTKRSNYTPI

An example nucleic acid sequence encoding DRB1*1501 is provided below:

(SEQ ID NO: 402)
GGAGACACAAGACCCCGATTCTTGTGGCAGCCCAAAAGGGAGTGCCATTT
TTTCAATGGGACGGAACGAGTTCGCTTCCTTGATCGGTACTTTTACAACC
AAGAAGAGAGTGTACGGTTCGACTCAGATGTCGGCGAGTTCCGAGCGGTT
ACGGAATTGGGGCGACCTGACGCGGAGTACTGGAACTCCCAAAAGGATAT
TTTGGAGCAGGCACGAGCAGCTGTGGACACCTATTGTCGACATAATTATG
GTGTGGTGGAATCCTTTACAGTTCAGCGGCGGGTGCAACCTAAAGTGACC
GTGTATCCATCTAAAACGCAACCCCTCCAACACCATAACCTCCTGGTGTG
TTCCGTAAGCGGCTTCTATCCCGGGTCAATTGAGGTCAGGTGGTTCCTCA
ACGGTCAGGAGGAGAAGGCCGGAATGGTAAGTACTGGTCTTATCCAGAAC
GGAGACTGGACCTTCCAAACTTTGGTAATGTTGGAAACGGTGCCGCGATC
CGGGGAGGTGTATACATGCCAAGTTGAACACCCGAGTGTTACGAGCCCCC
TGACG

An example nucleic acid sequence encoding DRA*0101 is provided below:

(SEQ ID NO: 415)
ATAAAAGAAGAGCACGTGATAATACAGGCGGAGTTTTATTTGAACCCGGA
CCAGAGCGGTGAGTTCATGTTCGATTTTGATGGCGACGAGATATTTCACG
TTGACATGGCAAAAAAGGAAACGGTGTGGAGACTTGAGGAGTTTGGACGA
TTCGCATCATTTGAGGCACAAGGAGCACTCGCCAATATCGCGGTGGACAA
GGCCAACCTGGAGATCATGACAAAACGCTCCAATTATACGCCTATCACTA
ATGTGCCCCCTGAGGTTACTGTGCTCACAAATTCTCCCGTAGAACTTAGG
GAACCTAACGTCCTCATATGTTTCATCGACAAGTTCACTCCTCCGGTGGT
CAATGTAACGTGGCTTCGGAATGGTAAGCCGGTCACCACGGGTGTCTCAG
AGACCGTATTTCTGCCCAGAGAAGACCACCTCTTCCGCAAATTTCATTAC
CTTCCCTTTCTTCCTTCAACGaAAGACGTTTACGACTGCAGGGTCGAACA
TTGGGGGCTTGACGAGCCACTTCTCAAGCATTGG

Any of MHC class I and MHC class II constructs described herein can be further fused to one or more signaling domains and optionally one or more of the additional components. In some instances, the MHC-CAR constructs described herein are free of singling domains.

Preferably, a MHC-CAR as described herein contains matched MHC moiety and antigenic peptide, e.g., a MHC molecule that would present the antigenic peptide or homologous analogs in natural state. In some instances, a MHC-CAR described herein may contain an alpha chain or a beta chain from HLA DRB1*1501 and an antigenic peptide associated with this HLA allele, e.g., those MBP peptides described herein and others as well. The association between antigenic peptides involved in an autoimmune disease and a specific HLA allele is well known in the art or can be identified via routine practice, for example, library screening.

One exemplary MHC-CAR may have the following formula (+/− means that the specific component is optional):
Single chain (MHC Class I or II+peptide) (+/−hinge)+single chain CD28/4-1BB (+/−dileucine motifs) (+/−cd3zeta)). (Additional short peptide linkers can be added between components as described previously.)

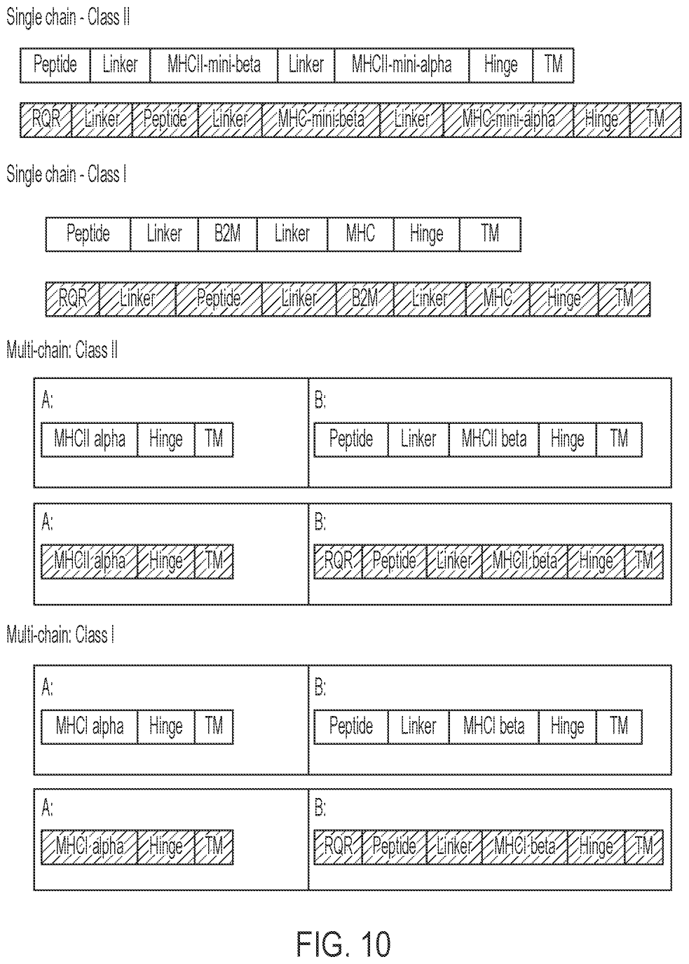

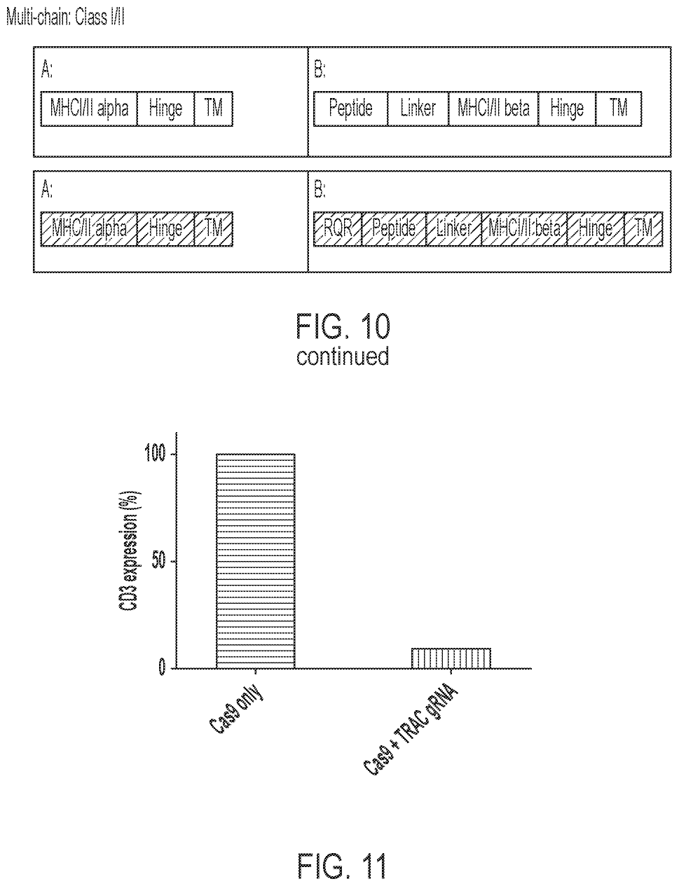

Other exemplary MHC-CAR designs (single chain and multi-chain) are illustrated in FIG. 10. In the case of multi-chain constructs, one or more short hinges may be used to enhance successful expression of the MHC-CAR. Further, it may be desirable to replace a portion of the structure with conserved domains from mice domains to prevent cross-reactivity. Note that in some cases, the internal domain may only be attached to one of the chains.

The amino acid sequence of a MHC-CAR binding (that displays MBP) TCR is provided below:

TCR alpha MBP:
(SEQ ID NO: 123)
METLLGVSLVILWLQLARVNSQQGEEDPQALSIQEGENATMNCSYKTSIN
NLQWYRQNSGRGLVHLILIRSNEREKHSGRLRVTLDTSKKSSSLLITASR
AADTASYFCATAAVGGFKTIFGAGTRLFVKANIQNPDPAVYQLRDSKSSD
KSVCLFTDFDSQTNVSQSKDSDVYITDKTVLDMRSMDFKSNSAVAWSNKS
DFACANAFNNSIIPEDTFFPSPESSCDVKLVEKSFETDTNLNFQNLSVIG
FRILLLKVAGFNLLMTLRLWSS
TCR beta MBP
(SEQ ID NO: 124)
MLLLLLLLGLAGSGLGAWSQHPSWVISKSGTSVKIECRSLDSFQATTMFW
YRQFPKQSLMLMATSNEGSKATYEQGVEKDKFLINHASLTLSTLTVTSAH
PEDSSFYICSARDLTSGANNEQFFGPGTRLTVLSDLKNVFPPEVAVFEPS
EAEISHTQKATLVCLATGFYPDHVELSWWVNGKEVHSGVSTDPQPLKEQP
ALNDSRYSLSSRLRVSATFWQNPRNHFRCQVQFYGLSENDEWTQDRAKPV
TQIVSAEAWGRADCGFTSESYQQGVLSATILYEILLGKATLYAVLVSALV
LMAMVKRKDSRG
TCR alpha class I
(SEQ ID NO: 125)
mamllgasvl ilwlqpdwvn sqqkndd
QQVKQNSPSLSVQEGRISILNCDYTNSMFDYFLWYKKYPAEGPTFLISIS
SIKDKNADGRFTVFLNKSAKHLSLHIVPSQPGDSAVYFCAAMEGAQKLVF
GQGTRLTINPNIQNPDPAVYQLRDSKSSDKSVCLFTDFDSQTNVSQSKDS
DVYITDKTVLDMRSMDFKSNSAVAWSNKSDFACANAFNNSIIPSDTFFPS
PESS
Cdvklve ksfetdtnln fqnlsvigfr illlkvagfn
llmclrlwss
TCR beta class I:
(SEQ ID NO: 126)
msigllccaa lsllwagpv
NAGVTQTPKFQVLKTGQSMTLQCAQDMNHEYMSWYRQDPGMGLRLIHYSV
GAGITDQGEVPNGYNVSRSTTEDFPLRLLSAAPSQTSVYFCASSYPGGGF
YEQYFGPGTRLTVTEDLKNVFPPEVAVFEPSEASISHTQKATLVCLATGF
YPDHVELSWWVNGKEVHSGVSTDPQPLKEQPALNDSRYALSSRLRVSATF
WQDPRNHFRCQVQFYGLSENDEWTQDRAKPVTQIVSAEAWGRAD
cgftse syqqgvlsat ilyeillgka tlyavlvsal vlmamvkrkd
srg

The amino acid sequences of exemplary CD19 targeting CAR constructs are provided below (note that these designs contain a 4-1BB domain which may be replaced with a cd28 domain):

4G7-CAR version 1:
(SEQ ID NO: 127)
MALPVTALLLPLALLLHAARPEVQLQQSGPELIKPGASVKMSCKASGYTF
TSYVMHWVKQKPGQGLEWIGYINPYNDGTKYNEKFKGKATLTSDKSSSTA
YMELSSLTSEDSAVYYCARGTYYYGSRVFDYWGQGTTLTVSSGGGGSGGG
GSGGGGSDIVMTQAAPSIPVTPGESVSISCRSSKSLLNSNGNGSGSGTAF
TLRISRVEAEDVGVYYCMQHLEYPFTAGTKLELKRSDPTTTPAPRPPTPA
PTIASQPLSLRPEACRPAAGGAVHTRGLDFACDIYIWAPLAGTCGVLLLS
LVITLYCKRGRKKLLYIFKQPFMRPVQTTQEEDGCSCRFPEEEEGGCELR
VKFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDTY
DALHMQALPPR
4G7-CAR version 2:
(SEQ ID NO: 128)
METDTLLLWV LLLWVPGSTG EVQLQQSGPE LIKPGASVKM
SCKASGYTFT SYVMHWVKOK PGOGLEWIGY INPYNDGTKY
NEKFKGKATL TSDKSSSTAY MELSSLTSED SAVYYCARGT
YYYGSRVEDY WGQGTTLTVS SGGGGSGGGG SGGGGSDIVM
TQAAPSIPVT PGESVSISCR SSKSLLNSNG NTYLYWFLQR
PGQSPQLLIY RMSNLABGVP DRFSGSGSGT AFTLRISRVE
AEDVGVYYCM QHLEYPFTFG AGTKLELKRS DPTTTPAPRP
PTPAPTIASQ PLSLRPEACR PAAGGAVHTR GLDEACDIYI
WAPLAGTCGV LLLSLVITLY CKRGRKKLLY IFKOPFMRPV
QTTQEEDGCS CRFPEEEEGG CELRVKFSRS ADAPAYQQGQ
NQLYNELNLG RREEYDVLDK RRGRDPEMGG KPRRKNPQEG
LYNELQKDKM ALAYSEIGMK GEPPRGKGHD GLYQGLSTAT
KDTYDALHMQ ALPPR

The nucleic acid sequences of exemplary CD19 targeting CAR constructs are provided below.

4G7-CAR version 2:
(SEQ ID NO: 390)
atggagacagacactcttctcctttgggtcttgctgctgtgggtt
cccggaagcacaggagaagtacagttgcaacagtctgggccagaa
ctcatcaaacccggagcttctgtaaaaatgtcatgcaaagctagt
ggatatacatttacttcttacgtgatgcactgggtaaaacagaaa
cctggtcaggggcttgagtggatcgggcacattaacccatataat
gacggcaccaaatataacgagaaattcaagggaaaggctacgctt
acascagataagtccagtagcaccgcttatatggaacccagcagc
cttacttccgaagattccgcggtgcactactgcgcgagagggact
tactactacgggagtcgagtattcgattattggggtcaaggcacg
acgctcacggtgagctcaggtggtggagggtctgggggtggcggc
agtggtggggggggctcagacatcgtgatgacccaggcagcacct
tctatcccggtaaccccaggcgagtctgtatctatcagstgtcgg
tccagcaagcctcttctcaacagtaacggcaatacatatctccac
tggttcctccaaaggcctgggcaaagtcctcaacttcttatatat
cggatgtccaatcttgcgagtggcgtacccgacaggttttcaggg
tctgggagcggaacagcttttacgttgagaatatccagggtagaa
gctgaggacgtcggtgtatattattgcatgcaacatctcgaatac
ccctttaccttcggcgctggtacaaagctcgaattgaaacgcagc
gatccaaccacgacgccagcgccacgaccacctacgcccgctcca
actattgcctcccagcccctgagtcttcggccagaagcgtgtaga
cctgctgccggcggggccgttcacacgcggggccttgactttgca
tgtgatatctatatatgggctcctttggcgggaacttgcggagtg
cttcttttgtcactcgtgataacgttgtattgtaaaaggggtcga
aagaaactcctctatatatttaagcagccctttatgaggcccgtg
caaacaacacaagaagaggacggatgctcttgtcgattcccggaa
gaggaggagggggggtgtgagctcagggtcaagttttctcgctct
gccgacgcgccagcctatcaacagggccaaaaccagctgtataac
gaactcaacctcgggcgccgggaagagtatgacgtccttgacaaa
cggcgcggtcgcgaccctgaaatgggtggaaaaccgaggcgaaag
aacccccaggagggactttacaacgaattgcaaaaagacaagatg
gccgaagcctattccgaaattggaatgaaaggcgagcggagacga
ggtaaggggcatgacggcctgtatcaagggctctctacggccacg
aaggatacttacgacgcccttcatatgcaagctcttccaccacgg

MHC-CAR1 containing MHC-CAR1 part B (MHC-DRB CAR)-HLA DRB1*1501 (signal peptide), MBP peptide, HLA DRB1*1501 (external, hinge, transmembrane) CD3ζ(cytoplasmic signaling domain) is provided below:

(SEQ ID NO 412)
MVCLKLPGGSCMTALTVTLMVLSSPLALASDENPVVH
FFKNIVTPRTPPGGGGSGGGGSGGSGDTRPRFLWQPK
RECHFFNGTERVRFLDRYFYNQSESVRFDSDVGEFRA
VTSLGRPDASYWNSQKDILEQARAAVDTYCRHNYGVV
ESFTVQRRVQPKVTVYPSKTQPLQHHNLLVCSVSGFY
PGSIEVRWFLNGQEEKAGMVSTGLIQNGDWTFQTLVM
LETVPRSGSVYTCQVEHPSVTSPLTVEWRARSSSAQS
KMLSGVGGFVLGLLFLGAGLFIYFRNQTSRVKFSRSA
DAPAYQQGQNQLYNELNLGRREEYDVLDKRRGRDPEM
GGKPRRKNPQEGLYNELQKDKMAEAYSEIGMKGERRR
GKGHDGLYQGLSTATKDTYDALHMQALPPR

An example nucleic acid sequence encoding a MHC_CAR1 containing MHC-CAR1 part B (MHC-DRB CAR)-HLA DRB1*1501 (signal peptide), MBP peptide. HLA DRB1*1501 (external, hinge, transmembrane) CD3ζ(cytoplasmic signaling domain) is provided below:

(SEQ ID NO: 413)
ATGGTATGCTTGAAGCTCCCGGGCGGGTCCTGCATGA
CCGCTCTCACTGTTACTCTTATGGTCCTTAGTTCACC
GCTTGCCCTGGCATCTGATGAGAATCCCGTGGTTCAT
TTTTTTAAGAACATCGTCACACCGCGCACCCCACCTG
GGGGAGGCGGATCTGGCGGAGGCGGGAGTGGAGGCTC
AGGAGACACAAGACCCCGATTCTTGTGGCAGCCCAAA
AGGGAGTGCCATTTTTTCAATGGGACGGAACGAGTTC
GCTTCCTTGATCGGTACTTTTACAACCAAGAAGAGAG
TGTACGGTTCGACTCAGATGTCGGCGAGTTCCGAGCG
GTTACGGAATTGGGGCGACCTGACGCGGAGTACTGGA
ACTCCCAAAAGGATATTTTGGAGCAGGCACGAGCAGC
TGTGGACACCTATTGTCGACATAATTATGGTGTGGTG
GAATCCTTTACAGTTCAGCGGCGGGTGCAACCTAAAG
TGACCGTGTATCCATCTAAAACGCAACCCCTCCAACA
CCATAACCTCCTGGTGTGTTCCGTAAGCGGCTTCTAT
CCCGGGTCAATTGAGGTCAGGTGGTTCCTCAACGGTC
AGGAGGAGAAGGCCGGAATGGTAAGTACTGGTCTTAT
CCAGAACGGAGACTGGACCTTCCAAACTTTGGTAATG
TTGGAAACGGTGCCGCGATCCGGGGAGGTGTATACAT
GCCAAGTTGAACACCCGAGTGTTACGAGCCCCCTGAC
GGTTGAGTGGAGGGCGCGGTCAGAGAGCGCACAATCT
AAAATGCTGTCAGGAGTAGGCGGATTTGTACTCGGAC
TCCTCTTTTTGGGCGCTGGGTTGTTTATCTACTTTAG
AAACCAAACAAGTAGAGTAAAGTTTTCCCGAAGTGCG
GACGCTCCCGCGTATCAGCAAGGTCAAAACCAGCTTT
ACAACGAACTGAACTTGGGACGACGCGAAGAGTACGA
TGTTCTTGATAAGCGGAGAGGGCGCGATCCCGAAATG
GGGGGAAAGCCTCGGAGGAAGAACCCACAAGAAGGCC
TTTATAATGAACTGCAGAAGGACAAGATGGCGGAGGC
GTATTCCGAAATAGGCATGAAGGGTGAACGGAGGAGA
GGAAAGGGACATGACGGACTTTATCAAGGATTGTCTA
CCGCAACTAAAGACACCTATGACGCGTTGCACATGCA
GGCTCTCCCTCCGAGA

MHC-CAR containing MHC-CAR1 part A (MHC-DRA CAR) HLA-DRA*1010 (signal peptide, external, hinge, transmembrane), CD3ζ(cytoplasmic signaling domain) is provided below:

(SEQ ID NO: 423)
MAISGVPVLGFFIIAVLMSAQESWAIKEEHVIIQAEFYLNPDQS
GSFMFDEDGDEIFHVDMAKKETVWRLEEFGRFASFEAQGALANI
AVDKANLEIMTKRSNYTPITNVPPEVTVLiTNSPVELREPNVLI
CFIDKFTPPWNVTWLRNGKPVTTGVSETVFLPREDHLFRKFHYL
PFLPSTEDVYDCRVEHWGLDEPLLKHVJEFDAPSPLPETTENWC
ALGLTVGLVGIIIGTIFIIKGLTSRVKFSRSADAPAYQQGQKQL
YNELNLGRREEYDVLDKRRGRDPEMGGKPRRKNPQEGLYNELQK
DKMAEAYSEIGMKGERRRGKGHDGLYQGLSTATKDTYDALHMQA
LPPR

An example nucleic acid sequence encoding a MHC-CAR1 part A (MHC-DRA CAR) HLA-DRA*1010 (signal peptide, external, hinge, transmembrane), CD3ζ (cytoplasmic signaling domain) is provided below:

(SEQ ID NO: 424)
ATGGCAATATCTGGTGTTCCTGTCCTCGGGTTTTTTATCATAGC
CGTACTGATGTCAGCACAGGAATCATGGGCGATAAAAGAAGAGC
ACGTGATAATACAGGCGGAGTTTTATTTGAACCCGGACCAGAGC
GGTGAGTTCATGTTCGATTTTGATGGCGACGAGATATTTCACGT
TGACATGGCAAAAAAGGAAACGGTGTGGAGACTTGAGGAGTTTG
GACGATTCGCATCATTTGAGGCACAAGGAGCACTCGCCAATATC
GCGGTGGACAAGGCCAACCTGGAGATCATGACAAAACGCTCCAA
TTATACGCCTATCACTAATGTGCCCCCTGAGGTTACTGTGCTCA
CAAATTCTCCCGTAGAACTTAGGGAACCTAACGTCCTCATATGT
TTCATCGACAAGTTCACTCCTCCGGTGGTCAATGTAACGTGGCT
TCGGAATGGTAAGCCGGTCACCACGGGTGTCTCAGAGACCGTAT
TTCTGCCCAGAGAAGACCACCTCTTCCGCAAATTTCATTACCTT
CCCTTTCTTCCTTCAACGGAAGACGTTTACGACTGCAGGGTCGA
ACATTGGGGGCTTGACGAGCCACTTCTCAAGCATTGGGAGTTCG
ACGCCCCATCACCGCTTCCAGAAACGACTGAAAACGTTGTCTGC
GCTCTTGGCCTGACAGTGGGCCTGGTAGGCATTATTATCGGGAC
CATCTTTATCATCAAAGGTTTGACTTCCCGGGTCAAATTTAGCA
GATCCGCTGACGCACCGGCCTACCAGCAGGGCCAGAACCAACTC
TACAACGAGCTGAATCTCGGCCGACGGGAAGAGTATGACGTACT
CGACAAGCGGAGAGGTCGAGACCCTGAGATGGGCGGTAAACCGA
GACGGAAAAATCCCCAAGAGGGTCTTTATAATGAACTCCAGAAG
GATAAGATGGCTGAAGCCTATTCTGAGATAGGGATGAAAGGCGA
GCGGCGGAGGGGTAAGGGCCATGATGGCCTTTACCAGGGACTCT
CCACGGCAACCAAAGATACTTACGACGCCCTTCACATGCAAGCC
CTCCCGCGACGC

Construct 1 (CD 19 CAR and CCR6 region) is provided below:

(SEQ ID NO: 425)
METDTLLLWVLLLWVPGSTGEVQLQQSGPELIKPGASVKMSCKA
SGYTFTSYVMHWVKQKPGQGLEWIGYINPYNDGTKYNEKFKGKA
TLTSDKSSSTAYMELSSLTSEDSAVYYCARGTYYYGSRVFDYWG
QGTTLTVSSGGGGSGGGGSGGGGSDIVMTQAAPSIPVTPGESVS
ISCRSSKSLLNSNGNTYLYWFLQRPGQSPQLLIYRMSNLASGVP
DRFSGSGSGTAFTLRISRVEAEDVGVYYCMQHLEYPFTFGAGTK
LELKRSDPTTTPAPRPPTPAPTIASQPLSLRPEACRPAAGGAVH
TRGLDFACDIYIWAPLAGTCGVLLLSLVITLYCKRGRKKLLYIF
KQPFMRPVQTTQEEDGCSCSFPEEEEGGCELRVKFSRSADAPAY
QQGQNQLYNELNLGRREEYDVLDKRRGRDPEMGGKPRRKNPQSG
LYNELQKDKMAEAYSEIGMKGERRRGKGHDGLYQGLSTATKDTY
DALHMQALPPRGSSGSGEGRGSLLTCGDVSENPGPMSGESMNFS
DVFDSSEDYFVSVNTSYYSVDSEMLLCSLQEVRQFSRLFVPIAY
SLICVFGLLGNILVVITFAFYKKARSMTDVYLLNMAIADILFVL
TLPFWAVSHATGAWVFSNATCKLLKGIYAINFNCGMLLLTCISM
DRYIAIVQATKSFRLRSRTLPRSKIICLVVWGLSVIXSSSTFVF
NQKYNTQGSDVCEPKYQTVSEPIRWKLLMLGLELLFGFFIPLMF
MIFCYTFIVKTLVQAQNSKRHKAIRVIIAVVLVFLACQIPHNMV
LLVTAANLGKMNRSCQSEKLIGYTKTVTEVLAFLHCCLNPVLYA
FIGQKFRNYFLKILKDLWCVRRKYKSSGFSCAGRYSENISRQTS
ETADNDNASSFTM

Construct 1 (CD 19 CAR, CCR6. GFP region) is provided below:

(SEQ ID NO: 429)
METDTLLLWVLLLWVPGSTGEVQLQQSGPELIKPGASVKMSCKASGYT
FTSYVMHWVKQKPGQGLEWIGYINPYNDGTKYNEKFKGKATLTSDKSS
STAYMELSSLTSEDSAVYYCARGTYYYGSRVFDYWGQGTTLTVSSGGG
GSGGGGSGGGGSDIVMTQAAPSIPVTPGESVSISCRSSKSLLNSNGNT
YLYWFLQRPGQSPQLLIYRMSNLASGVPDRFSGSGSGTAFTLRISRVE
AEDVGVYYCMQHLEYPFTFGAGTKLELKRSDPTTTPAPRPFTPAPTIA
SQPLSLRPEACRPAAGGAVHTRGLDFACDIYIWAPLAGTCGVLLLSLV
ITLYCKRGRKKLLYIFKQPFMRPVQTTQEEDGCSCRFPEEEEGGCELR
VKFSRSADAPAYQQGQNQLYNSLNLGRRESYDVLDKRRGRDPEMGGKP
RRKNPQEGLYNELQKDKMAEAYSEIGMKGERRRGKGHDGLYQGLSTAT
KDTYDALHMQALPPRGSSGSGEGRGSLLTCGDVEENPGPMSGESMNFS
DVFDSSEDYFVSVNTSYYSVDSEMLLCSLQEVRQFSRLFVPIAYSLIC
VFGLLGNILVVITFAFYKKARSMTDVYLLNMAIADILFVLTLPFWAVS
HATGAWVFSNATCKLLKGIYAINFNCGMLLLTCISMDRYIAIVQATKS
FRLRSRTLPRSKIICLVVWGLSVIISSSTFVFNQKYNTQGSDVCSPKY
QTVSEPIRWKLLMLGLELLFGFFIPLMFMIFCYTFIVKTLVQAQNSKR
HKAIRVIIAVVLVFLACQIPHNMVLLVTAANLGKMNRSCQSEKLIGYT
KTVTEVLAFLHCCLNPVLYAFIGQKFRNYFLKILKDLWCVRRKYKSSG
FSCAGRYSENISRQTSETADNDNASSFTMGSGATNFSLLKQAGDVEEN
PGPVSKGEELFTGVVPILVELDGDVNGHKFSVSGEGEGDATYGKLTLK
FICTTGKLPVPWPTLVTTLTYGVQCFSRYPDHMKQHDFFKSAMPSGYV
QERTIFFKDDGNYKTRAEVKFEGDTLVNRIELKGIDFKEDGNILGHKL
EYNYNSHNVYIMADKQKNGIKANFKIRHNIEDGSVQLADHYQQNTPIG
DGPVLLPDNHYLSTQSALSKDPNEKRDHMVLLEFVTAAGITLGMDELY
K

An example nucleic acid sequence encoding Construct 1 (CD19 CAR, CCR6, GFP region) is provided below:

(SEQ ID NO: 430)
atggagacagacactcttctcctttgggtcttgctgctgtgggttccc
ggaagcacaggagaagtacagttgcaacagtctgggccagaacccatc
aaacccggagcccctgtaaaaatgtcacgcaaagctagtggatacaca
tttacttcttacgtgatgcactgggtaaaacagaaacctggtcagggg
cttgagtggatcgggcacattaacccatataatgacggcaccaaatat
aacgagaaattcaagggaaaggctacgcttacatcagataagtccagt
agcaccgcttatatggaacttagcagccttacttccgaagattccgcg
gtgtattactgcgcgagagggacttactactacgggagtcgagtaccc
gattattggggtcaaggcacgacgctcacggcgagctcaggtggtgga
gggtctgggggtggcggcagtggtggggggggctcagacatcgtgatg
acccaggcagcaccttctatcccggtaaccccaggcgagtctgtatct
atcagttgtcggtccagcaagtctcttctcaacagtaatggcaataca
tatctttactggttcctccaaaggcctgggcaaagtcctcaacttctt
atatatcggatgtccaatcttgcgagtggcgtacccgacaggttttca
gggtctgggagcggaacagcttttacgttgagaatatccagggtagaa
gctgaggacgtcggtgtatattattgcatgcaacatctcgaatacccc
tttaccttcggcgctggtacaaagctcgaattgaaacgcaqcgatcca
accacgacgccagcgccacgaccacctacgcccgctccaactattgcc
tcccagcccctgagtcttcggccagaagcgtgtagacctgctgccggc
ggggccgttcatacgcggggccttgactttgcatgtgatatctatata
tgggctcctttggcgggaacttgcggagtgcttcttttgtcactcgtg
ataacgttgtattgtaaaaggggtcgaaagaaactcctctatatattt
aagcagccctttatgaggcccgtgcaaacaacacaagaagaggacgga
tgctcttgtcgattcccggaagaggaggagggggggtgtgagctcagg
gtcaagttttctcgctctgccgacgcgccagcctatcaacagggccaa
aaccagctgtataacgaactcaacctcgggcgccgggaagagtatgac
gtccttgacaaacggcgcggtcgcgaccctgaaatgggtggaaaaccg
aggcgaaagaacccccaggagggactttacaacgaattgcaaaaagac
aagatggccgaagcctattccgaaattggaatgaaaggcgagcggaga
cgaggtaaggggcacgacggcctgtatcaagggctctctacggccacg
aaggatacttacgacgcccttcatatgcaagctcttccaccacggggt
tcgagcggcagtggagagggcagaggaagtctgctaacatgcggtgac
gtcgaggagaatcctggcccaatgagtggggaaagtatgaacttcagc
gatgtatttgactcctccgaagattactttgtatctgtgaatacgagc
tattactccgtcgatagtgaaatgctgctctgtagtctccaagaagtc
cgccaattcagtcgcctcttcgttcccatcgcgtactcccttatttgt
gtttttggccttctgggtaacatcctggttgtaatcacattcgctttc
tataaaaaagctcggagtatgactgatgtttaccttcttaacatggct
atagcggacattctttttgtgcttactctcccattctgggctgtgagc
catgcaacaggggcgtgggttttttcaaatgccacatgtaagctgctt
aaagggatctatgcaataaacttcaattgcgggatgctcctgctgaca
tgcatcagtatggatcgatacatagctatagtacaggcgactaagtcc
ttccgcctgcgatcccgcacactgcctaggagcaaaattatttgcctc
gtcgtatgggggctctcagtgatcatctcctccagtacgtttgtcttt
aaccagaaatataacacacagggttctgatgtatgtgaaccaaagcat
cagacagtgagtgaaccaatacggtggaagttgcttatgttgggcttg
gagctgctttttgggtttttcatcccactgatgttcatgattttctgt
tatacatttattgttaagaccttggttcaggcgcaaaatagcaagaga
cataaggcaattcgagtcatcattgccgtggtgttggtcttcttggcc
tgtcagatcccccataatatggttctgctcgtcaccgccgctaacttg
gqtaagatgaatcgatcttgtcagtccgagaagttgatcggatacacc
aaaactgtgacagaagtgctggccttccttcactgttgtctgaaccca
gttttgtatgcttttataggacagaagtttcgaaattacttcttgaaa
atcctcaaggacctctggtgtgttcgaaggaagtacaagagctctggc
tttagttgcgctgggcgctacagtgagaatatatcccggcagacctcc
gagactgctgataatgacaacgcaagttccttcactatgggatccggc
gcaacaaacttctctctgctgaaacaagccggagatgtcgaagagaat
cctggaccggtgagcaagggcgaggagctgttcaccggggtggtgccc
atcctggtcgagctggacggcgacgtaaacggccacaagttcagcgtg
tctggcgagggcgagggcgatgccacctacggcaagctgaccctgaag
ttcatctgcaccaccggcaagctgcccgtgccctggcccaccctcgtg
accaccctgacctacggcgtgcagtgcttcagccgctaccccgaccac
atgaagcagcacgacttcttcaagtccgccatgcccgaaggctacgtc
caggagcgcaccatcttcttcaaggacgacggcaactacaagacccgc
gccgaggtgaagttcgagggcgacaccctggtgaaccgcatcgagctg
aagggcatcgacttcaaggaggacggcaacatcctggggcacaagctg
gagtacaactacaacagccacaacgtctatatcatggccgacaagcag
aagaacggcatcaaggcgaacttcaagatccgccacaacatcgaggac
ggcagcgtgcagctcgccgaccactaccagcagaacacccccatcggc
gacggccccgtgctgctgcccgacaaccactacctgagcacccagtcc
gccctgagcaaagaccccaacgagaagcgcgatcacatggtcctgctg
gagttcgtgaccgccgccgggatcactctcggcatggacgagctgtac
aagtaa

Construct 2 MHC CAR region (MHC-CAR1 part B, MHC-CAR 1 part A region) is provided below:

(SEQ ID NO: 431)
MVCLKLPGGSCMTALTVTLMVLSSPLALASDENPVVHFFKNIVTPRTP
PGGGGSGGGGSGGSGDTRPRFLWQPKRECHFFNGTERVRFLDRYFYNQ
SSSVRFDSDVGEFRAVTELGRPDAEYWNSQKDILSQARAAVDTYCRHN
YGVVESFTVQRRVQPKVTVYPSKTQPLQHHNLLVCSVSGFYPGSIEVR
WFLNGQEEKAGMVSTGLIQNGDWTFQTLVMLETVPRSGEVYTCQVEHP
SVTSPLTVEWRARSESAQSKMLSGVGGFVLGLLFLGAGLFIYFRNQTS
RVKFSRSADAPAYQQGQNQLYNELNLGRREEYDVLDKRRGRDPEMGGK
PRRKNPQEGLYNELQKDKMAEAYSEIGMKGERRRGKGHDGLYQGLSTA
TKDTYDALHMQALPPRGSSGSGEGRGSLLTCGDVEENPGPMAISGVPV
LGFFIIAVLMSAQESWAIKEEHVIIQAEFYLNPDQSGEFMFDFDGDEI
FHVDMAKKSTVWRLESFGRFASFEAQGALANIAVDKANLEIMTKRSNY
TPITNVPPEVTVLTNSPVELREPNVLICFIDKFTPPVVNVTWLRNGKP
VTTGVSETVFLPREDHLFRKFHYLPFLPSTEDVYDCRVEHWGLDEPLL
KHWEFDAPSPLPETTENVVCALGLTVGLVGIIIGTIFIIKGLTSRVKF
SRSADAPAYQQGQNQLYNELNLGRRSEYDVLDKRRGRDPEMGGKPRRK
NPQEGLYNELQKDKMAEAYSEIGMKGERRRGKGHDGLYQGLSTATKDT
YDALHMQALPPR

An example nucleic acid sequence encoding the Construct 2 MI-C CAR region (MHC-CAR1 part B, MHC-CAR1 part A) is provided below:

(SEQ ID NO: 71)
atggtatgcttgaagctcccgggcgggtcctgcatgaccgctctcact
gttactctcatggtccttagttcaccgccttgccctggcatctgatga
gaatcccgtggtccatttttttaagaacatcgtcacaccgcgcacccc
acctgggcggaggcggatctggcggaggcgggagtggaggctcaggag
acacaagaccccgattcttgtggcagcccaaaaggcgagtgccatttt
ttcaatgggacggaacgagttcgcttccttgatcggtacttttacaac
caagaagagagtgtaccggttcgactcagatgccggcgagttccgagc
ggttacggaattggggcgacctgacgcggagtactggaactccccaaa
aggatattttggagcaggcacgagcagctgtggacacctattgtcgac
ataaccatggtgtggtggaatccctttacagttcagcggcgggtgcaa
cctaaagtgaccgtgtatccatctaaaacgcaacccctccaacaccat
aaccctcctggtgtgttccgtaagcggcttctatcccgggtcaattga
ggtcaggtggttcctcaacggtcaggaggagcaaggccggaatggtaa
gtactggtcttatccagaacggagactggaccttccaaactttggtaa
tgttggaaacgcgtgccgcgatccggggaggtgtatacatgccaagtt
gaacacccgagtgttacgagccccctgacggttgagtggcagggcgcg
gtcagagagcgcacaatctaaaatgctgtcaggagtaggcggatttgt
actcggactcctctttttgcggcgctgggttgtttatctactttagaa
accaaacaagtagagtaaagttttcccgaagtgcggacgctcccgcgc
tatcagcaaggtcaaaaccagctttacaacgaactgaacttgggacga
cgcgaagagtacgatgttcttgataagccggagagggcgcgatcccga
aatggggggaaagcctcggaggaagaacccacaagaaggcctttataa
tgaaccgccagaaggacaagatggcqqaggcgtattccgaaataggca
tgaagggtgaacggaggagaggaaagqgacatgaccggactttatcaa
ggattgtctaccgcaactaaagacacctatgacgcgttgcacatgcag
gctctccctccgagacggttcgagcggcagtggagagggcagaggaag
tctgctaacatgcggtgacgtcgaggagaatcctggcccaatgcgcaa
tatctggtgttcctgtcctcgggttttttatcatagccgtactgatgt
cagcacaggaatcatgggcgatacaaagaagagcacgtgataatacag
gcggagttttatttgaacccggaccagagcggtgagttcatgttcgat
tttcgatggcgacgagatatctcacgttgacatggcaaaaaaggaaac
ggtgcggagacttgaggagtttggacgattccgcatcatttgaggcac
aaggagcactcgccaatatcgcggtggacaaggccaacctggagatca
tgacaaaacgcctccaattatacgcctatcactaatgtgccccctgag
gttactgtgctcacaaattctcccgtagaacttagggaaccctaacgt
cctcatatgtttcatcgacaagttcactcctccggtggtcaatqtaac
gtggcttcggaatggtaagcccggtcaccacgggtgtctcagagaccg
tatttctgcccagagaagaccacctcttccgcaaatttcattaccttc
ccctttcttccttcaacggaagacgtttacgactgcagggtcgaacat
tgggggcttgacgaaccacttctcaagccattgggagttcgacgcccc
atcaccgcttccagaaacgactgaaaacgttgtctgcgctcttggcct
gacagtgcggcctggtaggcattattatcgggaccatctttatcatca
aaggtttgacttcccgggtcaaatttagcagatcccgctgacgcaccg
gcccaccagcagggccagaaccaactctacaacgagctgaatctcggc
cgacgggaagagtatcgacgtactcgacaagcggagaggtcgagaccc
tgagatgggcggtaaaccgagacggaaaaatccccaagagggtccttt
ataatgaactccagaaggacaagatggctgaagcctattctgagatag
ggatgaaaggcgagcggcggaggcggtaagggccatgatggcctttac
cagggactctccacggcaaccaaagatacttacgacgcccttcacatg
caacgccctcccgccacgc

Construct 2 kill switch and MHC CAR region (RQR8, MHC-CAR1 part B. MHC-CAR 1 part A region) is provided below:

(SEQ ID NO: 211)
MGTSLLCWMALCLLGADHADACPYSNPSLCSGGGGSELPTQGTFSNVS
TNVSPAKPTTTACPYSNPSLCSGGGGSPAPRPPTPAPTIASQPLSLRP
EACRPAAGGAVHTRGLDFACDIYIWAPLAGTCGVLLLSLVITLYCNHR
NRRRVCKCPRPVVRSGSGQCTNYALLKLAGDVESNPGPPTGMVCLKLP
GGSCMTALTVTLMVLSSPLALASDENPVVHFFKNIVTPRTPPGGGGSG
GGGSGGSGDTRPRFLWQPKRECHFFNGTERVRFLDRYFYNQEESVRFD
SDVGEFRAVTELGRPDASYWNSQKDILEQARAAVDTYCRHNYGVVESF
TVQRRVQPKVTVYPSKTQPLQHHNLLVCSVSGFYPGSIEVRWFLNGQE
EKAGMVSTGLIQNGDWTFQTLVMLETVPRSGEVYTCQVEHPSVTSPLT
VEWRARSESAQSKMLSGVGGFVLGLLFLGAGLFIYFRNQTSRVKFSRS
ADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDA
LHMQALPPRGSSGSGEGRGSLLTCGDVEENPGPMAISGVPVLGFFIIA
VLMSAQESWAIKEEHVIIQAEFYLNPDQSGEFMFDFDGDEIFHVDMAK
KETVWRLEEFGRFASFSAQGALANIAVDKANLEIMTKRSNYTPITNVP
PEVTVLTNSPVELREPNVLICFIDKFTPPVVNVTWLRNGKPVTTGVSE
TVFLPREDHLFRKFHYLPFLPSTEDVYDCRVEHWGLDEPLLKHWEFDA
PSPLPETTENVVCALGLTVGLVGIIIGTIFIIKGLTSRVKFSRSADAP
AYQQGQNQLYNELNLGRREEYDVLDKRRGRDPEMGGKPRRKNPQEGLY
NELQKDKMAEAYSEIGMKGERRRGKGHDGLYQGLSTATKDTYDALHWQ
ALPPR

An example nucleic acid sequence encoding Construct 2 kill switch and MHC CAR region (RQR8, MHC-CAR1 part B, MHC-CAR1 part A region) is provided below:

(SEQ ID NO: 403)
atgggtacttcactgttgtgctggatggcactttgtcttttgggtgcc
gatcatgctgatgcatgtccgtactccaatcctagcctgtgctccggg
gggggagggagtgaactccctacacagggaaccttctctaatgtctcc
accaacgtctcccctgcaaaaccqaccacaacagcttgcccctatagt
aacccttccctctgtagtggaggggggggttcacctgctccacgccct
cctacccccgcgccaacgatcgcgccacaaccgctcagtcttaggccg
gaagcctgtaggccagcggctggcggtgcggttcatacgcggggattg
gattttgcctgcgacatttacatttgggctccgctggccggtacttgt
ggggtattgctgttgtctcttgttattacgctttattgcaatcacagg
aacaggcgacgagtatgcaaatgcccgcggcccgtcgtgagatctggg
tccggccaatgtactaactacgctttgttgaaactcgctggcgatgtt
gaaagtaaccccggtcctccaacaggtatggtatgcttgaagctcccg
ggcgggtcctgcatgaccgctctcactgttactcttatggtccttagt
tcaccgcttgccctggcatctgatgagaatcccgtggttcattttttt
aagaacatcgtcacaccgcgcaccccacctgggggaggcggatctggc
ggaggcgggagtggaggctcaggagacacaagaccccgattcttgtgg
cagcccaaaagggagtgccattttttcaatgggacggaacgagttcgc
ttccttgatcggtacctctacaaccaagaagagagtgtacggttcgac
tcagatgtcggcgagttccgagcggttacggaatcggggcgacctgac
gcggagtactggaactcccaaaaggatattttggagcaggcacgagca
gctgtggacacctattgtcgacataattatggtgtggtggaatccttt
acagttcagcggcgggtgcaacctaaagtgaccgtgtatccatctaaa
acgcaacccctccaacaccataacctcctggtgtgttccgtaagcggc
ttctatcccgggtcaattgaggtcaggtggttcctcaacggtcaggag
gagaaggccggaatggtaagtactggtcttatccagaacggagactgg
accttccaaactttggtaatgttggaaacggtgccgcgatccggggag
gtgtatacatgccaagttgaacacccgagtgttacgagccccctgacg
gttgagtggagggcgcggtcagagagcgcacaatctaaaatgctgtca
ggagtaggcggatttgtactcggactcctctttttgggcgctgggttg
tttatctactttagaaaccaaacaagtagagtaaagttttcccgaagt
gcggacgcccccgcgtatcagcaaggccaaaaccagctttacaacgaa
ctgaacttgggacgacgcgaagagtacgatgttcttgataagcggaga
gggcgcgatcccgaaatggggggaaagcctcggaggaagaacccacaa
gaaggcctttataatgaactgcagaaggacaagatggcggaggcgtat
tccgaaataggcatgaagggtgaacggaggagaggaaagggacatgac
ggactttatcaaggattgtctaccgcaactaaagacacctatgacgcg
ttgcacatgcaggctctccctccgagaggttcgagcggcagtggagag
ggcagaggaagtccgctaacatgcggtgacgtcgaggagaatcctggc
ccaatggcaatatctggtgttcctgtcctcgggttttttatcatagcc
gtactgatgtcagcacaggaatcatgggcgataaaagaagagcacgtg
ataatacaggcggagttttatttgaacccggaccagagcggtgagttc
atgttcgattttgatggcgacgagatatttcacgttgacatggcaaaa
aaggaaacggtgtggagacttgaggagtttggacgattcgcaccattt
gaggcacaaggagcactcgccaatatcgcggtggacaaggccaacctg
gagatcatgacaaaacgctccaattatacgcctatcactaatgtgccc
cctgaggttactgtgctcacaaattctcccgtagaacttagggaacct
aacgtcctcatatgtttcatcgacaagttcactcctccggtggtcaat
gtaacgtggcttcggaatggtaagccggtcaccacgggtgtctcagag
accgtatttctgcccagagaagaccacctcttccgcaaatttcattac
cttccctttcttccttcaacggaagacgtttacgactgcagggtcgaa
cattgggggcttgacgagccacttctcaagcattgggagttcgacgcc
ccatcaccgcttccagaaacgactgaaaacgttgtctgcgctcttggc
ctgacagtgggcctggtaggcattattatcgggaccatctttatcatc
aaaggtttgacttcccgggtcaaatttagcagatccgctgacgcaccg
gcctaccagcagggccagaaccaactctacaacgagctgaatctcggc
cgacgggaagagtatgacgtactcgacaagcggagaggtcgagaccct
gagatgggcggtaaaccgagacggaaaaatccccaagagggtctttat
aatgaactccagaaggataagatggctgaagcctattctgagataggg
atgaaaggcgagcggcggaggggtaagggccatgatggcctCCaccag
ggactctccacggcaaccaaagatacttacgacgcccttcacatgcaa
gccctcccgccacgc

Construct 2 (RQR8, MHC-CAR1 part B, MHC-CAR1 part A, GFP region) is provided below.

(SEQ ID NO: 405)
MGTSLLCWMALCLLGADHADACPYSNPSLCSGGGGSELPTQGTFSNVS
TNVSPAKPTTTACPYSNPSLCSGGGGSPAPRPPTPAPTIASQPLSLRP
EACRPAAGGAVHTRGLDFACDIYIWAPLAGTCGVLLLSLVITLYCNHR
NRRRVCKCPRPVVRSGSGQCTNYALLKLAGDVESNPGPPTGMVCLKLP
GGSCMTALTVTLMVLSSPLALASDENPVVHFFKNIVTPRTPPGGGGSG
GGGSGGSGDTRPRFLWQPKRECHFFNGTERVRFLDRYFYNQEESVRFD
SDVGEFRAVTELGRPDAEYWNSQKDILEQARAAVDTYCRHNYGVVESF
TVQRRVQPKVTVYPSKTQPLQHHNLLVCSVSGFYPGSIEVRWFLNGQE
EKAGMVSTGLIQNGDWTFQTLVMLETVPRSGEVYTCQVEHPSVTSPLT
VEWRARSESAQSKMLSGVGGFVLGLLFLGAGLFIYFRNQTSRVKFSRS
ADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDA
LHMQALPPRGSSGSGEGRGSLLTCGDVEENPGPMAISGVPVLGFFTIA
VLMSAQSSWAIKEEHVIIQAEFYLNPDQSGEFMFDFDGDEIFHVDMAK
KETVWRLEEFGRFASFEAQGALANIAVDKANLSIMTKRSNYTPITNVP
PEVTVLTNSPVELREPNVLICFIDKFTPPVVNVTWLRNGKPVTTGVSE
TVFLPREDHLFRKFHYLPFLPSTEDVYDCRVEHWGLDEPLLKHWEFDA
PSPLPETTENVVCALGLTVGLVGIIIGTIFIIKGLTSRVKFSRSADAP
AYQQGQNQLYNELNLGRREEYDVLDKRRGRDPEMGGKPRRKNPQEGLY
NELQKDKMAEAYSEIGMKGERRRGKGHDGLYQGLSTATKDTYDALHMQ
ALPPRGSGATNFSLLKQAGDVEENPGPVSKGEELFTGVVPILVELDGD
VNGHKFSVSGEGSGDATYGKLTLKFICTTGKLPVPWPTLVTTLTYGVQ
CFSRYPDHMKQHDFFKSAMPEGYVQERTIFFKDDGNYKTRAEVKFEGD
TLVNRIELKGIDFKEDGNILGHKLEYNYNSHNVYIMADKQKNGIKANF
KIRHNIEDGSVQLADHYQQNTPIGDGPVLLPDNHYLSTQSALSKDPNE
KRDHMVLLEFVTAAGITLGMDSLYK*

An example nucleic acid sequence encoding Construct 2 (RQR8, MHC-CAR 1 part B, MHC-CAR1 part A, GFP region) is provided below:

(SEQ ID NO: 409)
atgggtacttcactgttgtgctggatggcactttgtcttttgggtg
ccgatcatgctgatgcatgtccgtactccaatcctagcctgtgctc
cggggggggagggagtgaactccctacacagggaaccttctctaat
gtctccaccaacgtctcccctgcaaaaccgaccacaacagcttgcc
cctatagtaacccttccctctgcagtggaggggggggttcacctgc
tccacgccctcctacccccgcgccaacgatcgcgtcacaaccgctc
agtcttaggccggaagcctgtaggccagcggctggcggtgcggttc
atacgcggggattggattttgcctgcgacatttacatttgggctcc
gctggccggtacttgtggggtattgctgttgtctcttgttattacg
ctttattgcaatcacaggaacaggcgacgagtatgcaaatgcccgc
ggcccgtcgtgagatctgggtccggccaatgtactaactacgcttt
gttgaaacccgctggcgatgttgaaagcaaccccggtcctccaaca
ggtatggtatgctcgaagctcccgggcgggccctgcatgaccgctc
tcactgttactcttatggtccttagttcaccgcttgccctggcatc
tgatgagaatcccgtggttcatttttttaagaacatCgtcacaccg
cgcaccccacctgggggaggcggatctggcggaggcgggagtggag
gctcaggagacacaagaccccgattcttgtggcagcccaaaaggga
gtgccattttttcaatgggacggaacgagttcgcttccttgatcgg
tacttttacaaccaagaagagagtgtacggctcgactcagatgtcg
gcgagttccgagcggtcacggaattggggcgacctgacgcggagta
ctggaactcccaaaaggatattttggagcaggcacgagcagctgtg
gacacctattgtcgacataattatggtgtggtggaatcctttacag
ttcagcggcgggtgcaacctaaagtgaccgtgtatccacctaaaac
gcaacccccccaacaccataacctcctggtgtgttccgtaagcggc
ttctatcccgggtcaattgaggtcaggtggttcctcaacggtcagg
aggagaaggccggaatggtaagtactggtcttatccagaacggaga
ctggaccttccaaactttggtaatgttggaaacggtgccgcgatcc
ggggaggtgtatacatgccaagttgaacacccgagtgttacgagcc
ccctgacggttgagtggagggcgcggtcagagagcgcacaatctaa
aatgctgtcaggagtaggcggatttgtactcggactcctctttttg
ggcgctgggttgtttatctactttagaaaccaaacaagtagagtaa
agttttcccgaagtgcggacgctcccgcgtatcagcaaggtcaaaa
ccagctttacaacgaactgaacttgggacgacgcgaagagtacgat
gttctcgataagcggagagggcgcgatcccgaaatggggggaaagc
ctcggaggaagaacccacaagaaggcctttataatgaactgcagaa
ggacaagatggcggaggcgtattccgaaataggcatgaagggtgaa
cggaggagaggaaagggacatgacggactttatcaaggattgccta
ccgcaactaaagacacctatgacgcgttgcacatgcaggctctccc
tccgagaggttcgagcggcagtggagagggcagaggaagtctgcta
acatgcggtgacgtcgaggagaatcctggcccaatggcaatatctg
gtgttcctgtcctcgggttttttatcatagccgtactgatgtcagc
acaggaatcatgggcgataaaagaagagcacgtgataatacaggcg
gagttttatttgaacccggaccagagcggtgagttcatgttcgatt
ttgatggcgacgagatatttcacgttgacatggcaaaaaaggaaac
ggtgtggagacttgaggagtttggacgattcgcatcatttgaggca
caaggagcactcgccaataccgcggtggacaaggccaacctggaga
tcatgacaaaacgctccaattatacgcctatcactaatgtgccccc
tgaggttactgtgctcacaaattctcccgtagaacttagggaacct
aacgtcctcatatgtttcatcgacaagttcactcctccggtggtca
atgtaacgtggcttcggaatggtaagccggtcaccacgggtgtctc
agagaccgtatttctgcccagagaagaccaccccttccgcaaattt
cattaccttccctttcttccttcaacggaagacgtctacaactgca
gggtcgaacattgggggcttgacgagccacttctcaagcatcggga
gttcgacgccccatcaccgcttccagaaacgactgaaaacgttgtc
tgcgctcttggcctgacagtgggcctggtaggcattattatcggga
ccatctttatcatcaaaggtttgacttcccgggtcaaatttagcag
atccgctgacgcaccggcctaccagcagggccagaaccaactctac
aacgagctgaatctcggccgacgggaagagtatgacgtactcgaca
agcggagaggtcgagaccctgagatgggcggtaaaccgagacggaa
aaatccccaagagggtctttataatgaactccagaaggataagatg
gctgaagcctattctgagatagggatgaaaggcgagcggcggaggg
gtaagggccatgatggcctttaccagggactctccacggcaaccaa
agatacttacgacgcccttcacatgcaagccctcccgccacgcgga
tccggcgcaacaaacttctctctgctgaaacaagccggagatgtcg
aagagaatcctggaccggtgagcaagggcgaggagctgttcaccgg
ggtggtgcccatcctggtcgagctggacggcgacgtaaacggccac
aagttcagcgtgtctggcgagggcgagggcgatgccacctacggca
agctgaccctgaagttcatctgcaccaccggcaagctgcccgtgcc
ctagcccaccctcgtgaccaccctgacctacggcgtgcagtgcttc
agccgctaccccgaccacatgaagcagcacgacttcttcaagtccg
ccatgcccgaaggctacgtccaggagcgcaccatcttcttcaagga
cgacggcaactacaagacccgcgccgaggtgaagttcgagggcgac
accctggtgaaccgcatcgagctgaagggcatcgacttcaaggagg
acggcaacatcctggggcacaagctggagtacaactacaacagcca
caacgtctatatcatggccgacaagcagaagaacggcatcaaggcg
aacttcaagatccgccacaacatcgaggacggcagcgtgcagctcg
ccgaccactaccagcagaacacccccatcggcgacggccccgtgct
gctgcccgacaaccactacctgagcacccagtccgccctgagcaaa
gaccccaacgagaagcgcgatcacatggtcctgctggagttcgtga
ccgccgccgggatcactctcggcatggacgagctgtacaagtaa

The amino acid sequences of exemplary CS-1 targeting CAR constructs are provided below (note that these designs contain a 4-1BB domain which may be replaced with a CD28 domain):

Anti-CS1-CAR-v1 (Luc63-V1 CAR):
(SEQ ID NO: 129)
MALPVTALLLPLALLLHAARPEVKLLESGGGLVQPGGSLKLSCAASGFDFSRYWMSWVRQAPGKGLEWIGEINPD
SSTINYTPSLKDKFIISRDNAKNTLYLQMSKVRSEDTALYYCARPDGNYWYFDVWGAGTTVTVSSGGGGSGGGGS
GGGGSDIVMTQSHKFMSTSVGDRVSITCKASQDVGIAVAWYQQKPGQSPKLLIYWASTRHTGVPDRFTGSGSGTD
FTLTISNVQSEDLADYFCQQYSSYPYTFGGGTKLEIKGLAVSTISSFFPPGYQKRGRKKLLYIFKQPFMRPVQTT
QEEDGCSCRFPEEEEGGCELRVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQALPPR
Anti-CS1-CAR-v2 (Luc63-V2 CAR):
(SEQ ID NO: 130)
MALPVTALLLPLALLLHAARPEVKLLESGGGLVQPGGSLKLSCAASGFDFSRYWMSWVRQAPGKGLEWIGEINPD
SSTINYTPSLKDKFIISRDNAKNTLYLQMSKVRSEDTALYYCARPDGNYWYFDVWGAGTTVTVSSGGGGSGGGGS
GGGGSDIVMTQSHKFMSTSVGDRVSITCKASQDVGIAVAWYQQKPGQSPKLLIYWASTRHTGVPDRFTGSGSGTD
FTLTISNVQSEDLADYFCQQYSSYPYTFGGGTKLEIKTTTPAPRPPTPAPTIASQPLSLRPEACRPAAGGAVHTR
GLDFACDIYIWAPLAGTCGVLLLSLVITLYCKRGRKKLLYIFKQPFMRPVQTTQEEDGCSCRFPEEEEGGCELRV
KFSRSADAPAYQQGQNQLYNELNLGRREEYDVLDKRRGRDPEMGGKPRRKNPQEGLYNELQKDKMAEAYSEIGMK
GERRRGKGHDGLYQGLSTATKDTYDALHMQALPPR
Anti-CS1-CAR-v3 (Luc63-V3 CAR):
(SEQ ID NO: 131)
MALPVTALLLPLALLLHAARPEVKLLESGGGLVQPGGSLKLSCAASGFDFSRYWMSWVRQAPGKGLEWIGEINPD
SSTINYTPSLKDKFIISRDNAKNTLYLQMSKVRSEDTALYYCARPDGNYWYFDVWGAGTTVTVSSGGGGSGGGGS
GGGGSDIVMTQSHKFMSTSVGDRVSITCKASQDVGIAVAWYQQKPGQSPKLLIYWASTRHTGVPDRFTGSGSGTD
FTLTISNVQSEDLADYFCQQYSSYPYTFGGGTKLEIKEPKSPDKTHTCPPCPAPPVAGPSVFLFPPKPKDTLMIA
RTPEVTCVWDVSHEDPEVKFNVfYVDGVEVHNAKTKPREEQYNSTYRVVSVLTVLHQDWLNGKSYKCKVSNKALP
APIEKTISKAKGQPREPQVYTLPPSRDELTKNQVSLTCLVKGFYPSDIAVEWESNGQPENNYKTTPPVLDSDGSF
FLYSKLTVDKSRWQQGNVFSCSVMHEALHNHYTQKSLSLSPGKKRGRKKLLYIFKQPFMRPVQTTQEEDGCSCRF
PEEEEGGCELRVKFSRSADAPAYQQGQNQLYNELNLGRREEYDVLDKRRGRDPEMGGKPRRKNPQEGLYNSLQKD
KMAEAYSEIGMKGERRRGKGHDGLYQGLSTATKDTYDALHMQALPPR
Anti-CS1-CAR-v4 (Luc90-V1 CAR):
(SEQ ID NO: 132)
MALPVTALLLPLALLLHAARPQVQLQQPGASLVRPGASVKLSCKASGYSFTTYWMNWVKQRPGQGLEWIGMIHPS
DSETRLNQKFKDKATLTVDKSSSTAYMQLSSPTSEDSAVYYCARSTMIATRAMDYWGQGTSVTVSSGGGGSGGGG
SGGGGSDIVMTQSQKSMSTSVGDRVSITCKASQDVITGVAWYQQKPGQSPKLLIYSASYRYTGVPDRFTGSGSGT
DFTFTISNVQAEDLAVYYCQQHYSTPLTFGAGTKLELKGLAVSTISSFFPPGYQKRGRKKLLYIFKQPFMRPVQT
TQEEDGCSCREPESEEGGCELRVKFSRSADAPAYQQGQNQLYNELNLGRREEYDVLDKRRGRDPEMGGKPRRKNP
QEGLYNELQKDKMAEAYSEIGMKGERRRGKGHDGLYQGLSTATKDTYDALHMQALPPR
Anti-CS1-CAR-v5 (Luc90-V2 CAR):
(SEQ ID NO: 133)
MALPVTALLLPLALLLHAARPQVQLQQPGAELVRPGASVKLSCKASGYSFTTYWMNWVKQRPGQGLEWIGMIHPS
DSETRLNQKFKDKATLTVDKSSSTAYMQLSSPTSEDSAVYYCARSTMIATRAMDYWGQGTSVTVSSGGGGSGGGG
SGGGGSDIVMTQSQKSMSTSVGDRVSITCKASQDVITGVAWYQQKPGQSPKLLIYSASYRYTGVPDRFTGSGSGT
DFTFTISNVQAEDLAVYYCQQHYSTPLTFGAGTKLELKTTTPAPRPPTPAPTIASQPLSLRPSACRPAAGGAVHT
RGLDFACDIYIWAPLAGTCGVLLLSLVITLYCKRGRKKLLYIFKQPFMRPVQTTQSEDGCSCRFPEEEEGGCELR
VKFSRSADAPAYQQGQNQLYNSLNLGRREEYDVLDKRRGRDPEMGGKPRRKNPQEGLYNELQKDKMAEAYSEIGM
KGERRRGKGHDGLYQGLSTATKDTYDALHMQALPPR
Anti-CS1-CAR-v6 (Luc90-V3 CAR):
(SEQ ID NO: 134)
MALPVTALLLPLALLLHAARPQVQLQQPGAELVRPGASVKLSCKASGYSFTTYWMNWVKQRPGQGLEWIGMIHPS
DSETRLNQKFKDKATLTVDKSSSTAYMQLSSPTSEDSAVYYCARSTMIATRAMDYWGQGTSVTVSSGGGGSGGGG
SGGGGSDIVMTQSQKSMSTSVGDRVSITCKASQDVITGVAWYQQKPGQSPKLLIYSASYRYTGVPDRFTGSGSGT
DFTFTISNVQAEDLAVYYCQQHYSTPLTFGAGTKLELKSPKSPDKTHTCPPGPAPPVAGPSVFLFPPKPKDTLMI
ARTPEVTCVVVDVSHEDPEVKFNWYVDGVSVHNAKTKPREEQYNSTYRVVSVLTVLHQDWLNGKEYKCKVSNKAL
PAPIEKTISKAKGQPREPQVYTLPPSRDELTKNQVSLTCLVKGFYPSDIAVEWESNGQPENNYKTTPPVLDSDGS
FFLYSKLTVDKSRWQQGNVFSCSVMHEALHNHYTQKSLSLSPGKKRGRKKLLYIFKQPFMRPVQTTQEEDGCSCR
FPEEEEGGCELRVKFSRSADAPAYQQGQNQLYNELNLGRREEYDVLDKRRGRDPEMGGKPRRKNPQEGLYNSLQK
DKMAEAYSEIGMKGSRRRGKGHDGLYQGLSTATKDTYDALHMQALPPR
Anti-CA1-CAR-v7 (Luc34-V1 CAR):
(SEQ ID NO: 135)
MALPVTALLLPLALLLHAARPQVQLQQSGAELARPGASVKLSCKASGYTFTSYWMQWVKQRPGQGLEWIGAIYPG
DGDTRYTQKFKGKATLTADKSSSTAYMQLSSLASEDSAVYYCARGKVYYGSNPFAYWGQGTLVTVSAGGGGSGGG
GSGGGGSDIQMTQSSSYLSVSLGGRVTITCKASDHINNWLAWYQQKPGNAPRLLISGATSLETGVPSRFSGSGSG
KDYTLSITSLQTEDVATYYCQQYWSTPWTFGGGTKLEIKGLAVSTISSFFPPGYQKRGRKKLLYIFKQPFMRPVQ
TTQEEDGCSCRFPEEEEGGCELRVKFSRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYDALHMQALPPR
Anti-CS1-CAR-v8 (Luc34-V2 CAR):
(SEQ ID NO: 136)
MALPVTALLLPLALLLHAARPQVQLQQSGAELARPGASVKLSCKASGYTFTSYWMQWVKQRPGQGLEWIGAIYPG
DGDTRYTQKFKGKATLTADKSSSTAYMQLSSLASEDSAVYYCARGKVYYGSNPFAYWGQGTLVTVSAGGGGSGGG
GSGGGGSDIQMTQSSSYLSVSLGGRVTITCKASDHINNWLAWYQQKPGNAPRLLISGATSLETGVPSRFSGSGSG
KDYTLSITSLQTEDVATYYCQQYWSTPWTFGGGTKLEIKTTTPAPRPPTPAPTIASQPLSLRPEACRPAAGGAVH
TRGLDFACDIYIWAPLAGTCGVLLLSLVITLYCKRGRKKLLYIFKQPFMRPVQTTQEEDGCSCRFPEEEEGGCEL
RVKFSRSADAPAYQQGQNQLYNELNLGRREEYDVLDKRRGRDPEMGGKPRRKNPQEGLYNELQKDKMAEAYSEIG
MKGERRRGKGHDGLYQGLSTATKDTYDALHMQALPPR
Anti-CS1-CAR-v9 (Luc34-V3 CAR):
(SEQ ID NO: 137)
MALPVTALLLPLALLLHAARPQVQLQQSGAELARPGASVKLSCKASGYTFTSYWMQWVKQRPGQGLEWIGAIYPG
DGDTRYTQKFKGKATLTADKSSSTAYMQLSSLASEDSAVYYCARGKVYYGSNPFAYWGQGTLVTVSAGGGGSGGG
GSGGGGSDIQMTQSSSYLSVSLGGRVTITCKASDHINNWLAWYQQKPGNAPRLLISGATSLETGVPSRFSGSGSG
KDYTLSITSLQTEDVATYYCQQYWSTPWTFGGGTKLEIKEPKSPDKTHTCPPCPAPPVAGPSVFLFPPKPKDTLM
IARTPEVTCVVVDVSHEDPSVKFNWYVDGVEVHNAKTKPREEQYNSTYRVVSVLTVLHQDWLNGKEYKCKVSNKA
LPAPIEKTISKAKGQPREPQVYTLPPSRDELTKNQVSLTCLVKGFYPSDIAVEWESNGQPENNYKTTPPVLDSDG
SFFLYSKLTVDKSRWQQGNVFSCSVMHEALHNHYTQKSLSLSPGKKRGRKKLLYIFKQPFMRPVQTTQEEDGCSC
RFPEEEEGGCELRVKFSRSADAPAYQQGQNQLYNELNLGRREEYDVLDKRRGRDPEMGGKPRRKNPQEGLYNELQ
KDKMAEAYSEIGMKGERRRGKGHDGLYQGLSTATKDTYDALHMQALPPR
Anti-CS1-CAR-v10 (LucX1-V1 CAR)
(SEQ ID NO: 138)
MALPVTALLLPLALLLHAARPQVQLQQSGPELVKPGASVKISCKASGYAFSSSWMNWVKQRPGQGLEWIGRIYPG
DGDTKYNGKFKGKATLTADKSSSTAYMQLSSLTSVDSAVYFCARSTMIATGAMDYWGQGTSVTVSSGGGGSGGGG
SGGGGSETTVTQSPASLSMAIGEKVTIRCITSTDIDDDMNWYQQKPGEPPKLLISEGNTLRPGVPSRFSSSGYGT
DFVFTIENMLSEDVADYYCLQSDNLPLTFGGGTKLEIKGLAVSTISSFFPPGYQKRGRKKLLYIFKQPFMRPVQT
TQEEDGCSCRFPEEEEGGCELRVKFSRSADAPAYQQGQNQLYNELNLGRREEYDVLDKRRGRDPEMGGKPRRKNP
QEGLYNELQKDKMAEAYSEIGMKGERRRGKGHDGLYQGLSTATKDTYDALHMQALPPR
Anti-CS1-CAR-v11 (LucX1-V2 CAR)
(SEQ ID NO: 139)
MALPVTALLLPLALLLHAARPQVQLQQSGAELARPGASVKLSCKASGYTFTSYWMQWVKQRPGQGLEWIGAIYPG
DGDTRYTQKFKGKATLTADKSSSTAYMQLSSLASEDSAVYYCARGKVYYGSNPFAYWGQGTLVTVSAGGGGSGGG
GSGGGGSDIQMTQSSSYLSVSLGGRVTITCKASDHINNWLAWYQQKPGNAPRLLISCATSLETGVPSRFSGSGSG
KDYTLSITSLQTEDVATYYCQQYWSTPWTFGGGTKLEIKTTTPAPRPPTPAPTIASQPLSLRPFACRPAAGGAVH
TRGLDFACDIYIWAPLAGTCGVLLLSLVITLYCKRGRKKLLYIFKQPFMRPVQTTQEEDGCSCRFPEEEEGGCEL
RVKFSRSADPAYQQGQNQLYNELNLGRREEYDVLDKRRGRDPEMGGKPRRKNPQEGLYNELQKDKMAEAYSEIGM
KGERRRGKGHDGLYQGLSTATKDTYDALHMQALPPR
Anti-CS1-CAR-v12 (LucX1-V3 CAR):
{SEQ ID NO: 140)
MALPVTALLLPLALLLHAARPQVQLQQSGPELVKPGASVKISCKASGYAFSSSWMNWVKQRPGQGLEWIGRIYPG
DGDTKYNGKFKGKATLTADKSSSTAYMQLSSLTSVDSAVYFCARSTMIATGAMDYWGQGTSVTVSSGGGGSGGGG
SGGGGSETTVTQSPASLSMAIGEKVTIRCITSTDIDDDMNWYQQKPGEPPKLLISEGNTLRPGVPSRFSSSGYGT
DFVFTIENMLSEDVADYYCLQSDNLPLTFGGGTKLEIKEPKSPDKTHTCPPCPAPPVAGPSVFLFPPKPKDTLMI
ARTPEVTCVVVDVSHEDPEVKFNWYVDGVEVHNAKTKPREEQYNSTYRVVSVLTVLHQDWLNGKEYKCKVSNKAL
PAPIEKTISKAKGQPREPQVYTLPPSRDELTKNQVSLTCLVKGFYPSDIAVEWESNGQPENNYKTTPPVLDSDGS
FFLYSKLTVDKSRWQQGNVFSCSVMHEALHNHYTQKSLSLSPGKKRGRKKLLYIFKQPFMRPVQTTQEEDGCSCR
FPEEEEGGCELRVKFSRSADAPAYQQGQNQLYNELNLGRREEYDVLDKRRGRDPEMGGKPRRKNPQEGLYNELQK
DKMAEAYSEIGMKGSRRGKGHDGLYQGLSTATKDTYDALHMQALPPR
Anti-CS1-CAR-v13 (LucX2-V1 CAR):
(SEQ ID NO: 141)
MALPVTALLLPLALLLHAARPQVQLQQSGPELVKPGASVKISCKASGYAFSSSWMNWVKQRPGQGLEWIGRIYPG
DGDTKYNGKFKGKATLTADKSSSTAYMQLSSLTSVDSAVYFCARSTMIATGAMDYWGQGTSVTVSSGGGGSGGGG
SGGGSDIVMTQSHKFMSTSVGDRVSITCKASQDVSTAVAWYQQKPGQSPKLLIYSASYRYTGVPDRFTGSGSGTD
FTFTISSVQAEDLAVYYCQQHYSTPPYTFGGGTKLEIKGLAVSTISSFFPPGYQKRGRKKLLYIFKQPFMRPVQT
TQEEDGCCRFPEEEEGGCELRVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQALPPR
Anti-CS1-CAR-v14(LucX2-V2 CAR):
(SEQ ID NO: 142)
MALPVTALLLPLALLLHAARPQVQLQQSGPSLVKPGASVKISCKASGYAFSSSWMNWVKQRPGQGLEWIGRIYPG
DGDTKYNGKFKGKATLTADKSSSTAYMQLSSLTSVDSAVYFCARSTMIATGAMDYWGQGTSVTVSSGGGGSGGGG
SGGGGSDIVMTQSHKFMSTSVGDRVSITCKASQDVSTAVAWYQQKPGQSPKLLIYSASYRYTGVPDRFTGSGSGT
DFTFTISSVQAEDLAVYYCQQHYSTPPYTFGGGTKLEIKTTTPAPRPPTPAPTIASQPLSLRPEACRPAAGGAVH
TRGLDFADIYIWAPLAGTCGVLLLSLVITLYCKRGRKKLLYIFKQPFMRPVQTTQEEDGCSCRFPEEEEGGCELR
VKFSRSADAPAYQQGQNQLYNELNLGRREEYDVLDKRRGRDPEMGGKPRRKNPQEGLYNELQKDKMAEAYSSIGM
KGERRRGKGHDGLYQGLSTATKDTYDALHMQALPPR
Anti-CS1-CAR-v15 (LucX2-V3 CAR):
(SEQ ID NO: 143)
MALPVTALLLPLALLLHAARPQVQLQQSGPSLVKPGASVKISCKASGYAFSSSWMNWVKQRPGQGLEWIGRIYPG
DGDTKYNGKFKGKATLTADKSSSTAYMQLSSLTSVDSAVYFCARSTMIATGAMDYWGQGTSVTVSSGGGGSGGGG
SGGGGSDIVMTQSHKFMSTSVGDRVSITCKASQDVSTAVAWYQQKPGQSPKLLIYSASYRYTGVPDRFTGSGSGT
DFTFTISSVQAEDLAVYYCQQHYSTPPYTFGGGTKLEIKEPKSPDKTHTCPPCPAPPVAGPSVFLFPPKPKDTLM
IARTPEVCVVVDVSHEDPEVKFNWYVDGVEVHNAKTKPREEQYNSTYRVVSVLTVLHQDWLNGKEYKCKVSNKAL
PAPIEKTISKAKGQPREPQVYTLPPSRDELTKNQVSLTCLVKGFYPSDIAVEWESNGQPENNYKTTPPVLDSDGS
FFLYSKLTVDKSRWQQGNVFSCSVMHEALHNHYTQKSLSLSPGKKRGRKKLLYIFKQPFMRPVQTTQEEDGCSCR
FPEEEEGGCELRVKFSRSADAPAYQQGQNQLYNELNLGRREEYDVLDKRRGRDPEMGGKPRRKNPQEGLYNSLQK
DKMAEAYSEIGMKGERRRGKGHDGLYQGLSTATKDTYDALHMQALPPR

### (iii) Preparation of MHC-CARs

Any of the MHC-CAR constructs described herein can be prepared by a routine method, such as recombinant technology. Methods for preparing the chimeric receptors herein involve generation of a nucleic acid or a nucleic acid set that encodes or collectively encodes a MHC-CAR construct (including a single polypeptide or two subunits). In some embodiments, the nucleic acid also encodes a self-cleaving peptide (e.g., P2A, T2A, or E2A peptide) between the coding sequences for the two subunits of a MHC-CAR, or between the coding sequence for a MHC-CAR and the coding sequence for other genes to be co-expressed with the MHC-CAR in a host cell (see discussions below).

Sequences of each of the components of the MHC-CARs may be obtained via routine technology, e.g., PCR amplification from any one of a variety of sources known in the art. In some embodiments, sequences of one or more of the components of the MHC-CARs are obtained from a human cell. Alternatively, the sequences of one or more components of the MHC-CARs can be synthesized. Sequences of each of the components (e.g., domains) can be joined directly or indirectly (e.g., using a nucleic acid sequence encoding a peptide linker) to form a nucleic acid sequence encoding the MHC-CAR, using methods such as PCR amplification or ligation. Alternatively, the nucleic acid encoding the MHC-CAR may be synthesized. In some embodiments, the nucleic acid is DNA. In other embodiments, the nucleic acid is RNA.

Any of the MHC-CAR proteins, nucleic acid encoding such, and expression vectors carrying such nucleic acid, all of which are within the scope of the present disclosure, can be mixed with a pharmaceutically acceptable carrier to form a pharmaceutical composition, which is also within the scope of the present disclosure. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids: hydrophobic polymers; monosaccharides; disaccharides; and other carbohydrates; metal complexes; and/or non-ionic surfactants. See, e.g. Remington: The Science and Practice of Pharmacy 20th Ed. (2000) Lippincott Williams and Wilkins, Ed. K. E. Hoover.

### II. Genetically Engineered Immune Cells Expressing MHC-CARs

(i) MHC-CAR-Expressing Immune Cells

Immune cells expressing the MHC-CAR described herein provide a specific population of cells that can recognize pathogenic cells (e.g., autoreactive T cells) involved in autoimmune diseases via MHC/peptide-TCR engagement. The interaction between the MHC-peptide portion of the MHC-CAR and the cognate TCR on the pathogenic cells would activate the MHC-CAR expressing immune cells via the signaling domains(s) of the MHC-CAR (optionally by recruiting cell membrane signaling molecules of the immune cells), leading to proliferation and/or effector functions of the MHC-CAR-expressing immune cells, which in turn eliminate the pathogenic cells. The immune cells can be T cells, NK cells, macrophages, neutrophils, eosinophils, or any combination thereof. In some embodiments, the immune cells are T cells. In some embodiments, the immune cells are NK cells. Specific examples are provided in Examples below.

The population of immune cells can be obtained from any source, such as peripheral blood mononuclear cells (PBMCs), bone marrow, tissues such as spleen, lymph node, thymus, tumor tissue, or established cell lines. A source suitable for obtaining the type of immune cells desired would be evident to one of skill in the art. In some embodiments, the population of immune cells is derived from PBMCs. The type of immune cells desired (e.g., T cells, NK cells, macrophages, neutrophils, eosinophils, or any combination thereof) may be expanded within the population of cells obtained by co-incubating the cells with stimulatory molecules, for example, anti-CD3 and anti-CD28 antibodies may be used for expansion of T cells.

To construct the immune cells that express any of the MHC-CAR constructs described herein, expression vectors for stable or transient expression of the chimeric receptor construct may be constructed via conventional methods as described herein and introduced into immune host cells. For example, nucleic acids encoding the MHC-CAR may be cloned into a suitable expression vector, such as a viral vector (e.g., a lentiviral vector) in operable linkage to a suitable promoter. The nucleic acids and the vector may be contacted, under suitable conditions, with a restriction enzyme to create complementary ends on each molecule that can pair with each other and be joined with a ligase. Alternatively, synthetic nucleic acid linkers can be ligated to the termini of the nucleic acid encoding the chimeric receptors. The synthetic linkers may contain nucleic acid sequences that correspond to a particular restriction site in the vector. The selection of expression vectors/plasmids/viral vectors would depend on the type of host cells for expression of the chimeric receptors, but should be suitable for integration and replication in eukaryotic cells.

A variety of promoters can be used for expression of the MHC-CAR constructs described herein, including, without limitation, cytomegalovirus (CMV) intermediate early promoter, a viral LTR such as the Rous sarcoma virus LTR, HIV-LTR, HTLV-1 LTR, the simian virus 40 (SV40) early promoter, herpes simplex tk virus promoter. Additional promoters for expression of the chimeric receptors include any constitutively active promoter in an immune cell. Alternatively, any regulatable promoter may be used, such that its expression can be modulated within an immune cell.

Additionally, the vector may contain, for example, some or all of the following: a selectable marker gene, such as the neomycin gene for selection of stable or transient transfectants in host cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase, an inducible caspase such as iCasp9), and reporter gene for assessing expression of the MHC-CAR.

In some embodiments, the marker/sorting/suicide molecules for use in the present disclosure can be used for killing with rituximab and/or for sorting with QBEND. Philip et al., Blood 124(8):1277-87; 2014). One example is RQR8, which contains rituximab mimotope and QBEND-10 epitope. Exemplary sequences are provided below:

(SEQ ID NO: 144)
MGTSLLCWMALCLLGADHADACPYSNPSLCSGGGGSELPTQGTFSNVSTN
VSPAKPTTTACPYSNPSLCSGGGGSPAPPPTPAPTIASQPLSLRPEACRP
AAGGAVHTRGLDFACDIYIWAPLAGTCGVLLLSLVITLYCNHRNRRRVCK
CPRPVV
(SEQ ID NO: 394)
MGTSLLCWMALCLLGADHADACPYSNPSLCSGGGGSELPTQGTFSNVSTN
VSPAKPTTTACPYSNPSLCSGGGGSPAPRPPTPATIASQPLSLRPEACRP
AAGGAVHTRGLDFACDIYIWAPLAGTCGVLLLSLVITLYCNHRNRRRVCK
CPRPVV

An exemplary nucleic acid sequence encoding a RQR8 is provided below.

(SEQ ID NO: 395)
ATGGGTACTTCACTGTTGTGCTGGATGGCACTTTGTCTTTTGGGTGCCGA
TCATGCTGATGCATGTCCGTACTCCAATCCTAGCCTGTGCTCCGGGGGGG
GAGGGAGTGAACTCCCTACACAGGGAACCTTCTCTAATGTCTCCACCAAC
GTCTCCCCTGCAAAACCGACCACAACAGCTTGCCCCTATAGTAACCCTTC
CCTCTGTAGTGGAGGGGGGGGTTCACCTGCTCCACGCCCTCCTACCCCCG
CGCCAACGATCGCGTCACAACCGCTCAGTCTTAGGCCGGAAGCCTGTAGG
CCAGCGGCTGGCGGTGCGGTTCATACGCGGGGATTGGATTTTGCCTGCGA
CATTTACATTTGGGCTCCGCTGGCCGGTACTTGTGGGGTATTGCTGTTGT
CTCTTGTTATTACGCTTTATTGCAATCACAGaAACAGGCGACGAGTATGC
AAATGCCCGCGGCCCGTCTG

In another example, the following exemplary RQR sequence tag can be affixed to a MHC-CAR construct as disclosed herein:

(SEQ ID NO: 145)
ACPYSNPSLCSGGGGSELPTQGTFSNVSTNVSPAKPTTTACPYSNPSLCS
GGGGS

The boldfaced fragment is the rituximab minotope and the underlined/italicized fragment is the QBEND-10 epitope.

Suitable vectors and methods for producing vectors containing transgenes are well known and available in the art. Any of the vectors comprising a nucleic acid sequence that encodes a MHC-CAR construct described herein is also within the scope of the present disclosure. Such a vector may be delivered into host immune cells by a suitable method. Methods of delivering vectors to immune cells are well known in the art and may include DNA electroporation, RNA electroporation, transfection reagents such as liposomes, or viral transduction. In some embodiments, the vectors for expression of the MHC-CAR are delivered to host cells by viral transduction. Exemplary viral methods for delivery include, but are not limited to, recombinant retroviruses (see, e.g., PCT Publication Nos. WO 90/07936; WO 94/03622; WO 93/25698: WO 93/25234; WO 93/11230; WO 93/10218; WO 91/02805; U.S. Pat. Nos. 5,219,740 and 4,777,127; GB Patent No. 2,200,651; and EP Patent No. 0 345 242), alphavirus-based vectors, and adeno-associated virus (AAV) vectors (see, e.g., PCT Publication Nos. WO 94/12649, WO 93/03769; WO 93/19191: WO 94/28938; WO 95/11984 and WO 95/00655). In some embodiments, the vectors for expression of the chimeric receptors are retroviruses. In some embodiments, the vectors for expression of the chimeric receptors are lentiviruses.

In examples in which the vectors encoding chimeric receptors are introduced to the host cells using a viral vector, viral particles that are capable of infecting the immune cells and carry the vector may be produced by any method known in the art and can be found, for example in PCT Application No. WO 1991/002805A2, WO 1998/009271 A1, and U.S. Pat. No. 6,194,191. The viral particles are harvested from the cell culture supernatant and may be isolated and/or purified prior to contacting the viral particles with the immune cells.

Following introduction into the host cells a vector encoding any of the MHC-CAR provided herein, the cells are cultured under conditions that allow for expression of the chimeric receptor. In examples in which the nucleic acid encoding the MHC-CAR is regulated by a regulatable promoter, the host cells are cultured in conditions wherein the regulatable promoter is activated. In some embodiments, the promoter is an inducible promoter and the immune cells are cultured in the presence of the inducing molecule or in conditions in which the inducing molecule is produced. Determining whether the MHC-CAR is expressed will be evident to one of skill in the art and may be assessed by any known method, for example, detection of the chimeric receptor-encoding mRNA by quantitative reverse transcriptase PCR (qRT-PCR) or detection of the chimeric receptor protein by methods including Westem blotting, fluorescence microscopy, and flow cytometry. See also Examples below. Alternatively, expression of the MHC-CAR may take place in vivo after the immune cells are administered to a subject.

Alternatively, expression of a MHC-CAR construct in any of the immune cells disclosed herein can be achieved by introducing RNA molecules encoding the MHC-CAR constructs. Such RNA molecules can be prepared by in vitro transcription or by chemical synthesis. The RNA molecules can then introduced into suitable host cells such as immune cells (e.g., T cells, NK cells, macrophages, neutrophils, eosinophils, or any combination thereof) by, e.g., electroporation. For example, RNA molecules can be synthesized and introduced into host immune cells following the methods described in Rabinovich et al., Human Gene Therapy, 17:1027-1035 and WO WO2013/040557.

The methods of preparing host immune cells expressing any of the MHC-CARs described herein may comprise expanding the host immune cells ex vivo. Expanding host immune cells may involve any method that results in an increase in the number of cells expressing MHC-CAR, for example, allowing the host cells to proliferate or stimulating the host cells to proliferate. Methods for stimulating expansion of host cells will depend on the type of host cell used for expression of the chimeric receptors and will be evident to one of skill in the art. In some embodiments, the host immune cells expressing any of the MHC-CAR described herein can be expanded ex vivo prior to administration to a subject.

(ii) Additional Genetic Modifications

One or more additional genetic modifications can be introduced into host immune cells before, concurrently with, or after the transfection of the MHC-CAR construction. For example, one or more marker and/or suicide genes may be introduced into the host immune cells. Examples include green fluorescent protein (GFP), enhanced blue fluorescent protein (eBFP), and RQR genes, such as RQR8 (a compact marker/suicide gene for T cells which combines target epitopes from CD34 and CD20. Philip et al., Blood 124(8):1277-87; 2014). Such marker/suicide genes may be constructed in one expression cassette with the MHC-CAR components.

An example of an amino acid sequence of GFP is provided below:

(SEQ ID NO: 427)
VSKGEELFTGVVPILVELDGDVNGHKFSVSGEGEGDATYGKLTLKFICTT
GKLPVPWPTLVTTLTYGVQCFSRYPDHMKQHDFFKSAMPEGYVQERTIFF
KDDGNYKTRAEVKFEGDTLVNRIELKGIDFKEDGNILGHKLEYNYNSHNV
YIMADKQKNGIKANFKIRHNIEDGSVQLADHYQQNTPIGDGPVLLPDNHY
LSTQSALSKDPNEKRDHMVLLEFVTAAGITLGMDELYK

An example of a nucleic acid sequence encoding GFP is provided below:

(SEQ ID NO: 428)
gtgagcaagggcgaggagctgttcaccggggtggtgcccatcctggtgga
gctggacggcgacgtaaacggccacaagttcagcgtgtctggcgagggcg
agggcgatgccacctacggcaagctgaccctgaagttcatctgcaccacc
ggcaagctgcccgtgccctggcccaccctcgtgaccaccctgacctacgg
cgtgcagtgcttcagccgctaccccgaccacatgaagcagcacgacttct
tcaagtccgccatgcccgaaggctacgtccaggagcgcaccatcttcttc
aaggacgacggcaactacaagacccgcgccgaggtgaagttcgagggcga
caccctggtgaaccgcatcgagctgaagggcatcgacttcaaggaggacg
gcaacatcctggggcacaagctggagtacaactacaacagccacaacgtc
tatatcatggccgacaagcagaagaacggcatcaaggcgaacttcaagat
ccgccacaacatcgaggacggcagcgtgcagctcgccgaccactaccagc
agaacacccccatcggcgacggccccgtgctgctgcccgacaaccactac
ctgagcacccagtccgccctgagcaaagaccccaacgagaagcgcgatca
catggtcctgctggagttcgtgaccgccgccgggatcactctcggcatgg
acgagctgtacaagtaa

In some instances, the endogenous TCR (alpha chain, beta chain, or both) can be disrupted such that the host immune cells do not express the endogenous TCR. Deficiency in endogenous TCR could avoid undesired T cell activation. Alternative or in addition, certain cell surface receptors can be knocked out. Such surface receptors may be target receptors for disease treatment, for example, CD52, which is a target for MS treatment. Knock-out such target receptors from the MHC-CAR immune cells allows for the co-use of the MHC-CAR immune cells with therapeutic agents specific to the target receptor (e.g., and anti-CD52 antibodies such as alemtuzumab).

In some embodiments, the host immune cells may be modified with synthetic surface proteins to enhance their retention in a specific organ or tissue, for example, in the lymph nodes, in tertiary lymphoid organs, or at sites of inflammation. Doing so would allow the modified immune cells to access target pathogenic cells, while minimizing fatal off-target effects due to penetration of the blood brain barrier or free travel of the immune cells through peripheral blood. Cells early in the T cell differentiation pathway (e.g., naïve, stem cell memory, and central memory T cells) travel freely to the lymph nodes. As differentiation progresses, most effector T cells leave the lymph node. Pathologic immune cells can also travel to and accumulate at sites of inflammation. Treatment by activated CAR-T cells has a number of undesirable effects when they react with undesired targets. Interaction with heart tissue can be fatal to cardiac protein, and permeation of the brain can lead to fatal cerebral edema. Recent progress has been made in treatment of the brain cancer gliobastoma using lower doses of CAR T therapy than in systemic treatments for cancer using CD19 CAR T therapy. Brown et al., New England Journal of Medicine, 375(26):2561-2569, 2016. Resolution of inflammation has the potential to transform pathologic to protective environments. Gagliani et al., Nature, 523(7559):221-225, 2015.

Introducing one or more of lymph node retention proteins into the immune cells can enhance retention of the immune cells in the lymph node, wherein the immune cells still have access to target pathogenic cells, while undesired effects as noted above can be significantly reduced. Naïve lymphocytes enter the lymph node via high endothelial venules (HEVs). Thus, expressing or overexpressing proteins involved in HEV anchor ing and/or entry can facilitate the immune cells entering into lymph node. Exemplary lymph node retention proteins include, but are not limited to, CCR7 (a chemokine receptor), MECA79 (a peripheral lymph node addressin), vascular adhesion protein-1 (VAP-1) and CD62 (selectin, a family of the cell adhesion molecules). Azzi et al., Blood 124(4):476-477, 2016; Streeter et al., J. Cell. Biol. 107:1853-186; 1988: Michie et al., Amer, J. Path. 143:1688-1698; 1993; Berg et al., J. Cell. Biol. 114:343-349: 1991; Berg et al., Nature 366:695-698; 1993; and Hemmerich et al., J. Exp. Med. 180:2219-2226; 1994. Alternatively, genes encoding proteins (e.g., sphingosine-1-phosphate receptor-1 or SIP) involved in lymphocyte egress from the thymus and lymph organs can be knocked out from the immune cells.

Chemokine receptors and adhesion receptors that promote trafficking to sites of inflammation can also bring MHC-CAR immune cells in contact with pathogenic cells that propagate immune disease [Barreiro et al., Cardiovascular research, 86(2):174-182, 2010] see Table 3 and 4. Receptors involved in recruiting immune cells that propagate inflammation include receptors (i.e., CXCR5, CCR7, CCR6) that recruit to tertiary lymphoid organs (where CXCL13, CCL19, CCL2, CCL21 are expressed).

One or more genes encoding proteins involved in targeting other organs/tissues, for example brain/CNS, bone marrow, pancreas, intestine, liver, lungs, spleen, and/or thymus, may also be introduced into or knocked-out from the immune cells.

The genes (in Table 3 and 4), by means of virally induced or temporary RNA mediated expression (possibly combined with knockout of the endogenous gene) in the therapeutic cell, may be used to route either Treg or CTL cells to the desired location or to treat-remove the desired cells. Barreiro, et al. Cardiovascular research, 86(2):174-182, 2010. If mRNA transfection is utilized then it can allow expression of the chemokine or adhesion receptor for a week. Wang and Riviere. Molecular Therapy-Oncolytics, 3:16015 2016.

TABLE 3
Chemokine receptors and natural context

| | | GenBank | |
|---|---|---|---|
| | Typically | Accession | |
| Receptor | expressed on | number | Ligands |
| CXCR1 | Neutrophils | AAY21515.1 | CXCL8, CXCL6 |
| CXCR2 | Neutrophils | NP_001548.1 | CXCL1, 2, 3, 4, 5, 7 |
| CXCR3 | T cells and some | NP_001495.1 | CXCL9, 10, 11 |
| | B cells | | |
| CXCR4 | Most mature and | CAA12166.1 | CXCL12 |
| | immature | | |
| | hematopoietic cells | | |
| CXCR5 | B cells and | NP_001707.1 | CXCL13 |
| | Tfh cells | | |
| CXCR6 | Inflammation | NP_006555.1 | CXCL16 |
| | response T cells | | |
| | but weak | | |
| | chemotaxis | | |
| CXCR7 | Memory B cells, | NP_064707.1 | CXCL12 |
| | T cells | | |
| CCR1 | Peripheral | NP_001286.1 | CCL4, CCL5, CCL6, |
| | lymphocytes, | | CCL14, CCL15, |
| | memory T cells | | CCL16, CCL23 |
| CCR2 | Monocytes, | AAA19119.1 | CCL11, CCL26, |
| | activated memory | | CCL7, CCL13, |
| | T cells, B cells, | | CCL15, CCL24 & |
| | basophils | | CCL5, CCL28 |
| CCR3 | Plays a role in | NP_001828.1 | CCL11, CCL26, |
| | allergic reactions, | | CCL7, CCL13, |
| | B and T cells to | | CCL15, CCL24, |
| | mucous, eosinophils, | | CCL5 |
| | basophils | | |
| CCR4 | Th2 lymphocytes, | NP_005499.1 | CCL3, CCL5, |
| | dendritic cells | | CCL17, CCL22 |
| CCR5 | Peripheral blood | NP_000570.1 | CCL2, CCL3, CCL4, |
| | dendritic cells, | | CCL5, CCL11, |
| | CD34+ | | CCL13, CCL14, |
| | hematopoietic | | CCL16 |
| | progenitors, | | |
| | activated/memory | | |
| | Th1 | | |
| CCR6 | Inactivated memory | AAC51124.1 | CCL20 |
| | T cells, dendritic | | |
| | cells, Th17, | | |
| | downregulated on | | |
| | activated T cells | | |
| CCR7 | Trafficking of B, | AAH35343.1 | CCL19, CCL21 |
| | T, and dendritic | | |
| | cells across | | |
| | HEV and into T cell | | |
| | zone of lymph | | |
| | nodes | | |
| CCR8 | Th2 cells, thymus, | NP_005192.1 | CCL1, CCL16 |
| | lymph nodes, spleen, | | |
| | brain, monocytes | | |
| CCR9 | Thymus, gut | NP_112477.1 | CCL25 |
| CCR10 | Skin, mucous layers, | NP_057686.2 | CCL27, CCL28 |
| | regulatory T cells | | |
| XCR1 | | NP_005274.1 | XCL1, XCL2 |
| CX3CR1 | | NP_001164642.1 | CX3CL1 |

The amino acid sequence of CCR6, provided by AAC51124.1 is shown below:

(SEQ ID NO: 391)
MSGESMNFSDVFDSSEDYEVSVNTSYYSVDSEMLLCSLQEVROFSRLFVP
IAYSLICVFGLLGNILVVITFAFYKKARSMTDVYLLNMAIADILFVLTLP
FWAVSHATGAWVFSNATCKLLKGIYAINFNCGMLLLTCISMDRYIAIVQA
TKSFRLRSRTLPRSKIICLVVWGLSVIISSSTFVFNQKYNTQGSDVCEPK
YQTVSEPIRWKLLMLGLELLFGFFIPLMFMIFCYTFIVKTLVQAQNSKRH
KAIRVIIAVVLVFLACQIPHNMVLLVTAANLGKMNRSCQSEKLIGYTKTV
TEVLAFLHCCLNPVLYAFIGQKFRNYFLKILKDLWCVRRKYKSSGFSCAG
RYSENISRQTSETADNDNASSFTM

Example nucleic acid sequences of CCR6 are shown below:

(SEQ ID NO: 392)
atgagtggggaaagtatgaacttcagcgatgtatttgactcctccgaaga
ttactttgtatctgtgaatacgagctattactccgtcgatagtgaaatgc
tgctctgtagtctccaagaagtccgccaattcagtcgcctcttcgttccc
atcgcgtactcccttatttgtgtttttggccttctgggtaacatcctggt
tgtaatcacattcgctttctataaaaaagctcggagtatgactgatgttt
accttcttaacatggctatagcggacattctttttgtgcttactctccca
ttctgggctgtgagccatgcaacaggggcgtgggttttttcaaatgccac
atgtaagctgcttaaagggatctatgcaataaacttcaattgcgggatgc
tcctgctgacatgcatcagtatggatcgatacatagctatagtacaggcg
actaagtccttccgcctgcgatcccgcacactgcctaggagcaaaattat
ttgcctcgtcgtatgggggctctcagtgatcatctcctccagtacgtttg
tctttaaccagaaatataacacacagggttctgatgtatgtgaaccaaag
tatcagacagtgagtgaaccaatacggtggaagttgcttatgttgggctt
ggagctgctttttgggtttttcatcccactgatgttcatgattttctgtt
atacatttattgttaagaccttggttcaggcgcaaaatagcaagagacat
aaggcaattcgagtcatcattgccgtggtgttggtcttcttggcctgtca
gatcccccataatatggttctgctcgtcaccgccgctaacttgggtaaga
tgaatcgatcttgtcagtocgagaagttgatoggatacaccaaaactgtg
acagaagtgctggccttccttcactgttgtctgaacccagttttgtatgc
ttttataggacagaagtttcgaaattacttcttgaaaatcctcaaggacc
tctggtgtgttcgaaggaagtacaagagctctggctttagttgcgctggg
cgctacagtgagaatatatcccggcagacctccgagactgctgataatga
caacgcaagttccttcactatg
(SEQ ID NO: 393)
ATGAGCGGGGAATCAATGAATTTCAGCGATGTTTTCGACTCCAGTGAAGA
TTATTTTGTGTCAGTCAATACTTCATATTACTCAGTTGATTCTGAGATGT
TACTGTGCTCCTTGCAGGAGGTCAGGCAGTTCTCCAGGCTATTTGTACCG
ATTGCCTACTCCTTGATCTGTGTCTTTGGCCTCCTGGGGAATATTCTGGT
GGTGATCACCTTTGCTTTTTATAAGAAGGCCAGGTCTATGACAGACGTCT
ATCTCTTGAACATGGCCATTGCAGACATCCTCTTTGTTCTTACTCTCCCA
TTCTGGGCAGTGAGTCATGCCACTGGTGCGTGGGTTTTCAGCAATGCCAC
GTGCAAGTTGCTAAAAGGCATCTATGCCATCAACTTTAACTGCGGGATGC
TGCTCCTGACTTGCATTAGCATGGACCGGTACATCGCCATTGTACAGGCG
ACTAAGTCATTCCGGCTCCGATCCAGAACACTACCGCGCACGAAAATCAT
CTGCCTTGTTGTGTGGGGGCTGTCAGTCATCATCTCCAGCTCAACTTTTG
TCTTCAACCAAAAATACAACACCCAAGGCAGCGATGTCTGTGAACCCAAG
TACCAGACTGTCTCGGAGCCCATCAGGTGGAAGCTGCTGATGTTGGGGCT
TGAGCTACTCTTTGGTTTCTTTATCCCTTTGATGTTCATGATATTTTGTT
ACACGTTCATTGTCAAAACCTTGGTGCAAGCTCAGAATTCTAAAAGGCAC
AAAGCCATCCGTGTAATCATAGCTGTGGTGCTTGTGTTTCTGGCTTGTCA
GATTCCTCATAACATGGTCCTGCTTGTGACGGCTGCAAATTTGGGTAAAA
TGAACCGATCCTGCCAGAGCGAAAAGCTAATTGGCTATACGAAAACTGTC
ACAGAAGTCCTGGCTTTCCTGCACTGCTGCCTGAACCCTGTGCTCTACGC
TTTTATTGGGCAGAAGTTCAGAAACTACTTTCTGAAGATCTTGAAGGACC
TGTGGTGTGTGAGAAGGAAGTACAAGTCCTCAGGCTTCTCCTGTGCCGGG
AGGTACTCAGAAAACATTTCTCGGCAGACCAGTGAGACCGCAGATAACGA
CAATGCGTCGTCCTTCACTATG

TABLE 4
Adhesion receptors and natural context

| | Typically | Accession | |
|---|---|---|---|
| Receptor | expressed on | number | Ligands |
| VLA-1 or α₁β₁ | Many cell types | NP_852478.1, | Collagens, |
| | | AAH20057.1 | laminins |
| VLA-2 or α₂β₁ | Many cell types | NP_002194.2, | Collagens, |
| | | AAH20057.1 | laminins |
| VLA-3 or α₃β₁ | Many cell types | AAI50191.1, | Laminin-5 |
| | | AAH20057.1 | |
| VLA-4 or α₄β₁ | Hematopoietic | NP_000876.3, | Fibronectin and |
| | cells | AAH20057.1 | proteinases |
| VLA-5 or α₅β₁ | Many cell types | NP_002196.4, | Fibronectin, |
| | | AAH20057.1 | VCAM-1 |
| VLA-6 or α₆β₁ | Many cell types | AAI36456.1, | Laminins |
| | | AAH20057.1 | |
| α₄β₇ | Gut | NP_000876.3, | MADCAM1 |
| | | NP_00880.1 | |
| α₇β₁ | Muscle | AAQ89241.1, | Laminins |
| | | AAH20057.1 | |
| α_{L}β₂ | T lymphocytes | NP_002200.2, | I-CAM1, I-CAM2 |
| | | NP_000202.3 | |
| MAC-1 or | Neutrophils and | AAB24821.1, | I-CAM1 |
| α_{M}β₂ | monocytes | NP_000202.3 | |
| α_{IIb}β₃ | Platelets | AAI26443.1, | Fibrinogen, |
| | | AAI27668.1 | fibronectin |
| α_{V}β₁ | Melanocytes | AAA61631.1, | Vitronectin, |
| | | AAH20057.1 | fibrinogen |
| α_{V}β₃ | Activated | AAA61631.1, | Vitronectin, |
| | endothelial | AAI27668.1 | fibronectin, |
| | cells | | fibrinogen, |
| | | | osteopontin, |
| | | | Cyr61, tyroxine, |
| | | | Tetrac |
| α_{V}β₅ | Epithelial | AAA61631.1, | Vitronectin |
| | cells and | NP_002204.2 | |
| | fibroblasts | | |
| α_{V}β₆ | Lung, mammary | AAA61631.1, | Fibrinonectin and |
| | gland | NP_000879.2 | TGF-β 1, 3 |
| α_{V}β₈ | Neural tissues | AAA61631.1, | Fibrinonectin and |
| | | NP_002205.1 | TGF-β 1, 3 |
| α₆β₄ | Epithelial | AAA61631.1, | Laminin |
| | cells | CAB61345.1 | |

Alternatively or in addition, genes that may enhance immune cell functions, e.g. proliferation, cytotoxicity, etc., can also be introduced into or knocked-out from the immune cells. Examples include TNFTNFR2 overexpression (for short-lived but more effective CD8 T cells), gld (FasL mutant; for lymphoproliferation; CTLs do not kill via Fas-FasL pathway); lpr (Fas mutant, for upregulation of FasL—target cells resistant to FasL-mediated apoptosis)c Granzyme B* deficient (delayed nuclear apoptotic changes in target cells); Granzyme A & B* deficient (delayed nuclear apoptotic changes in target cells); Perform deficient (complete absence of granule-mediated apoptosis); Perforin and FasL deficient (defective granule-mediated and Fas-mediated apoptosis); Cathepsin C (dipeptidyl-peptidase) deficient (fails to produce active granzymes and some haematopoietic serine proteases); FAS (CD95) underexpression; and/or FASL overexpression.

Table 5 below lists additional genetic modifications of the MHC-CAR T cells or co-treatment described herein and the accompanying advantages arising therefrom.

TABLE 5
Genetic Modifications or Co-Treatment and Benefits Thereof

| Genetic Modifications | Advantages |
|---|---|
| TCR knockout | Reduce fraternal killing of |
| CIITA deletion (to remove | MHC-CAR T cells by natural |
| endogenous MHC | CD8⁺ T cells with an |
| class II expression) | affinity to the peptide-MHC |
| CS-1 (CD319) deletion (which | in the MHC-CAR, thereby |
| is present on T cells) | extending the life-span of |
| | the MHC-CAR T cells. (Without |
| | the genetic modifications, |
| | the MHC-CAR T cells would |
| | still be cytotoxic and |
| | effective, but would have a |
| | short life-span.) |
| Fas deletion, which | Enhance activity to eliminate |
| optionally can be in | CD8 cytotoxic T cells, |
| combination of FasL | antigen-presenting cells |
| overexpression | (APCs), and/or B cells |
| PD-1 deletion, which | |
| optionally can be in | |
| combination with | |
| PD-L1 and/or PD-L2 | |
| overexpression | |
| Co-express of CS-1 CAR | |
| (conventional CAR construct | |
| having an extracellular | |
| domain specific to CS-1) | |
| Co-express of CD19 CAR | |
| (conventional CAR construct | |
| having an extracellular | |
| domain specific to CD19) | |
| PD-L1 overexpression, which | Reduce the level of |
| may optionally be in | MHC-CAR T cell |
| combination with | elimination by other |
| PD-1 deletion | immune cells; |
| PD-L1 + CTLA4-Ig, which | Inclusion of hinge may |
| may optionally may be in | decrease/prevent killing |
| combination with PD-1 | with cell TCR by, e.g., |
| deletion | decreasing ability for |
| FasL overexpression, which | it to engage CD4 or CD8 |
| may optionally be in | |
| combination with Fas deletion | |
| Galectin 9 expression, which | |
| may optionally be in | |
| combination with | |
| Tim-3 deletion | |
| CS-1 deletion (when CS-1 CAR | |
| is used to reduce fraternal | |
| killing) | |
| Include a hinge in a MHC-CAR | |
| TCR knockout when allogenic | Reduce the risk of |
| cells are used (cells can be | graft-v-host disease |
| sorted to remove non-edited | |
| cells) | |
| Treg cells expressing MHC-CAR | Reduce humoral responses |
| PD-1 and/or PD-L1 knockout | to the peptide-MHC of |
| CXCR5 expression | interest (e.g., antibody |
| | responses), especially a |
| | B cell response |
| IL-35 expression | Reduce inflammation and/or |
| Inhibitors (e.g., antibodies) | enhance tolerogenic |
| targeting cytokine producing | environment |
| B cells (e.g., targeting | |
| CD10) and/or other activated | |
| immune cells (e.g., targeting CS-1) | |
| Relevent chemokine receptor | Routing to inflamed or |
| expression to either direct | antigen presenting or |
| to relevant organ (Schall et al., | antigen targeting |
| Nature Reviews Immunology, | environment |
| 11(5): 355-363, 2011) or to | |
| interact with relevant cell | |
| type (example, CXCR5 for | |
| B cell, CCR6 for Th17) | |
| Antigen targeting antibody | |
| scFv that contains a CD8 | |
| hinge, a transmembrane | |
| domain, and optional | |
| stimulatory and co-stimulatory | |
| domain (for Treg only). (for | |
| example an anti-MOG antibody). | |
| The relevant antibody sequence | |
| (for a subset of antigen targets | |
| in Table 1) can be generated | |
| from sequencing of commercially | |
| available human-targeted | |
| monoclonal variants using mass | |
| spectrometry. Tran et al. | |
| Scientific reports, 6: 31730, 2016. | |
| Genetically encoded kill-switches | Reduce cytokine crisis |

In some embodiments, genetic modification that lead to PD blockade can be introduced into the immune cells that express a MHC-CAR as described herein. Such modifications include one or more of PD-1 knockout, PD-L1 or L2 overexpression, or PD-L1 knockout. PD blockade may be combined with an immune-inhibitor (e.g., knockouts of CTLA-4, TIM-3, LAG3, TIGIT, IDO, or Arginase, or CTLA-41g secretion), an immunostimulator (e.g., anti-OX40, anti-CD137, IL-2, TLR ligands, or STING), and/or a kinase inhibitor (e.g., Braf inhibitor or MEK inhibitor)

Table 6 below provides exemplary genetic modifications for PD blockade, immune inhibitor, death receptor, immunostimulator, toll like receptor, kinase inhibition, master regulator, cytokine signaling, cell interaction reduction, and drug interaction related edits. The tables also provides target sequences for guide RNA using Cas9 in T cells as well as Genbank accession numbers for sequences that can be used for expression/overexpression. Genome editing using gRNAs is performed through transduction of lentivirus (lentiCRISPRv2) containing the desired gRNA and the Streptococcus pyogenes Cas9 nuclease. This can be perform as an alternative to delivery of TALEN RNA in the protocols. Sanjana, et al. Nature Methods (2014) 11(8):783-784.

TABLE 6
Exemplary Genes for Genetic Modification and Corresponding
Cas9-Mediated Edits

| | Genbank | Genomic sequences (gRNA) |
|---|---|---|
| PD blockade related edits | accession no. | for Cas9 targeting |
| Programmed cell death protein 1 | NP_005009.2 | TGACGTTACCTCGTGCGGCC |
| (PDCD1) | | (SEQ ID NO: 146), |
| | | CACGAAGCTCTCCGATGTGT |
| | | (SEQ ID NO: 147), |
| | | GCGTGACTTCCACATGAGCG |
| | | (SEQ ID NO: 148), |
| | | TTGGAACTGGCCGGCTGGCC |
| | | (SEQ ID NO: 149), |
| | | GTGGCATACTCCGTCTGCTC |
| | | (SEQ ID NO: 150), |
| | | GATGAGGTGCCCATTCCGCT |
| | | (SEQ ID NO: 151), |
| Programmed cell death 1 ligand 1 | NP_005009.2 | TACCGCTGCATGATCAGCTA |
| (CD274) | | (SEQ ID NO: 152), |
| | | AGCTACTATGCTGAACCTTC |
| | | (SEQ ID NO: 153), |
| | | GGATGACCAATTCAGCTGTA |
| | | (SEQ ID NO: 154), |
| | | ACCCCAAGGCCGAAGTCATC |
| | | (SEQ ID NO: 155), |
| | | TCTTTATATTCATGACCTAC |
| | | (SEQ ID NO: 156), |
| | | ACCGTTCAGCAAATGCCAGT |
| | | (SEQ ID NO: 157) |
| | Genbank | Genomic sequences for |
| Immune-inhibitor related edits | accession no. | Cas9 targeting |
| Cytotoxic T-lymphocyte protein 4 | NP_005205.2 | GTACCCACCGCCATACTACC |
| (CTLA4) | | (SEQ ID NO: 158), |
| | | TTGCCTATGCCCAGGTAGTA |
| | | (SEQ ID NO: 159), |
| | | CCTTGTGCCGCTGAAATCCA |
| | | (SEQ ID NO: 160), |
| | | ACCCCGAACTAACTGCTGCA |
| | | (SEQ ID NO: 161), |
| | | ACATAGACCCCTGTTGTAAG |
| | | (SEQ 1D NO: 162), |
| | | ATCCTTGCAGCAGTTAGTTC |
| | | (SEQ ID NO: 163) |
| CTLA4-Ig (Orencia) | APZ76727.1 | |
| Serine/threonine-protein phosphatase 2A | NP_002706.1 | ACATCGAACCTCTTGCACGT |
| catalytic subunit alpha isoform | | (SEQ ID NO: 164), |
| (PPP2CA) | | TACAGCTCACCTTCTCGCAG |
| | | (SEQ ID NO 165):, |
| | | GGTATATCTCCTCGAGGAGC |
| | | (SEQ ID NO: 166), |
| | | TACACTGCTTGTAGCTCTTA |
| | | (SEQ 1D NO: 167), |
| | | GAGCTCTAGACACCAACGTG |
| | | (SEQ ID NO: 168), |
| | | CAAGCAGCTGTCCGAGTCCC |
| | | (SEQ ID NO: 169) |
| Serine/threonine-protein phosphatase 2A | CAA31183.1 | AATGTGTAGCCAGCACCACG |
| catalytic subunit beta isoform (PPP2CB) | | (SEQ ID NO: 170), |
| | | GAACTTCCTGTAAACGATCC |
| | | (SEQ 1D NO: 171), |
| | | TACATACCTCCATTACAAGC |
| | | (SEQ ID NO: 172), |
| | | CCATCTACTAAAGCTGTAAG |
| | | (SEQ ID NO: 173), |
| | | CTCAATATTGTAATGCGTTC |
| | | (SEQ ID NO: 174), |
| | | CTCTCCATCCATAGACACAC |
| | | (SEQ ID NO: 175) |
| Protein tyrosine phosphatase, non- | AAA35963.1 | TAAGACCTACATCGCCAGCC |
| receptor type 6 (PTPN6) | | (SEQ ID NO: 176), |
| | | GAAGAACTTGCACCAGCGTC |
| | | (SEQ ID NO: 177), |
| | | GTCAGCCGCATTCACCCTCG |
| | | (SEQ ID NO: 178), |
| | | CTGCCAGAAGTCATTGACCG |
| | | (SEQ ID NO: 179), |
| | | CCCAGCCGTACTATGCCACG |
| | | (SEQ ID NO: 180), |
| | | GCCGCTGCCCTTCCAGACGC |
| | | (SEQ ID NO: 181) |
| Tyrosine-protein phosphatase non- | AAD00904.1 | GTAGCGGAATCCTCATCAG |
| receptor type 22 (PTPN22) | | (SEQ ID NO: 182), |
| | | CAAAACCTATCCTACAACTG |
| | | (SEQ ID NO: 183), |
| | | TTAGGGAGTTTATGGACCCA |
| | | (SEQ ID NO: 184), |
| | | CTCAGCCACAGTTGTAGGAT |
| | | (SEQ ID NO: 185), |
| | | TCACTGTACCTTAATGAAGT |
| | | (SEQ ID NO: 186), |
| | | TCCTTTATCTACAACCCTCC |
| | | (SEQ ID NO: 187) |
| Lymphocyte activation gene 3 protein | CAA36243.3 | TCCATAGGTCCCCAACGCTC |
| (LAG3) | | (SEQ ID NO: 188), |
| | | GTTCCGGAACCAATGCACAG |
| | | (SEQ ID NO: 189), |
| | | GCGAGAAGTCCCCGCGCTGC |
| | | (SEQ ID NO: 190), |
| | | TGACCCCTGCTCTTCGCAGA |
| | | (SEQ ID NO: 191), |
| | | CGCCGGCGAGTACCGCGCCG |
| | | (SEQ ID NO: 192), |
| | | TGGGCGGTCAGGGCGGCTGA |
| | | (SEQ ID NO: 193) |
| Hepatitis A virus cellular receptor 2 | AAM19100.1 | CTAAATGGGGATTTCCGCAA |
| (Tim3, HAVCR2) | | (SEQ ID NO: 194), |
| | | ATCCCCATTTAGCCAGTATC |
| | | (SEQ ID NO: 195), |
| | | GTGAAGTCTCTCTGCCGAGT |
| | | (SEQ ID NO: 196), |
| | | AGGTCACCCCTGCACCGACT |
| | | (SEQ ID NO: 197), |
| | | CTTACTGTTAGATTTATATC |
| | | (SEQ ID NO: 198), |
| | | TATAGCAGAGACACAGACAC |
| | | (SEQ ID NO: 199) |
| B- and T-lymphocyte attenuator (BTLA) | AAP44003.1 | GTGACTTGGTGCAAGCTCAA |
| | | (SEQ ID NO: 200), |
| | | TCTGCTTGCCATTTCGTCCT |
| | | (SEQ ID NO: 201), |
| | | CTGTTAGCACAGTATTTCAC |
| | | (SEQ ID NO: 202), |
| | | CCAAAGGAAGTAAACGATAC |
| | | (SEQ ID NO: 203), |
| | | ATGTTCCAGATGTCCAGATA |
| | | (SEQ ID NO: 204), |
| | | CTTCTTCTTAATCCCATATC |
| | | (SEQ ID NO: 205) |
| CD160 antigen (CD160) | AAC72302.1 | AGTTTAGTCGCGTTCCTTCC |
| | | (SEQ ID NO: 206), |
| | | CACTGTGCAACGGTGTGACT |
| | | (SEQ ID NO: 207), |
| | | GGATGTCCACAATTGCCAGC |
| | | (SEQ ID NO: 208), |
| | | AACTGAGAGTGCCTTCATTA |
| | | (SEQ ID NO: 209), |
| | | GACAGGGAACTACACAGTGA |
| | | (SEQ ID NO: 210), |
| | | GACAGGGAACTACACAGTGA |
| | | (SEQ ID NO: 210), |
| | | ATTGTGGACATCCAGTCTGG |
| | | (SEQ ID NO: 212) |
| T-cell immunoreceptor with Ig and ITIM | BAC04973.1 | TCGCTGACCGTGAACGATAC |
| domains (TIGIT) | | (SEQ ID NO: 213), |
| | | TGGGGCCACTCGATCCTTGA |
| | | (SEQ ID NO: 214), |
| | | GCAGATGACCACCAGCGTCG |
| | | (SEQ ID NO: 215), |
| | | TCAGGCCTTACCTGAGGCGA |
| | | (SEQ ID NO: 216), |
| | | CATCTGCACAGCAGTCATCG |
| | | (SEQ ID NO: 217), |
| | | ATTGAAGTAGTCATGCAGCT |
| | | (SEQ ID NO: 218) |
| T-cell surface protein tactile (CD96) | AAA36662.1 | AGGCACAGTAGAAGCCGTAT |
| | | (SEQ ID NO: 219), |
| | | GCTGTCTATCATCCCCAATA |
| | | (SEQ ID NO: 220), |
| | | ACTTACCACCGACCATGCAT |
| | | (SEQ ID NO: 221) |
| Cytotoxic and regulatory T-cell molecule | AAC80267.1 | CACACTTTAGAGTGAGCGTC |
| (CRTAM) | | (SEQ ID NO: 222), |
| | | CTCCAGTGGCTGACCCCCTC |
| | | (SEQ ID NO: 223), |
| | | CCACAGCAGCCCACCAGTAC |
| | | (SEQ ID NO: 224) |
| Leukocyte-associated immunoglobulin- | AF013249.1 | TTATAATAGATGCAGCGATA |
| like receptor 1 (LAIR1) | | (SEQ ID NO: 225), |
| | | TCATTGIGACTGTTGTCCGA |
| | | (SEQ ID NO: 226), |
| | | GCCAGGCACCGTGATCCCCC |
| | | (SEQ ID NO: 227) |
| Sialic acid-binding Ig-like lectin 7 | AF170485.1 | CATCCTTATCCCCGGTACCC |
| (SIGLEC7) | | (SEQ ID NO: 228), |
| | | CAGAGAGCTTCTGAGCTCGAC |
| | | (SEQ ID NO: 229), |
| | | AGTGTTGCTGGGGGCGGTCG |
| | | (SEQ ID NO: 230 |
| Sialic acid-binding Ig-like lectin 9 | AF135027.1 | GACGATGCAGAGTTCCGTGA |
| (SIGLEC9) | | (SEQ ID NO: 231), |
| | | ACTCACAGGACACGTTGAGA |
| | | (SEQ ID NO: 232), |
| | | TACCCTGGCCCAGTAGTTCA |
| | | (SEQ ID NO: 233) |
| Natural killer cell receptor 2B4 (CD244) | AF105261.1 | ACCTTCGTCTGTATGCTGTT |
| | | (SEQ ID NO: 234), |
| | | ACCAAACAGCATACAGACGA |
| | | (SEQ ID NO: 235), |
| | | CTACTCTATGATCCAGTCCC |
| | | (SEQ ID NO: 236) |
| Death receptors and pathway edits | | |
| Tumor necrosis factor ligand superfamily | AAC50332.1 | ACTCCGTCAGCTCGTTAGAA |
| member 10 (TRAIL) | | (SEQ ID NO: 237), |
| | | GTTCATACTCTCTTCGTCAT |
| | | (SEQ ID NO: 238), |
| | | AGAGTAGCAGCTCACATAAC |
| | | (SEQ ID NO: 239) |
| Tumor necrosis factor receptor | AF018657.1 | TTCCAGAGCTCACAACGACC |
| superfamily member 10B (TNFRSF10B) | | (SEQ ID NO: 240), |
| | | ATAGTCCTGTCCATATTTGC |
| | | (SEQ ID NO: 241), |
| | | AGATACTCACGATCTCATTG |
| | | (SEQ ID NO: 242) |
| Tumor necrosis factor receptor | AAC51226.1 | AGGTCAAGGATTGTACGCCC |
| superfamily member 10A (TNFRSF10A) | | (SEQ ID NO: 243), |
| | | GAAGTCCCTGCACCACGACC |
| | | (SEQ ID NO: 244), |
| | | TTTGGTTGTTCCGTTGCTGT |
| | | (SEQ ID NO: 245) |
| Caspase-8 (CASP8) | CAA66853.1 | TGATCGACCCTCCGCCAGAA |
| | | (SEQ ID NO: 246), |
| | | GGGTCGATCATCTATTAATA |
| | | (SEQ ID NO: 247), |
| | | TCCTTTGCGGAATGTAGTCC |
| | | (SEQ ID NO: 248) |
| Caspase-10 (CASP10) | AAC50644.1 | CTATGTATCCTTTCGGCATG |
| | | (SEQ ID NO: 249), |
| | | TCTTCTGCCGTATGATATAG |
| | | (SEQ ID NO: 250), |
| | | GTGAGACATGATCTCCCGAA |
| | | (SEQ ID NO: 251) |
| Caspase-3 (CASP3) | AAA65015.1 | ATGTCGATGCAGCAAACCTC |
| | | (SEQ ID NO: 252), |
| | | ATTATACATAAACCCATCTC |
| | | (SEQ ID NO: 253), |
| | | AATGGACTCTGGAATATCCC |
| | | (SEQ ID NO: 254) |
| Caspase-6 (CASP6) | AAC50168.1 | ATAGAGACAATCTTACCCGC |
| | | (SEQ ID NO: 255), |
| | | AAGATTGTCTCTATCTGCGC |
| | | (SEQ ID NO: 256), |
| | | AAATGTGATTGCCTTCGCCA |
| | | (SEQ ID NO: 257) |
| Caspase-7 (CASP7) | AAC50303.1 | CGTTTGTACCGTCCCTCTTC |
| | | (SEQ ID NO: 258), |
| | | TGCGATCCATCAAGACCACC |
| | | (SEQ ID NO: 259), |
| | | TTGATATTTAGGCTTGCCGA |
| | | (SEQ ID NO: 260) |
| FAS-associated death domain protein | AAA86517.1 | AGTCGTCGACGCGCCGCAGC |
| (FADD) | | (SEQ ID NO: 261), |
| | | AGCGGCCCATCAGGACGCTT |
| | | (SEQ ID NO: 262), |
| | | GCGGCGCGTCGACGACTTCG |
| | | (SEQ ID NO: 263) |
| Tumor necrosis factor receptor | AAA63174.1 | GTGTAACATACCTGGAGGAC |
| superfamily member 6 (FAS) | | (SEQ ID NO: 264), |
| | | TACATCTGCACTTGGTATTC |
| | | (SEQ ID NO: 265), |
| | | CTAAAACTTACTTGGTGCAA |
| | | (SEQ ID NO: 266) |
| IDO | AAA36081.1 | TCTCAACTCTTTCTCGAAGC |
| | | (SEQ ID NO: 267), |
| | | CTGCCTGATCTCATAGAGTC |
| | | (SEQ ID NO: 268), |
| | | CAGATACTTACTCATAAGTC |
| | | (SEQ ID NO: 269) |
| Arginase EIF2AK4 | AAH09350.2 | CGCTGAGAAATGACTGCACG |
| | | (SEQ ID NO: 270), |
| | | CATATACTTCTTCACCAGTT |
| | | (SEQ ID NO: 271), |
| | | ATGTACTCACACATCTGGAT |
| | | (SEQ ID NO: 272) |
| Immunostimulator edits | | |
| OX40 (TRAF2) | BAA14259.1 | ACCGAATGTCCCGCGTGCAA |
| | | (SEQ ID NO: 273), |
| | | GCCTTTGCACGCGGGACATT |
| | | (SEQ ID NO: 274), |
| | | GGGGACCCTGAAAGAATACG |
| | | (SEQ ID NO: 275) |
| CD137 (TNFRSF9) | TNFRSF9 | CCTGCGCTGGAGAAACTATT |
| | | (SEQ ID NO: 276), |
| | | CCTTGTAGTAACTGCCCAGC |
| | | (SEQ ID NO: 277), |
| | | CATAGTAGCCACTCTGTTGC |
| | | (SEQ ID NO: 278) |
| IL2 | CAA25292.1 | CAATATCAACGTAATAGTTC |
| | | (SEQ ID NO: 279), |
| | | GACTTAGTGCAATGCAAGAC |
| | | (SEQ ID NO: 280), |
| | | GATATTGCTGATTAAGTCCC |
| | | (SEQ ID NO: 281) |
| Stimulator of interferon genes protein | AC146648.1 | GCGGGCCGACCGCATTTGGG |
| (STING or TMEM173) | | (SEQ ID NO: 282), |
| | | CATATTACATCGGATATCTG |
| | | (SEQ ID NO: 283), |
| | | ACTCTTCTGCCGGACACTTG |
| | | (SEQ ID NO: 284) |
| Toll like receptor edits | | |
| TLR1 | AAC34137.1 | TTATAGAGGAACCCTACTAA |
| | | (SEQ ID NO: 285), |
| | | TTGTGGGCACCTTACTGAGT |
| | | (SEQ ID NO: 286), |
| | | CGAACACATCGCTGACAACT |
| | | (SEQ ID NO: 287) |
| TLR2 | AAC34377.1 | GTTAACGTTTCCACTTTACC |
| | | (SEQ ID NO: 288), |
| | | TTCCCGCTGAGCCTCGTCCA |
| | | (SEQ ID NO: 289), |
| | | TATCTAATTTATCGTCTTCC |
| | | (SEQ ID NO: 290) |
| TLR3 | AAC34134.1 | TTCGGAGCATCAGTCGTTGA |
| | | (SEQ ID NO: 291), |
| | | TTCAACGACTGATGCTCCGA |
| | | (SEQ ID NO: 292), |
| | | CATGCACTCTGTTTGCGAAG |
| | | (SEQ ID NO: 293) |
| TLR4 | AAC80227.1 | TTCTCCCAGAACCAAACGA |
| | | (SEQ ID NO: 294), |
| | | GATGATGTCTGCCTCGCGCC |
| | | (SEQ ID NO: 295), |
| | | ATGCCCCATCTTCAATTGTC |
| | | (SEQ ID NO: 296) |
| TLR5 | AAC34376.1 | TATTCGGCCATCAAAGGAGC |
| | | (SEQ ID NO: 297), |
| | | GACTAAGCCTCAACTCCAAC |
| | | (SEQ ID NO: 298), |
| | | TATACAACICTATTAGCTGCG |
| | | (SEQ ID NO: 299) |
| TLR6 | BAA78631.1 | GAACTACATCGCTGAGCTIC |
| | | (SEQ ID NO: 300), |
| | | GCCATCCTATTGTGAGTTTC |
| | | (SEQ ID NO: 301), |
| | | TGTCTCCAATTTAACTAACG |
| | | (SEQ ID NO: 302) |
| TLR7 | AAF60188.1 | AAGGAATAGTCACCTCCGTA |
| | | (SEQ ID NO: 303), |
| | | AATGGGGCATTATAACAACG |
| | | (SEQ ID NO: 304), |
| | | GGTGAGGTTCGTGGTGTTCG |
| | | (SEQ ID NO: 305) |
| TLR8 | AAF64061.1 | GTGCAGCAATCGTCGACTAC |
| | | (SEQ ID NO: 306), |
| | | AATCCCGGTATACAATCAAA |
| | | (SEQ ID NO: 307), |
| | | CTCGAGTTGCTTGACTTACG |
| | | (SEQ ID NO: 308) |
| TLR9 | AAF72189.1 | GGCTCACGGCTATTCGGCCG |
| | | (SEQ ID NO: 309), |
| | | GCGTCTCCGTGACAATTACC |
| | | (SEQ ID NO: 310), |
| | | CCGACAGGTCCACGTAGCGC |
| | | (SEQ ID NO: 311) |
| TLR10 | AAK26744.1 | CCCACATTTACGCCTATCCT |
| | | (SEQ ID NO: 312), |
| | | TAACATTAATAGCAGCTCGA |
| | | (SEQ ID NO: 313), |
| | | GACCCCAGCCACAACGACAC |
| | | (SEQ ID NO: 314) |
| Kinase inhibition edits | | |
| Serine/threonine-protein kinase B-raf | AAA35609.2 | CCCCACCAAATTTGTCCAAT |
| (BRAF) | | (SEQ ID NO: 315), |
| | | GAGGCCCTATTGGACAAATT |
| | | (SEQ ID NO: 316), |
| | | GTTGCTCCGTGCCACATCTG |
| | | (SEQ ID NO: 317) |
| Dual specificity mitogen-activated | AAA36318.1 | CCATACTTACTCCGCAGAGC |
| protein kinase kinase 1 (MAP2K or | | (SEQ ID NO: 318), |
| MEK) | | TATGGTGCGTTCTACAGCGA |
| | | (SEQ ID NO: 319), |
| | | CCCGACGGCTCTGCAGTTAA |
| | | (SEQ ID NO: 320) |
| Master regulator edits | | |
| FoxP3 | AAG53607.1 | |
| Cytokine signaling edits (The goal of | | |
| one or both of these edits is to prevent | | |
| or minimize conversion of therapeutic | | |
| Tregs into Th17 cells due to endogeonous | | |
| IL-6. | | |
| Gagliani et al. Nature, 523(7559): | | |
| 221-225 2015, | | |
| Korn et al. Proceedings of the National | | |
| Academy of Sciences, 105(47): 18460- | | |
| 18465, 2008. | | |
| Interleukin-6 receptor subunit alpha | CAA31312.1 | TCGGTGCAGCTCCACGACTC |
| (IL6R) | | (SEQ ID NO: 321), |
| | | AACTATTCATGCTACCGGGC |
| | | (SEQ ID NO: 322), |
| | | CGTGGTGCAGCTTCGTGCCC |
| | | (SEQ ID NO: 323) |
| Interleukin-6 receptor subunit beta | AAA59155.1 | AGATGCCTCAACTTGGAGCC |
| (IL6ST or GP130) | | (SEQ ID NO: 324), |
| | | TTTGAGTTGCATTGTGAACG |
| | | (SEQ ID NO: 325), |
| | | ATTCGCTGTATGAAGGAAGA |
| | | (SEQ ID NO: 326) |
| Cell interaction reducing edits | | |
| TCR alpha see preferred talen edit) | | |
| TCR beta (see preferred talen edit) | | |
| CIITA | CAA52354.1 | TTCCTACACAATGCGTTGCC |
| | | (SEQ ID NO: 327), |
| | | GATATTGGCATAAGCCTCCC |
| | | (SEQ ID NO: 328), |
| | | TCAACTGCGACCAGTTCAGC |
| | | (SEQ ID NO: 329) |
| B7-1(CD80) | AAA36045.1 | TCGTATGTGCCCTCGTCAGA |
| | | (SEQ ID NO: 330), |
| | | GAGTGAATCAGACCTTCAAC |
| | | (SEQ ID NO: 331), |
| | | TATGGCCCGAGTACAAGAAC |
| | | (SEQ ID NO: 332) |
| B7-2(CD86) | AAB03814.1 | GTAACCGTGTATAGATGAGC |
| | | (SEQ ID NO: 333), |
| | | ATACTCGATAGTTGAATTCT |
| | | (SEQ ID NO: 334), |
| | | CATCAGATCTTTCAGGTATA |
| | | (SEQ ID NO: 335) |
| b2m | AAA51811.1 | ACTCACGCTGGATAGCCTCC |
| | | (SEQ ID NO: 336), |
| | | GAGTAGCGCGAGCACAGCTA |
| | | (SEQ ID NO: 337), |
| | | CAGTAAGTCAACTTCAATGT |
| | | (SEQ ID NO: 338) |
| UL18 | CAA68399.1 | |
| PDL2 | AAK31105.1 | |
| FasL | AAC50071.1 | GGTTGTTGCAAGATTGACCC |
| | | (SEQ ID NO: 339), |
| | | GAGGAACTCTAAGTATCCCC |
| | | (SEQ ID NO: 340), |
| | | TCTGGTTGCCTTGGTAGGAT |
| | | (SEQ ID NO: 341) |
| Perforin (PRF1) | CAA31612.1 | CGCAGCCACAAGTTCGTGCC |
| | | (SEQ ID NO: 342), |
| | | GGAGCTGGGTGGCCGCATAT |
| | | (SEQ ID NO: 343), |
| | | CCCGAACAGCAGGTCGTTAA |
| | | (SEQ ID NO: 344) |
| Galectin 9 (LGALS9) | AA88922.1 | |
| PVT/CD155 | AAA36461.1 | |
| Drug interaction related edits | | |
| CD52 | CAA44323.1 | CTCTTACCTGTACCATAACC |
| | | (SEQ ID NO: 345), |
| | | AATGCCTCCGCTTATGTTGC |
| | | (SEQ ID NO: 346), |
| | | TGGCATTGGCCACGAAGAAA |
| | | (SEQ ID NO: 347) |
| tocilizumab-like heavy chain (for scfv) | BAJ21229.1 | |
| tocilizumab-like light chain (for scfv) | BAJ21230.1 | |
| Integrin alpha-4 (ITGA4) | CAA34852.1 | CGACTACTTCGGTAGTATGC |
| | | (SEQ 1D NO: 348), |
| | | CAGCATACTACCGAAGTAGT |
| | | (SEQ ID NO: 349), |
| | | GTGTTTGTGTACATCAACTC |
| | | (SEQ ID NO: 350) |

The table below provides target sequences for TALEN edits as well as protein sequences.

TABLE 7
Primarily TALEN mediated edits

| | Sequence or Genbank Accession no. |
|---|---|
| CS1 edit related | |
| CS1 | NP_067004.3 |
| CS1v1 TALEN target | tgacttccagagag caatatggct ggttccccaa catgcctca (SEQ ID NO: 351) |
| CS1v1 left TALEN target | tgacttccagagagcaa (SEQ ID NO: 352) |
| CS1v1 left TALEN protein | MDYKDHDGDYKDHDIDYKDDDDKMAPKKKRKVGIHGVPAAVDTATLGYSQQQQEKIKPK |
| | VRSTVAQHHEALVGHGFTHAHIVALSQHPAALGTVAVKYQDMIAALPEATHEAIVGVGKQ |
| | WSGARALEALLTVAGE-LRGPPLQLDTGQLLKIAKRGGVTAVEAVHAWRNALTGAPLNLTP |
| | EQVNAIASNNGGKQALETVQRLLPVLCQAHGLTPEQVVAIASNIGGKQALETVQLLPVLC |
| | QAHGLTPEQVVAIASHDGGKQALETVQRLLPVLCQAHGLTPEQVVAIASNGGGKQALETVQ |
| | RLLPVLCQAHGLTPEQVVAIASNGGGKQALETVQRLLPVLCQAHGLTPEQVVAIASHDGGK |
| | QALETQRLLPVLCQAHGLTPEQVVAIASHDGGKQALETVQRLLPVLCQAHGLTPEQVVAI |
| | ASNIGGKQALETVQRLLPVLCQAHGLTEQVVAIASNNGGKQALETVQRLLPVLCQAHGLT |
| | PEQVVAIASNIGGKQALETVQRLLPVLCQAHGLTPEQVVAIASNNGGKQALETVQRLLPVLC |
| | QAHGLTPEQVVAIASNIGGKQALETVQRLLPVLCQAHGLTPEQVVAIASNNGGKQALETVQ |
| | RLLPVLCQAHGLTPEQVVAIASHDGGKQALETVQRELLPVLCQAHGLTPEQVVAIASNIGGKQ |
| | ALETVQRLLPVLCQAHGLTPEQVVAIASNIGGRPALESIVQLSRPDPALAALTNDHLVALA |
| | CLGGRPALDAVKKGLPHAPALIKRTNRRIPERTSHRVAGSQLVKSELEEKKSELRHKLKYVP |
| | HEYIELIEIARNSQDRILEMKVMEFFMKVYGYRGKHLGGSRKPDGAIYTVGSPIDYGVIVDT |
| | KAYSGGYNLPIGQADEMQRYVEENQTRNKHINPNEWWKVYPSSVTEFKFLFVSGHFKGNY |
| | KAQLTRLNHITNCNGAVLSVEELLIGGEMIKAGTLTLEEVRRKFNNGEINFRS (SEQ ID |
| | NO: 353) |
| CS1v1 right TALEN target | aacatgcctc accctca (SEQ ID NO: 354) |
| CS1v1 right TALEN | MDYKDHDGDYKDHDIDYKDDDDKMAPKKKRKVGIHGVPAAVDLRTLGYSQQQQEKIKPK |
| protein | VRSTVAQHHEALVGHGFTHAHIVALSQHPAALGTVAVKYQDMIAALPEATHEAIVGVGKQ |
| | WSGARALEALLTVAGELRGPPLQLDTGQLLKIAKRGGVTAVEAVHAWRNALTGAPLNLTP |
| | EQVNAIASNNGGKQALETVQRLLPVLCQAHGLTPEQVVAIASNIGGKQALETVQRLLPVLC |
| | QNHGLTPEQVVAIASNNGGKQALETVQRLLPVLCQAHGLTPEQVVAIASNNGGKQALETVQ |
| | RLLPVLCQAHGLTPEQVVAIASNNGGKQALETVQRLLPVLCQAHGLTPEQVVAIASNGGGK |
| | QALETVQALLPVLCQAHGLTPEQVVAIASNNGGKQALETVQRLLPVLCQAHGLTPEQVVAI |
| | ASNIGGKQALETNQRLLPVLCQAHGLTPEQVVAIASNNGGKQALETVQRLLPVLCQAHGLT |
| | PEQVVAIASNNGGIKQALETVQRLLPVLCQAHGLTPEQVVAIASHDGGKQALETVQRLLPVL |
| | CQAHGLTPEQVVIVASNIGGKQALETVQRLLPVLCQAHGLTPEQVVAIASNGGGKQALETV |
| | QRLLPVLCQAHGLTPEQVVAIASNNGGKQALETVQRLLPVLCQAHGLTPEQVVAIASNGGG |
| | KQALETVQRLLPVLCQAHGLTPEQVVAIASNGGGRPALESIVAQLSRPDRALAALTNDHLVA |
| | LACLGGRPALDAVKKGLPHAPALIKRTNRRIPERTSHRVAGSQLVKSELEEKKSELRHKLKY |
| | VPHEYIELIEIARNSTQDRILEIMVMEFFMKVYGYRGKHLGGSRKPDGAIYTVGSPIDYGVIV |
| | DTKAYSGGYNLPIGQADEMQRYVEENQTRNKHINPNEWWKVYPSSVTEFKFLFVSGHFKG |
| | NYKAQLTRLNHITNCNGAVLSVEELLIGGEMIKAGTLTLEEVRRKFTNNGEINFRS (SEQ ID |
| | NO: 355) |
| CS1v2 TALEN target | ttccagagag caatatggct ggttccccaa catgcctcac cctcatcta (SEQ ID NO: |
| | 356) |
| CS1v2 left TALEN target | ttccagagag caatatg (SEQ ID NO: 357) |
| CS1v2 left TALEN protein | MDYKLMIDGDYKTYRDIDYKDDDDKMARKKKRKVGIEGVPAAVDERTLGYSQQQQEKIKPK |
| | VRSTVAQHHEALVGHGFTHAHIVALSQHPAALGTVAVKYQDMIAALPEATHEAIVGVGKQ |
| | WSGARALEALLTVAGELRGPPLQLDTGQLLKIAKRGGVTAVEAVHAWRNALTGAPLNLTP |
| | EQVVAIASNGGGKQALETNQRLLPVLCQAHGLTPEQVVAIASHDGGKQALETVQRLLPVLC |
| | QAHGLTPEQVVAIASHDGGKQALETVQRLLPVLCQAHGLTPEQVVAIASNIGGKQALETVQ |
| | RLLPVCQAHGLTPEQVVAIASNNGGKQALETVQRLLPVLCQAHGLTPEQVVAIATASNIGGKQ |
| | ALETVQRLLPVLCQAHGLTPEQVVAIASNNGGKQALETVQRLLPVLCQAHGLTPEQVVAIA |
| | SNIGGKQALETVQRLLPVLCQAHGLTPEQYVAIASNNGGKQALETVQRLLPVLCQAHGLTP |
| | EQVVAIASHDGGKQALETVQRLLPVLCQAHGLTPEQVVAIASNIGGKQALETVQRLLPVLC |
| | QAHGLTPEQVVAIASNIGGKQALETVQRLLPVLCQAHGLTPEQVVAIASNGGGKQALETVQ |
| | RLLPVLCQAHGLTPEQVVAIASNIGGKQALETVQRLLPVLCQAHGLTPEQVVAIASNGGGKQ |
| | ALETVQRLLPVLCQAHGLTPEQVVAIASNNGGKQALETVQRLLPVLCQAHGLTPEQVVALA |
| | CLGGRPALDAVKKGLPHAPALIKRTNRRIPERTSHRVAGSQLVKSELEEKKSELRHKLKYVP |
| | HEYIELIEIARNSTQDRTIEMKVMEFFMKVYGYRGKHLGGSRKPDGAIYTVGSPIDYGVIVDT |
| | KAYSGGYNLPIGQADEMQRYVEENQTRNKHINPNEWWKVVYPSSVTEFKFLFVSGHFKGY |
| | KAQLTRLNHTITNCNGAVLSVEELLIGGEMIKAGTLTLEEVRRKFNNGEINFRS (SEQ ID |
| | NO: 358) |
| CS1v2 right TALEN target | tgcctcaccc tcatcta (SEQ ID NO: 359) |
| CS1v2 right TALEN | MDYKDHDGDYKDHDIDYKDDDDKMAPKKKRKVGIHGVPAAVDLRTLGYSQQQQEKIKPK |
| protein | VRSTVAQHHEALVGHGFTHAHIVALSQHPAALGTVAYKYQDMIAALPEATHEAIVGVGKQ |
| | WSGARALEALLTVAGELRGPPLQLDTGQLLKIAKGGVTAVTAVEAVHAWRNALTGAPLNLTP |
| | EQVVAIASNIGGKQALETVQRLLPVLCQAHGLTPEQVVAIASNNGGKQALETVQRLLPVLC |
| | QAHGLTPEQVVAIASNIGGKQALETVQRLLPVCQAHGLTPEQVVAIASNGGGKQALETVQ |
| | RLLPVLCQAHGLTPEQVVAIASNNGGKQALETVQRLLPVLCQAHGLTPEQVVAIASNIGGKQ |
| | ALETVQRLLPVLCQAHGLTPEQVVAIASNNGGKQALETVQALLPVLCQAHGLTPEQVVAIA |
| | SNNGGKQALETVQRLLPVLCQAHGLTPEQVVAIASNNGGKQALETVQRLLPVLCQAHGLTP |
| | EQVVAIASNGGGKQALETVQRLLPVLCQAHGLTPEQVVAIASNNGGKQALETVQRLLPVLC |
| | QAHGLTPEQVVAIASNIGGKQALETVQRLLPVLCQAHGLTPEQVVAIASNNG |
| | GKQALETVQRLLPVLCQAHGLTPEQVVAIASNNGGKQALETVQRLLPVLCQAHGLTPEQVV |
| | AIASHDGGKQALETVQRLLPVLCQAHGLFPEQVVAIASNIGGRPALESIVAQLSRPDPALAAL |
| | TNDHLVALACLGGRPALDAVKKGLPHAPALIKRTNRRIPERTSHRVAGSQLVKSELEEKKSE |
| | LRHKLKYVPHEYIELIEIARNSTQDRILEMKVMEFFMKVYGYRGKHLGGSRKPDGAIYTVGS |
| | PIDYGVIVDTKAYSGCNLPIGQADEMQRYVEENQTRNKNHINPNEWWKYPSSVTEFKFLF |
| | VSGHFKGNYKAQLTRLNHITNCNGAVLSVEELLIGGEMIKAGTLTLEEVRRKFNNGEINFRS |
| | (SEQ ID NO: 360) |
| CS1v3 target | ttgactctat tgtctgacc ttcaacacaa cccctcttgt caccataca (SEQ ID NO: |
| | 361) |
| CS1v3 left TALEN target | ttgactctat tgtagg (SEQ ID NO: 362) |
| CS1v3 left TALEN protein | MDYKDHDGDYKDHDIDYKDDDDKMAPKKKRKVGIHGVPAAVDLRTLGYSQQQQEKIKPK |
| | VRSTVAQHHEALVGHGFTHAHIVALSQHPAALGTVAVKYQDMIAALPEATHEAIGVGKQ |
| | WSGARALEALLTVAGELRGPPLQLDTGQLLKIAKRGGVTAVEAVHAWRNALTGAPLNLTP |
| | EQVVAIASNGGGKQALETVQRLLPVLCQAHGLTPEQVVAIASNNGGKQALETVQRLLPVLC |
| | QAHGLTPEQVVAIASNIGGKQALETVQRLLPVLCQAHGLTPEQVVAIASHDGGKQALETVQ |
| | RLLPVLCQAHGLTPEQVVAIASNGGGKQALETVQRLLPVLCQAHGLTPEQVVAIASHDGGK |
| | QALETVQRLLPVLCQAHGLTPEQVVAIASNGGGKQALETVQRLLPVLCQAHGLTPEQVVAI |
| | ASNIGGKQALETVQRLLPVLCQAHGLTPEQVVAIASNGGGKQALETVQRLLPVLCQAHGLT |
| | PEQVVAIASNGGGKQALETVQRLLPVLCQAHGLTPEQVVAIASNNGGKQALETVQRLLPVL |
| | CQAHGLTPEQVVIVASNGGGKQALETVQRLLPVLCQAHGLTPEQVVAIASHDGGKQALETV |
| | QRLLPVLCQAHGLTPEQVVAIASNGGGKQALETVQRLLPVLCQAHGLTPEQVVAIASNNGG |
| | KQALETVQRLLPVLCQAHGLTPEQVVIASNNGGRPALESIVAQLSRPDPALAALTNDHLVA |
| | LACLGGRPALDAVKKGLPHAPALIKRTNRRIPERTSHRVAGSQLVKSELEEKKSELRHKLKY |
| | VPHEYIELIEIARNSTQDRILEMKVMEFFMKVYGYRGKHLGGSRKPDGAIYTVGSPIDYGVIV |
| | DTKAYSGGYNLPIGQADEMQRYVEENQTRNKHINPNEWWKVYPSSVTEFKFLFVSGHFKG |
| | NYKAQLTRLNHITNCNGAVLSVEELLIGGEMIKAGTLTLEEVRRKFNNGEINFRS (SEQ ID |
| | NO: 363) |
| CS1v3 right TALEN target | cacttgtca ccataca (SEQ ID NO: 364) |
| Cs1v3 right TALEN | MDYKDHDGDYKDHDIDYKDDDDKMAPKKKRKVGIHGVPAAVDLRTLGYSQQQQEKIKPK |
| protein | VRSTVAQHHEALVGHGFTHAHIVALSQHPAALGTVAVKYQDMIAALPEATHEAIVGVGKQ |
| | WSGARALEALLTVAGELRGPPLQLDTGQLLKIAKRGGVTAVEAVHAWRNALTGAPLNLTP |
| | EQVVAIASNNGGKQALETVQRLLPVLCQAHGLTPEQVVAIASNGGGKQALETVQRLLPVLC |
| | QAHGLTPEQVVAIASNIGGKQALETVQRLLPVLCQAHGLTPEQVVAIASNGGGKQALETVQ |
| | RLLPVLCQAHGLTPEQYVAIASNNGGKQALETVQRLLPVLCQAHGLTPEQVVAIASNNGGK |
| | QALETVQRLLPVLCQAHGLTPEQVVAIASNGGGKQALETVQRLLPVLCQAHGLTPEQVVAI |
| | ASNNGGKQALETVQRLLPVLCQAHGLTPEQVVAIASNIGGKQALETVQRLLPVLCQAHGLT |
| | PEQVVAIASHDGGKQALETVQRLLPVLCQAHGLTPEQVVAIASNIGGKQALETVQRLLPVLC |
| | QAHGLTPEQVVAIASNIGGKQALETVQRLLPVLCQAHGLTPEQVVAIASNNGGKQALETVQ |
| | RLLPVLCQAHGLTPEQVVAIASNIGGKQALETVQRLLPVLCQAHGLTPEQVVAIASNNGGKQ |
| | ALETVQRLLPVLCQAHGLTPEQVVAIASNNGGRPALESIVAQLSRPDPALAALTNDHLVALA |
| | CLGGRPALDAVKKGLPHAPALIKRTNRRIPERTSHRVAGSQLVKSELEEKKSELRLHKLKYVP |
| | HEYIELIEIARNSTQDRILEMKVMEFFMKVYGYRGKHLGGSRKPDGAIYTVGSPIDYGVIVDT |
| | KAYSGGYNLPIGQADEMQRYVEENQTRNKHINPNEWWKVYRSSVTEFKFLFVSGHFKGNY |
| | KAQLTRLNHITNCNGAVLSVEELLIGGEMIKAGTLTLEEVRRKFNNGEINFRS (SEQ ID |
| | NO: 365) |
| CIITA edit related | |
| CIITA | NP_001273331.1 |
| CIITA TALEN target | TTCCCTCCCAGGCAGCTCacagtgtgccaccaTGGAGTTGGGGCCCCT |
| | A (SEQ ID NO: 366) |
| CIITA left TALEN target | TTCCCTCCCAGGCAGCTC (SEQ ID NO: 367) |
| CITTA left TALEN protein | MDYKDHDGDYKDHDIDYKDDDDKMAPKKKRKVGIHGVPAAVDERTLGYSQQQQEKIKPK |
| | VRSTVAQHHEALVGHGFTHAHIVALSQHPAALGTVAVKYQDMIAALPEATHEAWGVGKQ |
| | WSGSRALEALLTVGELRGPPLQLDTGQLLKIAKRGGVTAVEAVHAWRNALTGAPLNLTP |
| | EQVVAIASNGGGKQALETVQRLLPVLCQAHGLTPEQVVAIASHDGGKQALTEVQRLLPVLC |
| | QAHGLTPEQVVAIASHDGGKQALETVQRLLPVLCQAHGLTPEQVVAIASHDGGKQALETVQ |
| | RLLPVLCQAHGLTPEQVVAIANGGGKQALETVQRLLPVLCQAHGLTPEQVVAIASHDGGK |
| | QALETVQRLLPVLCQAHGLTPEQVVAIASHDGGKQALETVQRLLPVLCQAHGLTPEQVVAI |
| | ASHDGGKQALETVQRLLPVLCQAHGLTPEQVVAIASNIGGKQALETVQRLLPVLCQAHGLT |
| | PEQVVAIASNNGGKQALETVQRLLPVLCQAHGLTPEQVVAIASNNGGKQALETVQRLLPVL |
| | CQAHGLTPEQVVAIASHDGGKQALETVQRLLPVLCQAHGLTPEQVVAIASNIGGKQALETV |
| | QRLLPVLCQAHGLTPEQVVAIASSNNGGKQALETVQRLLPVLCQAHGLTPEQVVAIASHDGG |
| | KQALETVQRLLPVLCQAHGLTPEQVVAIASNGGGKQALETVQRLLPVLCQAHGLTPEQVVA |
| | IASHDGGRPALESIVAQLSRPDPALAALTNDHLVALACLGGRPALDAYKKGLPHAPLIKRT |
| | NRRIPERTSHRVAGSQLVKSELEEKKSELRHKLKYVPHEYIELIEIARNSTQDRILEMKVMEFF |
| | MKVYGYRGKHLGGSRKPDGAIYTVGSPIDYGVIVDTKAYSGGYNLPIGQADEMQRYNEEN |
| | QTRNKHINPNEWWKVYPSSVTEFKFLFVSGHFKGNYKAQLTRLNHITNCNGAVLSVEELLIG |
| | GEMIKAGTLTLEEVRRKFNGEINFRS (SEQ ID NO: 368) |
| CIITA right target | TGGAGTTGGGGCCCCTA (SEQ ID NO: 369) |
| CIITA right protein | MDYKDHDGDYKDHDIDYKDDDDKMAPKKKRKVGIHGVPAAVDLRTLGYSQQQQEKIKPK |
| | VRSTVAQHEEALVGHGFTHIAHIVALSQHPAALGTVAVKYQDMIAALPEATHEAIVGVGKQ |
| | WSGARALEALLTVAGELRGPRLQLDTGQLLKIAKRGGVTAVEAVHAWRNALTGAPLNLTP |
| | EQVVAIASNIGGKQALETVQRLLPVLCQAHGLTPEQVVAIASNNGGKQALETVQRLLPVLC |
| | QAHGLTPEQVVAIASNNGGKQALETVQRLLPVLCQAHGLTPEQVVAIASNNGGKQALETVQ |
| | RLLPVLCQAHGLTPEQVVAIASNNGGKQALETVQRLLPVLCQAHGLTPEQVVAIASHDGGK |
| | QALETVQRLLPVLCQAHGLTPEQVVAIASHDGGKQALETVQRLLPVLCQAHGLTPEQVVAI |
| | ASHDGGKQALETVQRLLPVLCQAHGLTPEQVVAILASHDGGKQALETVQRLLPVLCQAHGLT |
| | PEQVVAIASNIGGKQALETVQRLLPVLCQAHGLTPEQVVAIASNIGGKQALETVQRLLPVLC |
| | QAHGLTPEQVVAIASHDGGKQALETVQRLLPVLCQAHGLTPEQVVAIASNGGGKQALETVQ |
| | RLLPVLCQAHGLTPEQVVAIASHDGGKQALETVQRLLPVLCQAHGLTPEQVVAIASHDGGK |
| | QALETVQRLLPVLCQAHGLTPEQVVAIASNIGGRPALESIVAQLSRPDPALAALTNDHLVAL |
| | ACLGGRPALDAVKKGLPHAPALIKRINRKPERTSHRVAGSQLVKSELEEKKSELRHKLKYV |
| | PHEYIELIEIARNSTQDRILEMIKVMEFFMKVYGYRGKHLGGSRKPDGAIYTVGSPIDYGVIVD |
| | TKAYSGGYNLPIGQADEMQRYVEENQTRNKHKNPNEWWKVYPSSVTEFKFLSGHFKGN |
| | YKAQLTRLNHITNCNGAVLSVEELLIGGEMIKAGTLTLEEVRRKFNNGEINFRS (SEQ ID |
| | NO: 370) |
| CD52 edit related | |
| CD52 | NP_001794.2 |
| CD52 target | TTCCTCCTACTCACCATcagcctcctggttatGGTACAGGTAAGAGCAA |
| | (SEQ ID NO: 371) |
| CD52 left target | TTCCTCCTACTCACCAT (SEQ ID NO: 372) |
| CD52 left protein | MDYKDHDGDYKDHDIDYKDDDDKMAPKKKRKVGIHGVPAAVDLRTLGYSQQQQEKIKPK |
| | VRSTVAQHHEALVGHGFTHAHIVALSQHPAALGTVAVKYQDMIAALPEATHAIVGVGKQ |
| | WSGARALEALLTVAGELRGPPLQLDTGQLLKIAKRGGVTAVEAVHAWRNALTGAPLNLTP |
| | EQVVAIASNGGGKQALETVQRLLPVLCQAHGLTPEQVVAIASHDGGKQALETVQRLLPVLC |
| | QAHGLTPEQVVAIAHDGGKQALETVQRLLPVLCQAHGLTPEQVVAIASNGGGKQALETVQ |
| | RLLPVLCQAHGLTPEQVVAIASHDGGKQALETVQRLLPVLCQAHGLTPEQVVAIASHDGGK |
| | QALETVQRLLPVLCQAHGLTPEQVVAIASNGGGKQALETVQRLLPVLCQAHGLTPEQVVAI |
| | ASNIGGKQALETVQRLLPVLCQAHGLTPEQVVAIASHDGGKQALETVQRLLPVLCQAHGLT |
| | PEQVVAIASNGGGKQALETVQRLLPVLCQAHGLTPEQVVAIASHDGGKQALETVQRLLPVL |
| | CQAHGLTPEQVVAIASNIGGKQALETVQRLLPVLCQAHGLTPEQVVAIASHDGGKQALETV |
| | QRLLPVLCQAHGLTPEQVVAIASHDGGKQALETVQRLLPVLCQAHGLTPEQVVAIASNIGGK |
| | QALETVQRLLPVLCQAHGLTPEQVVAIASNGGGRPALESIVAQLSRPDPALAALTNDHLVAL |
| | ACLGGRPALDAVKKGLPHAPALIKRTNRRIPERTSHRVAGSQLVKSELEEKKSELRHKLKYV |
| | PHEYIELEIARNSTQDRILEMKVMEFEMKVYGYRGKHLGGSRKPDGAIYINGSPIDYGVIND |
| | TKAYSGGYNLPIGQADEMQRYVLENQTRNKHINPNEWWKVYPSSVTEFKFLEVSGHFKGN |
| | YKAQLTRLNHITNCNGAVLSVEELLIGGEMIKAGTLTLEEVRRKFNNGEINFRS (SEQ ID |
| | NO: 373) |
| CD52 right target | GGTACAGGTAAGAGCAA (SEQ ID NO: 374) |
| CD52 right protein | MDYKDHDGDYKDHDIDYKDDDDKMAPKKKRKVGIHGVPAAVDLRTLGYSQQQQEKIKPK |
| | VRSTVAQHHEALVGHGFTHAHIVALSQHPAALGTVAVKYQDMIAALPEATHEAIVGVGKQ |
| | WSGARALEALLTVAGELRGPPLQLDTGQLLKIAKRGGVTAVEAVHAWRNALTGAPLNLTP |
| | EQVVAIASNGGGKQALETVQRLLPVLCQAHGLTPEQVVAIASNNGGKQALETVQRLLPVLC |
| | QAHGLTPEQVVAIASHDGGKQALETVQRLLPVLCQAHGLTPEQVVAIASNGGGKQALETVQ |
| | RLLPVLCQAHGLTPEQVVAIASHDGGKQALETVQRLLPVLCQAHGLFPEQVVAIASNGGGK |
| | QALETVQRLLPVLCQAHGLTPEQVVAIASNGGGKQALETVQRLLPVLCQAHGLTPEQVVAI |
| | ASNIGGKQALETVQRLLPVLCQAHGLTPEQVAIASHDGGKQALETVQRLLPVLCQAHGLT |
| | PEQVVAIASHDGGKQALETVQRLLPVLCQAHGLTPEQVVAIASNGGGKQALETVQRLLPVL |
| | CQAHGLTPEQVVAIASNNGGKQALETVQRLLPVLCQAHGLTPEQVVAIASNGGGKQALETV |
| | QRLLPVLCQAHGLTPEQVVAIASNIGGKQALETVQRILLPVLCQAHGLTPEQVVAIASHDGGK |
| | QALETVQRLLPVLCQAHGLTPEQVVAIASHDGGRPALESIVAQLSRPDPALAALTNDHLVAL |
| | ACLGGRPALDAVKKGLPHAPALIKRTNRRIPERTSHRVAGSQLVKSLLEEKKSELRHKLKYV |
| | PHEYIELEIARNSTQDRILEMKVMEFFMKVYGYRGKHLGGSRKPDGAIYTVGSPIDYGVIVD |
| | TKAYSGGYNLPIGQADEMQRYVEENQTRNKHINPNEWWKVYPSSVTEFKFLFVSGHFKGN |
| | YKAQLTRLNHITNCNGAVLSVEELLIGGEMIKAGTLTLEEVRRKFNNGEINFRS (SEQ ID |
| | NO: 375) |
| TRAC edit related | |
| TRAC | CAA26435.1 |
| TRAC TALEN target | TTGTCCCACAGATATCCagaaccctgaccctgCCGTGTACCAGCTGAGA |
| | (SEQ ID NO: 376) |
| TRAC left TALEN target | TTCTTCCCACAGATATCC (SEQ ID NO: 377) |
| TRAC left protein | MDYKDHDGDYKDHDIDYKDDDDKMAPKKKRKVGIHGVPAAVDLRTLGYSQQQQEKIKPK |
| | VRSTVAQHHEALVGHGFTHAHIVALSQHPAALGTVAVKYQDMIAALPEATHEAIVGVGKQ |
| | WSGARALEALLTVAGELRGPPLQLDTGQLLKIAKRGGVTAVEAVHAWRNALTGAPLNLTP |
| | EQVVAIASNGGGKQALETVQRLLPVLCQAHGLTPEQVVAIASNNGGKQALETVQRLLPVLC |
| | QAHGLTPEQVVAIASNGGGKQALETVQRLLPVLCQAHGLTPEQVVAIASHDGGKQALETVQ |
| | RLLPVLCQAHGLTPEQVVAIASHDGGKQALETVQRLLPVLCQAHGLTPEQVVAIASHDGGK |
| | QALETVQRLLPVLCQAHGLTPEQVVAIASNIGGKQALETVQRLLPVLCQAHGLTPEQVVAIA |
| | SHDGGKQALETVQRLLPVLCQAHGLTPEQVVAIASNIGGKQALETVQRLLPVLCQAHGLTP |
| | EQVVAIASNNGGKQALETVQRLLPVLCQAHGLTPEQVVAIASNIGGKQALETVQRLLPVLC |
| | QAHGLTPEQVVAIASNGGGKQALETVQRLLPVLCQAHGLTPEQVVAIASNIGGKQALETVQ |
| | RLLPVLCQAHGLTPEQVVAIASNGGGKQALETVQRELPVLCQAHGLTPEQVVAIASHDGGK |
| | QALETVQRLLPVLCQAHGLTPEQVVAIASHDGGRPALESIVAQLSRPDPALAALTNDHLVAL |
| | ACLGGRPALDAVKKGLHNPALIKRTNRRIPERTSHRVAGSQLVKSELEEKKSELRHKLKYV |
| | PHEYIELIEIARNSTQDRILEMKVMEFFMKVYGYRGKHLGGSRKPDGAIYTVGSPIDYGVIVD |
| | TKAYSGGYNLPIGQADEMQRYVEENQTRNKHINPNEWWKVYPSSVTEFKFLFVSGHFKGN |
| | YKAQLTRLNTNUGAVLSVEELLIGGEMIKAGTLTLEEVRRKFNNGEINFRS (SEQ ID |
| | NO: 378) |
| TRAC right TALEN target | CCGTGTACCAGCTGAGA (SEQ ID NO: 379) |
| TRAC TALEN right | MDYKDHDGDYKDRDIDYKDDDDKMAPKKKRKVGIHGVPAAVDLRELGYSQQQQEKIKPK |
| | VRSTVAQHEEALVGHGFTHAHIVALSQHPAALGTVAVKYQDMIAALPEATHEAIVGVGKQ |
| | WSGARALEALLTVAGELRGPPLQLDTGQLLKIAKRGGVTAVEAVHAWRNALTGAPLNTP |
| | EQVVAIASHDGGKQALETVQRLLVVLCQAHGLITEQVVATASNGGGKQALETVQRLLPVLC |
| | QAHGLTPEQVVAIASHDGGKQALETVQRLLPVLCQAHGLTPEQVVAIASNIGGKQALETVQ |
| | RLLPVLCQAHGLTPEQVVAIASNNGGKQALETVQRLLPVLCQAHGLTPEQVVAIASHDGGK |
| | QALETVQRLLPVLCQAHGLTPEQVVAIASNGGGKQALETVQRLLPVLCQAHGLTPEQVAAI |
| | ASNNGGKQALETVQRLLPVLCQAHGLTPEQVVAIASNNGGKQALETVQRLLPVLCQAHGLT |
| | PEQVVAIASNGGGKQALETVQRLLPVLCQAHGLTPEQVVAIASNIGGKQALETVQRLLPVLC |
| | QAHGLTPEQVVAIASHDGGKQALETVQRLLPVLCQAHGLTPEQVVAIASNIGGKQALETVQ |
| | RLLPVLCQAHGLTPEQVVAIASHDGGKQALETVQRELLPVLCQAHGLTPEQVVAIASNNGGK |
| | QALETVQRLLPVLQAHGLTPEQVVAIASNNGGRPALESIVAQLSRPDPALAALTNDHLVAL |
| | ACLGGRPALDAVKKGLPHAPALIKRTNRRIPERTSHRVAGSQLVKSELEEKKSELRHKLKYV |
| | PHEYIELIEIARNSTQDRILEMKVMEFFMKVYGYRGKHLGGSRKPDGAINTVGSPIDYGVIVD |
| | TKAYSGGYNLPIGQADEMQRYVEENQTRNKHINPNEWWKVYPSSVTEFKFLFVSGHFKGN |
| | YKAQLTRLNHITNCNGAVLSVEELLIGGEMIKAGTLTLEEVRRKFNNGEINFRS (SEQ ID |
| | NO: 380) |
| PDCD1 edit related | |
| PDCD1 | NG_012110:1 |
| PDCD1 talen target | ttctccccag ccctgctcgt ggtgaccgaa ggggacaacg ccaccttca (SEQ ID |
| | NO: 381) |
| PDCD1 talen left | MGDPKKKRKVIDYPYDVPDYAIDIADLRTLGYSQQQQEKIKPKVRSTVAQHHEALVGHGFT |
| | HAHIVALSQHPAALGTVAVKYQDMIAALPEATHEAIVGVGKQWSGARALEALLTVAGELR |
| | GPPLQLDTGQLLKIAKRGGVTAVEAVHAWRNALTGARLNLTPQQVVAIASNGGGKQALET |
| | VQRLLPVLCQAHGLTPEQVVAIASHDGGKQALETVQRLLPVLCQAGLTPQQVVAIASNGG |
| | GKQALETVQRLLPVLCQAHGLTEQVVAIASHDGGKQALETVQRLLPVLCQAHGLTPEQVV |
| | AIASHDGGKQALETVQRLLPVLCQAHGLTPEQVVAIASHDGGKQALETVQRLLPVLCQAHG |
| | LTPEQVVAIASHDGGKQALETVQRLLPVLCQAHGLTPEQVVAIASNIGGKQALETVQALLTV |
| | LCQAHGLTPQQVVAIASNNGGKQALETVQRLLPVLCQAHGLTPEQVVAIASHDGGKQALET |
| | VQRLLPVLCQAHGLTPEQVVAIASHDGGKQALETVQRLLPVLCQAHGLTPEQVVAIASHDG |
| | GKQALETVQRLLPVLCQAHGLTPQQVVAIASNGGGKQALETVQRLLPVLCQAHGLTPQQV |
| | VAIASNNGGKQALETVQRLLPVLCQAHGLTPEQVVAIASHDGGKQALETVQRLLPVLCQAH |
| | GLTPQQVVAIASNGGGRPALESIVAQLRPDPALAALTNDHLVALACLGGRPALDAVKKGL |
| | GDPISRSQLVKSELEEKKSELRHKLKYVPHEYIELIEIARNSTQDRILEMKVMEFFMKVYGYR |
| | GKHLGGSRKPDGAIYTVGSPIDYGVIVDTKAYSGGYNLPIGQADEMQRYVEENQTRNKHIN |
| | PNEWWKVYPSSVTEFKFLFVSGHFKGNYKAQLTRLNHITNCNGAVLSVEELLIGGEMIKAG |
| | TLTLEEVRRKFNNGEINFAAD (SEQ ID NO: 382) |
| PDCD1 talen right | MGDPKKKRKVIDKETAAAKFERQHMDSIDIADLRTLGYSQQQQEKIKPKVRSTVAQHHEAL |
| | VGHGFTHAHIVALSQHPAALGTVAVKYQDMIAALPEATHEAIVGVGKQWSGARALEALLT |
| | VAGELRGPPLQLDTGQLLKIAKRGGVTAVEAVHAWRNALTGAPLNLTPEQVVAIASHDGG |
| | KQALETVQRLLPVLCQAHGLTPQQVVAIASNGGGKQALETVQRLLPVLCQAHGLTPEQVVA |
| | IASHDGGKQALETVQRLLPVLCQAHGLTPQQVVALASNGGGKQALETVQRLLPVLCQAHGL |
| | TPQQVVAIASNGGGKQALETVQRLLPVLCQAHGLTPQQAVVAIASNGGGKQALETVQRLLPV |
| | LCQAHGLTPQQVVAIASNNGGKQALETVQRLLPVLCQAHGLTPEQVVAIASNIGGKQALET |
| | VQALLPVLCQAHGLTPQQVVVAIASNGGGKQALETVQRLLPVLCQAHGLTPEQVVAIASHDG |
| | GKQALETVQRLLPVLCQAHGLTPQQVVAIASNGGGKQALETVQRLLPVLCQAHGLTPQQV |
| | VAIASNNGGKQALETVQRLLPVLCQAHGLTPEQVVAIASNGGKQALETVQRLLPVLCQAHG |
| | LTPQQVVAIASNNGGKQALETVQRLLPVLCQAHGLTPEQVVAIASHDGGKQALETVQRLLP |
| | VLCQAHGLTPQQVVAIASNGGGRPALESIVAQLSRPDPALAALTNDHLVALACLGGRPALD |
| | AVKKGLGDPISRSQLVKSELEEKKSELRHKLKYVPHEYIELIEIARNSTQDRILEMKVMEFFM |
| | KVYTYRGKHLGGSRKPDGAIYTVGSPIDYGVIVDTKAYSGGYNLPIGQADEMQRYVEENQT |
| | RNKHINPNEWWKVYPSSVTEFKFLFVSGHFKGNYKAQLTRLNHITNCNGAVLVEELLIGGE |
| | MIKAGTLTLEEVRRKFNNGEINFAAD (SEQ ID NO: 383) |

(iii) Exemplary Genetic Modification Approaches

Any conventional genetic modification approaches can be used to genetically modify the immune cells in a manner as described herein. In some embodiments, the genetic modification is performed using genome editing. “Genome editing” refers to a method of modifying the genome, including any protein-coding or non-coding nucleotide sequence, of an organism to knock out the expression of a target gene. In general, genome editing methods involve use of an endonuclease that is capable of cleaving the nucleic acid of the genome, for example at a targeted nucleotide sequence. Repair of the double-stranded breaks in the genome may be repaired introducing mutations and/or exogenous nucleic acid may be inserted into the targeted site.

Genome editing methods are generally classified based on the type of endonuclease that is involved in generating double stranded breaks in the target nucleic acid. These methods include use of zinc finger nucleases (ZFN), transcription activator-like effector-based nuclease (TALEN), meganucleases, and CRISPR/Cas systems.

In some instances, genetic modification of the immune cells as described herein is performed using the TALEN technology known in the art. TALENs are engineered restriction enzymes that can specifically bind and cleave a desired target DNA molecule. A TALEN typically contains a Transcriptional Activator-Like Effector (TALE) DNA-binding domain fused to a DNA cleavage domain. The DNA binding domain may contain a highly conserved 33-34 amino acid sequence with a divergent 2 amino acid RVD (repeat variable dipeptide motif) at positions 12 and 13. The RVD motif determines binding specificity to a nucleic acid sequence and can be engineered according to methods known to those of skill in the art to specifically bind a desired DNA sequence (see, e.g., Juillerat, et al. (January 2015). Scientific reports, 5; Miller et. al. (February 2011). Nature Biotechnology 29 (2): 143-8; Zhang et. al. (February 2011). Nature Biotechnology 29 (2): 149-53; Geipler, et al., Boch, (2011). PLoS ONE 6 (5): e19509; Boch (February 2011). Nature Biotechnology 29 (2): 135-6; Boch, et. al. (December 2009). Science 326 (5959): 1509-12; and Moscou et al, (December 2009). Science 326 (5959): 1501. The DNA cleavage domain may be derived from the FokI endonuclease, which is active in many different cell types. The FokI domain functions as a dimer, requiring two constructs with unique DNA binding domains for sites in the target genome with proper orientation and spacing. Both the number of amino acid residues between the TALE DNA binding domain and the FokI cleavage domain and the number of bases between the two individual TALEN binding sites appear to be important parameters for achieving high levels of activity. Miller et al. (2011) Nature Biotech. 29: 143-8.

TALENs specific to sequences in a target gene of interest (e.g., TCR, CD52, MHC, and others described herein) can be constructed using any method known in the art, including various schemes using modular components. Zhang et al. (2011) Nature Biotech. 29: 149-53: Geibler et al. (2011) PLoS ONE 6: e19509.

A TALEN specific to a target gene of interest can be used inside a cell to produce a double-stranded break (DSB). A mutation can be introduced at the break site if the repair mechanisms improperly repair the break via non-homologous end joining. For example, improper repair may introduce a frame shift mutation. Alternatively, a foreign DNA molecule having a desired sequence can be introduced into the cell along with the TALEN. Depending on the sequence of the foreign DNA and chromosomal sequence, this process can be used to correct a defect or introduce a DNA fragment into a target gene of interest, or introduce such a defect into the endogenous gene, thus decreasing expression of the target gene.

In some instances, genetic modification of the immune cells as described herin is performed using CRISPR technology as known in the art (CRISPR/Cas systems). Such modification may include the deletion or mutation of a sequence in a target gene of interest can be constructed using a CRISPR-Cas system, where the Clustered Regularly Interspaced Short Palindromic Repeats (CRISPR)-Cas system is an engineered, non-naturally occurring CRISPR-Cas system. The present disclosure utilizes the CRISPR/Cas system that hybridizes with a target sequence in a target gene of interest, where the CRISPR/Cas system comprises a Cas endonuclease and an engineered crRNA/tracrRNA (or single guide RNA (“sgRNA”). In some embodiments, the CRISPR-Cas system includes a crRNA and does not include a tracrRNA sequence. CRISPR/Cas complex can bind to the lineage specific protein polynucleotide and allow the cleavage of the protein polynucleotide, thereby modifying the polynucleotide.

The CRISPR/Cas system of the present disclosure may bind to and/or cleave the region of interest within a target gene of interest, within or adjacent to the gene, such as, for example, a leader sequence, trailer sequence or intron, or within a non-transcribed region, either upstream or downstream of the coding region. The guide RNAs (gRNAs) used in the present disclosure may be designed such that the gRNA directs binding of the Cas enzyme-gRNA complexes to a pre-determined cleavage sites (target site) in a genome. The cleavage sites may be chosen so as to release a fragment that contains a region of unknown sequence, or a region containing a SNP, nucleotide insertion, nucleotide deletion, rearrangement, etc. Cleavage of a gene region may comprise cleaving one or two strands at the location of the target sequence by the Cas enzyme. In one embodiment, such, cleavage can result in decreased transcription of a target gene. In another embodiment, the cleavage can further comprise repairing the cleaved target polynucleotide by homologous recombination with an exogenous template polynucleotide, wherein the repair results in an insertion, deletion, or substitution of one or more nucleotides of the target polynucleotide.

The terms “gRNA,” “guide RNA” and “CRISPR guide sequence” may be used interchangeably throughout and refer to a nucleic acid comprising a sequence that determines the specificity of a Cas DNA binding protein of a CRISPR/Cas system. A gRNA hybridizes to (complementary to, partially or completely) a target nucleic acid sequence in the genome of a host cell. The gRNA or portion thereof that hybridizes to the target nucleic acid may be between 15-25 nucleotides, 18-22 nucleotides, or 19-21 nucleotides in length. In some embodiments, the gRNA sequence that hybridizes to the target nucleic acid is 15, 16, 17, 18, 19, 20, 21, 22, 23, 24, or 25 nucleotides in length. In some embodiments, the gRNA sequence that hybridizes to the target nucleic acid is between 10-30, or between 15-25, nucleotides in length.

In addition to a sequence that binds to a target nucleic acid, in some embodiments, the gRNA also comprises a scaffold sequence. Expression of a gRNA encoding both a sequence complementary to a target nucleic acid and scaffold sequence has the dual function of both binding (hybridizing) to the target nucleic acid and recruiting the endonuclease to the target nucleic acid, which may result in site-specific CRISPR activity. In some embodiments, such a chimeric gRNA may be referred to as a single guide RNA (sgRNA).

As used herein, a “scaffold sequence,” also referred to as a tracrRNA, refers to a nucleic acid sequence that recruits a Cas endonuclease to a target nucleic acid bound (hybridized) to a complementary gRNA sequence. Any scaffold sequence that comprises at least one stem loop structure and recruits an endonuclease may be used in the genetic elements and vectors described herein. Exemplary scaffold sequences will be evident to one of skill in the art and can be found, for example, in Jinek, et al. Science (2012) 337(6096):816-821, Ran, et al. Nature Protocols (2013) 8:2281-2308, PCT Application No. WO2014/093694, and PCT Application No. WO2013/176772. In some embodiments, the CRISPR-Cas system does not include a tracrRNA sequence.

In some embodiments, the gRNA sequence does not comprise a scaffold sequence and a scaffold sequence is expressed as a separate transcript. In such embodiments, the gRNA sequence further comprises an additional sequence that is complementary to a portion of the scaffold sequence and functions to bind (hybridize) the scaffold sequence and recruit the endonuclease to the target nucleic acid.

In some embodiments, the gRNA sequence is at least 50%, 55%, 60%, 65%. 70%, 75%, 80%, 85%, 900%, 95%, 96%, 97%, 98%, 99%, or at least 100% complementary to a target nucleic acid (see also U.S. Pat. No. 8,697,359, which is incorporated by reference for its teaching of complementarity of a gRNA sequence with a target polynucleotide sequence). It has been demonstrated that mismatches between a CRISPR guide sequence and the target nucleic acid near the 3′ end of the target nucleic acid may abolish nuclease cleavage activity (Upadhyay, et al. Genes Genome Genetics (2013) 3(12):2233-2238). In some embodiments, the gRNA sequence is at least 50%, 55%, 60%, 65%, 70%, 75%, 80%, 85%. 90%, 95%, 96%, 97%, 98%, 990%, or at least 1000% complementary to the 3′ end of the target nucleic acid (e.g., the last 5, 6, 7, 8, 9, or 10 nucleotides of the 3′ end of the target nucleic acid).

Example sgRNA sequences, including both modified and unmodified sgRNAs, targeting the T cell receptor alpha constant (TRAC) gene are provided herein. As will be evident to one of ordinary skill in the art, selection of sgRNA sequences may depend on factors such as the number of predicted on-target and/or off-target binding sites. In some embodiments, the sgRNA sequence is selected to maximize potential on-target and minimize potential off-target sites.

In some embodiments, the Cas endonuclease is a Cas9 nuclease (or variant thereof) or a Cpf1 nuclease (or variant thereof). Cas9 endonucleases cleave double stranded DNA of a target nucleic acid resulting in blunt ends, whereas cleavage with Cpf1 nucleases results in staggered ends of the nucleic acid.

In general, the target nucleic acid is flanked on the 3′ side or 5′ side by a protospacer adjacent motif (PAM) that may interact with the endonuclease and be further involved in targeting the endonuclease activity to the target nucleic acid. It is generally thought that the PAM sequence flanking the target nucleic acid depends on the endonuclease and the source from which the endonuclease is derived. For example, for Cas9 endonucleases that are derived from Streptococcus pyogenes, the PAM sequence is NGG, although the PAM sequences NAG and NGA may be recognized with lower efficiency. For Cas9 endonucleases derived from Staphylococcus aureus, the PAM sequence is NNGRRT. For Cas9 endonucleases that are derived from Neisseria meningitidis, the PAM sequence is NNNNGATT. Cas9 endonucleases derived from Streptococcus thermophilus, StlCas9 an dSt3Cas9, the PAM sequences are NNAGAAW and NGGNG, respectively. For Cas9 endonuclease derived from Treponema denticola, the PAM sequence is NAAAAC. In some embodiments, the Cas endonuclease is a Cpf1 nuclease. In contrast to Cas9 endonucleases, Cpf1 endonuclease generally do not require a tracrRNA sequence and recognize a PAM sequence located at the 5′ end of the target nucleic acid. For a Cpf1 nuclease, the PAM sequence is TYTN. In some embodiments, the Cas endonuclease is MAD7 (also referred to as Cpf1 nuclease from Eubacterium rectale) and the PAM sequence is YTiTN.

In some embodiments, genetically engineering a cell also comprises introducing a Cas endonuclease, or nucleic acid sequence encoding such (e.g., mRNA encoding a Cas endonuclease), into the cell. In some embodiments, the Cas endonuclease and the nucleic acid encoding the gRNA are provided on the same nucleic acid (e.g., a vector). In some embodiments, the Cas endonuclease and the nucleic acid encoding the gRNA are provided on different nucleic acids (e.g., different vectors). In some embodiments, the Cas endonuclease is provided as an mRNA encoding the Cas endonuclease and the gRNA is provided as a modified gRNA molecule. Alternatively or in addition, the Cas endonuclease may be provided or introduced into the cell in protein form.

In some embodiments, the Cas endonuclease is a Cas9 enzyme or variant thereof. In some embodiments, the Cas9 endonuclease is derived from Streptococcus pyogenes, Staphylococcus aureus, Neisseria meningitidis, Streptococcus thermophilus, or Treponema denticola. In some embodiments, the nucleotide sequence encoding the Cas endonuclease may be codon optimized for expression in a host cell. In some embodiments, the endonuclease is a Cas9 homolog or ortholog.

In some embodiments, the nucleotide sequence encoding the Cas9 endonuclease is further modified to alter the activity of the protein. In some embodiments, the Cas9 endonuclease has been modified to inactivate one or more catalytic resides of the endonuclease. In some embodiments, the Cas9 endonuclease has been modified to inactivate one of the catalytic residues of the endonuclease, referred to as a “nickase” or “Cas9n”. Cas9 nickase endonucleases cleave one DNA strand of the target nucleic acid. In some embodiments, the methods described herein involve two distinct cleavage reactions, in which one Cas9 nickase is directed to cleave one DNA strand of the target nucleic acid and a Cas9 nickase is directed to cleave the second DNA strand of the target nucleic acid.

(iv) MHC-CAR Regulatory T cells (Treg)

Any of the MHC-CAR-expressing T cells disclosed herein can be regulatory T cells (Treg), which may mimic the immune modulation activity of follicular regulatory cells. As used herein, regulatory T cells or Treg cells, which are also known as suppressor T cells, refer to a subpopulation of T cells that modulate the immune system, maintain tolerance to self-antigens, and/or prevent autoimmune disease. Treg cells function as immunesuppressors to suppress or downregulate induction and/or proliferation of effector T cells, such as pathologic CD4+ and/or CD8+cells involved in autoimmune diseases.

The genetically modified Treg cells disclosed herein express one or more of the biomarkers associated with Treg cells in nature, for example, CD4, FOXP3, CD25, CD45R (e.g., CD45RA or CD45RO), or a combination thereof. The Treg cells may be prepared using (derived from) peripheral blood mononuclear cells (PBMCs) isolated from a suitable donor (e.g., the human patient subject to the treatment). Methods for isolating the subpopulation of Treg cells from PBMCs are well known in the art, for example, cell sorting. Expression vectors for a suitable MHC-CAR construct, as well as other genetic modification (e.g., those described herein) can be introduced into the Treg subpopulation via methods as described herein, or other methods known in the art. Alternatively, the genetically modified Treg cells may be prepared by introducing a transgene coding for CD25 and/or other Treg cell markers into suitable T cells, which can be further modified to introduce the expression cassette for the MHC-CAR and optionally other genetic modification as described herein.

In some embodiments, the genetically modified Treg cells may be further modified to display (e.g., surface express or surface attach) molecules targeting a specific type of pathologic cells (e.g., CD4+cells or CD8+cells) and/or display molecules targeting a specific tissue site (e.g., lymph node or an inflammation site).

In some examples, the genetically modified Treg cells further express a chimeric receptor (CAR) comprising an extracellular domain such as a single-chain antibody (scFv) specific to a B cell surface marker, for example, CD19. Alternatively or in addition, the Treg cells may further express a chimeric receptor comprising an extracellular domain (e.g., scFv) specific to a T cell surface marker, for example, CS-1. Such a chimeric receptor can be a cell-surface receptor comprising an extracellular domain, a transmembrane domain, and a cytoplasmic domain (e.g., comprising a co-stimulatory domain, a cytoplasmic signaling domain such as CD3ζ, or a combination thereof) in a combination that is not naturally found together on a single protein.

The Treg cell may further display a molecule targeting lymph nodes and/or germinal center, for example, CXCR5, and/or display a molecule targeting an inflammation site, for example, CCR6. Targeting germinal center B cells (GC B cells) may be mediated, at least in part, by a specialized helper T cell subset, the CXCR5highPD-lhigh T follicular helper (TFH) cells. Foxp3+Treg can be diverted to become TFH repressors via expression of Bcl6 and SAP-mediated interaction with B cells. The resulting follicular regulatory T cells (TFR) are expected to share features of both TFH and Treg cells, localize to germinal centers, and regulate the size of the TFH cell population and germinal centers in vivo.

Further, the Treg cells disclosed herein may include one or more of the additional genetic modification as described herein, for example, checkpoint molecule knock out.

The Treg cells expressing B-cell or T-cell specific CAR may target pathologic B cells and/or T cells involved in an autoimmune disease. For example, the genetically modified Treg cells as described herein would be expected to exhibit functions similar to follicular regulatory cells, e.g., targeting B cells, T cells, and/or dendritic cells, thereby. e.g., downregulating B cell stimulation, secreting suppressive cytokines that can inhibit activation of germinal center (GCB cells (such as I1-10 and TGF-Beta), inducing cytolysis of Tfh (through MHC CAR) and GC) B (e.g., through the CD19 CAR), and/or mechanical disrupting signaling transduction to GC B cells or to T follicular helper (Tfh) cells (e.g., through binding to GC B and MHC-peptide Tfh). Alternatively or in addition, the Treg cells may potentially engage both helper T cells, B cells, and/or antigen presenting cells, or in some instances, physically blocking the engagement.

### III. Application of Immune Cells Expressing MHC-CAR in Immunotherapy

Host immune cells expressing MHC-CAR (the encoding nucleic acids or vectors comprising such) described herein are useful for targeting and eliminating pathogenic cells involved in autoimmune diseases, such as MS, type I diabetes, lupus, rheumatoid arthritis, etc. In some embodiments, the subject is a mammal, such as a human, monkey, mouse, rabbit, or domestic mammal. In some embodiments, the subject is a human, for example, a human patient having, suspected of having, or at risk for an autoimmune disease (e.g., MS).

The MHC-CAR-expressing immune cells can be mixed with a pharmaceutically acceptable carrier to form a pharmaceutical composition, which is also within the scope of the present disclosure. To perform the methods described herein, an effective amount of the immune cells expressing any of the MHC-CAR constructs described herein can be administered into a subject in need of the treatment. The immune cells may be autologous to the subject, i.e., the immune cells are obtained from the subject in need of the treatment, genetically engineered for expression of the MHC-CAR constructs and optionally contains one or more of the additional genetic modifications as described herein, and then administered to the same subject. Administration of autologous cells to a subject may result in reduced rejection of the immune cells as compared to administration of non-autologous cells. Alternatively, the immune cells are allogeneic cells, i.e., the cells are obtained from a first subject, genetically engineered for expression of the MHC-CAR construct, and administered to a second subject that is different from the first subject but of the same species. For example, allogeneic immune cells may be derived from a human donor and administered to a human recipient who is different from the donor.

In some embodiments, the immune cells are co-used with a therapeutic agent for the target immune disease, for example, Alemtuzumab for treating MS. Such immunotherapy is used to treat, alleviate, or reduce the symptoms of the target immune disease for which the immunotherapy is considered useful in a subject.

The efficacy of the MHC-CAR immunotherapy may be assessed by any method known in the art and would be evident to a skilled medical professional. For example, the efficacy of the immunotherapy may be assessed by survival of the subject and/or reduction of disease symptoms in the subject.

In some embodiments, the immune cells expressing any of the MHC-CAR disclosed herein are administered to a subject who has been treated or is being treated with a therapeutic agent for an autoimmune disease. The immune cells expressing any one of the MHC-CAR disclosed herein may be co-administered with the therapeutic agent. For example, the immune cells may be administered to a human subject simultaneously with the therapeutic agent. Alternatively, the immune cells may be administered to a human subject during the course of a treatment involving the therapeutic agent. In some examples, the immune cells and the therapeutic agent can be administered to a human subject at least 4 hours apart, e.g., at least 12 hours apart, at least 1 day apart, at least 3 days apart, at least one week apart, at least two weeks apart, or at least one month apart.

To practice the method disclosed herein, an effective amount of the immune cells expressing MHC-CAR or compositions thereof can be administered to a subject (e.g., a human MS patient) in need of the treatment via a suitable route, such as intravenous administration. Any of the immune cells expressing MHC-CAR or compositions thereof may be administered to a subject in an effective amount. As used herein, an effective amount refers to the amount of the respective agent (e.g., the immune cells expressing MHC-CAR or compositions thereof) that upon administration confers a therapeutic effect on the subject. Determination of whether an amount of the cells or compositions described herein achieved the therapeutic effect would be evident to one of skill in the art. Effective amounts varv, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human. In some embodiments, the subject is a human cancer patient.

In some embodiments, the subject is a human patient suffering from an autoimmune disease, which is characterized by abnormal immune responses attacking a normal body part. Examples of autoimmune diseases include multiple sclerosis, systemic lupus erythematosus, rheumatoid arthritis, juvenile idiopathic arthritis (also known as juvenile idiopathic arthritis), Sjögren's syndrome, systemic sclerosis, ankylosing spondylitis, Type 1 diabetes, autoimmune thyroid diseases (Grave's and Hashimoto's), multiple sclerosis myasthenia gravis, inflammatory bowel disease (Crohn's or ulcerative colitis), Psoriasis, or a diseases mentioned in Table 1.

There are numerous stages in the immune cascade where, in general, an autoimmune disease can be impacted. There is a continuum of interventions made possible by the combination of edits that the methods disclosed herein comprises. For example, Treg cells displaying a distinct set of surface molecules, in addition to the MHC-CAR, can be used for treating the autoimmune disease at different stages.

In the early stage of many immune disorders, including MS, there exist unexplained deficits in regulatory mechanisms and/or tolerance induction exists in MS and there begin repeated attacks on the nervous system by T cells. Treg cells expressing a suitable MHC-CAR and anti-CD19 CAR (optionally with other genetic modifications as described herein) may be used for intervention.

An advantage of autoreactive Treg cells is their ability to act as “bystander” suppressors, to dampen inflammation at a site-specific manner in response to cognate antigen expressed locally by affected tissues. The induction of regulatory T cells (by autoantigens) can suppress disease progression even when there are a variety of autoantigens (or when the initiating/primary) autoantigen is unknown. Tregs can travel relatively freely, and inhibit T cells and B cells and prevent return to an inflammatory environment. These autoreactive Tregs are advantageous in their ability to act as bystander suppressors and dampen inflammation in a site-specific manner in response to cognate antigen expressed locally by affected tissues.

Thus, the genetically modified Treg cells may be designed to mimic the suppressor function of the autoreactive Treg cells. Such Treg cells may be modified, for example, to have PD-L1/PD-1 knocked out, to display CCR6 and/or scFv targeting MOG to route to the site of inflammation, to express a suitable MHC-CAR and/or anti-CD19-CAR. Alternatively, the Treg cells may be modified, for example, to have PD-L1/PD-1 knocked out, to display CXCR5 to route to germinal centers and/or ectopic lymph nodes, to express a suitable MHC-CAR and/or anti-CD19-CAR. These types of Treg cells may interact with pathogenic cells at the site of inflammation, block pathogenic interactions, and/or calm inflammatory environment. They can be used at an early disease stage (to inhibit pathogenicity) or after cytotoxic therapy (to prevent return to an inflammatory environment).

Relapsing-remitting MS (mid-stage) naturally regulates itself, and treatments which augment these natural regulatory mechanisms will help control the disease process. In successful disease treatment, there is a shift from Th1 cells to Th2 and Th3 cells, and the appearance of other regulatory cells. At this stage, therapeutic targets will include both pathogenic B and pathogenic T cells. Treg cells for treating such mid-stage disease may express a suitable MHC-CAR as described herein, and an additional CAR targeting B cells (e.g., an anti-CD19 CAR) or targeting T cells such as CD8+cells. The Treg cells may further display CXCR5 or free of CXCR5 targeting. Treg cells expressing anti-CD19 CAR may be used to eliminate B cells in the germinal center.

When MS changes from relapsing remitting to the chronic progressive form (late-stage), T cells enter a state of chronic activation and degenerative processes occur. Aggressive treatment against cytotoxic CD8+ T cells requires a CAR augmentation that is sufficiently cytotoxic. At this point, a treatment may shift from one primarily driven by Treg cells to one driven by MHC-CAR CD8+ T cells and even MHC-CAR CS-1 cells. The ultimate goal remains the same: to suppress pathology through cytotoxicity enhanced by bystander effect. Genetically engineered T cells for use at this disease stage may express a suitable MHC-CAR, and an additional CAR targeting pathologic T cells involved in the late stage of the disease, for example, CD8+ T cells. In some examples, the additional CAR may target CS-1 (also known as SLAMF7), which is a glycoprotein expressed on CD8+ T cells. CS1 is a promising antigen that can be used to target and kill CD8+ T cells and plasma cells. CS1-CAR T cells secrete more IFN-gamma as well as IL-2, expressing higher levels of activation marker CD69, higher capacity for degranulation, and display enhanced cytotoxicity. Anti-CS1 CAR will target CD8+ T-cells. The genetically modified T cells may further display a molecule for bone-marrow targeting of plasma cells, such as CXCR4, and their targeting to inflamed tissues, with CXCR3.

Hiepe et al., Nature Reviews Rheumatology, 7(3):170-178, 2011. Examples include targeting of plasma cells in lupus.

### IV. Kits for Therapeutic Uses

The present disclosure also provides kits for use of the MHC-CAR-expressing immune cells for use in suppressing pathogenic immune cells such as autoreactive T cells in autoimmunity. Such kits may include one or more containers comprising compositions comprising immune cells expressing MAR-CAR such as those described herein), and a pharmaceutically acceptable carrier.

In some embodiments, the kit can comprise instructions for use in any of the methods described herein. The included instructions can comprise a description of administration of the MHC-CAR-expressing immune cells to a subject who needs the treatment, e.g., an MS patient. The kit may further comprise a description of selecting a subject suitable for treatment based on identifying whether the subject is in need of the treatment. In some embodiments, the instructions comprise a description of administering the immune cells to a subject who is in need of the treatment.

The instructions relating to the use of the immune cells expressing the MHC-CAR described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device. A kit may have a sterile access port (for example, the container may be an intravenous solution bag or a vial having a stopper pierceable by a hypodermic injection needle). The container may also have a sterile access port. At least one active agent in the pharmaceutical composition is immune cells expressing MHC-CAR as described herein.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Examples

The instant examples focuses on the development of a cellular immunotherapy utilizing chimeric receptors to selectively redirect therapeutic T cells against myelin basic protein (MBP)-specific T lymphocytes implicated in MS [16]. The results of this program can support the further development of redirected therapeutic T cells able to counteract pathologic, self-specific T lymphocytes, and specifically validate humanized MBP-DR2-chimeric receptors as a therapeutic target in MS [29].

### Example 1: Construction of Modified T-Cells Specific to Myelin Basic Protein

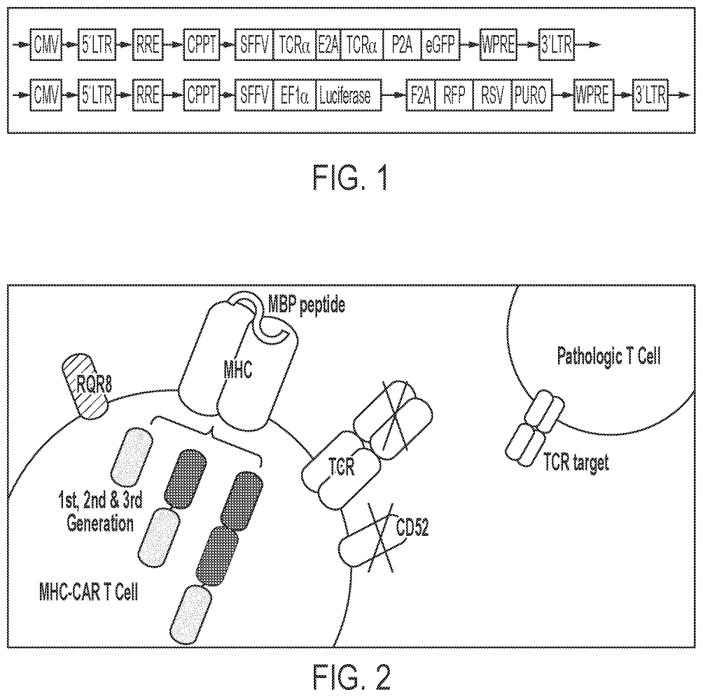

A construct for an antigen-specific T-cell receptor (TCR) that targets a MBP-loaded major histocompatibility complex-chimeric antigen receptor (MHC-CAR) is designed for reintroduction into cells with TCR knockouts for assays. Design of the TCR constructs is based on an antigen-specific TCR with a published structure and TCR expression constructs validated in human cell lines [17, 46, 51, 52]. Green fluorescent protein (eGFP) or luciferase is genetically encoded for labeling [28]. Exemplary lentiviral expression vectors comprising the TCR construct and reporting gene are provided in FIG. 1.

Several cell lines are selected for testing TCR expression and activity, including Jurkat E6-4 (a control strain that expresses TCR), Jurkat J.RT3-T3.5 (a strain that lacks TCRb), SupT1 (a strain with damaged TCRa), and primary human T cells (which contain a diverse population of TCR clonotypes).

Jurkat E6-1 cells are an established human T lymphocyte cell line from peripheral blood. It is used as a control cell line expressing TCR [15, 18].

Jurkat RT3-T3.5 lacks TCRO due to a mutation that precludes expression of the TCR β-chain. It also fails to express surface CD3 or produce the T-cell receptor α,β heterodimer. It is therefore used for validating T-cell receptor gene transfer [1, 6, 21, 49].

SupT1 is a human lymphoblast line expressing multiple T lineage markers and is used because it encodes a non-functional receptor and fails to express TCRα 1491.

PMBC-derived primary human T cells contain a diverse repertoire of TCR clonotypes.

Lentiviral vectors containing the antigen-specific TCR as illustrated in FIG. 1 are used to transduce cancer cell lines lacking at least one TCR chain, and are subsequently assessed for expression using fluorescence-activated cell sorting (FACs). In cancer cell lines containing the antigen-specific TCR, luciferase is added to the cell line following successful stable expression to enable its use in mouse studies.

TCR constructs for mRNA, multicistronic mRNA, and lentiviral transduction proceed straightforwardly, via a screen based on the genetically encoded eGFP and labeling with anti-TCR or anti-CD3 antibody [3, 19].

### Example 2: Construction of MHC-Based Chimeric Receptors (MHC-CAR) and T-cells Expressing Such

(i) Design of MHC-CAR Constructs

Receptors for adoptive cell therapy that genetically link the MBP 84-102 epitope to human leukocyte antigen HLADR2 are generated and, either incorporate or lack chimeric intracellular signaling domains [29]. The antigen-major histocompatibility complex (Ag-MHC) domain serves as receptor, binding the TCR of MBP-specific target cells. The Ag-MHC-CAR has been validated in preclinical mouse models with CD3-((i.e. a first-generation signaling domain), which may optionally be in combination with additional co-stimulatory signaling domains (i.e. second- or third-generation signaling domains) for efficacy in humans, following the methodology provided in [9, 25]. A schematic illustration of the various designs of MHC-CAR is provided in FIG. 2.

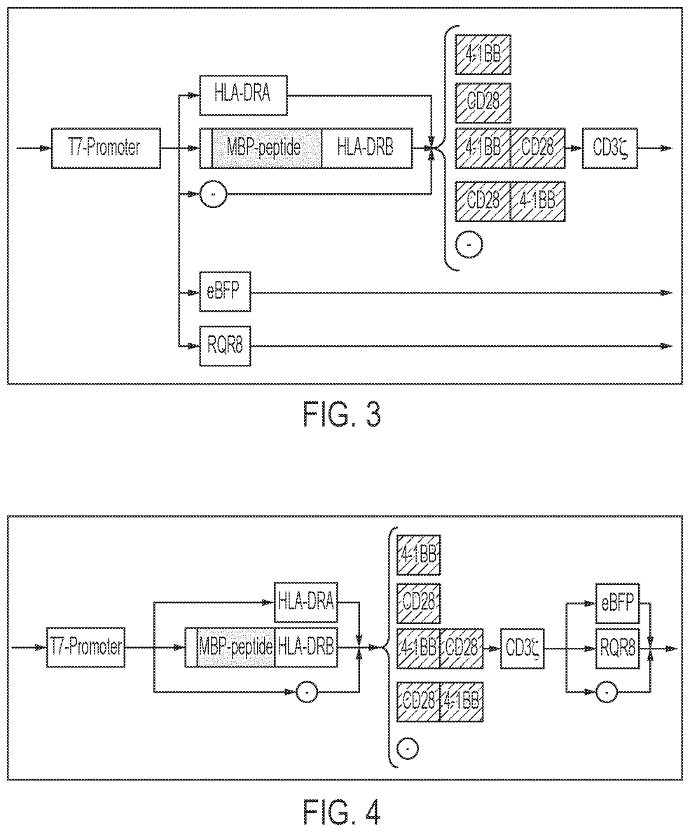

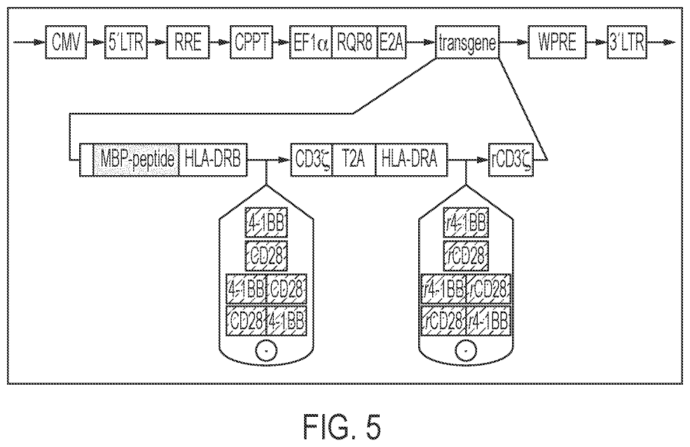

MHC-CARs are designed based on the structure of HLA-DR, and combined with a variety of internal cytoplasmic costimulatory domains. The MHC-CAR has two subunits: (i) an α-chain that contains the leader sequence, DRA*1010 domain, and a cytoplasmic domain; and (ii) a β-chain that contains a leader sequence (from HLA-DRB1*1501), a peptide (DENPVVHFFKNIVTPRTPP (SEQ ID NO: 15) from myelin basic protein), a domain (from HLA-DRB1*1501), and a cytoplasmic signal domain, for example, CD3z [291. FIG. 3. The DNA used to create mRNA contains either single chains (shown as one embodiment in FIG. 4) or are multicistronic and separated by orthogonal 2A sequences as illustrated in FIG. 5. Genetically encoded eBFP or RQR8 are introduced into the cells for cell-labeling or to provide a mechanism for depletion [4, 5, 37, 40]. FIGS. 3 and 5.

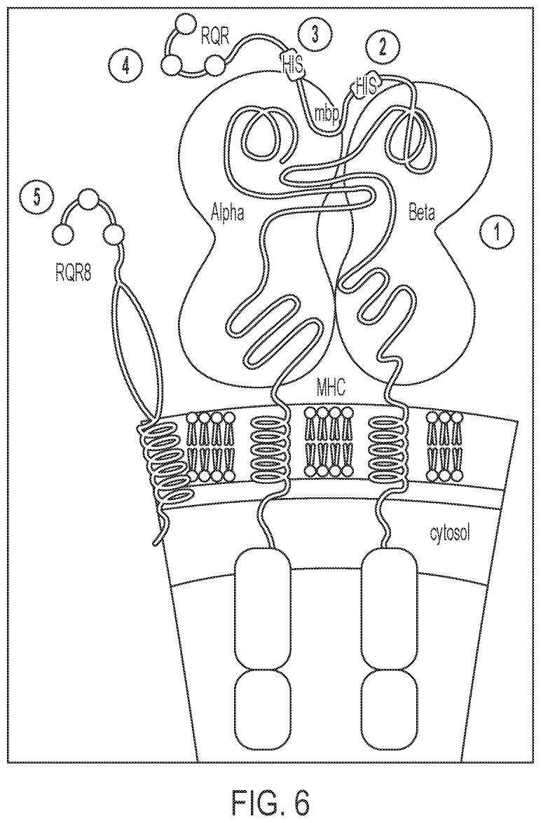

As illustrated in FIG. 6, the designed MHC-CAR has a number of sites for tagging. Site 1 is an HLA-DR antibody binding site for cases where native HLA-DR is either not expressed due to CIITA editing 126, 38]. Sites 2 and 3 are potential insertion sites for polyhistidine-tag motifs 1241. Sites 4 and 5 represent RQR and RQR8, respectively [37]. Multiple sclerosis is shown to affect blood brain barrier permeability, so in order to enhance the safety of therapeutic MHC-CAR, genetically encoded RQR would allow for rapid depletion of MHC-CAR T cells upon administration of rituximab, a chimeric monoclonal antibody treatment used off label in the treatment of severe MS [42].

### (ii) Construction of T Cell Lines Expressing MHC-CAR

A number of cell lines discussed below are selected for testing the MHC-CAR construct. Assays are then continued in human T cells in order to establish clinically translatable protocols.

K56 cells lack Class I and Class II MHC, allowing for tagless verification of HLA-DR expression using antibodies (HLA-DR is a component of the MHC-CAR) [45]. Expression assays will allow assessment of the MHC-CAR expression relative to RQR8 expression using flow cytometry, and the same lentiviral construct is then used in PMBC-derived T cells [23].

KM-H2 is a human Hodgkin's lymphoma line that can be used as an HLA-DR positive control line [34, 44]. Jurkat E6-1, as noted above, conditionally express HLA-DR upon delivery of CIITA and can be used to evaluate CIITA TALEN if CIITA knockout is used [33].

In addition, primary human T cells can be used. PMBC-derived primary human T cells are purified and enriched from whole blood, and then activated. Transduction follows enrichment. The cells are incubated with recombinant human interleukin-2 (and/or IL-7 and/or IL-15) [7, 12, 43]. Based on preclinical studies and the anticipated therapeutic course, the desired cell type is CD8+ T cells with the molecular and functional features of stem cell memory TSCM, central memory TCM, and naïve cells TN [11, 22, 32, 35]. Antibody staining allows for cell immunotyping [27].

Initial constructs, including a permutation of signaling domains (CD3z, 41BB, CD28) are expressed using mRNA, multicistronic mRNA, and lentiviral strategies in K562 cells. The cell line's lack of MHC Class I and II allow the tagless verification of MHC-CAR expression using the HLA-DR antibodies, following a successfully employed strategy [29]. Quantification of the expression efficiency in more clinically relevant cell lines, including therapeutic T cells, depends on construct, and will be measured using genetically encoded fluorescent reporters (BFP) or antibody staining of the RQR site, a polyhistidine-(HIS)-tag, or the MHCCAR (with an HLA-DR antibody if CIITA is inactivated using TALEN). Labeling sites are indicated in FIG. 6. To test rates of MHC-CAR delivery as a potential therapeutic vector, BFP is removed from constructs while RQR is retained to provide for depletion control. Expression rates of the clinically relevant construct are measured using antibody staining post-editing.

The transcription activator-like effector technology (TALEN) can also be used for preparing T cells expressing MHC-CAR. Human T cell lines are activated, transfected with TALEN, and either transfected or transduced with MHC-CAR They are then stained and analyzed by flow cytometry to assess TALEN gene inactivation and MHC-CAR expression. Upon verification of construct expression in desired cell lines, TALEN transduction (TCRα and CD52 or CIITA as discussed below) into human T cell lines is performed. TALEN is introduced into activated human T cell lines, and the MHC-CAR is subsequently introduced into the same T cell lines for evaluation. The modification of the human T cells can be performed in this order to prevent incidental fratricidal killing of MHC modified cells due to the native TCR [10]. The activity of TALEN-edited MHC-CAR T-cells is to be confirmed.

(iii) Modification of MHC-CAR-Expressing T Cells via Transcription activator-like effectors (TALENs)

Human T cells are selected to confirm inactivation of TCRa, and CD52 or CIITA genes by TALEN, followed by evaluation in combination with MHC-CAR, using TALENs. Transcription activator-like effectors (TALENs) bind DNA in a sequence-specific manner. The DNA binding domain contains a highly conserved 33-34 amino acid sequence with a divergent 2 amino acid RVD (repeat variable dipeptide motif) conferring specific nucleotide recognition [23, 31].

Mutation of either a or P chain of the TCR is sufficient for disruption of surface TCR expression [15, 36]. TALEN is used to inhibit expression of two genes in the therapeutic cell without introducing a proliferative advantage through undesired translocations [39][47]. TCR expression is inhibited through a TRAC-targeted TALEN to prevent graft versus host disease (GvHD) and allow for the creation of an allogeneic therapy [15, 39, 47].

CD52 deletion can be made for alemtuzumab compatibility. Alemtuzumab is a humanized anti-CD52 IgG1 monoclonal antibody that targets and depletes circulating T and B lymphocytes [41]. Alemtuzumab can be used as rescue therapy or as first line drug in severe-onset MS 1501, and will be co-administered in the human patient population. TALEN knockout will make MHC-CAR compatible with concurrent treatment in patients [14, 39]. Further, CIITA deletion can be made for characterization of HLA-DR in MHC-CAR CIITA is a protein coding gene essential for the transcriptional activity of HLA Class 11 promoter [26]. Knockout would allow for the direct measurement of HLA-DR using antibodies for characterization of MHC-CAR expression [38, 53]. The inhibition of CD52 expression allows for concurrent treatment with Lemtrada R (alemtuzumab), an FDA-approved treatment for multiple sclerosis 1421. Alemtuzumab is also used as a lympho-depleting/lympho-suppressive agent that aids in the engraftment of CAR T therapies [39]. A CAR T-cell therapy modified with TRAC- and CD52-targeted TALENs is currently being tested in clinical trials 138, 40, 431.

TALEN to inactivate MHC Class II transactivator (CIITA) in place of CD52, as inactivation of CIITA is expected to inhibit HLA-DR expression [26][38], thus allowing for the direct identification of MHC-CAR-containing cells by HLA-DR antibody staining.

Validated TALENs (TRAC:
TTGTCCCACAGATATCCagaacectgacectgCCGTGTACCAGCTGAGA
(SEQ ID NO: 376), CD52: TTCCTCCTACTCACCATcagcctcc
tggttatGGTACAGGTAAGAGCAA (SEQ ID NO: 371), CIITA:
TTCCCTCCCAGGCAGCTCacagtgtgccaccaTGGAGTTGGGGCCCCTA
(SEQ ID NO: 366))

are obtained from Cellectis or designed to target previously validated sites [38, 43].

Human T cells are activated and electroporated with mRNA encoding variants of 3 different TALEN: TCR-alpha constant chain, CD52, and CIITA. Cells are surface stained with anti-CD3 or anti-TCR (TCR-alpha constant chain), anti-CD52 (CD52), or anti-HLA-DR (CIITA), and then analyzed by flow cytometry.

In the TALEN expression assays, the ability of previously validated TALENs to inactive target genes are re-validated [39]. Translocation studies and off-target studies are reperformed, and several whole-genome sequences confirm results. GUIDE-seq can be used as an alternative to whole genome sequencing to confirm on- and off-target editing [8].

One of more of the following endogenous genes are to be edited to reduce interaction with other cells: TCR (through TCR alpha or beta chain: to reduce targeting to undesired cells), CIITA (regulates expression of MHC Class II genes; target cells for taster deletion), B7-1(CD80) and/or B7-2(CD86) knockout, and b2m (regulates expression of MHC Class I genes) with NKG2D ligands or UL18.

Further, one or more of the following genes can be edited to modify the function of an interacting cell: PD-L1+1—CTLA4-Ig overexpression; PD-L1/2 overexpression +/−PD-1 knockout; FasL overexpression +/−Fas knockout; Galectin 9 overexpression +/−TIM3 knockout; and/or PVT/CD155 overexpression +/−TIGIT knockout. Defects in PD-1, Fas, TIm3, TIGIT predispose patients to autoimmunity. Some drugs can restore function (for example: Tim3, glatiramer acetate and IFN-beta). If autologous cells from a patient suffering autoimmune disease are utilized they may require patient specific correction of defective genes that influence CTL and Treg function. Their personal mutation set may also determine whether CTL or Treg cells will be the most therapeutically relevant, and whether some cellular modification will be effective, if either autologous or allogeneic cells are used.

Further, the following edits may modify the location and/or function of the cells (for example, to make it more like a follicular regulatory cell):
MHC-CAR-(FOX3P)-(CSI or CD19 CAR)/(CS-1 knockout required for CS-1 CAR)
MHC-CAR-(FOX3P)-(CCR7 or CXCR5)

Moreover, the following edits can enable combination therapies for autoimmune diseases (e.g., MS specific therapeutics)
RQR tag: engineered T cells can be removed with rituximab (kill switch). A tag for the newly approved anti-CD20 antibody Ocrevus could be generated.
Rapamycin switch: CARs only in/active when patient is treated with rapamycin (tacrolimus)
CD52 knockout: allows pretreatment with Lemtrada (alemtuzumab) to decrease number of immune cells present
VLA-4 knockout: can treat with tysabri to move pathogenic immune cells to periphery but engineered cells are forced to stay there (may not be ideal for patients with established MS as cells that are stuck in the brain spinal cord; however, simultaneous mRNA expression of VLA-4 can provide temporary access to those locations)
IL-6 antibody (Toclizumab) secretion from engineered T cell: helpful when the engineered T cell must access the brain and spinal cord, but this drug cannot access the locations due to the BBB

### Example 3: Investigation of MHC-CAR Activities

(i) Preparation of Primary T Cells Expressing MHC-CAR

Primary T cells can be prepared as follows. T cells are isolated from peripheral blood mononuclear cells (EasySep Human T Cell Enrichment Kit, Stemcell Technologies) and activated (Dynabeads Human T-Activator CD3/CD28, Life Technologies) with (X-Vivo 15 medium. Lonza: 20 mg/ml 11-2, Miltenyi; 5% human AB serum, Seralab). A suitable MHC-CAR construct containing a MBP antigenic peptide is introduced into the primary T cells using a conventional method. Surface expression of the MHC-CAR construct is verified by FACS and antibody staining.

(ii) MHC-CAR Activity Tests In Vitro

Upon verification of construct trafficking and expression (with mRNA, multicistronic mRNA, lentivirus), activity tests are conducted in vitro. All tests are conducted at different effector:target (E:F) cell concentrations. The in vitro tests provide an initial evaluation of MHC-CAR signaling domains and T cell subsets.

(iii) Signaling Domain Assessment by IL-2 Production

24 hours post electroporation, human T cells transiently engineered with MHC-CAR are stimulated with plate-bound HLA-DR antibody, to determine whether MHC-CAR (containing various signaling domains) is functional. 11-2 production is measured 24 hours later using a StemCell IL-2 ELISA kit. This test provides a quick assay as to whether variants should be reengineered or abandoned [29].

(iv) Interaction of MHC-CAR Cells and Pathogenic TCR Cells Through Proliferation Assay

Target cell lines transiently expressing TCR are magnetically sorted for TCR expression 24 hours after electroporation and irradiated, in order to test whether engagement of the MHC-CAR with antigen-specific TCR stimulates proliferation of MHC-CAR containing T cells. Alternatively, target cell lines that stably express TCR are irradiated. The irradiated cells displaying (+/−antigen-specific) TCR are incubated with CFSE-labeled MHCCAR cells and proliferation is measured after culture at different T:E ratios [30].

(v) Degranulation Assay

CAR T-cells are labeled through epitopes on RQR (which are not being expressed on the target cells used) or eBFP instead of T cell markers. The assay is performed for cell lines with transient or stable expression (the example of transient expression is described). 24 hours post-electroporation, MHC-CAR human T-cells with either RQR8 or BFP are co-cultured with target (antigen-specific TCR SupT1 or Jurkat) or control (+1—TCR Jurkat or TCR-SupT1) cells for 6 hours. Transiently expressed (and later stably expressing) target cells are electroporated with antigen-specific TCR and sorted with CD3 magnetic beads post-electroporation. The RQR+ or BFP+ MHC-CAR T-cells (as identified with anti-rituximab antibody. QBEnd10 antibody) are analyzed by flow cytometry to detect the expression of the degranulation marker CD107a on their surface [2].

(vi) Cytokine Secretion Assay

The assay is performed for cell lines with transient or stable expression (transient expression is described). The human T cells transiently expressing the MHC-CARs are assessed for cytokine secretion following co-culture with target cells 24 hours post electroporation. Human T cells transiently expressing the MHC-CARs are co-cultured with target (antigen-specific TCR containing Jurkat or SupT1 cells) or control (+/−non-antigen-specific Jurkat or SupT1) cells for 24 hours. The antigen-specific TCR is then killed by irradiation before the assay. The supernatants are harvested and analyzed using the TH1/TH2 cytokine cytometric bead array kit to quantify the cytokines produced by T cells [13]. MHC-CAR T-cells produce IFN and other cytokines in co-culture with antigen-specific TCR expressing target cells but not in co-culture with control cells.

(vii) IFNγ release assay

Various levels of MHC-CAR expressing cells are incubated with irradiated TCR T-cells 24 hours after transfection. Co-cultures are maintained for 24 hours. After incubation and centrifugation, supernatants are tested with IFNγ detection by ELISA.

(viii) Cytotoxicity Assay

TCR T-cells are incubated with therapeutic MHC-CAR as well as control cells. Target and control cells are labeled with fluorescent intracellular dyes (CFSE or Cell Trace Violet) before co-culturing with MHC-CAR T-cells. The co-cultures are incubated for 4 hours. After this incubation period, cells are labeled with a fixable viability dye and analyzed by flow cytometry. Viability of each cell population (target or negative control) is determined and the percentage of specific cell lysis is calculated. Cytotoxicity assays are carried out 48 hours after transduction.

(ix) Inhibition Assay

PBMCs are co-cultured with irradiated or mitomycin-treated engineered T cells expressing the MHC-CAR construct. As control, PBMCs are co-cultured with irradiated or mitomycin-treated engineered T cells that do not express the MHC-CAR construct. 7 days later, cell proliferation from a human patient donor A is measured by XTT colorimetric assay or by CFSE dilution (FACS analysis). Although cell proliferation would be observed in control, no or limited cell proliferation is expected when engineered T cells express secreted FP. The results from this experiment aim to show that alloreactive T cells proliferation is inhibited when the MHC-CAR expressing T cells express FP.

(x) Proliferation

To test whether engagement of the MHC-CAR with antigen-specific TCR stimulates proliferation of MHC-CAR containing T cells, target cell lines transiently expressing TCR are magnetically sorted for TCR expression 24 hours after electroporation and irradiated. Alternatively, target cell lines that stably express TCR are irradiated. The irradiated cells displaying (+/−antigen-specific) TCR are incubated with CFSE-labeled MHC-CAR cells and proliferation is measured after culture at different T:E ratios.

(xi) In Vivo Tests

MHC-CAR (with mouse and human MHC) in mouse T cells has previously shown therapeutic efficacy in experimental allergic encephalomyelitis, the mouse model for multiple sclerosis [25, 29]. Here we test whether MHC-CAR in human cells can target human TCR in human T cell lines, using an in vivo mouse model analogous to that in prior CAR T preclinical studies 1391. The in vivo tests allow further evaluation of MHC-CAR signaling domains and T cell subsets.

The In vivo activity of MHC-CAR T-cells can be verified in a mouse xenograft model as illustrated in FIG. 7.

Immunodefficient NOG mice are intravenously injected with antigen-specific TCR-luciferase expressing T-cells as an MS xenograft mouse model. Mice then receive intravenous doses of MHC-CAR T-cells tested at different doses, either 2 or 7 days post-injection with tumor cell line. Intravenous injection with T-cells that are not transduced with the CAR lentiviral vector serve as control. Bioluminescent signals are determined at the day of T-cell injection (DO), at D7, 14, 21, 28 and 40 after T-cell injection in order to follow the expansion of TCR-luciferase expressing cells in different animals [39].

CAR T-cells with similar background modifications (TALEN to inactivate native TCR and minimize graft vs host, TALEN to inactivate CD52 and allow simultaneous treatment with alemtuzumab, and RQR8 to allow depletion) to the ones shown here have been previously validated in mouse models and are now used in a UCART19 clinical trial 1391.

(xii) Kill-switch verification

Transduced T cells are exposed to 25% baby-rabbit complement (AbD Serotec) for 4 hours with or without inclusion of pharmaceutical complements (rituximab, tysabri, or alemtuzumab) to examine complement-dependent cytotoxicity (CDC)-mediated sensitivity. Miltenyi CD34 magnetic bead-selected-transduced RQR8 T-cells are compared against a similarly treated population of Q8-transduced T cells to demonstrate specificity of CDC-mediated deletion. Further examination of CDC assay parameters was achieved through time-course/dose-titration assays using RQR8-transduced T cells incubated with pharmaceutical complement at 12.5, 25, 50, and 100 mg/mL and time-point assessments ranging between 1 to 120 minutes.

### Example 4: Regulatory T Cells (Treg) Expressing MHC-CAR

Therapies that antigen specifically target pathologic T lymphocytes responsible for multiple sclerosis and other autoimmune diseases are expected to have improved therapeutic indices compared with antigen-nonspecific therapies. This example provides an exemplary cellular immunotherapy that uses chimeric antigen receptors to selectively redirect therapeutic T cells against myelin basic protein-specific T lymphocytes implicated in MS

Treg Cell Sorting. Transduction, and Expansion

CD4+ T cells are isolated from PMBC via RosetteSep (STEMCELL Technologies) and enriched for CD25+cells (Miltenyi Biotec) prior to sorting into live CD4+CD45RO-CD45RA+CD25+Tregs and CD4+CD45RO-CD45RA+CD25—control T cells by FACS. Sorted T cells are stimulated with artificial APCs (aAPCs) loaded with aCD3 mAbs in 1,000 U/ml or 100 U/ml of IL-2, for Tregs or non-reg control T, respectively. One day later, cells are transduced with lentivirus. At day 7, ΔNGFR+cells were purified with magnetic selection (Miltenyi Biotec), then re-stimulated with aAPCs as above and expanded for 6 to 7 days.

### Flow Cytometry:

For phenotypic analysis, cells were stained with fixable viability dye (FVD) (65-0865-14 and 65-0866-14, eBioscience) and for surface markers before fix/perm with FOXP3′Transcription Factor Staining Buffer Set (eBioscience), followed by staining for intracellular proteins. For analysis of cytokine production, cells were stimulated with 10 ng/ml PMA and 500 ng/ml ionomycin, in the presence of brefeldin A (10 μg/ml) (all Sigma-Aldrich) for 4 hours. Samples were analyzed by flow cytometry.

### Microscopv:

PBMCs are labeled with PKH26 or PKH67 (Sigma-Aldrich, PKH26GL-1KT and PKH67GL-1KT), and Tregs are labeled with cell proliferation dye (CPD) eFluor450 (eBiosciences, 65-0842-85) and then suspended in a 3D gel of 1.5% rat tail collagen type I (Ibidi) composed of 1×DMEM and 100% FCS per the manufacturer's general 3D gel protocol. The cell suspension is pipetted into a β-Slide Chemotaxis3D and allowed to polymerize for 30 minutes in a humidified incubator at 35° C. and 5% CO2 (Tokai Hit) on a Leica TCS SP8 confocal microscope. The outer chambers are filled with 1×DMEM and images recorded using a ×10/0.30 objective every 2 minutes for 3 hours, eFluor450, PKH67, and PKH26 were excited at 405 nm, 488 nm, and 561 nm, and the fluorescence emission is collected at 415-470 nm, 495-525 nm, and 570-650 nm, respectively. The number of interactions between CAR-Tregs and either target or control cells is quantified every 20 minutes. Cells that do not move were excluded from the analysis. The total numbers of each labeled cell type per field of view can be counted using the analyze particles function in ImageJ (imagej.nih.gov/ij/).

### Treg-Specific Demethylated Region (TSDR) Analysis

Treg stable expression of stable Foxp3 is associated with selective demethylation of TSDR within the Foxp3 locus. In order to test for stable expression, DNA from frozen T cell pellets is was isolated with the DNeasy Blood and Tissue Kit (QIAGEN) and bisulfite converted with the EZ Direct Kit (Zymo Research). PCR of BisDNA was performed with the Human FOXP3 Kit (Epigen DX) and prepared for pyrosequencing using PyroMark buffers (QIAGEN), then assayed on a Biotage PyroMark Q96 MD pyrosequencer (QIAGEN). Results were calculated with Pyro Q-CpG software (Biotage).

### Cytokine Production

To measure cytokine production, T cell lines are stimulated with K562 cells (1 K562:2 T cells) for 48 hours. Supematants are collected and cytokine concentration was determined by the Human ThlIh2/Th17 Cytokine Kit (BD Biosciences) and analyzed.

### Suppression of MHC CAR-Specific Proliferation

To test whether Treg specific for target were also capable of suppressing CD4+ T cell proliferation, MHC CAR-specific CD4+T clones are isolated. An Epstein Barr Virus-tranformed B lymphablastoid cell line was transduced with MHC-CAR using lentivirus. EBV cell lines were grown overnight, irradiated at 150 Gy, and cocultured with CPD-labeled MHC CAR-specific CD4+T clones in the absence or presence of CAR-expressing Tregs or conventional T cells. Proliferation is determined after 4 days, and percentage of suppression of MHC CAR-specific clones calculated using percentage of proliferation as follows: (100−[(% proliferated MHC CAR+test)/(% proliferated MHC CAR alone)]—100).

Upon verification of construct trafficking and expression (with mRNA, multicistronic mRNA, and/or lentivirus), activity tests will begin in vitro. All tests are conducted at different effector:target (E:F) cell concentrations. The in vitro tests are expected to provide an initial evaluation of MHC-CAR signaling domains and T cell subsets.

Transient or Lentiviral Expression of Chemokine and Adhesion Receptors in T Cells Receptors are expressed in human T cells after electroporation of mono/polycistronic mRNA or lentiviral transduction. Expression of the receptor is analyzed using flow cytometry. In summary: 5×10⁶ T cells preactivated several days (3-5) with anti CD3/CD28 coated beads and IL2 were re-suspended in cytoporation buffer T, and electroporated with 45 μg of mRNA. Twenty-four hours after electroporation, human T cells engineered using polycistronic mRNAs encoding the multi-chain CARs were labeled with a fixable viability dye eFluor-780 and a PE-conjugated goat anti mouse IgG F(ab′)2 fragment specific, and analyzed by flow cytometry. Alternately the receptors were vectorized in lentivirus, expressed, and analyzed similarly.

### In Vitro Chemotaxis Assay

Transduced T cells were used in chemotaxis assays as previously described [Bürkle et al., Blood, 110(9):3316-3325, 2007: Wu and Hwang, Journal of immunology, 168(10):5096, 2002.: Singh et al., Journal of immunology. 180(J):214-221, 2008.; Ryu et al., Molecules and Cells. 39.12:898-908, 2016.1. Cells (-20.000 cells in medium, one million cells, 5×10⁶/mL) were placed on top of the 5-μm pore size filters in duplicate, whereas medium with and without chemokines were placed into the lower chamber. After 30 min, 1 h, 3 h, 5 hr, 24 hr at 37° C., migrated cells that had fallen to the bottom of the plate were:
A, photographed using a 4×objective. Three random views from each of two wells were counted using Image Pro Plus (Media Cybernetics, Silver Spring, MD). Three independent experiments were performed with similar results.
B. 400 μL of the cell suspension was added to 100 μL of a solution containing 4×10-7 M FITC-labeled phalloidin, 0.5 mg/mL 1-alpha-lysophosphatidylcholine (both from Sigma. St Louis, MO), and 7% formaldehyde in phosphate-buffered saline (PBS). The fixed cells were analyzed by flow cytometry on a FACSCalibur. and all time points are plotted relative to the mean relative fluorescence of the sample before addition of the chemokine.
C. the cells in the lower chamber were counted using Countess II FL (Thermo Fisher Scientific, USA) or the O.D, value at 450 nm was measured using a Versamax microplate reader (Molecular Devices).

### Example 5: Expression of MHC-Based Chimeric Receptors (MHC-CAR) in HEK293 Cells

Constructs encoding MHC-CARs were constructed as discussed in Example 2 and assessed for expression in HEK293 cells. Briefly. Construct 1 includes an EF1alpha short promoter, CD19 CAR (4G7-CAR), CCR6, and GFP (provided by SEQ ID NO: 426); and Construct 2 includes a EF1alpha short promoter, RQR8, MHC-CAR1 part B MHC-CAR1 part A, and GFP (provided by SEQ ID NO: 409).

Constructs 1, 2, or media control (non-transfected) was transfected into HEK293 cells and cultured. The cells were assessed for expression by microscopy based on GFP expression. Populations of GF-positive cells were observed in the groups that were transfected with Construct 1 or Construct 2. The cells were also assessed for expression of the components encoded by the constructs by flow cytometry. Tables 8 and 9.

### Construct 1 Expression

For detection of CCR6, cells were incubated with an anti-CCR6 monoclonal antibody conjugated to APC (17-1969-42, eBioscience); and for detection of CD19, cells were incubated with a biotinylated CD19 (Acro CD9—H8259, Acro Biosystems) followed by a streptavidin-PE (405203, BioLegend@).

TABLE 8
Expression of Construct 1

| | FITC (GFP) | APC (CCR6) | PE (CD19) |
|---|---|---|---|
| CCR6 Expression | | | |
| Unstained | 71.9 | 0.1 | 17.4 |
| CCR6 | 71.3 | 81.9 | 16.9 |
| CD19 Expression | | | |
| Unstained | 71.9 | 0.1 | 17.4 |
| CD19 | 71.3 | 0.1 | 33.3 |
| streptavidin-PE only | 71.5 | 0.1 | 18.5 |

### Construct 2 Expression

For detection of CD34 expression from the epitope included in RQR8, cells were incubated with an anti-CD34 APC-conjugated monoclonal antibody; and for detection of MHC-CAR expression, the cells were incubated an anti-HLA-DR antibody.

TABLE 9
Construct 2 Expression

| GFP Expression | FITC (GFP) | APC (CD34) | PE |
|---|---|---|---|
| Unstained | 62.3 | 0.1 | 2.4 |
| RQR Expression | FITC (GFP) | APC (CD34) | PE |
| Unstained | 62.3 | 0.0 | 2.4 |
| CD34 | 61.8 | 58.0 | 2.5 |

| MHC CAR Expression | FITC (GFP) | APC (HLA-DR) | PE |
|---|---|---|---|
| Unstained | 62.3 | 0.0 | 2.4 |
| HLA-DR | 63.9 | 98.1 | 2.1 |

Nucleic acid sequence of Construct 1
(SEQ ID NO: 426)
atggagacagacactcttctcctttgggtcttgctgctgtgggttcccggaagcacaggagaagcacagttgcaa
cagtctgggccagaactcatcaaacccggagcttctgtaaaaatgtcatgcaaagctagtggatatacatttact
tcttacgtgatgcactgggtaaaacagaaacctggtcaggggcttgagtggatcgggtacattaacccatataat
gacggcaccaaatataacgagaaattcaagggaaaggctacgcttacatcagataagtccagtagcaccgcttat
atggaacttagcagccttacttccgaagattccgcggtgtattactgcgcgagagggacttactactacgggagt
cgagtattcgattattggggtcaaggcacgacgctcacggtgagctcaggtggtggagggtctgggggtggcggc
agtggtggggggggctcagacatcgtgatgacccaggcagcaccttctatcccggtaaccccaggcgagtctgta
tctatcagttgtcggtccagcaagtctcttctcaacagtaatggcaatacatatctttactggttcctccaaagg
cctgggcaaagtcctcaacttcttatatatcggatgtccaatcttgcgagtggcgtaccagacaggttttcaggg
tctgggagcggaacagcttttacgttgagaatatccagggtagaagctgaggacgtcggtgtatattattgcatg
caacatctcgaatacccctttaccttcggcgctggtacaaagctcgaattgaaacgcagcgatccaaccacgacg
ccagcgccacgaccacctacgcccgctccaactattgcctcccagcccctgagtcttcggccagaagcgtgtaga
cctgctgccggcggggccgttcatacgcggggccttgactttgcatgtgatatctatatatgggctcctttggcg
ggaacttgcggagtgcttcttttgtcactcgtgataacgttgtattgtaaaaggggtcgaaagaaactcctctat
atatttaagcagccctttatgaggcccgtgcaaacaacacaagaagaggacggatgctcttgtcgattcccggaa
gaggaggaagggggggtgtgagcttagggtcaagttttctcgctctgccgacgcgccagccctcaacagggccaa
aaccagctgtataacgaactcaacctcgggcgccgggaagagtatgacgtccttgacaaacggcgcggtcgcgac
cctgaaatgggtggaaaaccgaggcgaaagaacccccaggagggactttacaacgaattgcaaaaagacaagatg
gccgaagcctattccgaaattggaatgaaaggcgagcggagacgaggtaaggggcatgacggcctgtatcaaggg
ctctctacggccacgaaggatacttacgacgcccttcatatgcaagctcttccaccacggggttcgagcggcagt
ggagagggcagaggaagtctgctaacatgcggtgacgtcgaggagaatcctggcccaatgagtggggaaagtatg
aacttcagcgatgtatttgactcctccgaagattactttgtatctgtgaatacgagccattactccgtcgatagt
gaaatgctgctctgtagtctccaagaagtccgccaattcagtcgcctcctcgttcccatcgcgtactcccttatt
tgtgtttttggccttctgggtaacatcctggttgtaatcacattcgctttctataaaaaagctcggagtatgact
gatgtttaccttcttaacatggctatagoggacattctttttgtgcttactctcccattctgggctgtgagccat
gcaacaggggcgtgggttttttcaaatgccacatgtaagctgcttaaagggatctatgcaataaacttcaattgc
gggatgctcctgctgacatgcatcagtatggatcgatacatagctatagtacacaggactaagtccttccgcctg
cgatcccgcacactgcctaggagcaaaattatttgcctcgtcgtatgggggctctcagtgatcatctcctccagt
acgtttgtctttaaccagaaatataacacacagggttctgatgtatgtgaaccaaagtatcagacagtgagtgaa
ccaatacggtggaagttgcttatgttgggcttggagctgctttttgggtttttcatcccactgatgttcatgatt
ttctgttatacatttattgttaagaccttggttcaggcgcaaaatagcaagagacataaggcaattcgagtcacc
attgccgtggtgttggtcttcttggcctgtcagatcccccataatatggttctgctcgtcaccgccgctaacttg
ggtaagatgaatcgatcttgtcagtccgagaagttgatcggatacaccaaaactgtgatagaagtgctggccttc
cttcactgttgtctgaacccagttttgtatgcttttataggacagaagtttcgaaattacttcttgaaaatcctc
aaggacctctggtgtgttcgaaggaagtacaagagctctggctttagttgcgctgggcgctacagtgagaatata
tcccggcagacctccgagactgctgataatgacaacgcaagttccttcactatg
Nucleic acid sequence of Construct 2
(SEQ ID NO: 409)
atgggtacttcactgttgtgctggatggcactttgtcttttgggtgccgatcatgctgatgcatgtccgtactcc
aatcctagcctgtgctccggggggggagggagtgaactccctacacagggaaccttctctaatgtctccaccaac
gtctcccctgcaaaaccgatcacaatagcttgcccctatagtaacccttccctctgtagtggaggggggggttca
cctgctccacgccctcctacccccgcgccaacgatcgcgtcacaaccgctcagtcttaggccggaagcctgtagg
ccagcggctggcggtgcggttcatacgcggggattggattttgcctgcgacatttacatttgggctccgctggcc
ggtacttgtggggtattgctgttgtctottgttattacgctttattgcaatcacaggaacaggcgacgagtatgc
aaatgcccgcggcccgtcgtgagatctgggtccggccaatgtactaactacgctttgttgaaactcgctggcgat
gttgaaagtaaccccggtcctccaacaggtatggtatgcttgaagctcccgggcgggtcctgcatgaccgctctc
actgttactcttatggtccttagttcaccgcttgccctggcatctgatgagaatcccgtggttcatttttttaag
aacatcgtcacaccgcgcaccccacctgggggaggcggatctggcggaggcgggagtggaggctcaggagacaca
agaccccgattcttgtggcagcccaaaagggagtgccattttttcaatgggacggaacgagttcgcttccttgat
gggcgacctgacgcggagtactggaactcccaaaaggatattttggagcaggcacgagcagctgtggacacctat
tgtcgacataattatggtgtggtggaatcctttacagttcagcggcgggtgcaacctaaagtgaccgtgtatcca
tctaaaacgcaacccctccaacaccataacctcctggtgtgttccgtaagcggcttctatcccgggtcaattgag
gtcaggtggttcctcaacggtcaggaggagaaggccggaatggtaagtactggtcttatccagaacggagactgg
accttccaaactttggtaatgttggaaacggtgccgcgatccggggaggtgtatacatgccaagttgaacacccg
agtgttacgagccccctgacggttgagtggagggcgcggtcagagagcgcacaatctaaaatgctgtcaggagta
ggcggatttgtactcggactcctctttttgggcgctgggttgtttatctactttagaaaccaaacaagtagagta
aagtttccccgaagtgcggacgcccccgcgtatcagcaaggtcaaaaccagctttataacgaactgaacttggga
cgacgcgaagagtacgatgttcttgataagcggagagggcgcgatcccgaaatggggggaaagcctcggaggaag
aacccacaagaaggcctttataatgaactgcagaaggacaagatggcggaggcgtattccgaaataggcatgaag
ggtgaacggaggagaggaaagggacatgacggactttatcaaggattgtctaccgcaactaaagacacctatgac
gcgttgcacatgcaggctctccctccgagaggttcgagcggcagtggagagggcagaggaagtctgctaacatgc
ggtgacgtcgaggagaatcctggcccaatggcaatatctggtgttcctgtcctcgggttttttatcatagccgta
ctgatgtcagcacaggaatcatgggcgataaaagaagagcacgtgataatacaggcggagttttatttgaacccg
gaccagagcggtgagttcatgttcgattttgatggegacgagatatttcacgttgacatggcaaaaaaggaaacg
gtgtggagacttgaggagtttggacgattcgcatcatttgaggcacaaggagcactcgccaatatcgcggtggac
aaggccaacctggagatcatgacataacgctccaattatacgcctatcactaatgtgccccctgaggttactgtg
ctcacaaattctcccgtagaacttagggaacctaacgtcctcatatgtttcatcgacaagttcactcctccggtg
gtcaatgtaacgtggcttcggaatggtaagccggtcaccacgggtgtctcagagaccgtatttctgcccagagaa
gaccacctcttccgcaaatttcattaccttccctttcttccttcaacggaagacgtttacgactgcagggtcgaa
cattgggggcttgacgagccacttctcaagcattgggagttcgacgccccatcaccgcttccagaaacgactgaa
aacgttgtctgcgctcttggcctgacagtgggcctggtaggcattattatcgggaccatctttatcatcaaaggt
ttgacttcccgggtcaaatttagcagatccgctgacgcaccggcctaccagcagggccagaaccaactctacaac
gagctgaatctcggccgacgggaagagtatgacgtactcgacaagcggagaggtcgagaccctgagatgggcggt
aaaccgagacggaaaaatccccaagagggtctttataatgaactccagaaggataagatggctgaagcctattct
gagatagggatgaaaggcgagcggcggaggggtaagggccatgatggcctttaccagggactctccacggcaacc
aaagatacttacgacgcccttcacatgcaagccctcccgccacgcggatccggcgcaacaaacttctctctgctg
aaacaagccggagatgtcgaagagaatcctggaccggtgagcaagggcgaggagctgttcaccggggtggtgccc
atcctggtcgagctggacggcgacgtaaacggccacaagttcagcgtgtctggcgagggcgagggcgatgccacc
tacggcaagctgaccctgaagttcatctgcaccaccggcaagctgcccgtgccctggcccaccctcgtgaccacc
ctgacctacggcgtgcagtgcttcagccgctaccccgaccacatgaagcagcacgacttcttcaagtccgccatg
cccgaaggctacgtccaggagcgcaccatcttcttcaaggacgacggcaactacaagacccgcgccgaggtgaag
ttcgagggcgacaccctggtgaaccgcatcgagctgaagggcatcgacttcaaggaggacggcaacatcctgggg
cacaagctggagtacaactacaacagccacaacgtctatatcatggccgacaagcagaagaacggcatcaaggcg
aacttcaagatccgccacaacatcgaggacggcagcgtgcagctcgccgaccactaccagcagaacacccccatc
ggcgacggccccgtgctgctgcccgacaaccactacctgagcacccagtccgccctgagcaaagaccccaacgag
aagcgcgatcacatggtcctgctggagttcgtgaccgccgccgggatcactctcggcatggacgagctgtacaag
taa′

### Example 6: Expression of CD3

Expression of CD3 was assessed to determine the efficacy of CRISPR methods targeting the T cell receptor alpha constant (TRAC) gene. Briefly, sgRNAs targeting the TRAC gene were generated by amplifying the target site using forward primer (5′-AGCGCTCTCGTACAGAGTTGG-3′ (SEQ ID NO: 385)) and reverse primer (5′-AAAAAAAGCACCGACTCGGTGCC-3′ (SEQ ID NO: 386).

The unmodified sgRNA is provided by the nucleic acid sequence:

(SEQ ID NO: 384)
5′- GAG AAU CAA AAU CGG UGA AUG UUU UAG AGC UAG AAA
UAG CAA GUU AAA AUA AGG CUA GUC CGU UAU CAA CUU GAA
AAA GUG GCA CCG AGU CGG UGC UUU U -3.

The modified sgRNA is provided by the nucleic acid sequence:

(SEQ ID NO: 337)
5′- 2′OMe(G(ps)A(ps)G(ps)) AAU CAA AAU CGG UGA AUG
UUU UAG AGC UAG AAA UAG CAA GUU AAA AUA AGG CUA
GUC CGU UAU CAA CUU GAA AAA GUG GCA CCG AGU CGG
UGC 2′OMe(U(ps)U(ps)U(ps) U -3′.
2′OMe = 2′O) -methyl RNA and ps = phosphorothioate.

Primary human stimulated CD3+ T-cells were transfected with an mRNA encoding Cas9 (Cas9 only) or both an mRNA encoding Cas9 and sgRNAs targeting the TRAC gene. After 7 days post-transfection, expression of CD3 was assessed by flow cytometry. The cells were incubated with a 1:100 dilution of an anti-CD3-APC antibody (clone OKT3; BioLegend®, cat. no. 317318). As shown in FIG. 11, transfection of an mRNA encoding Cas9 and sgRNAs targeting the TRAC gene resulted in a substantial reduction in CD3 expression.

### Example 7: Kill Switch Verification

Efficacy of the RQR8 kill switch encoded in example Construct 2 was assessed using a cell viability assay. Briefly, HEK cells were transfected with media only, Construct 1 (SEQ ID NO: X), Construct 2 (which encodes the rituximab-mediated RQR8 kill switch, SEQ ID NO: X), or both Construct 1 and Construct 2. The transfected HEK293 cells were harvested, counted, and resuspended at 1×10\hat{ }6 cells/mL. 300 uL of the cellular suspension was transferred into each of 4 wells of α48-well tissue culture plate. 100 uL of complete medium and 4 uL of Rituximab were added to the second well, and 100 uL of freshly prepared baby rabbit complement and 4 uL of Rituximab were added to the fourth well. The plates were incubated for 2, 4, or 24 hours. The assay was terminated by the adding 1 uL of chilled Annexin V buffer (150 mMNaCl. 10 mM HEPES, 10 mM CaCl), and then the sample was transferred into a pre-prepared flow cytometry tube containing 3 mL of Annexin V buffer.

Samples were harvested by centrifugation and any residual buffer was blotted with paper towels. The samples were then stained with 1 uL of Annexin V APC, vortexed, and placed in subdued lighting for 15 minutes. The samples were then washed with Annexin V buffer and supplemented with 5 uL of propridium iodide/mL buffer and placed on ice pending flow cytometry performed immediately following final suspension.

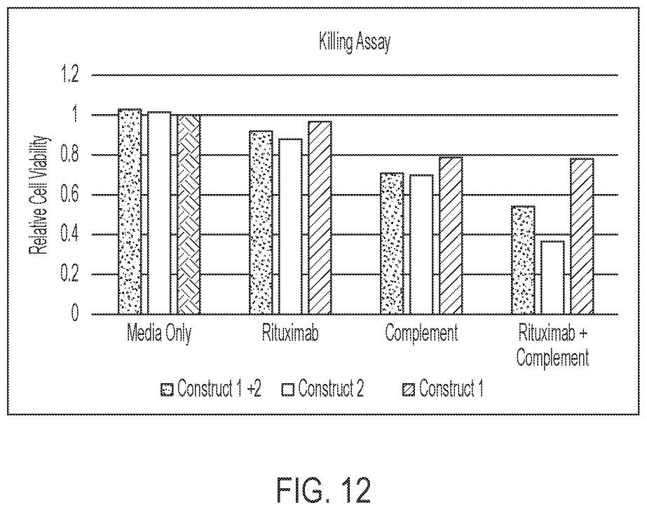

The percentage of GFP-positive cells was quantified to quantify the killing induced by the RQR8 kill switch. As shown in FIG. 12, incubation with complement alone resulted in some cell death, however this reduction in viability was observed in all of the groups of cells. Cell death was observed in cells that expressed Construct 2 or both Constructs 1 and 2 the presence of the combination of rituximab and complement, indicating specific RQR8-mediated cell death.

### REFERENCES

[1] Corlien et al., International journal of cancer, 99(1):7-13, 2002.
[2] Betts et Ia., Methods in cell biology, 75:497-512, 2004.
[3] Bimbaum et al. Proceedings of the National Academy of Sciences of the United States of America, 1 11(49):17576, 2014.
[4] Boissel et al., Nucleic acids research, 42(4):2591-2601, 2014.
[5] Cai et al., Blood, 128(22):4039, 2016.
[6] Chung et al., Proceedings of the National Academy of Sciences, 92(9):3712-3716, 1995.
[7] Cieri et al., Blood, 121(4):573-584, 2013.
[8] Corrigan-Curay et al., Molecular Therapy, 23(5):796-806, 2015.
[9] Dotti et al., Immunological reviews. 257(1):107-126, 2014.
[10] WO2015121454
[11] Farber et al., Nature Reviews Immunology, 14(1):24-35, 2014.
[12] US20170183413
[13] Galetto. Publication No. EP3137498 A1, 2017.
[14] US20140120905
[15] Galetto et al., Molecular Therapy-Methods & Clinical Development, 1:14021, 2014.
[16] Goebels et al., Brain, 123(3):508-518, 2000.
[17] Hahn et al., Nature immunology. 6(5):490-496, 2005.
[18] Hall et al., International Immunology, 3(4):359-368, 1991.
[19] Hart et al., Gene Therapy, 15:625-631, 2008.
[20] Hohlfeld et al., Proceedings of the National Academy of Sciences, 101(suppl 2):14599-14606, 2004.
[21] Holst et al., Nature Protocols Electronic Edition, l(1):406, 2006.
[22] Jensen et al., Immunological reviews, 257(1):127-144, 2014.
[23] Juillerat et al., Scientific reports. 5:8150, 2015.
[24] Justesen et al., Immunome research, 5(1):2, 2009.
[25] Jyothi et al., Nature biotechnology, 20(12):1215-1220, 2002.
[26] Mach et al., Immunological reviews, 138(1):207-221, 1994.
[27] Maecker et al., Nature Reviews Immunology, 12(3):191-200, 2012.
[28] Mamonkin et al., Blood, 126(8):983-992, 2015.
[29] Moisini et al., The Journal of Immunology, 180(5):3601-3611, 2008.
[30] Morris et al., Science translational medicine, 7(272):272rα10-272rα10, 2015.
[31] Moscou et al., Science, 326(5959):1501-1501, 2009.
[32] Mueller et al., Nature reviews Immunology, 16(2):79-89, 2016.
[33] Nagarajan et al., The Journal of Immunology, 168(4):1780-1786, 2002.
[34] Nagy et al., Nature medicine, 8(8):801-807, 2002.
[35] Ophir et al., Blood, 121(7):1220-1228, 2013.
[36] Osborn et al., Molecular therapy, 24(3):570-581, 2016.
[37] Philip et al., Blood, 124(8):1277-1287, 2014.
[38] US20170016025
[39] Poirot et al., Cancer research, 75(18):3853-3864, 2015.
[40] Qasim et al., Science translational medicine, 9(374):eaaj2013, 2017.
[41] Ruck et al., International journal of molecular sciences, 16(7):16414-16439, 2015.
[42] Salzer et al., Neurology, 87(20):2074-2081, 2016.
[43] Smith et al., U.S. patent application Ser. No. 14/018,021, 2013.
[44] Steidl et al., Nature, 471(7338):377-381, 2011.
[45] Suhoski et al., Molecular Therapy, 15(5):981-988, 2007.
[46] Taylor et al., Cell, 169(1):108-119, 2017.
[47] Valton et al., Molecular Therapy, 23(9):1507-1518, 2015.
[48] Vanderlugt et al., Nature Reviews Immunology, 2(2):85-95, 2002.
[49] Walchli et al., PloS one, 6(11):e27930, 2011.
[50] Wingerchuk et al., Mayo Clinic Proceedings, 89:225-240, 2014.
[51] Yang et al., Gene Therapy, 15(21):1411-1423, 2008.
[52] Yang, Journal of Immunotherapy, 31(9):830, 2008.
[53] Zhou et al., Immunology, 122(4):476-485, 2007.
Jiang et al., International immunology, 25(4):235-246, 2013.
Busch et al., The EMBO joumal, 15(2):418, 1996.
Akalin et al., Molecular Therapy, 17:S25, 2009.
Jin et al., Proceedings of the National Academy of Sciences, 111(10):3787-3792, 2014.
Tanimura et al., Blood, 125(18):2835-2844, 2015.
Hamano et al., Alveolar Macrophage Biology B32:A3147-A3147, 2016.
Arase et al., Journal of Dermatological Science, 84(1):e87, 2016.
Tanimura et al., Placenta, 46:108, 2016.
Hiwa et al., Arthritis & Rheumatology, 69(10):2069-2080, 2017.
Hansen et al. Trends in immunology, 31(10):363, 2010.
Hacohen et al., Cancer immunology research, 1(1):11-15, 2013.
Owens et al., Annals of neurology, 65(60):639-649, 2009.
Chastre et al., New England Journal of Medicine, 374(15):1495-1496, 2016.
Housley et al., Clinical Immunology, 161(1):51-58, 2015.
Larman et al., 2013. Journal of autoimmunity, 43:1-9, 2013.
Wu et al., Science, 350(6258):aab4077, 2015
Tran et al. Scientific reports, 6:31730, 2016
Schall et al., Nature Reviews Immunology, 11(5):355-363, 2011.
Tabarkiewicz et al., Archivum immunologiae et therapiae experimentalis, 63(6):435-449, 2015.
Brown et al., New England Journal of Medicine, 375(26):2561-2569, 2016.
Gagliani et al., Nature. 523(7559):221-225, 2015.
Barreiro, et al. Cardiovascular research, 86(2):174-182, 2010
Wang and Rivière. Molecular Therapy-Oncolytics, 3:16015 2016.
Birkle et al., Blood, 110(9):3316-3325, 2007.
Wu and Hwang, Journal of immunology. 168(10):5096, 2002.
Singh et al., Journal of immunology, 180(1):214-221, 2008.
Ryu et al., Molecules and Cells, 39.12:898-908, 2016.
Hiepe et al., Nature Reviews Rheumatology, 7(3):170-178, 2011
Garboczi, et al. The Journal of Immunology, 157(12):5403-5410, 1996.
Quaresma, et al., 2015. Viruses, 8(1):5 2015.
Sanjana, et al. Nature methods, 11(8):783-784, 2014.
Korn et al. Proceedings of the National Academy of Sciences, 105(47):18460-18465, 2008.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

1. A major histocompatibility complex (MHC)-based chimeric receptor, comprising:
(i) an extracellular domain of a MHC molecule conjugated to an antigenic peptide from an antigen involved in an autoimmune disease; and
(ii) a cytoplasmic signaling domain, at least one co-stimulatory domain, or a combination thereof;
wherein the MHC molecule is a class I MHC.

2. The MHC-based chimeric receptor of claim 1, which comprises the at least one co-stimulatory domain.

3. The MHC-based chimeric receptor of claim 2, wherein the at least one co-stimulatory domain is a co-stimulatory domain from 4-1BB (CD137), a co-stimulatory domain from CD28, or a combination thereof.

4. The MHC-based chimeric receptor of claim 2, wherein the MHC-based chimeric receptor is free of a cytoplasmic signaling domain.

5. The MHC-based chimeric receptor of claim 1, which further comprises a hinge domain located between (i) and (ii).

6. The MHC-based chimeric receptor of claim 1, which comprises a cytoplasmic signaling domain of CD3ζ.

7. The MHC-based chimeric receptor of claim 1, wherein the antigenic peptide is from myelin basic protein (MBP), myelin oligodendrocyte glycoprotein (MOG), proteolipid protein (PLP), insulin, glutamate decarboxylase, or described in Table 1.

8. (canceled)

9. The MHC-based chimeric receptor of claim 1, wherein the class I MHC is a human class I MHC.

10. The MHC-based chimeric receptor of claim 1 wherein the extracellular domain of the chimeric receptor comprises an extracellular domain of the alpha chain of the class I MHC, which is fused to the antigenic peptide.

11. The MHC-based chimeric receptor of claim 10, wherein the chimeric receptor is a fusion polypeptide comprising (i) the extracellular domain of the class I MHC molecule, and (ii) the cytoplasmic domain, the at least one co-stimulatory domain, or the combination thereof.

12. The MHC-based chimeric receptor of claim 11, wherein the chimeric receptor is a fusion polypeptide, which comprises, from N-terminus to C-terminus, a signal peptide, a first peptide linker, the antigenic peptide, a second peptide linker, an extracellular domain of macroglobulin, a third peptide linker, the class I MHC molecule, a transmembrane domain, the at least one co-stimulatory domain, and CD3ζ.

13-20. (canceled)

21. A genetically modified immune cell, which expresses a MHC-based chimeric receptor of claim 1.

22. The genetically modified immune cell of claim 21, which is a T cell.

23. The genetically modified immune cell of claim 22,
wherein the activity of the endogenous T cell receptor (TCR) is suppressed;
wherein expression of the endogenous CD52 is disrupted;
wherein the genetically modified immune cell further express a suicide gene, a marker gene or both;
wherein the immune cell is further modified for lymph node delivery and retention;
wherein the expression of endogenous sphingosine-1-phosphate receptor 1 is disrupted in the immune cell;
wherein the immune cell is further modified to express one or more surface molecules for tertiary lymph node or ectopic lymph node delivery and retention;
wherein the immune cell is modified with IL6ST knockout, IL6R knockout, or both;
wherein the immune cell is further modified to express or overly express a chemokine receptor;
wherein the immune cell is further modified to express or overly express an adhesion receptor; and/or
wherein the immune cell further comprises a genetic modification that results in blockade of PD-1 signaling.

24-41. (canceled)

42. The genetically modified immune cell of claim 21, which is a regulatory T cell, wherein the regulatory T cell is CD25+.

43. (canceled)

44. The genetically modified immune cell of claim 42, wherein the regulatory T cell comprises a transgene coding for CD25; wherein the regulatory T cell comprises a transgene coding for FoxP3; and/or wherein the regulatory T cell further expresses a chimeric receptor specific to CD19, a chimeric receptor specific to CS-1, or both.

45-48. (canceled)

49. The genetically modified immune cell of claim 42, wherein the regulatory T cell displays an antibody specific to an autoantigen.

50-53. (canceled)

54. The genetically modified immune cell of claim 21, which is a cytotoxic lymphocyte, wherein the cytotoxic T cell is CD8+.

55-57. (canceled)

58. A method for suppressing autoreactive immune cells in a subject having an autoimmune disease, comprising administering to the subject an effective amount of genetically modified immune cells set forth in claim 21.

59-76. (canceled)


SEQUENCE LISTING (431 sequences)

SEQ ID NO: 1 - protein, 203 aa, Homo sapiens [sequence omitted]

SEQ ID NO: 2 - protein, 40 aa, Homo sapiens
GSKYLATASTMDHARHGFLPRHRDTGILDSIGRFFGGDRG

SEQ ID NO: 3 - protein, 20 aa, Homo sapiens
KYLATASTMDHARHGFLPRH

SEQ ID NO: 4 - protein, 23 aa, Homo sapiens
ATASTMDHARHGFLPRHRDTGIL

SEQ ID NO: 5 - protein, 20 aa, Homo sapiens
RDTGILDSIGRFFGGDRGAP

SEQ ID NO: 6 - protein, 29 aa, Homo sapiens
IGRFFGGDRGAPKRGSGKDSHHPARTAHY

SEQ ID NO: 7 - protein, 19 aa, Homo sapiens
APKRGSGKDSHHAARTAHY

SEQ ID NO: 8 - protein, 20 aa, Homo sapiens
GSGKDSHHPARTAHYGSLPQ

SEQ ID NO: 9 - protein, 19 aa, Homo sapiens
HHPARTAHYGSLPQKSHGR

SEQ ID NO: 10 - protein, 19 aa, Homo sapiens
HAARTAHYGSLPQKSQGHR

SEQ ID NO: 11 - protein, 19 aa, Homo sapiens
SLPQKSHGRTQDENPVVHF

SEQ ID NO: 12 - protein, 20 aa, Homo sapiens
PQDENPVVHFFKNIVTPRTP

SEQ ID NO: 13 - protein, 20 aa, Homo sapiens
TQDENPVVHFFKNIVTPRTP

SEQ ID NO: 14 - protein, 19 aa, Homo sapiens
QDENPVVHFFKNIVTPRTP

SEQ ID NO: 15 - protein, 19 aa, Homo sapiens
DENPVVHFFKNIVTPRTPP

SEQ ID NO: 16 - protein, 15 aa, Homo sapiens
ENPVVHFFKNIVTPR

SEQ ID NO: 17 - protein, 17 aa, Homo sapiens
ENPVVHFFKNIVTPRTP

SEQ ID NO: 18 - protein, 14 aa, Homo sapiens
ENPVVHFFKNIVTP

SEQ ID NO: 19 - protein, 20 aa, Homo sapiens
NPVVHFFKNIVTPRTPPPSQ

SEQ ID NO: 20 - protein, 13 aa, Homo sapiens
VVHFFKNIVTPRT

SEQ ID NO: 21 - protein, 20 aa, Homo sapiens
VVHFFKNIVTPRTPPPSQGK

SEQ ID NO: 22 - protein, 19 aa, Homo sapiens
KNIVTPRTPPPSQGKGRGL

SEQ ID NO: 23 - protein, 19 aa, Homo sapiens
PSQGKGRGLSLSRFSWGAE

SEQ ID NO: 24 - protein, 20 aa, Homo sapiens
GKGRGLSLSRFSWGAEGQRP

SEQ ID NO: 25 - protein, 19 aa, Homo sapiens
LSRFSWGAEGQRPGFGYGG

SEQ ID NO: 26 - protein, 19 aa, Homo sapiens
QRPGFGYGGRASDYKSAHK

SEQ ID NO: 27 - protein, 19 aa, Homo sapiens
ASDYKSAHKGFKGVDAQGT

SEQ ID NO: 28 - protein, 19 aa, Homo sapiens
FKGVDAQGTLSKIFKLGGR

SEQ ID NO: 29 - protein, 20 aa, Homo sapiens
VDAQGTLSKIFKLGGRDSRS

SEQ ID NO: 30 - protein, 20 aa, Homo sapiens
SKIFKLGGRDSRSGSPMARR

SEQ ID NO: 31 - protein, 247 aa, Homo sapiens
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLVFLCLQYRLRGKLRAEIENLHRTFDPHFLRVPCWKITLFVIVPVLGPLVALIICYNWLHRRLAGQFLEELRNPF

SEQ ID NO: 32 - protein, 277 aa, Homo sapiens [sequence omitted]

SEQ ID NO: 33 - protein, 582 aa, Homo sapiens
MIFLTALPLFWIMISASRGGHWGAWMPSSISAFEGTCVSIPCRFDFPDELRPAVVHGVWYFNSPYPKNYPPVVFKSRTQVVHESFQGRSRLLGDLGLRNCTLLLSNVSPELGGKYYFRGDLGGYNQYTFSEHSVLDIVNTPNIVVPPEVVAGTEVEVSCMVPDNCPELRPELSWLGHEGLGEPAVLGRLREDEGTWVQVSLLHFVPTREANGHRLGCQASFPNTTLQFEGYASMDVKYPPVIVEMNSSVEAIEGSHVSLLCGADSNPPPLLTWMRDGTVLREAVAESLLLELEEVTPAEDGVYACLAENAYGQDNRTVGLSVMYAPWKPTVNGTMVAVEGETVSILCSTQSNPDPILTIFKEKQILSTVIYESELQLELPAVSPEDDGEYWCVAENQYGQRATAFNLSVEFAPVLLLESHCAAARDTVQCLCVVKSNPEPSVAFELPSRNVTVNESEREFVYSERSGLVLTSILTLRGQAQAPPRVICTARNLYGAKSLELPFQGAHRLMWAKIGPVGAVVAFAILIAIVCYITQTRRKKNVTESPSFSAGDNPPVLFSSDFRISGAPEKYESKEVSTLESH

SEQ ID NO: 34 - protein, 20 aa, synthetic construct [sequence omitted]

SEQ ID NO: 35 - protein, 31 aa, synthetic construct
  REGION 1..31: note = Synthetic
LLECCARCLVGAPFASLVATGLCFFGVALFC

SEQ ID NO: 36 - protein, 20 aa, synthetic construct
  REGION 1..20: note = Synthetic
LVGAPFASLVATGLCFFGVA

SEQ ID NO: 37 - protein, 31 aa, synthetic construct
  REGION 1..31: note = Synthetic
FGVALFCGCEVEALTGTEKLIETYFSKNYQD

SEQ ID NO: 38 - protein, 20 aa, synthetic construct
  REGION 1..20: note = Synthetic
LFCGCGHEALTGTEKLIETY

SEQ ID NO: 39 - protein, 20 aa, synthetic construct
  REGION 1..20: note = Synthetic
TGTEKLIETYFSKNYQDYEY

SEQ ID NO: 40 - protein, 21 aa, synthetic construct
  REGION 1..21: note = Synthetic
TGTEKLIETYFSKNYQDYEYL

SEQ ID NO: 41 - protein, 32 aa, synthetic construct
  REGION 1..32: note = Synthetic
YFSKNYQDYEYLINVIHAFQYVIYGTASFFFL

SEQ ID NO: 42 - protein, 32 aa, synthetic construct
  REGION 1..32: note = Synthetic
GTASFFFLYGALLLAYGFYTTGAVRQIFGDYK

SEQ ID NO: 43 - protein, 20 aa, synthetic construct
  REGION 1..20: note = Synthetic
LYGALLLAEGFYTTGAVRQI

SEQ ID NO: 44 - protein, 21 aa, synthetic construct
  REGION 1..21: note = Synthetic
FYYTTGAVRQIFGDYKTTICG

SEQ ID NO: 45 - protein, 22 aa, synthetic construct
  REGION 1..22: note = Synthetic
AVRQIFGDYKTTICGKGLSATV

SEQ ID NO: 46 - protein, 31 aa, synthetic construct
  REGION 1..31: note = Synthetic
RQIFGDYKTTCGKGLSATVTGGQKGRGSRGQ

SEQ ID NO: 47 - protein, 20 aa, synthetic construct
  REGION 1..20: note = Synthetic
KGLSATVTGGQKGRGYRGQH

SEQ ID NO: 48 - protein, 20 aa, synthetic construct
  REGION 1..20: note = Synthetic
QKGRGSRGQHQAHSLERVCH

SEQ ID NO: 49 - protein, 32 aa, synthetic construct
  REGION 1..32: note = Synthetic
KGRGSRGQHQAHSLERVCHCLGCWLGHPDKFV

SEQ ID NO: 50 - protein, 32 aa, synthetic construct
  REGION 1..32: note = Synthetic
LGHPDKFVGITYALTVVWLLVFACSAVPVYIY

SEQ ID NO: 51 - protein, 32 aa, synthetic construct
  REGION 1..32: note = Synthetic
SAVPVYIYFNTWTTCQSIAAPCKTSASIGTLC

SEQ ID NO: 52 - protein, 20 aa, synthetic construct
  REGION 1..20: note = Synthetic
AVPVYIYFNTWTTCQSIAFP

SEQ ID NO: 53 - protein, 20 aa, synthetic construct
  REGION 1..20: note = Synthetic
WTTCQSIAFPSKTSASIGSL

SEQ ID NO: 54 - protein, 32 aa, synthetic construct
  REGION 1..32: note = Synthetic
SASIGTLCADARMYGVLPWNAFFGKVCGSNLL

SEQ ID NO: 55 - protein, 32 aa, synthetic construct
  REGION 1..32: note = Synthetic
KVCGSNLLSICKTAEFQMTFHLFIAAFVGAAA

SEQ ID NO: 56 - protein, 32 aa, synthetic construct
  REGION 1..32: note = Synthetic
AAFVGAAATLVSLLTFMIAATYNFAVLKLMGR

SEQ ID NO: 57 - protein, 20 aa, synthetic construct
  REGION 1..20: note = Synthetic
MIAATYNFAVLKLMGRGTKF

SEQ ID NO: 58 - protein, 19 aa, synthetic construct
  REGION 1..19: note = Synthetic
MAATYNFAVLKLMGRFTKF

SEQ ID NO: 59 - protein, 42 aa, synthetic construct
  REGION 1..42: note = Synthetic
KRGRKKLLYIFKQPFMRPVQTTQEEDGCSCRFPEEEEGGCEL

SEQ ID NO: 60 - protein, 47 aa, synthetic construct
  REGION 1..47: note = Synthetic
PGKRGRKKLLYIFKQPFMRPVQTTQEEDGCSCRFPEEEEGGCELAHA

SEQ ID NO: 61 - protein, 41 aa, synthetic construct
  REGION 1..41: note = Synthetic
RSKRSRGGHSDYMNMTPRRPGPTRKHYQPYAPPRDFAAYRS

SEQ ID NO: 62 - protein, 46 aa, synthetic construct
  REGION 1..46: note = Synthetic
PGRSKRSRGGHSDYMNMTPRRPGPTRKHYQPYAPPRDFAAYRSAHA

SEQ ID NO: 63 - protein, 87 aa, synthetic construct
  REGION 1..87: note = Synthetic
EMWHEGLEEASRLYFGERNVKGMFEVLEPLHAMMERGPQTLKETSFNQAYGRDLMEAQEWCRKYMKSGNVKDLLQAWDLYYHVFRRI

SEQ ID NO: 64 - protein, 115 aa, synthetic construct
  REGION 1..115: note = Synthetic
GSSSGSSSGSSSGSSSEMWHEGLEEASRLYFGERNVKGMFEVLEPLHAMMERGPQTLKETSFNQAYGRDLMEAQEWCRKYMKSGNVKDLLQAWDLYYHVFRRIGSSSGSSSGSSS

SEQ ID NO: 65 - protein, 107 aa, synthetic construct
  REGION 1..107: note = Synthetic
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 66 - protein, 135 aa, synthetic construct
  REGION 1..135: note = Synthetic
GSSSGSSSGSSSGSSSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGSSSGSSSGSSS

SEQ ID NO: 67 - protein, 112 aa, synthetic construct
  REGION 1..112: note = Synthetic
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - protein, 15 aa, synthetic construct
  REGION 1..15: note = Synthetic
GGGGSGGGGSGGGGS

SEQ ID NO: 69 - protein, 15 aa, synthetic construct
  REGION 1..15: note = Synthetic
GGGGGGSGGSGGSGG

SEQ ID NO: 70 - protein, 20 aa, synthetic construct
  REGION 1..20: note = Synthetic
GGGGSGGGGSGGGGSGGGGS

SEQ ID NO: 71 - DNA, 2340 bp, synthetic construct
  misc_feature 1..2340: note = Synthetic
  source 1..2340: mol_type = other DNA
atggtatgcttgaagctcccgggcgggtcctgcatgaccgctctcactgttactcttatggtccttagttcaccgcttgccctggcatctgatgagaatcccgtggttcatttttttaagaacatcgtcacaccgcgcaccccacctgggggaggcggatctggcggaggcgggagtggaggctcaggagacacaagaccccgattcttgtggcagcccaaaagggagtgccattttttcaatgggacggaacgagttcgcttccttgatcggtacttttacaaccaagaagagagtgtacggttcgactcagatgtcggcgagttccgagcggttacggaattggggcgacctgacgcggagtactggaactcccaaaaggatattttggagcaggcacgagcagctgtggacacctattgtcgacataattatggtgtggtggaatcctttacagttcagcggcgggtgcaacctaaagtgaccgtgtatccatctaaaacgcaacccctccaacaccataacctcctggtgtgttccgtaagcggcttctatcccgggtcaattgaggtcaggtggttcctcaacggtcaggaggagaaggccggaatggtaagtactggtcttatccagaacggagactggaccttccaaactttggtaatgttggaaacggtgccgcgatccggggaggtgtatacatgccaagttgaacacccgagtgttacgagccccctgacggttgagtggagggcgcggtcagagagcgcacaatctaaaatgctgtcaggagtaggcggatttgtactcggactcctctttttgggcgctgggttgtttatctactttagaaaccaaacaagtagagtaaagttttcccgaagtgcggacgctcccgcgtatcagcaaggtcaaaaccagctttacaacgaactgaacttgggacgacgcgaagagtacgatgttcttgataagcggagagggcgcgatcccgaaatggggggaaagcctcggaggaagaacccacaagaaggcctttataatgaactgcagaaggacaagatggcggaggcgtattccgaaataggcatgaagggtgaacggaggagaggaaagggacatgacggactttatcaaggattgtctaccgcaactaaagacacctatgacgcgttgcacatgcaggctctccctccgagaggttcgagcggcagtggagagggcagaggaagtctgctaacatgcggtgacgtcgaggagaatcctggcccaatggcaatatctggtgttcctgtcctcgggttttttatcatagccgtactgatgtcagcacaggaatcatgggcgataaaagaagagcacgtgataatacaggcggagttttatttgaacccggaccagagcggtgagttcatgttcgattttgatggcgacgagatatttcacgttgacatggcaaaaaaggaaacggtgtggagacttgaggagtttggacgattcgcatcatttgaggcacaaggagcactcgccaatatcgcggtggacaaggccaacctggagatcatgacaaaacgctccaattatacgcctatcactaatgtgccccctgaggttactgtgctcacaaattctcccgtagaacttagggaacctaacgtcctcatatgtttcatcgacaagttcactcctccggtggtcaatgtaacgtggcttcggaatggtaagccggtcaccacgggtgtctcagagaccgtatttctgcccagagaagaccacctcttccgcaaatttcattaccttccctttcttccttcaacggaagacgtttacgactgcagggtcgaacattgggggcttgacgagccacttctcaagcattgggagttcgacgccccatcaccgcttccagaaacgactgaaaacgttgtctgcgctcttggcctgacagtgggcctggtaggcattattatcgggaccatctttatcatcaaaggtttgacttcccgggtcaaatttagcagatccgctgacgcaccggcctaccagcagggccagaaccaactctacaacgagctgaatctcggccgacgggaagagtatgacgtactcgacaagcggagaggtcgagaccctgagatgggcggtaaaccgagacggaaaaatccccaagagggtctttataatgaactccagaaggataagatggctgaagcctattctgagatagggatgaaaggcgagcggcggaggggtaagggccatgatggcctttaccagggactctccacggcaaccaaagatacttacgacgcccttcacatgcaagccctcccgccacgc

SEQ ID NO: 72 - protein, 8 aa, synthetic construct
  REGION 1..8: note = Synthetic
GSGSGSGS

SEQ ID NO: 73 - protein, 21 aa, synthetic construct
  REGION 1..21: note = Synthetic
IYIWAPLAGTCGVLLLSLVIT

SEQ ID NO: 74 - protein, 21 aa, synthetic construct
  REGION 1..21: note = Synthetic
IWAPLAGICVALLLSLIITLI

SEQ ID NO: 75 - protein, 267 aa, synthetic construct
  REGION 1..267: note = Synthetic
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEEAAAREAAAREAAAREAAARGRVAILWHEMWHEGLEEASRLYFGERNVKGMFEVLEPLHAMMERGPQTLKETSFNQAYGRDLMEAQEWCRKYMKSGNVKDLLQAWDLYYHVFRRITTTPAPRPPTPAPTIASQPLSLRPEACRPAAGGAVHTRGLDFACD

SEQ ID NO: 76 - protein, 45 aa, synthetic construct
  REGION 1..45: note = Synthetic
GGGGSGGGGSGGGGSGGGGSGGGGSGGGGSGGGGSGGGGSGGGGS

SEQ ID NO: 77 - protein, 13 aa, synthetic construct
  REGION 1..13: note = Synthetic
LRWEPPPSTVSNM

SEQ ID NO: 78 - protein, 13 aa, synthetic construct
  REGION 1..13: note = Synthetic
LRWEPSSQPTIPI

SEQ ID NO: 79 - protein, 13 aa, synthetic construct
  REGION 1..13: note = Synthetic
LRWELSSQPTIPI

SEQ ID NO: 80 - protein, 30 aa, synthetic construct
  REGION 1..30: note = Synthetic
QTTPGERSSLPAFYPGTSGSCSGCGSLSLP

SEQ ID NO: 81 - protein, 46 aa, synthetic construct
  REGION 1..46: note = Synthetic
GSSSGSSSGSSSGSSSQTTPGERSSLPAFYPGTSGSCSGCGSLSLP

SEQ ID NO: 82 - protein, 19 aa, synthetic construct
  REGION 1..19: note = Synthetic
LRPVQAQAQSDCSCSTVSP

SEQ ID NO: 83 - protein, 35 aa, synthetic construct
  REGION 1..35: note = Synthetic
GSSSGSSSGSSSGSSSLRPVQAQAQSDCSCSTVSP

SEQ ID NO: 84 - protein, 16 aa, synthetic construct
  REGION 1..16: note = Synthetic
GLAVSTISSFFPPGYQ

SEQ ID NO: 85 - protein, 45 aa, synthetic construct
  REGION 1..45: note = Synthetic
TTTPAPRPPTPAPTIASQPLSLRPEACRPAAGGAVHTRGLDFACD

SEQ ID NO: 86 - protein, 231 aa, synthetic construct
  REGION 1..231: note = Synthetic
EPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 13 aa, synthetic construct
  REGION 1..13: note = Synthetic
EFDAPSPLPETTE

SEQ ID NO: 88 - protein, 13 aa, synthetic construct
  REGION 1..13: note = Synthetic
VEWRARSESAQSK

SEQ ID NO: 89 - protein, 24 aa, synthetic construct
  REGION 1..24: note = Synthetic
IYIWAPLAGTCGVLLLSLVITLYC

SEQ ID NO: 90 - protein, 24 aa, synthetic construct
  REGION 1..24: note = Synthetic
VGIIAGLVLFGAVITGAVVAAVMW

SEQ ID NO: 91 - protein, 24 aa, synthetic construct
  REGION 1..24: note = Synthetic
VGIIAGLVLLGAVITGAVVAAVMW

SEQ ID NO: 92 - protein, 21 aa, synthetic construct
  REGION 1..21: note = Synthetic
LCYLLDGILFIYGVILTALFL

SEQ ID NO: 93 - protein, 21 aa, synthetic construct
  REGION 1..21: note = Synthetic
MLSGVGGFVLGLLFLGAGLFI

SEQ ID NO: 94 - protein, 26 aa, synthetic construct
  REGION 1..26: note = Synthetic
MLSGVGGFVLGLLFLGAGLFIYFRNQ

SEQ ID NO: 95 - protein, 326 aa, synthetic construct
  REGION 1..326: note = Synthetic
ILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRR

SEQ ID NO: 96 - protein, 363 aa, synthetic construct
  REGION 1..363: note = Synthetic
PCVGSNPCYNQGTCEPTSENPFYRCLCPAKFNGLLCHILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRR

SEQ ID NO: 97 - protein, 18 aa, synthetic construct
  REGION 1..18: note = Synthetic
MLRLLLALNLFPSIQVTG

SEQ ID NO: 98 - protein, 20 aa, synthetic construct
  REGION 1..20: note = Synthetic
MSRSVALAVLALLSLSGLEA

SEQ ID NO: 99 - protein, 24 aa, synthetic construct
  REGION 1..24: note = Synthetic
MAVMAPRTLLLLLSGALALTQTWA

SEQ ID NO: 100 - protein, 25 aa, synthetic construct
  REGION 1..25: note = Synthetic
MAISGVPVLGFFIIAVLMSAQESWA

SEQ ID NO: 101 - protein, 28 aa, synthetic construct
  REGION 1..28: note = Synthetic
MVCLKLPGGSCMTALTVTLMVLSSPLAL

SEQ ID NO: 102 - protein, 29 aa, synthetic construct
  REGION 1..29: note = Synthetic
MVCLKLPGGSYMAKLTVTLMVLSSPLALA

SEQ ID NO: 103 - protein, 100 aa, synthetic construct
  REGION 1..100: note = Synthetic
  REGION 16..100: note = MISC_FEATURE - may be absent
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 104 - protein, 30 aa, synthetic construct
  REGION 1..30: note = Synthetic
GGGGSGGGGSGGGGSGGGGSGGGGSGGGGS

SEQ ID NO: 105 - protein, 60 aa, synthetic construct
  REGION 1..60: note = Synthetic
GGGGSGGGGSGGGGSGGGGSGGGGSGGGGSGGGGSGGGGSGGGGSGGGGSGGGGSGGGGS

SEQ ID NO: 106 - protein, 75 aa, synthetic construct
  REGION 1..75: note = Synthetic
GGGGSGGGGSGGGGSGGGGSGGGGSGGGGSGGGGSGGGGSGGGGSGGGGSGGGGSGGGGSGGGGSGGGGSGGGGS

SEQ ID NO: 107 - protein, 9 aa, synthetic construct
  REGION 1..9: note = Synthetic
KLIETYFSK

SEQ ID NO: 108 - protein, 9 aa, synthetic construct
  REGION 1..9: note = Synthetic
LLFGYPVYV

SEQ ID NO: 109 - protein, 271 aa, synthetic construct
  REGION 1..271: note = Synthetic
GSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLT

SEQ ID NO: 110 - protein, 271 aa, synthetic construct
  REGION 1..271: note = Synthetic
GSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLT

SEQ ID NO: 111 - protein, 4 aa, synthetic construct
  REGION 1..4: note = Synthetic
LRWE

SEQ ID NO: 112 - protein, 271 aa, synthetic construct
  REGION 1..271: note = Synthetic
GSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDSPKAHVTHHSRPEDKVTLRCWALGFYPADITLTWQLNGEELIQDMELVETRPAGDGTFQKWASVVVPLGKEQYYTCHVYHQGLPEPLT

SEQ ID NO: 113 - protein, 271 aa, synthetic construct
  REGION 1..271: note = Synthetic
GSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHEAEQLRAYLDGTCVEWLRRYLENGKETLQRTDSPKAHVTHHSRPEDKVTLRCWALGFYPADITLTWQLNGEELIQDMELVETRPAGDGTFQKWASVVVPLGKEQYYTCHVYHQGLPEPLT

SEQ ID NO: 114 - protein, 99 aa, Homo sapiens [sequence omitted]

SEQ ID NO: 115 - protein, 99 aa, Mus musculus [sequence omitted]

SEQ ID NO: 116 - DNA, 149 bp, synthetic construct
  misc_feature 1..149: note = Synthetic
  source 1..149: mol_type = other DNA
agcgctctcgtacagagttggcattataatacgactcactataggggagaatcaaaatcggtgaatgttttagagctagaaatagcaagttaaaataaggctagtccgttatcaacttgaaaaagtggcaccgagtcggtgcttttttt

SEQ ID NO: 117 - protein, 185 aa, synthetic construct
  REGION 1..185: note = Synthetic
GDTRPRFLWQPKRECHFFNGTERVRFLDRYFYNQEESVRFDSDVGEFRAVTELGRPDAEYWNSQKDILEQARAAVDTYCRHNYGVVESFTVQRRVQPKVTVYPSKTQPLQHHNLLVCSVSGFYPGSIEVRWFLNGQEEKAGMVSTGLIQNGDWTFQTLVMLETVPRSGEVYTCQVEHPSVTSPLT

SEQ ID NO: 118 - protein, 178 aa, synthetic construct
  REGION 1..178: note = Synthetic
IKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHW

SEQ ID NO: 119 - protein, 185 aa, synthetic construct
  REGION 1..185: note = Synthetic
IKEEHVIIQAESYLNPDQSGEFKFDFDGDEIFHVDMAKKETVWRLEEFGRFASFEAQGALANIAVDKANLEIMTKRSNYTPIEETEVPTSLRRLEQPNVAISLSRTEALNHHNTLVCSVTDFYPAKIKVRWFRNGQEETVGVSSTQLIRNGDWTFQVLVMLEMTPHQGEVYTCHVEHPSLKSPIT

SEQ ID NO: 120 - protein, 183 aa, synthetic construct
  REGION 1..183: note = Synthetic
IKEEHVIIQAESYLNPDQSGEFKFDFDGDEIFHVDMAKKETVWRLEEFGRFASFEAQGALANIAVDKANLEIMTKRSNYTPIATNEAPQATVFPKSPVLLGQPHTLICFVDNIFPPVINITWLRNSKSVTDGVYETSFLVNRDHSFHKLSYLTFIPSDDDIYDCKVEHWGLEEPVLKHWEPEI

SEQ ID NO: 121 - protein, 90 aa, synthetic construct
  REGION 1..90: note = Synthetic
RPRFLWQSKRECHFFNGTERVRFLDRYFYNQEESVRFDSDVGEFRAVTELGRPDAEYWNSQKDILEQARAAVDTYCRHNYGVVESFTVQR

SEQ ID NO: 122 - protein, 82 aa, synthetic construct
  REGION 1..82: note = Synthetic
IKEEHVIIQAESYLNPDQSGEFKFDFDGDEIFHVDMAKKETVWRLEEFGRFASFEAQGALANIAVDKANLEIMTKRSNYTPI

SEQ ID NO: 123 - protein, 272 aa, synthetic construct [sequence omitted]

SEQ ID NO: 124 - protein, 312 aa, synthetic construct
  REGION 1..312: note = Synthetic
MLLLLLLLGLAGSGLGAVVSQHPSWVISKSGTSVKIECRSLDFQATTMFWYRQFPKQSLMLMATSNEGSKATYEQGVEKDKFLINHASLTLSTLTVTSAHPEDSSFYICSARDLTSGANNEQFFGPGTRLTVLEDLKNVFPPEVAVFEPSEAEISHTQKATLVCLATGFYPDHVELSWWVNGKEVHSGVSTDPQPLKEQPALNDSRYSLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 125 - protein, 278 aa, synthetic construct
  REGION 1..278: note = Synthetic
MAMLLGASVLILWLQPDWVNSQQKNDDQQVKQNSPSLSVQEGRISILNCDYTNSMFDYFLWYKKYPAEGPTFLISISSIKDKNADGRFTVFLNKSAKHLSLHIVPSQPGDSAVYFCAAMEGAQKLVFGQGTRLTINPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 126 - protein, 312 aa, synthetic construct
  REGION 1..312: note = Synthetic
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYPGGGFYEQYFGPGTRLTVTEDLKNVFPPEVAVFEPSEAEISHTQKATLVCLATGFYPDHVELSWWVNGKEVHSGVSTDPQPLKEQPALNDSRYALSSRLRVSATFWQDPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 127 - protein, 461 aa, synthetic construct
  REGION 1..461: note = Synthetic
MALPVTALLLPLALLLHAARPEVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSSGGGGSGGGGSGGGGSDIVMTQAAPSIPVTPGESVSISCRSSKSLLNSNGNGSGSGTAFTLRISRVEAEDVGVYYCMQHLEYPFTAGTKLELKRSDPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 128 - protein, 495 aa, synthetic construct
  REGION 1..495: note = Synthetic
METDTLLLWVLLLWVPGSTGEVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSSGGGGSGGGGSGGGGSDIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKRSDPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 129 - protein, 432 aa, synthetic construct
  REGION 1..432: note = Synthetic
MALPVTALLLPLALLLHAARPEVKLLESGGGLVQPGGSLKLSCAASGFDFSRYWMSWVRQAPGKGLEWIGEINPDSSTINYTPSLKDKFIISRDNAKNTLYLQMSKVRSEDTALYYCARPDGNYWYFDVWGAGTTVTVSSGGGGSGGGGSGGGGSDIVMTQSHKFMSTSVGDRVSITCKASQDVGIAVAWYQQKPGQSPKLLIYWASTRHTGVPDRFTGSGSGTDFTLTISNVQSEDLADYFCQQYSSYPYTFGGGTKLEIKGLAVSTISSFFPPGYQKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 130 - protein, 485 aa, synthetic construct
  REGION 1..485: note = Synthetic
MALPVTALLLPLALLLHAARPEVKLLESGGGLVQPGGSLKLSCAASGFDFSRYWMSWVRQAPGKGLEWIGEINPDSSTINYTPSLKDKFIISRDNAKNTLYLQMSKVRSEDTALYYCARPDGNYWYFDVWGAGTTVTVSSGGGGSGGGGSGGGGSDIVMTQSHKFMSTSVGDRVSITCKASQDVGIAVAWYQQKPGQSPKLLIYWASTRHTGVPDRFTGSGSGTDFTLTISNVQSEDLADYFCQQYSSYPYTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 131 - protein, 647 aa, synthetic construct
  REGION 1..647: note = Synthetic
MALPVTALLLPLALLLHAARPEVKLLESGGGLVQPGGSLKLSCAASGFDFSRYWMSWVRQAPGKGLEWIGEINPDSSTINYTPSLKDKFIISRDNAKNTLYLQMSKVRSEDTALYYCARPDGNYWYFDVWGAGTTVTVSSGGGGSGGGGSGGGGSDIVMTQSHKFMSTSVGDRVSITCKASQDVGIAVAWYQQKPGQSPKLLIYWASTRHTGVPDRFTGSGSGTDFTLTISNVQSEDLADYFCQQYSSYPYTFGGGTKLEIKEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 132 - protein, 433 aa, synthetic construct
  REGION 1..433: note = Synthetic
MALPVTALLLPLALLLHAARPQVQLQQPGAELVRPGASVKLSCKASGYSFTTYWMNWVKQRPGQGLEWIGMIHPSDSETRLNQKFKDKATLTVDKSSSTAYMQLSSPTSEDSAVYYCARSTMIATRAMDYWGQGTSVTVSSGGGGSGGGGSGGGGSDIVMTQSQKSMSTSVGDRVSITCKASQDVITGVAWYQQKPGQSPKLLIYSASYRYTGVPDRFTGSGSGTDFTFTISNVQAEDLAVYYCQQHYSTPLTFGAGTKLELKGLAVSTISSFFPPGYQKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 133 - protein, 486 aa, synthetic construct
  REGION 1..486: note = Synthetic
MALPVTALLLPLALLLHAARPQVQLQQPGAELVRPGASVKLSCKASGYSFTTYWMNWVKQRPGQGLEWIGMIHPSDSETRLNQKFKDKATLTVDKSSSTAYMQLSSPTSEDSAVYYCARSTMIATRAMDYWGQGTSVTVSSGGGGSGGGGSGGGGSDIVMTQSQKSMSTSVGDRVSITCKASQDVITGVAWYQQKPGQSPKLLIYSASYRYTGVPDRFTGSGSGTDFTFTISNVQAEDLAVYYCQQHYSTPLTFGAGTKLELKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 134 - protein, 648 aa, synthetic construct
  REGION 1..648: note = Synthetic
MALPVTALLLPLALLLHAARPQVQLQQPGAELVRPGASVKLSCKASGYSFTTYWMNWVKQRPGQGLEWIGMIHPSDSETRLNQKFKDKATLTVDKSSSTAYMQLSSPTSEDSAVYYCARSTMIATRAMDYWGQGTSVTVSSGGGGSGGGGSGGGGSDIVMTQSQKSMSTSVGDRVSITCKASQDVITGVAWYQQKPGQSPKLLIYSASYRYTGVPDRFTGSGSGTDFTFTISNVQAEDLAVYYCQQHYSTPLTFGAGTKLELKEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 135 - protein, 434 aa, synthetic construct
  REGION 1..434: note = Synthetic
MALPVTALLLPLALLLHAARPQVQLQQSGAELARPGASVKLSCKASGYTFTSYWMQWVKQRPGQGLEWIGAIYPGDGDTRYTQKFKGKATLTADKSSSTAYMQLSSLASEDSAVYYCARGKVYYGSNPFAYWGQGTLVTVSAGGGGSGGGGSGGGGSDIQMTQSSSYLSVSLGGRVTITCKASDHINNWLAWYQQKPGNAPRLLISGATSLETGVPSRFSGSGSGKDYTLSITSLQTEDVATYYCQQYWSTPWTFGGGTKLEIKGLAVSTISSFFPPGYQKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 136 - protein, 487 aa, synthetic construct
  REGION 1..487: note = Synthetic
MALPVTALLLPLALLLHAARPQVQLQQSGAELARPGASVKLSCKASGYTFTSYWMQWVKQRPGQGLEWIGAIYPGDGDTRYTQKFKGKATLTADKSSSTAYMQLSSLASEDSAVYYCARGKVYYGSNPFAYWGQGTLVTVSAGGGGSGGGGSGGGGSDIQMTQSSSYLSVSLGGRVTITCKASDHINNWLAWYQQKPGNAPRLLISGATSLETGVPSRFSGSGSGKDYTLSITSLQTEDVATYYCQQYWSTPWTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 137 - protein, 649 aa, synthetic construct
  REGION 1..649: note = Synthetic
MALPVTALLLPLALLLHAARPQVQLQQSGAELARPGASVKLSCKASGYTFTSYWMQWVKQRPGQGLEWIGAIYPGDGDTRYTQKFKGKATLTADKSSSTAYMQLSSLASEDSAVYYCARGKVYYGSNPFAYWGQGTLVTVSAGGGGSGGGGSGGGGSDIQMTQSSSYLSVSLGGRVTITCKASDHINNWLAWYQQKPGNAPRLLISGATSLETGVPSRFSGSGSGKDYTLSITSLQTEDVATYYCQQYWSTPWTFGGGTKLEIKEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 138 - protein, 433 aa, synthetic construct
  REGION 1..433: note = Synthetic
MALPVTALLLPLALLLHAARPQVQLQQSGPELVKPGASVKISCKASGYAFSSSWMNWVKQRPGQGLEWIGRIYPGDGDTKYNGKFKGKATLTADKSSSTAYMQLSSLTSVDSAVYFCARSTMIATGAMDYWGQGTSVTVSSGGGGSGGGGSGGGGSETTVTQSPASLSMAIGEKVTIRCITSTDIDDDMNWYQQKPGEPPKLLISEGNTLRPGVPSRFSSSGYGTDFVFTIENMLSEDVADYYCLQSDNLPLTFGGGTKLEIKGLAVSTISSFFPPGYQKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 139 - protein, 486 aa, synthetic construct
  REGION 1..486: note = Synthetic
MALPVTALLLPLALLLHAARPQVQLQQSGAELARPGASVKLSCKASGYTFTSYWMQWVKQRPGQGLEWIGAIYPGDGDTRYTQKFKGKATLTADKSSSTAYMQLSSLASEDSAVYYCARGKVYYGSNPFAYWGQGTLVTVSAGGGGSGGGGSGGGGSDIQMTQSSSYLSVSLGGRVTITCKASDHINNWLAWYQQKPGNAPRLLISGATSLETGVPSRFSGSGSGKDYTLSITSLQTEDVATYYCQQYWSTPWTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 140 - protein, 647 aa, synthetic construct
  REGION 1..647: note = Synthetic
MALPVTALLLPLALLLHAARPQVQLQQSGPELVKPGASVKISCKASGYAFSSSWMNWVKQRPGQGLEWIGRIYPGDGDTKYNGKFKGKATLTADKSSSTAYMQLSSLTSVDSAVYFCARSTMIATGAMDYWGQGTSVTVSSGGGGSGGGGSGGGGSETTVTQSPASLSMAIGEKVTIRCITSTDIDDDMNWYQQKPGEPPKLLISEGNTLRPGVPSRFSSSGYGTDFVFTIENMLSEDVADYYCLQSDNLPLTFGGGTKLEIKEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRGKGHDGLYQGLSTATKDTYDALHMQALPPR

SEQ ID NO: 141 - protein, 432 aa, synthetic construct
  REGION 1..432: note = Synthetic
MALPVTALLLPLALLLHAARPQVQLQQSGPELVKPGASVKISCKASGYAFSSSWMNWVKQRPGQGLEWIGRIYPGDGDTKYNGKFKGKATLTADKSSSTAYMQLSSLTSVDSAVYFCARSTMIATGAMDYWGQGTSVTVSSGGGGSGGGGSGGGSDIVMTQSHKFMSTSVGDRVSITCKASQDVSTAVAWYQQKPGQSPKLLIYSASYRYTGVPDRFTGSGSGTDFTFTISSVQAEDLAVYYCQQHYSTPPYTFGGGTKLEIKGLAVSTISSFFPPGYQKRGRKKLLYIFKQPFMRPVQTTQEEDGCCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 142 - protein, 486 aa, synthetic construct
  REGION 1..486: note = Synthetic
MALPVTALLLPLALLLHAARPQVQLQQSGPELVKPGASVKISCKASGYAFSSSWMNWVKQRPGQGLEWIGRIYPGDGDTKYNGKFKGKATLTADKSSSTAYMQLSSLTSVDSAVYFCARSTMIATGAMDYWGQGTSVTVSSGGGGSGGGGSGGGGSDIVMTQSHKFMSTSVGDRVSITCKASQDVSTAVAWYQQKPGQSPKLLIYSASYRYTGVPDRFTGSGSGTDFTFTISSVQAEDLAVYYCQQHYSTPPYTFGGGTKLEIKTTTPAPRPPTPAPTIASQPLSLRPEACRPAAGGAVHTRGLDFADIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 143 - protein, 648 aa, synthetic construct
  REGION 1..648: note = Synthetic
MALPVTALLLPLALLLHAARPQVQLQQSGPELVKPGASVKISCKASGYAFSSSWMNWVKQRPGQGLEWIGRIYPGDGDTKYNGKFKGKATLTADKSSSTAYMQLSSLTSVDSAVYFCARSTMIATGAMDYWGQGTSVTVSSGGGGSGGGGSGGGGSDIVMTQSHKFMSTSVGDRVSITCKASQDVSTAVAWYQQKPGQSPKLLIYSASYRYTGVPDRFTGSGSGTDFTFTISSVQAEDLAVYYCQQHYSTPPYTFGGGTKLEIKEPKSPDKTHTCPPCPAPPVAGPSVFLFPPKPKDTLMIARTPEVCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 144 - protein, 156 aa, synthetic construct
  REGION 1..156: note = Synthetic
MGTSLLCWMALCLLGADHADACPYSNPSLCSGGGGSELPTQGTFSNVSTNVSPAKPTTTACPYSNPSLCSGGGGSPAPPPTPAPTIASQPLSLRPEACRPAAGGAVHTRGLDFACDIYIWAPLAGTCGVLLLSLVITLYCNHRNRRRVCKCPRPVV

SEQ ID NO: 145 - protein, 55 aa, synthetic construct
  REGION 1..55: note = Synthetic
ACPYSNPSLCSGGGGSELPTQGTFSNVSTNVSPAKPTTTACPYSNPSLCSGGGGS

SEQ ID NO: 146 - DNA, 20 bp, Homo sapiens
  source 1..20: mol_type = other DNA
tgacgttacctcgtgcggcc

SEQ ID NO: 147 - DNA, 20 bp, Homo sapiens
  source 1..20: mol_type = other DNA
cacgaagctctccgatgtgt

SEQ ID NO: 148 - DNA, 20 bp, Homo sapiens
  source 1..20: mol_type = other DNA
gcgtgacttccacatgagcg

SEQ ID NO: 149 - DNA, 20 bp, Homo sapiens
  source 1..20: mol_type = other DNA
ttggaactggccggctggcc

SEQ ID NO: 150 - DNA, 20 bp, Homo sapiens
  source 1..20: mol_type = other DNA
gtggcatactccgtctgctc

SEQ ID NO: 151 - DNA, 20 bp, Homo sapiens
  source 1..20: mol_type = other DNA
gatgaggtgcccattccgct

SEQ ID NO: 152 - DNA, 20 bp, Homo sapiens
  source 1..20: mol_type = other DNA
taccgctgcatgatcagcta

SEQ ID NO: 153 - DNA, 20 bp, Homo sapiens
  source 1..20: mol_type = other DNA
agctactatgctgaaccttc

SEQ ID NO: 154 - DNA, 20 bp, Homo sapiens
  source 1..20: mol_type = other DNA
ggatgaccaattcagctgta

SEQ ID NO: 155 - DNA, 20 bp, Homo sapiens
  source 1..20: mol_type = other DNA
accccaaggccgaagtcatc

SEQ ID NO: 156 - DNA, 20 bp, Homo sapiens
  source 1..20: mol_type = other DNA
tctttatattcatgacctac

SEQ ID NO: 157 - DNA, 20 bp, Homo sapiens
  source 1..20: mol_type = other DNA
accgttcagcaaatgccagt

SEQ ID NO: 158 - DNA, 20 bp, Homo sapiens
  source 1..20: mol_type = other DNA
gtacccaccgccatactacc

SEQ ID NO: 159 - DNA, 20 bp, Homo sapiens
  source 1..20: mol_type = other DNA
ttgcctatgcccaggtagta

SEQ ID NO: 160 - DNA, 20 bp, Homo sapiens
  source 1..20: mol_type = other DNA
ccttgtgccgctgaaatcca

SEQ ID NO: 161 - DNA, 20 bp, Homo sapiens
  source 1..20: mol_type = other DNA
accccgaactaactgctgca

SEQ ID NO: 162 - DNA, 20 bp, Homo sapiens
  source 1..20: mol_type = other DNA
acatagacccctgttgtaag

SEQ ID NO: 163 - DNA, 20 bp, Homo sapiens
  source 1..20: mol_type = other DNA
atccttgcagcagttagttc

SEQ ID NO: 164 - DNA, 20 bp, Homo sapiens
  source 1..20: mol_type = other DNA
acatcgaacctcttgcacgt

SEQ ID NO: 165 - DNA, 20 bp, Homo sapiens
  source 1..20: mol_type = other DNA
tacagctcaccttctcgcag

SEQ ID NO: 166 - DNA, 20 bp, Homo sapiens
  source 1..20: mol_type = other DNA
ggtatatctcctcgaggagc

SEQ ID NO: 167 - DNA, 20 bp, Homo sapiens
  source 1..20: mol_type = other DNA
tacactgcttgtagctctta

SEQ ID NO: 168 - DNA, 20 bp, Homo sapiens
  source 1..20: mol_type = other DNA
gagctctagacaccaacgtg

SEQ ID NO: 169 - DNA, 20 bp, Homo sapiens
  source 1..20: mol_type = other DNA
caagcagctgtccgagtccc

SEQ ID NO: 170 - DNA, 20 bp, Homo sapiens
  source 1..20: mol_type = other DNA
aatgtgtagccagcaccacg

SEQ ID NO: 171 - DNA, 20 bp, Homo sapiens
  source 1..20: mol_type = other DNA
gaacttcctgtaaacgatcc

SEQ ID NO: 172 - DNA, 20 bp, Homo sapiens
  source 1..20: mol_type = other DNA
tacatacctccattacaagc

SEQ ID NO: 173 - DNA, 20 bp, Homo sapiens
  source 1..20: mol_type = other DNA
ccatctactaaagctgtaag

SEQ ID NO: 174 - DNA, 20 bp, Homo sapiens
  source 1..20: mol_type = other DNA
ctcaatattgtaatgcgttc

SEQ ID NO: 175 - DNA, 20 bp, Homo sapiens
  source 1..20: mol_type = other DNA
ctctccatccatagacacac

SEQ ID NO: 176 - DNA, 20 bp, Homo sapiens
  source 1..20: mol_type = other DNA
taagacctacatcgccagcc

SEQ ID NO: 177 - DNA, 20 bp, Homo sapiens
  source 1..20: mol_type = other DNA
gaagaacttgcaccagcgtc

SEQ ID NO: 178 - DNA, 20 bp, Homo sapiens
  source 1..20: mol_type = other DNA
gtcagccgcattcaccctcg

SEQ ID NO: 179 - DNA, 20 bp, Homo sapiens
  source 1..20: mol_type = other DNA
ctgccagaagtcattgaccg

SEQ ID NO: 180 - DNA, 20 bp, Homo sapiens
  source 1..20: mol_type = other DNA
cccagccgtactatgccacg

SEQ ID NO: 181 - DNA, 20 bp, Homo sapiens
  source 1..20: mol_type = other DNA
gccgctgcccttccagacgc

SEQ ID NO: 182 - DNA, 20 bp, Homo sapiens
  source 1..20: mol_type = other DNA
gtagctggaatcctcatcag

SEQ ID NO: 183 - DNA, 20 bp, Homo sapiens
  source 1..20: mol_type = other DNA
caaaacctatcctacaactg

SEQ ID NO: 184 - DNA, 20 bp, Homo sapiens
  source 1..20: mol_type = other DNA
ttagggagtttatggaccca

SEQ ID NO: 185 - DNA, 20 bp, Homo sapiens
  source 1..20: mol_type = other DNA
ctcagccacagttgtaggat

SEQ ID NO: 186 - DNA, 20 bp, Homo sapiens
  source 1..20: mol_type = other DNA
tcactgtaccttaatgaagt

SEQ ID NO: 187 - DNA, 20 bp, Homo sapiens
  source 1..20: mol_type = other DNA
tcctttatctacaaccctcc

SEQ ID NO: 188 - DNA, 20 bp, Homo sapiens
  source 1..20: mol_type = other DNA
tccataggtgcccaacgctc

SEQ ID NO: 189 - DNA, 20 bp, Homo sapiens
  source 1..20: mol_type = other DNA
gttccggaaccaatgcacag

SEQ ID NO: 190 - DNA, 20 bp, Homo sapiens
  source 1..20: mol_type = other DNA
gcgagaagtccccgcgctgc

SEQ ID NO: 191 - DNA, 20 bp, Homo sapiens
  source 1..20: mol_type = other DNA
tgacccctgctcttcgcaga

SEQ ID NO: 192 - DNA, 20 bp, Homo sapiens
  source 1..20: mol_type = other DNA
cgccggcgagtaccgcgccg

SEQ ID NO: 193 - DNA, 20 bp, Homo sapiens
  source 1..20: mol_type = other DNA
tgggcggtcagggcggctga

SEQ ID NO: 194 - DNA, 20 bp, Homo sapiens
  source 1..20: mol_type = other DNA
ctaaatggggatttccgcaa

SEQ ID NO: 195 - DNA, 20 bp, Homo sapiens
  source 1..20: mol_type = other DNA
atccccatttagccagtatc

SEQ ID NO: 196 - DNA, 20 bp, Homo sapiens
  source 1..20: mol_type = other DNA
gtgaagtctctctgccgagt

SEQ ID NO: 197 - DNA, 20 bp, Homo sapiens
  source 1..20: mol_type = other DNA
aggtcacccctgcaccgact

SEQ ID NO: 198 - DNA, 20 bp, Homo sapiens
  source 1..20: mol_type = other DNA
cttactgttagatttatatc

SEQ ID NO: 199 - DNA, 20 bp, Homo sapiens
  source 1..20: mol_type = other DNA
tatagcagagacacagacac

SEQ ID NO: 200 - DNA, 20 bp, Homo sapiens
  source 1..20: mol_type = other DNA
gtgacttggtgcaagctcaa

SEQ ID NO: 201 - DNA, 20 bp, Homo sapiens
  source 1..20: mol_type = other DNA
tctgcttgccatttcgtcct

SEQ ID NO: 202 - DNA, 20 bp, Homo sapiens
  source 1..20: mol_type = other DNA
ctgttagcacagtatttcac

SEQ ID NO: 203 - DNA, 20 bp, Homo sapiens
  source 1..20: mol_type = other DNA
ccaaaggaagtaaacgatac

SEQ ID NO: 204 - DNA, 20 bp, Homo sapiens
  source 1..20: mol_type = other DNA
atgttccagatgtccagata

SEQ ID NO: 205 - DNA, 20 bp, Homo sapiens
  source 1..20: mol_type = other DNA
cttcttcttaatcccatatc

SEQ ID NO: 206 - DNA, 20 bp, Homo sapiens
  source 1..20: mol_type = other DNA
agtttagtcgcgttccttcc

SEQ ID NO: 207 - DNA, 20 bp, Homo sapiens
  source 1..20: mol_type = other DNA
cactgtgcaacggtgtgact

SEQ ID NO: 208 - DNA, 20 bp, Homo sapiens
  source 1..20: mol_type = other DNA
ggatgtccacaattgccagc

SEQ ID NO: 209 - DNA, 20 bp, Homo sapiens
  source 1..20: mol_type = other DNA
aactgagagtgccttcatta

SEQ ID NO: 210 - DNA, 20 bp, Homo sapiens
  source 1..20: mol_type = other DNA
gacagggaactacacagtga

SEQ ID NO: 211 - protein, 965 aa, synthetic construct
  REGION 1..965: note = Synthetic
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRSGSGQCTNYALLKLAGDVESNPGPPTGMVCLKLPGGSCMTALTVTLMVLSSPLALASDENPVVHFFKNIVTPRTPPGGGGSGGGGSGGSGDTRPRFLWQPKRECHFFNGTERVRFLDRYFYNQEESVRFDSDVGEFRAVTELGRPDAEYWNSQKDILEQARAAVDTYCRHNYGVVESFTVQRRVQPKVTVYPSKTQPLQHHNLLVCSVSGFYPGSIEVRWFLNGQEEKAGMVSTGLIQNGDWTFQTLVMLETVPRSGEVYTCQVEHPSVTSPLTVEWRARSESAQSKMLSGVGGFVLGLLFLGAGLFIYFRNQTSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSSGSGEGRGSLLTCGDVEENPGPMAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGLTSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 212 - DNA, 20 bp, Homo sapiens
  source 1..20: mol_type = other DNA
attgtggacatccagtctgg

SEQ ID NO: 213 - DNA, 20 bp, Homo sapiens
  source 1..20: mol_type = other DNA
tcgctgaccgtgaacgatac

SEQ ID NO: 214 - DNA, 20 bp, Homo sapiens
  source 1..20: mol_type = other DNA
tggggccactcgatccttga

SEQ ID NO: 215 - DNA, 20 bp, Homo sapiens
  source 1..20: mol_type = other DNA
gcagatgaccaccagcgtcg

SEQ ID NO: 216 - DNA, 20 bp, Homo sapiens
  source 1..20: mol_type = other DNA
tcaggccttacctgaggcga

SEQ ID NO: 217 - DNA, 20 bp, Homo sapiens
  source 1..20: mol_type = other DNA
catctgcacagcagtcatcg

SEQ ID NO: 218 - DNA, 20 bp, Homo sapiens
  source 1..20: mol_type = other DNA
attgaagtagtcatgcagct

SEQ ID NO: 219 - DNA, 20 bp, Homo sapiens
  source 1..20: mol_type = other DNA
aggcacagtagaagccgtat

SEQ ID NO: 220 - DNA, 20 bp, Homo sapiens
  source 1..20: mol_type = other DNA
gctgtctatcatccccaata

SEQ ID NO: 221 - DNA, 20 bp, Homo sapiens
  source 1..20: mol_type = other DNA
acttaccaccgaccatgcat

SEQ ID NO: 222 - DNA, 20 bp, Homo sapiens
  source 1..20: mol_type = other DNA
cacactttagagtgagcgtc

SEQ ID NO: 223 - DNA, 20 bp, Homo sapiens
  source 1..20: mol_type = other DNA
ctccagtggctgaccccctc

SEQ ID NO: 224 - DNA, 20 bp, Homo sapiens
  source 1..20: mol_type = other DNA
ccacagcagcccaccagtac

SEQ ID NO: 225 - DNA, 20 bp, Homo sapiens
  source 1..20: mol_type = other DNA
ttataatagatgcagcgata

SEQ ID NO: 226 - DNA, 20 bp, Homo sapiens
  source 1..20: mol_type = other DNA
tcattgtgactgttgtccga

SEQ ID NO: 227 - DNA, 20 bp, Homo sapiens
  source 1..20: mol_type = other DNA
gccaggcaccgtgatccccc

SEQ ID NO: 228 - DNA, 20 bp, Homo sapiens
  source 1..20: mol_type = other DNA
catccttatccccggtaccc

SEQ ID NO: 229 - DNA, 20 bp, Homo sapiens
  source 1..20: mol_type = other DNA
cagagagttctgagctcgac

SEQ ID NO: 230 - DNA, 20 bp, Homo sapiens
  source 1..20: mol_type = other DNA
agtgttgctgggggcggtcg

SEQ ID NO: 231 - DNA, 20 bp, Homo sapiens
  source 1..20: mol_type = other DNA
gacgatgcagagttccgtga

SEQ ID NO: 232 - DNA, 20 bp, Homo sapiens
  source 1..20: mol_type = other DNA
actcacaggacacgttgaga

SEQ ID NO: 233 - DNA, 20 bp, Homo sapiens
  source 1..20: mol_type = other DNA
taccctggcccagtagttca

SEQ ID NO: 234 - DNA, 20 bp, Homo sapiens
  source 1..20: mol_type = other DNA
accttcgtctgtatgctgtt

SEQ ID NO: 235 - DNA, 20 bp, Homo sapiens
  source 1..20: mol_type = other DNA
accaaacagcatacagacga

SEQ ID NO: 236 - DNA, 20 bp, Homo sapiens
  source 1..20: mol_type = other DNA
ctactctatgatccagtccc

SEQ ID NO: 237 - DNA, 20 bp, Homo sapiens
  source 1..20: mol_type = other DNA
actccgtcagctcgttagaa

SEQ ID NO: 238 - DNA, 20 bp, Homo sapiens
  source 1..20: mol_type = other DNA
gttcatactctcttcgtcat

SEQ ID NO: 239 - DNA, 20 bp, Homo sapiens
  source 1..20: mol_type = other DNA
agagtagcagctcacataac

SEQ ID NO: 240 - DNA, 20 bp, Homo sapiens
  source 1..20: mol_type = other DNA
ttccagagctcacaacgacc

SEQ ID NO: 241 - DNA, 20 bp, Homo sapiens
  source 1..20: mol_type = other DNA
atagtcctgtccatatttgc

SEQ ID NO: 242 - DNA, 20 bp, Homo sapiens
  source 1..20: mol_type = other DNA
agatactcacgatctcattg

SEQ ID NO: 243 - DNA, 20 bp, Homo sapiens
  source 1..20: mol_type = other DNA
aggtcaaggattgtacgccc

SEQ ID NO: 244 - DNA, 20 bp, Homo sapiens
  source 1..20: mol_type = other DNA
gaagtccctgcaccacgacc

SEQ ID NO: 245 - DNA, 20 bp, Homo sapiens
  source 1..20: mol_type = other DNA
tttggttgttccgttgctgt

SEQ ID NO: 246 - DNA, 20 bp, Homo sapiens
  source 1..20: mol_type = other DNA
tgatcgaccctccgccagaa

SEQ ID NO: 247 - DNA, 20 bp, Homo sapiens
  source 1..20: mol_type = other DNA
gggtcgatcatctattaata

SEQ ID NO: 248 - DNA, 20 bp, Homo sapiens
  source 1..20: mol_type = other DNA
tcctttgcggaatgtagtcc

SEQ ID NO: 249 - DNA, 20 bp, Homo sapiens
  source 1..20: mol_type = other DNA
ctatgtatcctttcggcatg

SEQ ID NO: 250 - DNA, 20 bp, Homo sapiens
  source 1..20: mol_type = other DNA
tcttctgccgtatgatatag

SEQ ID NO: 251 - DNA, 20 bp, Homo sapiens
  source 1..20: mol_type = other DNA
gtgagacatgatctcccgaa

SEQ ID NO: 252 - DNA, 20 bp, Homo sapiens
  source 1..20: mol_type = other DNA
atgtcgatgcagcaaacctc

SEQ ID NO: 253 - DNA, 20 bp, Homo sapiens
  source 1..20: mol_type = other DNA
attatacataaacccatctc

SEQ ID NO: 254 - DNA, 20 bp, Homo sapiens
  source 1..20: mol_type = other DNA
aatggactctggaatatccc

SEQ ID NO: 255 - DNA, 20 bp, Homo sapiens
  source 1..20: mol_type = other DNA
atagagacaatcttacccgc

SEQ ID NO: 256 - DNA, 20 bp, Homo sapiens
  source 1..20: mol_type = other DNA
aagattgtctctatctgcgc

SEQ ID NO: 257 - DNA, 20 bp, Homo sapiens
  source 1..20: mol_type = other DNA
aaatgtgattgccttcgcca

SEQ ID NO: 258 - DNA, 20 bp, Homo sapiens
  source 1..20: mol_type = other DNA
cgtttgtaccgtccctcttc

SEQ ID NO: 259 - DNA, 20 bp, Homo sapiens
  source 1..20: mol_type = other DNA
tgcgatccatcaagaccacc

SEQ ID NO: 260 - DNA, 20 bp, Homo sapiens
  source 1..20: mol_type = other DNA
ttgatatttaggcttgccga

SEQ ID NO: 261 - DNA, 20 bp, Homo sapiens
  source 1..20: mol_type = other DNA
agtcgtcgacgcgccgcagc

SEQ ID NO: 262 - DNA, 20 bp, Homo sapiens
  source 1..20: mol_type = other DNA
agcggcccatcaggacgctt

SEQ ID NO: 263 - DNA, 20 bp, Homo sapiens
  source 1..20: mol_type = other DNA
gcggcgcgtcgacgacttcg

SEQ ID NO: 264 - DNA, 20 bp, Homo sapiens
  source 1..20: mol_type = other DNA
gtgtaacatacctggaggac

SEQ ID NO: 265 - DNA, 20 bp, Homo sapiens
  source 1..20: mol_type = other DNA
tacatctgcacttggtattc

SEQ ID NO: 266 - DNA, 20 bp, Homo sapiens
  source 1..20: mol_type = other DNA
ctaaaacttacttggtgcaa

SEQ ID NO: 267 - DNA, 20 bp, Homo sapiens
  source 1..20: mol_type = other DNA
tctcaactctttctcgaagc

SEQ ID NO: 268 - DNA, 20 bp, Homo sapiens
  source 1..20: mol_type = other DNA
ctgcctgatctcatagagtc

SEQ ID NO: 269 - DNA, 20 bp, Homo sapiens
  source 1..20: mol_type = other DNA
cagatacttactcataagtc

SEQ ID NO: 270 - DNA, 20 bp, Homo sapiens
  source 1..20: mol_type = other DNA
cgctgagaaatgactgcacg

SEQ ID NO: 271 - DNA, 20 bp, Homo sapiens
  source 1..20: mol_type = other DNA
catatacttcttcaccagtt

SEQ ID NO: 272 - DNA, 20 bp, Homo sapiens
  source 1..20: mol_type = other DNA
atgtactcacacatctggat

SEQ ID NO: 273 - DNA, 20 bp, Homo sapiens
  source 1..20: mol_type = other DNA
accgaatgtcccgcgtgcaa

SEQ ID NO: 274 - DNA, 20 bp, Homo sapiens
  source 1..20: mol_type = other DNA
gcctttgcacgcgggacatt

SEQ ID NO: 275 - DNA, 20 bp, Homo sapiens
  source 1..20: mol_type = other DNA
ggggaccctgaaagaatacg

SEQ ID NO: 276 - DNA, 20 bp, Homo sapiens
  source 1..20: mol_type = other DNA
cctgcgctggagaaactatt

SEQ ID NO: 277 - DNA, 20 bp, Homo sapiens
  source 1..20: mol_type = other DNA
ccttgtagtaactgcccagc

SEQ ID NO: 278 - DNA, 20 bp, Homo sapiens
  source 1..20: mol_type = other DNA
catagtagccactctgttgc

SEQ ID NO: 279 - DNA, 20 bp, Homo sapiens
  source 1..20: mol_type = other DNA
caatatcaacgtaatagttc

SEQ ID NO: 280 - DNA, 20 bp, Homo sapiens
  source 1..20: mol_type = other DNA
gacttagtgcaatgcaagac

SEQ ID NO: 281 - DNA, 20 bp, Homo sapiens
  source 1..20: mol_type = other DNA
gatattgctgattaagtccc

SEQ ID NO: 282 - DNA, 20 bp, Homo sapiens
  source 1..20: mol_type = other DNA
gcgggccgaccgcatttggg

SEQ ID NO: 283 - DNA, 20 bp, Homo sapiens
  source 1..20: mol_type = other DNA
catattacatcggatatctg

SEQ ID NO: 284 - DNA, 20 bp, Homo sapiens
  source 1..20: mol_type = other DNA
actcttctgccggacacttg

SEQ ID NO: 285 - DNA, 20 bp, Homo sapiens
  source 1..20: mol_type = other DNA
ttatagaggaaccctactaa

SEQ ID NO: 286 - DNA, 20 bp, Homo sapiens
  source 1..20: mol_type = other DNA
ttgtgggcaccttactgagt

SEQ ID NO: 287 - DNA, 20 bp, Homo sapiens
  source 1..20: mol_type = other DNA
cgaacacatcgctgacaact

SEQ ID NO: 288 - DNA, 20 bp, Homo sapiens
  source 1..20: mol_type = other DNA
gttaacgtttccactttacc

SEQ ID NO: 289 - DNA, 20 bp, Homo sapiens
  source 1..20: mol_type = other DNA
ttcccgctgagcctcgtcca

SEQ ID NO: 290 - DNA, 20 bp, Homo sapiens
  source 1..20: mol_type = other DNA
tatctaatttatcgtcttcc

SEQ ID NO: 291 - DNA, 20 bp, Homo sapiens
  source 1..20: mol_type = other DNA
ttcggagcatcagtcgttga

SEQ ID NO: 292 - DNA, 20 bp, Homo sapiens
  source 1..20: mol_type = other DNA
ttcaacgactgatgctccga

SEQ ID NO: 293 - DNA, 20 bp, Homo sapiens
  source 1..20: mol_type = other DNA
catgcactctgtttgcgaag

SEQ ID NO: 294 - DNA, 19 bp, Homo sapiens
  source 1..19: mol_type = other DNA
ttctcccagaaccaaacga

SEQ ID NO: 295 - DNA, 20 bp, Homo sapiens
  source 1..20: mol_type = other DNA
gatgatgtctgcctcgcgcc

SEQ ID NO: 296 - DNA, 20 bp, Homo sapiens
  source 1..20: mol_type = other DNA
atgccccatcttcaattgtc

SEQ ID NO: 297 - DNA, 20 bp, Homo sapiens
  source 1..20: mol_type = other DNA
tattcggccatcaaaggagc

SEQ ID NO: 298 - DNA, 20 bp, Homo sapiens
  source 1..20: mol_type = other DNA
gactaagcctcaactccaac

SEQ ID NO: 299 - DNA, 20 bp, Homo sapiens
  source 1..20: mol_type = other DNA
tatacaagctattagctgcg

SEQ ID NO: 300 - DNA, 20 bp, Homo sapiens
  source 1..20: mol_type = other DNA
gaactacatcgctgagcttc

SEQ ID NO: 301 - DNA, 20 bp, Homo sapiens
  source 1..20: mol_type = other DNA
gccatcctattgtgagtttc

SEQ ID NO: 302 - DNA, 20 bp, Homo sapiens
  source 1..20: mol_type = other DNA
tgtctccaatttaactaacg

SEQ ID NO: 303 - DNA, 20 bp, Homo sapiens
  source 1..20: mol_type = other DNA
aaggaatagtcacctccgta

SEQ ID NO: 304 - DNA, 20 bp, Homo sapiens
  source 1..20: mol_type = other DNA
aatggggcattataacaacg

SEQ ID NO: 305 - DNA, 20 bp, Homo sapiens
  source 1..20: mol_type = other DNA
ggtgaggttcgtggtgttcg

SEQ ID NO: 306 - DNA, 20 bp, Homo sapiens
  source 1..20: mol_type = other DNA
gtgcagcaatcgtcgactac

SEQ ID NO: 307 - DNA, 20 bp, Homo sapiens
  source 1..20: mol_type = other DNA
aatcccggtatacaatcaaa

SEQ ID NO: 308 - DNA, 20 bp, Homo sapiens
  source 1..20: mol_type = other DNA
ctcgagttgcttgacttacg

SEQ ID NO: 309 - DNA, 20 bp, Homo sapiens
  source 1..20: mol_type = other DNA
ggctcacggctattcggccg

SEQ ID NO: 310 - DNA, 20 bp, Homo sapiens
  source 1..20: mol_type = other DNA
gcgtctccgtgacaattacc

SEQ ID NO: 311 - DNA, 20 bp, Homo sapiens
  source 1..20: mol_type = other DNA
ccgacaggtccacgtagcgc

SEQ ID NO: 312 - DNA, 20 bp, Homo sapiens
  source 1..20: mol_type = other DNA
cccacatttacgcctatcct

SEQ ID NO: 313 - DNA, 20 bp, Homo sapiens
  source 1..20: mol_type = other DNA
taacattaatagcagctcga

SEQ ID NO: 314 - DNA, 20 bp, Homo sapiens
  source 1..20: mol_type = other DNA
gaccccagccacaacgacac

SEQ ID NO: 315 - DNA, 20 bp, Homo sapiens
  source 1..20: mol_type = other DNA
ccccaccaaatttgtccaat

SEQ ID NO: 316 - DNA, 20 bp, Homo sapiens
  source 1..20: mol_type = other DNA
gaggccctattggacaaatt

SEQ ID NO: 317 - DNA, 20 bp, Homo sapiens
  source 1..20: mol_type = other DNA
gttgctccgtgccacatctg

SEQ ID NO: 318 - DNA, 20 bp, Homo sapiens
  source 1..20: mol_type = other DNA
ccatacttactccgcagagc

SEQ ID NO: 319 - DNA, 20 bp, Homo sapiens
  source 1..20: mol_type = other DNA
tatggtgcgttctacagcga

SEQ ID NO: 320 - DNA, 20 bp, Homo sapiens
  source 1..20: mol_type = other DNA
cccgacggctctgcagttaa

SEQ ID NO: 321 - DNA, 20 bp, Homo sapiens
  source 1..20: mol_type = other DNA
tcggtgcagctccacgactc

SEQ ID NO: 322 - DNA, 20 bp, Homo sapiens
  source 1..20: mol_type = other DNA
aactattcatgctaccgggc

SEQ ID NO: 323 - DNA, 20 bp, Homo sapiens
  source 1..20: mol_type = other DNA
cgtggtgcagcttcgtgccc

SEQ ID NO: 324 - DNA, 20 bp, Homo sapiens
  source 1..20: mol_type = other DNA
agatgcctcaacttggagcc

SEQ ID NO: 325 - DNA, 20 bp, Homo sapiens
  source 1..20: mol_type = other DNA
tttgagttgcattgtgaacg

SEQ ID NO: 326 - DNA, 20 bp, Homo sapiens
  source 1..20: mol_type = other DNA
attcgctgtatgaaggaaga

SEQ ID NO: 327 - DNA, 20 bp, Homo sapiens
  source 1..20: mol_type = other DNA
ttcctacacaatgcgttgcc

SEQ ID NO: 328 - DNA, 20 bp, Homo sapiens
  source 1..20: mol_type = other DNA
gatattggcataagcctccc

SEQ ID NO: 329 - DNA, 20 bp, Homo sapiens
  source 1..20: mol_type = other DNA
tcaactgcgaccagttcagc

SEQ ID NO: 330 - DNA, 20 bp, Homo sapiens
  source 1..20: mol_type = other DNA
tcgtatgtgccctcgtcaga

SEQ ID NO: 331 - DNA, 20 bp, Homo sapiens
  source 1..20: mol_type = other DNA
gagtgaatcagaccttcaac

SEQ ID NO: 332 - DNA, 20 bp, Homo sapiens
  source 1..20: mol_type = other DNA
tatggcccgagtacaagaac

SEQ ID NO: 333 - DNA, 20 bp, Homo sapiens
  source 1..20: mol_type = other DNA
gtaaccgtgtatagatgagc

SEQ ID NO: 334 - DNA, 20 bp, Homo sapiens
  source 1..20: mol_type = other DNA
atactcgatagttgaattct

SEQ ID NO: 335 - DNA, 20 bp, Homo sapiens
  source 1..20: mol_type = other DNA
catcagatctttcaggtata

SEQ ID NO: 336 - DNA, 20 bp, Homo sapiens
  source 1..20: mol_type = other DNA
actcacgctggatagcctcc

SEQ ID NO: 337 - DNA, 20 bp, Homo sapiens
  source 1..20: mol_type = other DNA
gagtagcgcgagcacagcta

SEQ ID NO: 338 - DNA, 20 bp, Homo sapiens
  source 1..20: mol_type = other DNA
cagtaagtcaacttcaatgt

SEQ ID NO: 339 - DNA, 20 bp, Homo sapiens
  source 1..20: mol_type = other DNA
ggttgttgcaagattgaccc

SEQ ID NO: 340 - DNA, 20 bp, Homo sapiens
  source 1..20: mol_type = other DNA
gaggaactctaagtatcccc

SEQ ID NO: 341 - DNA, 20 bp, Homo sapiens
  source 1..20: mol_type = other DNA
tctggttgccttggtaggat

SEQ ID NO: 342 - DNA, 20 bp, Homo sapiens
  source 1..20: mol_type = other DNA
cgcagccacaagttcgtgcc

SEQ ID NO: 343 - DNA, 20 bp, Homo sapiens
  source 1..20: mol_type = other DNA
ggagctgggtggccgcatat

SEQ ID NO: 344 - DNA, 20 bp, Homo sapiens
  source 1..20: mol_type = other DNA
cccgaacagcaggtcgttaa

SEQ ID NO: 345 - DNA, 20 bp, Homo sapiens
  source 1..20: mol_type = other DNA
ctcttacctgtaccataacc

SEQ ID NO: 346 - DNA, 20 bp, Homo sapiens
  source 1..20: mol_type = other DNA
aatgcctccgcttatgttgc

SEQ ID NO: 347 - DNA, 20 bp, Homo sapiens
  source 1..20: mol_type = other DNA
tggcattggccacgaagaaa

SEQ ID NO: 348 - DNA, 20 bp, Homo sapiens
  source 1..20: mol_type = other DNA
cgactacttcggtagtatgc

SEQ ID NO: 349 - DNA, 20 bp, Homo sapiens
  source 1..20: mol_type = other DNA
cagcatactaccgaagtagt

SEQ ID NO: 350 - DNA, 20 bp, Homo sapiens
  source 1..20: mol_type = other DNA
gtgtttgtgtacatcaactc

SEQ ID NO: 351 - DNA, 43 bp, Homo sapiens
  source 1..43: mol_type = other DNA
tgacttccagagagcaatatggctggttccccaacatgcctca

SEQ ID NO: 352 - DNA, 17 bp, Homo sapiens
  source 1..17: mol_type = other DNA
tgacttccagagagcaa

SEQ ID NO: 353 - protein, 969 aa, Homo sapiens
MDYKDHDGDYKDHDIDYKDDDDKMAPKKKRKVGIHGVPAAVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNIGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFRS

SEQ ID NO: 354 - DNA, 17 bp, Homo sapiens
  source 1..17: mol_type = other DNA
aacatgcctcaccctca

SEQ ID NO: 355 - protein, 969 aa, Homo sapiens
MDYKDHDGDYKDHDIDYKDDDDKMAPKKKRKVGIHGVPAAVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASNGGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFRS

SEQ ID NO: 356 - DNA, 49 bp, Homo sapiens
  source 1..49: mol_type = other DNA
ttccagagagcaatatggctggttccccaacatgcctcaccctcatcta

SEQ ID NO: 357 - DNA, 17 bp, Homo sapiens
  source 1..17: mol_type = other DNA
ttccagagagcaatatg

SEQ ID NO: 358 - protein, 969 aa, Homo sapiens
MDYKDHDGDYKDHDIDYKDDDDKMAPKKKRKVGIHGVPAAVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNNGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFRS

SEQ ID NO: 359 - DNA, 17 bp, Homo sapiens
  source 1..17: mol_type = other DNA
tgcctcaccctcatcta

SEQ ID NO: 360 - protein, 969 aa, Homo sapiens
MDYKDHDGDYKDHDIDYKDDDDKMAPKKKRKVGIHGVPAAVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNIGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFRS

SEQ ID NO: 361 - DNA, 49 bp, Homo sapiens
  source 1..49: mol_type = other DNA
ttgactctattgtctggaccttcaacacaacccctcttgtcaccataca

SEQ ID NO: 362 - DNA, 17 bp, Homo sapiens
  source 1..17: mol_type = other DNA
ttgactctattgtctgg

SEQ ID NO: 363 - protein, 969 aa, Homo sapiens
MDYKDHDGDYKDHDIDYKDDDDKMAPKKKRKVGIHGVPAAVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNGGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNNGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFRS

SEQ ID NO: 364 - DNA, 17 bp, Homo sapiens
  source 1..17: mol_type = other DNA
cctcttgtcaccataca

SEQ ID NO: 365 - protein, 969 aa, Homo sapiens
MDYKDHDGDYKDHDIDYKDDDDKMAPKKKRKVGIHGVPAAVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNNGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFRS

SEQ ID NO: 366 - DNA, 49 bp, Homo sapiens
  source 1..49: mol_type = other DNA
ttccctcccaggcagctcacagtgtgccaccatggagttggggccccta

SEQ ID NO: 367 - DNA, 18 bp, Homo sapiens
  source 1..18: mol_type = other DNA
ttccctcccaggcagctc

SEQ ID NO: 368 - protein, 1003 aa, Homo sapiens
MDYKDHDGDYKDHDIDYKDDDDKMAPKKKRKVGIHGVPAAVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFRS

SEQ ID NO: 369 - DNA, 17 bp, Homo sapiens
  source 1..17: mol_type = other DNA
tggagttggggccccta

SEQ ID NO: 370 - protein, 969 aa, Homo sapiens
MDYKDHDGDYKDHDIDYKDDDDKMAPKKKRKVGIHGVPAAVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFRS

SEQ ID NO: 371 - DNA, 49 bp, Homo sapiens
  source 1..49: mol_type = other DNA
ttcctcctactcaccatcagcctcctggttatggtacaggtaagagcaa

SEQ ID NO: 372 - DNA, 17 bp, Homo sapiens
  source 1..17: mol_type = other DNA
ttcctcctactcaccat

SEQ ID NO: 373 - protein, 969 aa, Homo sapiens
MDYKDHDGDYKDHDIDYKDDDDKMAPKKKRKVGIHGVPAAVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFRS

SEQ ID NO: 374 - DNA, 17 bp, Homo sapiens
  source 1..17: mol_type = other DNA
ggtacaggtaagagcaa

SEQ ID NO: 375 - protein, 969 aa, Homo sapiens
MDYKDHDGDYKDHDIDYKDDDDKMAPKKKRKVGIHGVPAAVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNGGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASHDGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFRS

SEQ ID NO: 376 - DNA, 49 bp, Homo sapiens
  source 1..49: mol_type = other DNA
ttgtcccacagatatccagaaccctgaccctgccgtgtaccagctgaga

SEQ ID NO: 377 - DNA, 17 bp, Homo sapiens
  source 1..17: mol_type = other DNA
ttgtcccacagatatcc

SEQ ID NO: 378 - protein, 969 aa, Homo sapiens
MDYKDHDGDYKDHDIDYKDDDDKMAPKKKRKVGIHGVPAAVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNGGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFRS

SEQ ID NO: 379 - DNA, 17 bp, Homo sapiens
  source 1..17: mol_type = other DNA
ccgtgtaccagctgaga

SEQ ID NO: 380 - protein, 969 aa, Homo sapiens
MDYKDHDGDYKDHDIDYKDDDDKMAPKKKRKVGIHGVPAAVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNNGGKQALETVQRLLPVLCQAHGLTPEQVVAIASNNGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFRS

SEQ ID NO: 381 - DNA, 49 bp, Homo sapiens
  source 1..49: mol_type = other DNA
ttctccccagccctgctcgtggtgaccgaaggggacaacgccaccttca

SEQ ID NO: 382 - protein, 936 aa, Homo sapiens
MGDPKKKRKVIDYPYDVPDYAIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 383 - protein, 941 aa, Homo sapiens
MGDPKKKRKVIDKETAAAKFERQHMDSIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 384 - RNA, 100 nt, synthetic construct
  misc_feature 1..100: note = Synthetic
  source 1..100: mol_type = other RNA
gagaatcaaaatcggtgaatgttttagagctagaaatagcaagttaaaataaggctagtccgttatcaacttgaaaaagtggcaccgagtcggtgctttt

SEQ ID NO: 385 - DNA, 21 bp, synthetic construct
  misc_feature 1..21: note = Synthetic
  source 1..21: mol_type = other DNA
agcgctctcgtacagagttgg

SEQ ID NO: 386 - DNA, 23 bp, synthetic construct
  misc_feature 1..23: note = Synthetic
  source 1..23: mol_type = other DNA
aaaaaaagcaccgactcggtgcc

SEQ ID NO: 387 - RNA, 100 nt, synthetic construct; identical to SEQ ID NO: 384
  misc_feature 1..100: note = Synthetic
  misc_feature 1: note = 2'OMe
  misc_feature 1..2: note = phosphorothioate
  misc_feature 2..3: note = phosphorothioate
  misc_feature 3..4: note = phosphorothioate
  misc_feature 96: note = 2'OMe
  misc_feature 97..98: note = phosphorothioate
  misc_feature 98..99: note = phosphorothioate
  misc_feature 99..100: note = phosphorothioate
  source 1..100: mol_type = other RNA

SEQ ID NO: 388 - protein, 20 aa, synthetic construct
  REGION 1..20: note = Synthetic
MDPKGLLSLTFVLFLSLAFG

SEQ ID NO: 389 - protein, 495 aa, synthetic construct; identical to SEQ ID NO: 128
  REGION 1..495: note = Synthetic

SEQ ID NO: 390 - DNA, 1485 bp, synthetic construct
  misc_feature 1..1485: note = Synthetic
  source 1..1485: mol_type = other DNA
atggagacagacactcttctcctttgggtcttgctgctgtgggttcccggaagcacaggagaagtacagttgcaacagtctgggccagaactcatcaaacccggagcttctgtaaaaatgtcatgcaaagctagtggatatacatttacttcttacgtgatgcactgggtaaaacagaaacctggtcaggggcttgagtggatcgggtacattaacccatataatgacggcaccaaatataacgagaaattcaagggaaaggctacgcttacatcagataagtccagtagcaccgcttatatggaacttagcagccttacttccgaagattccgcggtgtattactgcgcgagagggacttactactacgggagtcgagtattcgattattggggtcaaggcacgacgctcacggtgagctcaggtggtggagggtctgggggtggcggcagtggtggggggggctcagacatcgtgatgacccaggcagcaccttctatcccggtaaccccaggcgagtctgtatctatcagttgtcggtccagcaagtctcttctcaacagtaatggcaatacatatctttactggttcctccaaaggcctgggcaaagtcctcaacttcttatatatcggatgtccaatcttgcgagtggcgtacccgacaggttttcagggtctgggagcggaacagcttttacgttgagaatatccagggtagaagctgaggacgtcggtgtatattattgcatgcaacatctcgaatacccctttaccttcggcgctggtacaaagctcgaattgaaacgcagcgatccaaccacgacgccagcgccacgaccacctacgcccgctccaactattgcctcccagcccctgagtcttcggccagaagcgtgtagacctgctgccggcggggccgttcatacgcggggccttgactttgcatgtgatatctatatatgggctcctttggcgggaacttgcggagtgcttcttttgtcactcgtgataacgttgtattgtaaaaggggtcgaaagaaactcctctatatatttaagcagccctttatgaggcccgtgcaaacaacacaagaagaggacggatgctcttgtcgattcccggaagaggaggagggggggtgtgagcttagggtcaagttttctcgctctgccgacgcgccagcctatcaacagggccaaaaccagctgtataacgaactcaacctcgggcgccgggaagagtatgacgtccttgacaaacggcgcggtcgcgaccctgaaatgggtggaaaaccgaggcgaaagaacccccaggagggactttacaacgaattgcaaaaagacaagatggccgaagcctattccgaaattggaatgaaaggcgagcggagacgaggtaaggggcatgacggcctgtatcaagggctctctacggccacgaaggatacttacgacgcccttcatatgcaagctcttccaccacgg

SEQ ID NO: 391 - protein, 374 aa, Homo sapiens
MSGESMNFSDVFDSSEDYFVSVNTSYYSVDSEMLLCSLQEVRQFSRLFVPIAYSLICVFGLLGNILVVITFAFYKKARSMTDVYLLNMAIADILFVLTLPFWAVSHATGAWVFSNATCKLLKGIYAINFNCGMLLLTCISMDRYIAIVQATKSFRLRSRTLPRSKIICLVVWGLSVIISSSTFVFNQKYNTQGSDVCEPKYQTVSEPIRWKLLMLGLELLFGFFIPLMFMIFCYTFIVKTLVQAQNSKRHKAIRVIIAVVLVFLACQIPHNMVLLVTAANLGKMNRSCQSEKLIGYTKTVTEVLAFLHCCLNPVLYAFIGQKFRNYFLKILKDLWCVRRKYKSSGFSCAGRYSENISRQTSETADNDNASSFTM

SEQ ID NO: 392 - DNA, 1122 bp, synthetic construct
  misc_feature 1..1122: note = Synthetic
  source 1..1122: mol_type = other DNA
atgagtggggaaagtatgaacttcagcgatgtatttgactcctccgaagattactttgtatctgtgaatacgagctattactccgtcgatagtgaaatgctgctctgtagtctccaagaagtccgccaattcagtcgcctcttcgttcccatcgcgtactcccttatttgtgtttttggccttctgggtaacatcctggttgtaatcacattcgctttctataaaaaagctcggagtatgactgatgtttaccttcttaacatggctatagcggacattctttttgtgcttactctcccattctgggctgtgagccatgcaacaggggcgtgggttttttcaaatgccacatgtaagctgcttaaagggatctatgcaataaacttcaattgcgggatgctcctgctgacatgcatcagtatggatcgatacatagctatagtacaggcgactaagtccttccgcctgcgatcccgcacactgcctaggagcaaaattatttgcctcgtcgtatgggggctctcagtgatcatctcctccagtacgtttgtctttaaccagaaatataacacacagggttctgatgtatgtgaaccaaagtatcagacagtgagtgaaccaatacggtggaagttgcttatgttgggcttggagctgctttttgggtttttcatcccactgatgttcatgattttctgttatacatttattgttaagaccttggttcaggcgcaaaatagcaagagacataaggcaattcgagtcatcattgccgtggtgttggtcttcttggcctgtcagatcccccataatatggttctgctcgtcaccgccgctaacttgggtaagatgaatcgatcttgtcagtccgagaagttgatcggatacaccaaaactgtgacagaagtgctggccttccttcactgttgtctgaacccagttttgtatgcttttataggacagaagtttcgaaattacttcttgaaaatcctcaaggacctctggtgtgttcgaaggaagtacaagagctctggctttagttgcgctgggcgctacagtgagaatatatcccggcagacctccgagactgctgataatgacaacgcaagttccttcactatg

SEQ ID NO: 393 - DNA, 1122 bp, Homo sapiens
  source 1..1122: mol_type = other DNA
atgagcggggaatcaatgaatttcagcgatgttttcgactccagtgaagattattttgtgtcagtcaatacttcatattactcagttgattctgagatgttactgtgctccttgcaggaggtcaggcagttctccaggctatttgtaccgattgcctactccttgatctgtgtctttggcctcctggggaatattctggtggtgatcacctttgctttttataagaaggccaggtctatgacagacgtctatctcttgaacatggccattgcagacatcctctttgttcttactctcccattctgggcagtgagtcatgccactggtgcgtgggttttcagcaatgccacgtgcaagttgctaaaaggcatctatgccatcaactttaactgcgggatgctgctcctgacttgcattagcatggaccggtacatcgccattgtacaggcgactaagtcattccggctccgatccagaacactaccgcgcacgaaaatcatctgccttgttgtgtgggggctgtcagtcatcatctccagctcaacttttgtcttcaaccaaaaatacaacacccaaggcagcgatgtctgtgaacccaagtaccagactgtctcggagcccatcaggtggaagctgctgatgttggggcttgagctactctttggtttctttatccctttgatgttcatgatattttgttacacgttcattgtcaaaaccttggtgcaagctcagaattctaaaaggcacaaagccatccgtgtaatcatagctgtggtgcttgtgtttctggcttgtcagattcctcataacatggtcctgcttgtgacggctgcaaatttgggtaaaatgaaccgatcctgccagagcgaaaagctaattggctatacgaaaactgtcacagaagtcctggctttcctgcactgctgcctgaaccctgtgctctacgcttttattgggcagaagttcagaaactactttctgaagatcttgaaggacctgtggtgtgtgagaaggaagtacaagtcctcaggcttctcctgtgccgggaggtactcagaaaacatttctcggcagaccagtgagaccgcagataacgacaatgcgtcgtccttcactatg

SEQ ID NO: 394 - protein, 157 aa, synthetic construct
  REGION 1..157: note = Synthetic
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 395 - DNA, 471 bp, synthetic construct
  misc_feature 1..471: note = Synthetic
  source 1..471: mol_type = other DNA
atgggtacttcactgttgtgctggatggcactttgtcttttgggtgccgatcatgctgatgcatgtccgtactccaatcctagcctgtgctccggggggggagggagtgaactccctacacagggaaccttctctaatgtctccaccaacgtctcccctgcaaaaccgaccacaacagcttgcccctatagtaacccttccctctgtagtggaggggggggttcacctgctccacgccctcctacccccgcgccaacgatcgcgtcacaaccgctcagtcttaggccggaagcctgtaggccagcggctggcggtgcggttcatacgcggggattggattttgcctgcgacatttacatttgggctccgctggccggtacttgtggggtattgctgttgtctcttgttattacgctttattgcaatcacaggaacaggcgacgagtatgcaaatgcccgcggcccgtcgtg

SEQ ID NO: 396 - protein, 28 aa, synthetic construct
  REGION 1..28: note = Synthetic
MVCLKLPGGSCMTALTVTLMVLSSPLAL

SEQ ID NO: 397 - DNA, 84 bp, synthetic construct
  misc_feature 1..84: note = Synthetic
  source 1..84: mol_type = other DNA
atggtatgcttgaagctcccgggcgggtcctgcatgaccgctctcactgttactcttatggtccttagttcaccgcttgccctg

SEQ ID NO: 398-399 - skipped (2 entries)

SEQ ID NO: 400 - protein, 13 aa, synthetic construct
  REGION 1..13: note = Synthetic
GGGGSGGGGSGGS

SEQ ID NO: 401 - DNA, 39 bp, synthetic construct
  misc_feature 1..39: note = Synthetic
  source 1..39: mol_type = other DNA
gggggaggcggatctggcggaggcgggagtggaggctca

SEQ ID NO: 402 - DNA, 555 bp, synthetic construct
  misc_feature 1..555: note = Synthetic
  source 1..555: mol_type = other DNA
ggagacacaagaccccgattcttgtggcagcccaaaagggagtgccattttttcaatgggacggaacgagttcgcttccttgatcggtacttttacaaccaagaagagagtgtacggttcgactcagatgtcggcgagttccgagcggttacggaattggggcgacctgacgcggagtactggaactcccaaaaggatattttggagcaggcacgagcagctgtggacacctattgtcgacataattatggtgtggtggaatcctttacagttcagcggcgggtgcaacctaaagtgaccgtgtatccatctaaaacgcaacccctccaacaccataacctcctggtgtgttccgtaagcggcttctatcccgggtcaattgaggtcaggtggttcctcaacggtcaggaggagaaggccggaatggtaagtactggtcttatccagaacggagactggaccttccaaactttggtaatgttggaaacggtgccgcgatccggggaggtgtatacatgccaagttgaacacccgagtgttacgagccccctgacg

SEQ ID NO: 403 - DNA, 2895 bp, synthetic construct
  misc_feature 1..2895: note = Synthetic
  source 1..2895: mol_type = other DNA
atgggtacttcactgttgtgctggatggcactttgtcttttgggtgccgatcatgctgatgcatgtccgtactccaatcctagcctgtgctccggggggggagggagtgaactccctacacagggaaccttctctaatgtctccaccaacgtctcccctgcaaaaccgaccacaacagcttgcccctatagtaacccttccctctgtagtggaggggggggttcacctgctccacgccctcctacccccgcgccaacgatcgcgtcacaaccgctcagtcttaggccggaagcctgtaggccagcggctggcggtgcggttcatacgcggggattggattttgcctgcgacatttacatttgggctccgctggccggtacttgtggggtattgctgttgtctcttgttattacgctttattgcaatcacaggaacaggcgacgagtatgcaaatgcccgcggcccgtcgtgagatctgggtccggccaatgtactaactacgctttgttgaaactcgctggcgatgttgaaagtaaccccggtcctccaacaggtatggtatgcttgaagctcccgggcgggtcctgcatgaccgctctcactgttactcttatggtccttagttcaccgcttgccctggcatctgatgagaatcccgtggttcatttttttaagaacatcgtcacaccgcgcaccccacctgggggaggcggatctggcggaggcgggagtggaggctcaggagacacaagaccccgattcttgtggcagcccaaaagggagtgccattttttcaatgggacggaacgagttcgcttccttgatcggtacttttacaaccaagaagagagtgtacggttcgactcagatgtcggcgagttccgagcggttacggaattggggcgacctgacgcggagtactggaactcccaaaaggatattttggagcaggcacgagcagctgtggacacctattgtcgacataattatggtgtggtggaatcctttacagttcagcggcgggtgcaacctaaagtgaccgtgtatccatctaaaacgcaacccctccaacaccataacctcctggtgtgttccgtaagcggcttctatcccgggtcaattgaggtcaggtggttcctcaacggtcaggaggagaaggccggaatggtaagtactggtcttatccagaacggagactggaccttccaaactttggtaatgttggaaacggtgccgcgatccggggaggtgtatacatgccaagttgaacacccgagtgttacgagccccctgacggttgagtggagggcgcggtcagagagcgcacaatctaaaatgctgtcaggagtaggcggatttgtactcggactcctctttttgggcgctgggttgtttatctactttagaaaccaaacaagtagagtaaagttttcccgaagtgcggacgctcccgcgtatcagcaaggtcaaaaccagctttacaacgaactgaacttgggacgacgcgaagagtacgatgttcttgataagcggagagggcgcgatcccgaaatggggggaaagcctcggaggaagaacccacaagaaggcctttataatgaactgcagaaggacaagatggcggaggcgtattccgaaataggcatgaagggtgaacggaggagaggaaagggacatgacggactttatcaaggattgtctaccgcaactaaagacacctatgacgcgttgcacatgcaggctctccctccgagaggttcgagcggcagtggagagggcagaggaagtctgctaacatgcggtgacgtcgaggagaatcctggcccaatggcaatatctggtgttcctgtcctcgggttttttatcatagccgtactgatgtcagcacaggaatcatgggcgataaaagaagagcacgtgataatacaggcggagttttatttgaacccggaccagagcggtgagttcatgttcgattttgatggcgacgagatatttcacgttgacatggcaaaaaaggaaacggtgtggagacttgaggagtttggacgattcgcatcatttgaggcacaaggagcactcgccaatatcgcggtggacaaggccaacctggagatcatgacaaaacgctccaattatacgcctatcactaatgtgccccctgaggttactgtgctcacaaattctcccgtagaacttagggaacctaacgtcctcatatgtttcatcgacaagttcactcctccggtggtcaatgtaacgtggcttcggaatggtaagccggtcaccacgggtgtctcagagaccgtatttctgcccagagaagaccacctcttccgcaaatttcattaccttccctttcttccttcaacggaagacgtttacgactgcagggtcgaacattgggggcttgacgagccacttctcaagcattgggagttcgacgccccatcaccgcttccagaaacgactgaaaacgttgtctgcgctcttggcctgacagtgggcctggtaggcattattatcgggaccatctttatcatcaaaggtttgacttcccgggtcaaatttagcagatccgctgacgcaccggcctaccagcagggccagaaccaactctacaacgagctgaatctcggccgacgggaagagtatgacgtactcgacaagcggagaggtcgagaccctgagatgggcggtaaaccgagacggaaaaatccccaagagggtctttataatgaactccagaaggataagatggctgaagcctattctgagatagggatgaaaggcgagcggcggaggggtaagggccatgatggcctttaccagggactctccacggcaaccaaagatacttacgacgcccttcacatgcaagccctcccgccacgc

SEQ ID NO: 404 - DNA, 39 bp, synthetic construct
  misc_feature 1..39: note = Synthetic
  source 1..39: mol_type = other DNA
gttgagtggagggcgcggtcagagagcgcacaatctaaa

SEQ ID NO: 405 - protein, 1225 aa, synthetic construct
  REGION 1..1225: note = Synthetic
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRSGSGQCTNYALLKLAGDVESNPGPPTGMVCLKLPGGSCMTALTVTLMVLSSPLALASDENPVVHFFKNIVTPRTPPGGGGSGGGGSGGSGDTRPRFLWQPKRECHFFNGTERVRFLDRYFYNQEESVRFDSDVGEFRAVTELGRPDAEYWNSQKDILEQARAAVDTYCRHNYGVVESFTVQRRVQPKVTVYPSKTQPLQHHNLLVCSVSGFYPGSIEVRWFLNGQEEKAGMVSTGLIQNGDWTFQTLVMLETVPRSGEVYTCQVEHPSVTSPLTVEWRARSESAQSKMLSGVGGFVLGLLFLGAGLFIYFRNQTSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSSGSGEGRGSLLTCGDVEENPGPMAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGLTSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSTQSALSKDPNEKRDHMVLLEFVTAAGITLGMDELYK

SEQ ID NO: 406 - DNA, 78 bp, synthetic construct
  misc_feature 1..78: note = Synthetic
  source 1..78: mol_type = other DNA
atgctgtcaggagtaggcggatttgtactcggactcctctttttgggcgctgggttgtttatctactttagaaaccaa

SEQ ID NO: 407-408 - skipped (2 entries)

SEQ ID NO: 409 - DNA, 3678 bp, synthetic construct
  misc_feature 1..3678: note = Synthetic
  source 1..3678: mol_type = other DNA
atgggtacttcactgttgtgctggatggcactttgtcttttgggtgccgatcatgctgatgcatgtccgtactccaatcctagcctgtgctccggggggggagggagtgaactccctacacagggaaccttctctaatgtctccaccaacgtctcccctgcaaaaccgaccacaacagcttgcccctatagtaacccttccctctgtagtggaggggggggttcacctgctccacgccctcctacccccgcgccaacgatcgcgtcacaaccgctcagtcttaggccggaagcctgtaggccagcggctggcggtgcggttcatacgcggggattggattttgcctgcgacatttacatttgggctccgctggccggtacttgtggggtattgctgttgtctcttgttattacgctttattgcaatcacaggaacaggcgacgagtatgcaaatgcccgcggcccgtcgtgagatctgggtccggccaatgtactaactacgctttgttgaaactcgctggcgatgttgaaagtaaccccggtcctccaacaggtatggtatgcttgaagctcccgggcgggtcctgcatgaccgctctcactgttactcttatggtccttagttcaccgcttgccctggcatctgatgagaatcccgtggttcatttttttaagaacatcgtcacaccgcgcaccccacctgggggaggcggatctggcggaggcgggagtggaggctcaggagacacaagaccccgattcttgtggcagcccaaaagggagtgccattttttcaatgggacggaacgagttcgcttccttgatcggtacttttacaaccaagaagagagtgtacggttcgactcagatgtcggcgagttccgagcggttacggaattggggcgacctgacgcggagtactggaactcccaaaaggatattttggagcaggcacgagcagctgtggacacctattgtcgacataattatggtgtggtggaatcctttacagttcagcggcgggtgcaacctaaagtgaccgtgtatccatctaaaacgcaacccctccaacaccataacctcctggtgtgttccgtaagcggcttctatcccgggtcaattgaggtcaggtggttcctcaacggtcaggaggagaaggccggaatggtaagtactggtcttatccagaacggagactggaccttccaaactttggtaatgttggaaacggtgccgcgatccggggaggtgtatacatgccaagttgaacacccgagtgttacgagccccctgacggttgagtggagggcgcggtcagagagcgcacaatctaaaatgctgtcaggagtaggcggatttgtactcggactcctctttttgggcgctgggttgtttatctactttagaaaccaaacaagtagagtaaagttttcccgaagtgcggacgctcccgcgtatcagcaaggtcaaaaccagctttacaacgaactgaacttgggacgacgcgaagagtacgatgttcttgataagcggagagggcgcgatcccgaaatggggggaaagcctcggaggaagaacccacaagaaggcctttataatgaactgcagaaggacaagatggcggaggcgtattccgaaataggcatgaagggtgaacggaggagaggaaagggacatgacggactttatcaaggattgtctaccgcaactaaagacacctatgacgcgttgcacatgcaggctctccctccgagaggttcgagcggcagtggagagggcagaggaagtctgctaacatgcggtgacgtcgaggagaatcctggcccaatggcaatatctggtgttcctgtcctcgggttttttatcatagccgtactgatgtcagcacaggaatcatgggcgataaaagaagagcacgtgataatacaggcggagttttatttgaacccggaccagagcggtgagttcatgttcgattttgatggcgacgagatatttcacgttgacatggcaaaaaaggaaacggtgtggagacttgaggagtttggacgattcgcatcatttgaggcacaaggagcactcgccaatatcgcggtggacaaggccaacctggagatcatgacaaaacgctccaattatacgcctatcactaatgtgccccctgaggttactgtgctcacaaattctcccgtagaacttagggaacctaacgtcctcatatgtttcatcgacaagttcactcctccggtggtcaatgtaacgtggcttcggaatggtaagccggtcaccacgggtgtctcagagaccgtatttctgcccagagaagaccacctcttccgcaaatttcattaccttccctttcttccttcaacggaagacgtttacgactgcagggtcgaacattgggggcttgacgagccacttctcaagcattgggagttcgacgccccatcaccgcttccagaaacgactgaaaacgttgtctgcgctcttggcctgacagtgggcctggtaggcattattatcgggaccatctttatcatcaaaggtttgacttcccgggtcaaatttagcagatccgctgacgcaccggcctaccagcagggccagaaccaactctacaacgagctgaatctcggccgacgggaagagtatgacgtactcgacaagcggagaggtcgagaccctgagatgggcggtaaaccgagacggaaaaatccccaagagggtctttataatgaactccagaaggataagatggctgaagcctattctgagatagggatgaaaggcgagcggcggaggggtaagggccatgatggcctttaccagggactctccacggcaaccaaagatacttacgacgcccttcacatgcaagccctcccgccacgcggatccggcgcaacaaacttctctctgctgaaacaagccggagatgtcgaagagaatcctggaccggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 410 - DNA, 336 bp, synthetic construct
  misc_feature 1..336: note = Synthetic
  source 1..336: mol_type = other DNA
agagtaaagttttcccgaagtgcggacgctcccgcgtatcagcaaggtcaaaaccagctttacaacgaactgaacttgggacgacgcgaagagtacgatgttcttgataagcggagagggcgcgatcccgaaatggggggaaagcctcggaggaagaacccacaagaaggcctttataatgaactgcagaaggacaagatggcggaggcgtattccgaaataggcatgaagggtgaacggaggagaggaaagggacatgacggactttatcaaggattgtctaccgcaactaaagacacctatgacgcgttgcacatgcaggctctccctccgaga

SEQ ID NO: 411 - DNA, 58 bp, synthetic construct
  misc_feature 1..58: note = Synthetic
  source 1..58: mol_type = other DNA
gatgagaatcccgtggttcatttttttaagaacatcgtcacaccgcgcaccccacctg

SEQ ID NO: 412 - protein, 400 aa, synthetic construct
  REGION 1..400: note = Synthetic
MVCLKLPGGSCMTALTVTLMVLSSPLALASDENPVVHFFKNIVTPRTPPGGGGSGGGGSGGSGDTRPRFLWQPKRECHFFNGTERVRFLDRYFYNQEESVRFDSDVGEFRAVTELGRPDAEYWNSQKDILEQARAAVDTYCRHNYGVVESFTVQRRVQPKVTVYPSKTQPLQHHNLLVCSVSGFYPGSIEVRWFLNGQEEKAGMVSTGLIQNGDWTFQTLVMLETVPRSGEVYTCQVEHPSVTSPLTVEWRARSESAQSKMLSGVGGFVLGLLFLGAGLFIYFRNQTSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 413 - DNA, 1200 bp, synthetic construct
  misc_feature 1..1200: note = Synthetic
  source 1..1200: mol_type = other DNA
atggtatgcttgaagctcccgggcgggtcctgcatgaccgctctcactgttactcttatggtccttagttcaccgcttgccctggcatctgatgagaatcccgtggttcatttttttaagaacatcgtcacaccgcgcaccccacctgggggaggcggatctggcggaggcgggagtggaggctcaggagacacaagaccccgattcttgtggcagcccaaaagggagtgccattttttcaatgggacggaacgagttcgcttccttgatcggtacttttacaaccaagaagagagtgtacggttcgactcagatgtcggcgagttccgagcggttacggaattggggcgacctgacgcggagtactggaactcccaaaaggatattttggagcaggcacgagcagctgtggacacctattgtcgacataattatggtgtggtggaatcctttacagttcagcggcgggtgcaacctaaagtgaccgtgtatccatctaaaacgcaacccctccaacaccataacctcctggtgtgttccgtaagcggcttctatcccgggtcaattgaggtcaggtggttcctcaacggtcaggaggagaaggccggaatggtaagtactggtcttatccagaacggagactggaccttccaaactttggtaatgttggaaacggtgccgcgatccggggaggtgtatacatgccaagttgaacacccgagtgttacgagccccctgacggttgagtggagggcgcggtcagagagcgcacaatctaaaatgctgtcaggagtaggcggatttgtactcggactcctctttttgggcgctgggttgtttatctactttagaaaccaaacaagtagagtaaagttttcccgaagtgcggacgctcccgcgtatcagcaaggtcaaaaccagctttacaacgaactgaacttgggacgacgcgaagagtacgatgttcttgataagcggagagggcgcgatcccgaaatggggggaaagcctcggaggaagaacccacaagaaggcctttataatgaactgcagaaggacaagatggcggaggcgtattccgaaataggcatgaagggtgaacggaggagaggaaagggacatgacggactttatcaaggattgtctaccgcaactaaagacacctatgacgcgttgcacatgcaggctctccctccgaga

SEQ ID NO: 414 - DNA, 75 bp, synthetic construct
  misc_feature 1..75: note = Synthetic
  source 1..75: mol_type = other DNA
atggcaatatctggtgttcctgtcctcgggttttttatcatagccgtactgatgtcagcacaggaatcatgggcg

SEQ ID NO: 415 - DNA, 534 bp, synthetic construct
  misc_feature 1..534: note = Synthetic
  source 1..534: mol_type = other DNA
ataaaagaagagcacgtgataatacaggcggagttttatttgaacccggaccagagcggtgagttcatgttcgattttgatggcgacgagatatttcacgttgacatggcaaaaaaggaaacggtgtggagacttgaggagtttggacgattcgcatcatttgaggcacaaggagcactcgccaatatcgcggtggacaaggccaacctggagatcatgacaaaacgctccaattatacgcctatcactaatgtgccccctgaggttactgtgctcacaaattctcccgtagaacttagggaacctaacgtcctcatatgtttcatcgacaagttcactcctccggtggtcaatgtaacgtggcttcggaatggtaagccggtcaccacgggtgtctcagagaccgtatttctgcccagagaagaccacctcttccgcaaatttcattaccttccctttcttccttcaacggaagacgtttacgactgcagggtcgaacattgggggcttgacgagccacttctcaagcattgg

SEQ ID NO: 416 - protein, 23 aa, synthetic construct
  REGION 1..23: note = Synthetic
NVVCALGLTVGLVGIIIGTIFII

SEQ ID NO: 417 - DNA, 39 bp, synthetic construct
  misc_feature 1..39: note = Synthetic
  source 1..39: mol_type = other DNA
gagttcgacgccccatcaccgcttccagaaacgactgaa

SEQ ID NO: 418 - protein, 26 aa, synthetic construct
  REGION 1..26: note = Synthetic
NVVCALGLTVGLVGIIIGTIFIIKGL

SEQ ID NO: 419 - DNA, 78 bp, synthetic construct
  misc_feature 1..78: note = Synthetic
  source 1..78: mol_type = other DNA
aacgttgtctgcgctcttggcctgacagtgggcctggtaggcattattatcgggaccatctttatcatcaaaggtttg

SEQ ID NO: 420-421 - skipped (2 entries)

SEQ ID NO: 422 - DNA, 336 bp, synthetic construct
  misc_feature 1..336: note = Synthetic
  source 1..336: mol_type = other DNA
cgggtcaaatttagcagatccgctgacgcaccggcctaccagcagggccagaaccaactctacaacgagctgaatctcggccgacgggaagagtatgacgtactcgacaagcggagaggtcgagaccctgagatgggcggtaaaccgagacggaaaaatccccaagagggtctttataatgaactccagaaggataagatggctgaagcctattctgagatagggatgaaaggcgagcggcggaggggtaagggccatgatggcctttaccagggactctccacggcaaccaaagatacttacgacgcccttcacatgcaagccctcccgccacgc

SEQ ID NO: 423 - protein, 356 aa, synthetic construct
  REGION 1..356: note = Synthetic
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGLTSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 424 - DNA, 1068 bp, synthetic construct
  misc_feature 1..1068: note = Synthetic
  source 1..1068: mol_type = other DNA
atggcaatatctggtgttcctgtcctcgggttttttatcatagccgtactgatgtcagcacaggaatcatgggcgataaaagaagagcacgtgataatacaggcggagttttatttgaacccggaccagagcggtgagttcatgttcgattttgatggcgacgagatatttcacgttgacatggcaaaaaaggaaacggtgtggagacttgaggagtttggacgattcgcatcatttgaggcacaaggagcactcgccaatatcgcggtggacaaggccaacctggagatcatgacaaaacgctccaattatacgcctatcactaatgtgccccctgaggttactgtgctcacaaattctcccgtagaacttagggaacctaacgtcctcatatgtttcatcgacaagttcactcctccggtggtcaatgtaacgtggcttcggaatggtaagccggtcaccacgggtgtctcagagaccgtatttctgcccagagaagaccacctcttccgcaaatttcattaccttccctttcttccttcaacggaagacgtttacgactgcagggtcgaacattgggggcttgacgagccacttctcaagcattgggagttcgacgccccatcaccgcttccagaaacgactgaaaacgttgtctgcgctcttggcctgacagtgggcctggtaggcattattatcgggaccatctttatcatcaaaggtttgacttcccgggtcaaatttagcagatccgctgacgcaccggcctaccagcagggccagaaccaactctacaacgagctgaatctcggccgacgggaagagtatgacgtactcgacaagcggagaggtcgagaccctgagatgggcggtaaaccgagacggaaaaatccccaagagggtctttataatgaactccagaaggataagatggctgaagcctattctgagatagggatgaaaggcgagcggcggaggggtaagggccatgatggcctttaccagggactctccacggcaaccaaagatacttacgacgcccttcacatgcaagccctcccgccacgc

SEQ ID NO: 425 - protein, 893 aa, synthetic construct
  REGION 1..893: note = Synthetic
METDTLLLWVLLLWVPGSTGEVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSSGGGGSGGGGSGGGGSDIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKRSDPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSSGSGEGRGSLLTCGDVEENPGPMSGESMNFSDVFDSSEDYFVSVNTSYYSVDSEMLLCSLQEVRQFSRLFVPIAYSLICVFGLLGNILVVITFAFYKKARSMTDVYLLNMAIADILFVLTLPFWAVSHATGAWVFSNATCKLLKGIYAINFNCGMLLLTCISMDRYIAIVQATKSFRLRSRTLPRSKIICLVVWGLSVIISSSTFVFNQKYNTQGSDVCEPKYQTVSEPIRWKLLMLGLELLFGFFIPLMFMIFCYTFIVKTLVQAQNSKRHKAIRVIIAVVLVFLACQIPHNMVLLVTAANLGKMNRSCQSEKLIGYTKTVTEVLAFLHCCLNPVLYAFIGQKFRNYFLKILKDLWCVRRKYKSSGFSCAGRYSENISRQTSETADNDNASSFTM

SEQ ID NO: 426 - DNA, 2679 bp, synthetic construct
  misc_feature 1..2679: note = Synthetic
  source 1..2679: mol_type = other DNA
atggagacagacactcttctcctttgggtcttgctgctgtgggttcccggaagcacaggagaagtacagttgcaacagtctgggccagaactcatcaaacccggagcttctgtaaaaatgtcatgcaaagctagtggatatacatttacttcttacgtgatgcactgggtaaaacagaaacctggtcaggggcttgagtggatcgggtacattaacccatataatgacggcaccaaatataacgagaaattcaagggaaaggctacgcttacatcagataagtccagtagcaccgcttatatggaacttagcagccttacttccgaagattccgcggtgtattactgcgcgagagggacttactactacgggagtcgagtattcgattattggggtcaaggcacgacgctcacggtgagctcaggtggtggagggtctgggggtggcggcagtggtggggggggctcagacatcgtgatgacccaggcagcaccttctatcccggtaaccccaggcgagtctgtatctatcagttgtcggtccagcaagtctcttctcaacagtaatggcaatacatatctttactggttcctccaaaggcctgggcaaagtcctcaacttcttatatatcggatgtccaatcttgcgagtggcgtacccgacaggttttcagggtctgggagcggaacagcttttacgttgagaatatccagggtagaagctgaggacgtcggtgtatattattgcatgcaacatctcgaatacccctttaccttcggcgctggtacaaagctcgaattgaaacgcagcgatccaaccacgacgccagcgccacgaccacctacgcccgctccaactattgcctcccagcccctgagtcttcggccagaagcgtgtagacctgctgccggcggggccgttcatacgcggggccttgactttgcatgtgatatctatatatgggctcctttggcgggaacttgcggagtgcttcttttgtcactcgtgataacgttgtattgtaaaaggggtcgaaagaaactcctctatatatttaagcagccctttatgaggcccgtgcaaacaacacaagaagaggacggatgctcttgtcgattcccggaagaggaggagggggggtgtgagcttagggtcaagttttctcgctctgccgacgcgccagcctatcaacagggccaaaaccagctgtataacgaactcaacctcgggcgccgggaagagtatgacgtccttgacaaacggcgcggtcgcgaccctgaaatgggtggaaaaccgaggcgaaagaacccccaggagggactttacaacgaattgcaaaaagacaagatggccgaagcctattccgaaattggaatgaaaggcgagcggagacgaggtaaggggcatgacggcctgtatcaagggctctctacggccacgaaggatacttacgacgcccttcatatgcaagctcttccaccacggggttcgagcggcagtggagagggcagaggaagtctgctaacatgcggtgacgtcgaggagaatcctggcccaatgagtggggaaagtatgaacttcagcgatgtatttgactcctccgaagattactttgtatctgtgaatacgagctattactccgtcgatagtgaaatgctgctctgtagtctccaagaagtccgccaattcagtcgcctcttcgttcccatcgcgtactcccttatttgtgtttttggccttctgggtaacatcctggttgtaatcacattcgctttctataaaaaagctcggagtatgactgatgtttaccttcttaacatggctatagcggacattctttttgtgcttactctcccattctgggctgtgagccatgcaacaggggcgtgggttttttcaaatgccacatgtaagctgcttaaagggatctatgcaataaacttcaattgcgggatgctcctgctgacatgcatcagtatggatcgatacatagctatagtacaggcgactaagtccttccgcctgcgatcccgcacactgcctaggagcaaaattatttgcctcgtcgtatgggggctctcagtgatcatctcctccagtacgtttgtctttaaccagaaatataacacacagggttctgatgtatgtgaaccaaagtatcagacagtgagtgaaccaatacggtggaagttgcttatgttgggcttggagctgctttttgggtttttcatcccactgatgttcatgattttctgttatacatttattgttaagaccttggttcaggcgcaaaatagcaagagacataaggcaattcgagtcatcattgccgtggtgttggtcttcttggcctgtcagatcccccataatatggttctgctcgtcaccgccgctaacttgggtaagatgaatcgatcttgtcagtccgagaagttgatcggatacaccaaaactgtgacagaagtgctggccttccttcactgttgtctgaacccagttttgtatgcttttataggacagaagtttcgaaattacttcttgaaaatcctcaaggacctctggtgtgttcgaaggaagtacaagagctctggctttagttgcgctgggcgctacagtgagaatatatcccggcagacctccgagactgctgataatgacaacgcaagttccttcactatg

SEQ ID NO: 427 - protein, 238 aa, synthetic construct
  REGION 1..238: note = Synthetic
VSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSTQSALSKDPNEKRDHMVLLEFVTAAGITLGMDELYK

SEQ ID NO: 428 - DNA, 717 bp, synthetic construct
  misc_feature 1..717: note = Synthetic
  source 1..717: mol_type = other DNA
gtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 429 - protein, 1153 aa, synthetic construct
  REGION 1..1153: note = Synthetic
METDTLLLWVLLLWVPGSTGEVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSSGGGGSGGGGSGGGGSDIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKRSDPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSSGSGEGRGSLLTCGDVEENPGPMSGESMNFSDVFDSSEDYFVSVNTSYYSVDSEMLLCSLQEVRQFSRLFVPIAYSLICVFGLLGNILVVITFAFYKKARSMTDVYLLNMAIADILFVLTLPFWAVSHATGAWVFSNATCKLLKGIYAINFNCGMLLLTCISMDRYIAIVQATKSFRLRSRTLPRSKIICLVVWGLSVIISSSTFVFNQKYNTQGSDVCEPKYQTVSEPIRWKLLMLGLELLFGFFIPLMFMIFCYTFIVKTLVQAQNSKRHKAIRVIIAVVLVFLACQIPHNMVLLVTAANLGKMNRSCQSEKLIGYTKTVTEVLAFLHCCLNPVLYAFIGQKFRNYFLKILKDLWCVRRKYKSSGFSCAGRYSENISRQTSETADNDNASSFTMGSGATNFSLLKQAGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSTQSALSKDPNEKRDHMVLLEFVTAAGITLGMDELYK

SEQ ID NO: 430 - DNA, 3462 bp, synthetic construct
  misc_feature 1..3462: note = Synthetic
  source 1..3462: mol_type = other DNA
atggagacagacactcttctcctttgggtcttgctgctgtgggttcccggaagcacaggagaagtacagttgcaacagtctgggccagaactcatcaaacccggagcttctgtaaaaatgtcatgcaaagctagtggatatacatttacttcttacgtgatgcactgggtaaaacagaaacctggtcaggggcttgagtggatcgggtacattaacccatataatgacggcaccaaatataacgagaaattcaagggaaaggctacgcttacatcagataagtccagtagcaccgcttatatggaacttagcagccttacttccgaagattccgcggtgtattactgcgcgagagggacttactactacgggagtcgagtattcgattattggggtcaaggcacgacgctcacggtgagctcaggtggtggagggtctgggggtggcggcagtggtggggggggctcagacatcgtgatgacccaggcagcaccttctatcccggtaaccccaggcgagtctgtatctatcagttgtcggtccagcaagtctcttctcaacagtaatggcaatacatatctttactggttcctccaaaggcctgggcaaagtcctcaacttcttatatatcggatgtccaatcttgcgagtggcgtacccgacaggttttcagggtctgggagcggaacagcttttacgttgagaatatccagggtagaagctgaggacgtcggtgtatattattgcatgcaacatctcgaatacccctttaccttcggcgctggtacaaagctcgaattgaaacgcagcgatccaaccacgacgccagcgccacgaccacctacgcccgctccaactattgcctcccagcccctgagtcttcggccagaagcgtgtagacctgctgccggcggggccgttcatacgcggggccttgactttgcatgtgatatctatatatgggctcctttggcgggaacttgcggagtgcttcttttgtcactcgtgataacgttgtattgtaaaaggggtcgaaagaaactcctctatatatttaagcagccctttatgaggcccgtgcaaacaacacaagaagaggacggatgctcttgtcgattcccggaagaggaggagggggggtgtgagcttagggtcaagttttctcgctctgccgacgcgccagcctatcaacagggccaaaaccagctgtataacgaactcaacctcgggcgccgggaagagtatgacgtccttgacaaacggcgcggtcgcgaccctgaaatgggtggaaaaccgaggcgaaagaacccccaggagggactttacaacgaattgcaaaaagacaagatggccgaagcctattccgaaattggaatgaaaggcgagcggagacgaggtaaggggcatgacggcctgtatcaagggctctctacggccacgaaggatacttacgacgcccttcatatgcaagctcttccaccacggggttcgagcggcagtggagagggcagaggaagtctgctaacatgcggtgacgtcgaggagaatcctggcccaatgagtggggaaagtatgaacttcagcgatgtatttgactcctccgaagattactttgtatctgtgaatacgagctattactccgtcgatagtgaaatgctgctctgtagtctccaagaagtccgccaattcagtcgcctcttcgttcccatcgcgtactcccttatttgtgtttttggccttctgggtaacatcctggttgtaatcacattcgctttctataaaaaagctcggagtatgactgatgtttaccttcttaacatggctatagcggacattctttttgtgcttactctcccattctgggctgtgagccatgcaacaggggcgtgggttttttcaaatgccacatgtaagctgcttaaagggatctatgcaataaacttcaattgcgggatgctcctgctgacatgcatcagtatggatcgatacatagctatagtacaggcgactaagtccttccgcctgcgatcccgcacactgcctaggagcaaaattatttgcctcgtcgtatgggggctctcagtgatcatctcctccagtacgtttgtctttaaccagaaatataacacacagggttctgatgtatgtgaaccaaagtatcagacagtgagtgaaccaatacggtggaagttgcttatgttgggcttggagctgctttttgggtttttcatcccactgatgttcatgattttctgttatacatttattgttaagaccttggttcaggcgcaaaatagcaagagacataaggcaattcgagtcatcattgccgtggtgttggtcttcttggcctgtcagatcccccataatatggttctgctcgtcaccgccgctaacttgggtaagatgaatcgatcttgtcagtccgagaagttgatcggatacaccaaaactgtgacagaagtgctggccttccttcactgttgtctgaacccagttttgtatgcttttataggacagaagtttcgaaattacttcttgaaaatcctcaaggacctctggtgtgttcgaaggaagtacaagagctctggctttagttgcgctgggcgctacagtgagaatatatcccggcagacctccgagactgctgataatgacaacgcaagttccttcactatgggatccggcgcaacaaacttctctctgctgaaacaagccggagatgtcgaagagaatcctggaccggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 431 - protein, 780 aa, synthetic construct
  REGION 1..780: note = Synthetic
MVCLKLPGGSCMTALTVTLMVLSSPLALASDENPVVHFFKNIVTPRTPPGGGGSGGGGSGGSGDTRPRFLWQPKRECHFFNGTERVRFLDRYFYNQEESVRFDSDVGEFRAVTELGRPDAEYWNSQKDILEQARAAVDTYCRHNYGVVESFTVQRRVQPKVTVYPSKTQPLQHHNLLVCSVSGFYPGSIEVRWFLNGQEEKAGMVSTGLIQNGDWTFQTLVMLETVPRSGEVYTCQVEHPSVTSPLTVEWRARSESAQSKMLSGVGGFVLGLLFLGAGLFIYFRNQTSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSSGSGEGRGSLLTCGDVEENPGPMAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGLTSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR